SEQUENCE LISTING (55 sequences)

SEQ ID NO: 1 - RNA, 88009 nt, Homo sapiens
  source 1..88009: mol_type = other RNA
cctgcgtgcgctgccgtcccggatccaccgtgcctctgcggcctgcgtgcccggagtccccgcctgtgtcgtctctgtcgccgtccccgtctcctgccaggcgcggagccctgcgagccgcgggtgggccccaggcgcgcagacatgggctgctccgccaaagcgcgctgggctgccggggcgctgggcgtcgcggggctactgtgcgctgtgctgggcgctgtcatgatcgtgatggtgccgtcgctcatcaagcagcaggtccttaaggtgggtgagggagaccccagggggtccgcgcacggacccgggctgttgggcgctgggcgccgggaggacccgcgcgttgcggtgggtgggcgaccgcagcggaatcggcgcccgggcctggcgccgcagagcacgagggaggccaggcgcttcgggaggggctgctgcccgcctccccaccaccctcaccgccccagactgaggctaggggggaacccaggggctttgctggaggcgcaggccagggttggggaggcctggacgggagaccacccgggcgcctgacccgccctaaagggtctgggcgcccacgacggatcaggagtgtggacaggttgcttccgtggtgccgcccgaaggagcccaagagtgtgtgaacgtctgctttcacagcagccggtagcagggtccggtaaaattagagctgcactttgcttgtatcctgtgagcccaggaggacaggaaccttctgtgagacccacgtgggtgcccagctcacatggtaggtgctcagctgagcatcctccactgcccagaatctccccataggaaccctctcccagaggcagaatgcgcgactctgttctccctgctcactgcctccatgattcagtctggaaactataagccgcttttctccccagattcatgcagaggcaggcagcagcccaaggtcctctgagtgcacgcagccttaggtctttcctctgatcctaactttgcataccattgccctttcaaccagggagacagattaggcggtcagctggtctgcagacggacagactctgccctgggtgcctgggtggcctgtttgaatgagcctggagttagggaagggctgggagattgcagagtatcacatttgaactaaaagccatttagagttttgaagccctgggaacccacacatgcagtggcggggaggggaaactggggctcactcttatcaaagttctagccagatgcagagctggggtgaagatgtggacattgtcactctaatggacgtcttttccccttggcctgcctctttggactgggtttcttaagaatggttccttgtccacatttcatgtctgttccctcattcacagagcgaatgaatgagtgaatgaatgaaggatctgcttagatctggctgccacacttcttgggacccagctagctggccttaaagatggagcaacatatggtctctctcaaggtctctgggtgagcagttgtctagggcatcttagccacgttgtgtctatttttgatttttcaaatcttgtcccgccaaagcctgtaatctgactgcaaagaggtcccggtggatggtgacactgtctctggggatggcaggaaaatcagcagaatggcctcttctcccccatgacagctcagaggctttctctgttacactgagggagctttatttttctgtggcctactcatactgaaacgtgtgtgtcagtgacagccttgtggctgctgcagtgccaggaccccactgtgtgtatgcagggtccctagaggggctatcactcaggcagttcaagcatgtgaccctgtgctgtgagtgagcacctggtctcttgctatgggagtgtggggacttatgccaggtaaagagtcttgccaactcagttattcgggggcttatttcaaggtgttttctttctcaggatgtcatcaggttaagtatgtcagtaggatgtgtgtgtgtgtgtgtgcaccacacatgcatgcaccaccaaatttccccactcctctcctgggcttctaaagaggaaaaccacattgtcctcaattgtcctggagctaggagcatctggcttaaagttgaacaggagcctcccagcatccttctgtggatatcccaaaggcgattggtttggggccgagtggtgtgaaggcaggaagacacgcccagcccaagacggccacttctggaaccgcccaggctggagcagcggccaagtgccttagcatggaagaagagagcctgtgttcaggggaccaggcgggtgagacacacgcataagcagggactgcccgctggtgttttctttggctacatgagtctgtgtgcaacttgaatgggaggccttgtgcttttattttttttttttaatgtgctttttggttaacaacagaagtccctacagttggccctctgtgtctgcaggttctgcatccgtggattcaatcaactgcagatgaaaagttttcagaaaaaatccccccaaaataacaatacaactaaaaaaagacataaaaacaatactgtataacaactattgacatagcatttacatcgtattaggtattataagtaacctagagatgatttaaagaatacgggaggatgcgcataggtgatatgcaaataccacagcattttatgtcggggacttgagcatctgcagatcttggtatctatgggggtcctgaaaccaattcccctctgacattgagggacgactttatataaaatatatgaatacttgctcgtaaaaaaaagttcaaatggaggaaaagagagaaagtttcccccattttattcccctcctgagctattatcaccatcattgatgtggatctttctagacccttttctgtgtaccaggagcctgtatgtatacatattaatatatattttacataacttgaactaagtgtaggtgaacttgaacatgtataattatcttgcaatttgtttttcaattcaaagataccagctgaaaaattttccatgtctcagcaataagtctaccccgcgttttccttcatcactgctggtattcctttgaatggttatagacttgtttacctgtttctccattgactgatcattttctctgtcacaaatgacagcgatatcccgacacatccctcttcgtgtggttcatgaacatttccatagggtggatttctagaagtggggccttgggagcaggtgtgctcagtttgagttttgatgattacggccaaattgcccttctgcacagctctgttgctgcatccacccacaaggtatgggactgtgctacttcccctatctctgccatcactggtaattactgatttatcattaccattttttggtagccaagttcccgtgtgtctctggtcctccaaggaaggcccagctggtggacatgggggagaggcccctcactgcagctgggctgcaaaaaagggatgctcatcttccgaggctgctgtcttcaatgatgagctggggtgaagatgtggatattgtcacactagttgacatctttttccctttggcctgcttctttagactgaatttcttaagaatggcttcttgtccacatttcatgtctatgtccttttcacagaacttggatgtgtgcccccagggatctgaaccatgtcttcattattcaccggatcacctgaggtcaggagtttgagaccagcctggccaacatggctaaaccctgtctctactaaaaaatacaaaaactagctggacatggtggtgcacgcctgtaatcccagctattcgggacgctgaagcaggagaatagcttgaacctggaaggctgaggttgcagtgaactgagatcacgccactgccctccagcctgcgtaatagagtgagactccatctcaaaaaaaaaaaaaaaaaaatgaaggaacttagaaatatgtctcagcatgggtaggccataaaaacacattactaattgaaaaaagctctggtgctatttatgtttaaaaaaaacccaccaaacaatattctatattttctttccatttttatttttattttcgtagagacaaggtctcactatgttacccaggctggtctcgaactcctggccttaagtgatccttctgccttggccttccaaaacactgggatgataggtgtgaaccaccgcgccctgccattattttcaatggcttaatattagaatatgttaaagtttgggaaaaggtgagactaacagaagtggctactctaatgagagaaggcaggacactgggggtgtggccaaaggaaacctaatactttatctgtaatgctatttttttttttcttttttgagatggagtctcgctctgtcgcccaggctggagtgcagtggcacgatctcggctcactgcagcctctgcctcctgggttcacaccattctcctgcctcagcctcctgaggagctgggactgcaggtgcctgccaccacacccagctaattttttttttgtatttttagtagagatggggtttcaccgtgttagccgggatggtcttgatctcctgaccttgtgatcttcccctctcggcctcccaaagtgctggaattacaggcgtgagccaccatgcccggccattatttttgttttaacagagaaaatgtatccacatgctacgtgtttgattaaaaattacaaggccaagcatggtggctcaaacttgcgatcccagcactttatggggctgaggtgggagggtcacttgaagccctgagtttaagactagcgtgggcaacatagtgagacccccatctctacaaaacaaaacaacaacaaaactaagctggtgcatggtggcatgcacctatagtcctagctacttgagaggctgaggtgggaggatatcttgagcccaggagctcgaggctgcagtgagctgtgtttgtgccactgtactccaacctgggcaacaaagtgaaatcccatctcttttttttttttttgagacggagtctggctctgtcgcccaggctggagtgcagtggtgcgatctcggctcactgcaagctccaccttccgggttcacgccattctcctgcctcagcctcctgagtagctgggactacaggcgcccgccaccgcacccacctagttttttgtatttttagtggagacggggtttcactgttttagccaggatggtctcaatctcctgacctcgtgatccgcccgtctcggcctcccaaagtgctgggattacaggtgtgagccaccgcgcccggccaaaatcccatctcttaaaaaaaaaattacgaatgaacagatacatagttttcctcaaagactggccagatgaatgcgccacacagaccgatgaggcacactgcaaaggggccctgtggtcgaggttgcttggagaggctttggggaagcgcaactcatttcttttcccttctgccagtgggtaggattggagagaggtgtttggacaggcacactaagggccgcgattgtgaacttcatggcatccattcatgggcagcttactgtgcaccaggcagggttcaaagcacctttgtgaaaattgtctcatttagtccctgcagagggccgtgacatatgatttattgtcttcagtgtccgttaaggagacaagctgaagagagtaatttgccccaagtgacctcgctagtgtatgtgtgtgcgtgcatgtgtgcacgtgtgtgtgtgtgtgaaggtgtctgtctgtcccagaggcagatgccatgagaatggggactcttgctcccattccctggatctgagcctggcatgcagctgtgctcaggtatgtgggtgagtaacgaaggcctggttcaggtcctggggggcacacatccaagttcccgtatgtctctggtcctccaaggaaggcccagctggtggacgtggggagaggcccctcactactgctgggctgcagggaaggggtcatcttccaagaccaccatcttcaatgataggcctcatggccttcttccatgtccaggagagaacctgggtcccctctgctgtctttaagttggttttctagatggactcaacctcggttgccttgtcctgctgctgggcttcccccacctatgatccctgtctcaataaacggcacctccttttgtttcccctggggtagaggcaaaaaccttcggtcatctttgcctgcatgcttttttcctgatacccctgtatctgctgaggcaggggctttcccatcactgtcactgcagcgtccccgagggccatagcagcgcctgagctccttgccaccccttcaacaccagtcaggttacagaatgaatgaggcgggcgcgacccccagccctggcctcaggctctttagctcacccctgcctccctcagccagagccacacagtgagccttggggagccccggcaccagggccagccagggatgtcaccggctgtccctgagtttgtgccttttgggaaatctttgtcaatcagttaaaagatgggggttgatgggagataacagtcaatgggcactcgttgtggccggatactgttccaagtgtttatgtgcattattcattttagtatctaaaataggtactattgtgtcttattttattgagggaaatggatgcacagaggcggaaagtaacttgccccatgtcacatagctccaagcggttgaatcaggatttcaagtctgggggccgactccacagctggtcccggtggcctctgcgcactgcgtcctcccggttctccgtgtgtctgagcaggtcgactgcatgcttaaggcgggcggcggggggtgcccttcttatattctcttcctgggctgccccttccttttctgtgcatcaccagggaacagcagggtcccttcgaggctttgtcctaatcatcaccccgccaaactttgctggtgcagagtgcaccctggcctctcccttccgggggtagtctgagaccagggacatgttctccaatcaaaggcaccaagtgaggctcccggggaggccacctgttcctagcccagggctcccagtgaccccgggggctggactcccacccatcctgcaggggccgcccgctctgcgcctttatcagcccccctgcggaacctgggggggaggcccagtgtctccacctaccgggcgtttcctgtaggacccccttgtttggacatgtggggcaaagccctagaactgatcactgggcacaggtggggagggggctcctcgaggacagcttggatcacaggtgtgtgccaattctgttttgtttttcttcctctttcctgccattgaacttcctactccttctagcatgtgagagacagcttcaacttactccctggcgacggctgaaaaacatgggctatttttagtttgtgtgagtctagtttatttgctcctggtagatttgcctctccctgtcttcgtgagtgtgtgtgtgtttgcacacacgtgtgttggtgtgtgtacacaagtgctgatggtgaaaacagtgacacacacacctgtgtaggcatgcacacacctgccttgatgtgtgacgacgtgatgatgtgtatgcagacacctgtgttgatgtgtgcctacatatgttgacacaggcacacatggtgtgtctgtacatgcaaatggacacacatgaacacacgtgtttatgcatgtccttgtgtgttgatgtgtctgcacatacatgttgatgtgtcccgctgtggttggcctttctggggccaggcccagcacctggggtttccaggaaacattccctgtcccttcccggaatggccggtacttgctgtgcctccgccgggagatgcaccctgattaactagaacgtggcgaagctcagccacctggaatcgccttaccctggcctttggtttctagcaaatgggatggaagtgagtccccatggagggcttgctccatgaaggtgtttttcctggatgagtcttggccccagtggcgatttgctgcaggcttgttggcactgtcattcgtgggtcctgcatggaggttccttagtgtccagagggtgattccatgtctgtggtcagcaaaagagaggcccctggggccataagccacaccccccaaggctgaacccagagagtttgtggtttaaatcttgggcctggaggattctgtggaggagagttcggtccaggccttctgctccaggaggcgctggcagctccgacaccacagacacattaattgtgcggagaattcaacgctagggctcagtcaaaaatcaaaaacgcaaaacaaaaagagaatcccttttatttacctcgtctccctccccctgtttggacatgaccttgagctctgctggcccacaagtggccaggctggagctgtcttgcaaagagaaacaaaaacaacacaaaaagctgcgttcttgactttgaagccttaaagggcccaggggctggctccctgtgggtgttgggggcgattttcctcccaaagcctgcgtgagatatggagacactccgcctgctcagaagctccccaggtcccgggttccaggaagcctcatggcccaaggcctggccgtcttaattttcttgttttctttccctccactccactgtttcttcctgttcactgattgcctgtgctggggagggggaaggaacgggaggggcctgaccttgttcacttcctggcattttgggtgaagttccttacaccttctctggaagcttctagaaggacccttgaggcgctttaaatctccattctctgggttttcctcccaccttcctggctgctctgtcttcttgcctgctgatgcctcctcatctccctggcctcctgcctcctgctggtggagctccaggcatcctttctgtgtgtcctcatccctcatccctggggtgggctcctccagccttgcgcctgaatggcatctctgtcctgaccactcccaaattggtatccctgggcccagacctttcccctgaaccccacccccgagtgtccaacagcagagtccgtatctcaactcggggtcaaaggcagctcacgctcgccccggctggctcctcatcaccctcggccacacctgtggctcccaccagcttcccactccatcatagcccccccatcttccagttgcccgggccaacatcttgcactcagccttgactctgttctctccacttccctcagagaacccgtcagaaagttttctagcttcatcatctaaatatacccagattccaactacttctcaccacgcccgaggcgccatcacctctggtccaaggcaccatcacctctcccccagattcctgcatccgcctcctcactggcctccctgtgctcatccttgctccccagtggggtttctcaataagcagccatgggagcccatcaagattgcacttagcacctcctcccctcaaagccctgacatggccagacccgtcccctaggagtcacagccaggtcctaacctggcccagggcctgtgccatccacccctgccacttttctggcgacgtccgcattcccttttcccttgcacaatggcctcctggccattcctcagccccatgggcccaatcgccttagctctcctgacacaagccctctcatctcccgtaggtctctgttcagctctcacttgctcacggagaccctcccctgattccgtttgtgcatcatgcaggttcccccttgtccccctgtgctgccagccctccttacccgctctgcttagacctgtcagccactgtagataccctggaacttccctgggtatcatgttgattgtttattgtctatttctccccatgagaatgtcggactgtgagggcagggacctttgtgttattcgttgccgtgacccaagcccctggaacgatgcctagcacataatcgcaggttctcaaatatttattgaaaggatgtttaaaagacaagggttgtttcacaggatggttcgtgaccagtggcaggttgtggtcctgtgcagggcaatggggcctactcctccacctgccgtggccgtcgccccagaccgagctgccaccgttgtttcccagtggatgctggagtgtcatcatggggctgtttgctctgctgatttataccacactgttgtcacttctccccatcaaagccaggatgctctttaaaacatgcaaattgggccgggcacagtggctcatgcctgtaatcccagcactttgggaggccgaagtgggtgggtcacctgaggtcaggagttcaagaccagcctggccaacatggtgaaacctcatctctactaaaaatacaaaaattagccaggcgtggtggcatgtgcctgtaattccagctactcgggaggttgaggcaggagaattgtttgaacccgggaggtggaggttgcagtgagccgagattgcaccattgcactccagcctgggtgacagagcgagactccgtctcccaaaaaaaaaaaaaaaaaaaacaaaactatgcaaactggatcatttcaagaagctgaataaaatcttcctgcatttggtatcaaagctgtgagcctcattttcgtgggccctgcccatctcaactcagcattctcaccccactgcaacctcacaggccttccttttcctccaacgtgtcacatgctgtcctgcctcagggcctttggaagtgcttttctttctgcctggaccgtctttttgtctccctggctcttatgcttggttcctcttttatgtttaaatgtaaccttttgaagcttaaatgtaaccttttgaagaagacaccccgtgattgcccaccccacccaatggacagttgccccccacgtccactgtctgtgccatctgtgtggtttttcagctggagggcaatggccccatctcggctcactgcaacctcctcctccccggttcaaacgactctccagccttgcctcttgagtagctgggattacaggcgtgcaccactatgcccagctaatttggtatttttagtagagatggggtctcaccatgttagccaggctggtctcgaactcctgacctcaggtgatccacctgtcttagcttcccaaagtgcttggattacaggcatgagccaccgcgcctggcttgcgccatctgtgttataaccacaccatgctattcccttctgagcccttaatagtctctgaaattatctcatccacttgtgtgagttccatgaggacagagactttatctgtctcgttcatggctgcattccatagttggagacccttgtccagggcagagaaccacctagcgtaggaccttgggctaaggcagagaggccccagtgggggtcctccgcagcgtggactttggaaccagactccctgagttcagcagtggctcttgcacttcctgtctctgtggctgtgggtgagttgcataaccctgccgggcgtgtttctccgggcctccctcctcgggctgtagcgaggggtaagggaggtcatacgccctggggaacaccccaaagcctgggtccctgggatgcatccacagattgtaggcctgagtcttaacacactccatctggatttcaatgacttctgcctggttccagtgctgggctgtttccctgtctgagctttggtggcttttgattgacccctttcctgccccagcatgaaggtggagtagccagaaggaagggatgcgggtgtataaagatgtcatcggggctgtctgtggatggtgggtaaagagacagcacctgtcatggtggaagggccacaggaagcgtggaggctgtttctggggccacctcctgcctccaggaggaagcagggatcctcctgttggcatcttggtcccatcaggtgtcttggatggggctggggctgggagtgtttctctccctgagcatttaagccaaggttcattatttagaatggaaccatttataggccgggcgccattattacattattcctaaagtgttaggattacaggtgtcatgcctgtaatcccggcactttaggaggctgaggtgggtggatcacttgaagtcaggagttcgagaccagcctggccaacatggtgaaaccccatctctactaaaaacacaaaaattagctgggcatggtggcacacacctgtagtcccagctacttggaaggctgaggcggaggactgcttgaactcgggaggtggaggttgcagtgagctgagatcgcgccactgcacttcagcctaggtgacagtgagactccatctcaaaaaaaaaaaaaaaaaaatggaatcctttatgtttcactttgttctcattagcaggactgtgcgaggctgagtcaggagaccgaggtccattctgcttgtgattttgctccgtgcaggaatgtggaggtgtggcccataagcatgtcattcatgcaaaatcatctacatgcattcaattagaaaaaaaaaggaaaacagcagtgaccctcataagtagacttggttagtgtcaatgtagggagtgagtttcttgtgaccctgctgttcttcctgggtcccgtgacagggcatctggacaggctgaatgcgattctagaattagattcctatgtttttatatgtacgccccagtctcacttgccagtggcttcctctgacgcttagaaaataaatggcatctgcaccacgtcagggagaaactgactgctttggaattcccagaagctcttggaagggaaccttcctgagggatattgaaggagaattaaagaattttctcagtggctggcacaacttatggagtcaatgctcattggctgtgattggctgagctgggatcacatgtgcatccttggaccaattgctgtggctcgagtaatggactgctgtgattggtcagccttgggtcgcctgcccatttggagccagggagggaagtagttcttgcccacagagactcaggtaggagtagggtggttcccaaaaggcaaaaaggaggggctgttgtcgggggaagaggcggccataggcagcaaaaatggggctgagcccctttgcccccaggtagcttcccacttcatccccacattgcctttcactgtctctggctgttacagtattagagattattttgtttggctgactattctgtttaccaaggcctcttgaggaggtagtagcttaccagctgtttattatgtatgtatttacttatatttacttacctatttattttgagacggagtttctctcttgttgcccaggctggagtgcaatggtgtgatctcggctcaccgcaacctttgcctcccaggttcaagcgattctctcacctcagcctcccaagtagctgggattacaggctgcgccactgtgcccagctaattttgtatttttagtagagacagggtttctccatgttggccaggctggtctcgaactcctgacctcaggtgatccgcccacctcgacctcccaaagtgctgggattacaggtgtgagccacgaagcccggcctatttatttatttttttgagacatggtctcagtctgtcgcccgggctggagtgcagtggcatgatcttggctcactgcaacctccgcctcccgggttcaagtgattctcctgcctcagcctcctgagtagttgggattacaggtgcccaccactacacccggctaatttttgtatttttagtagaggcgaggtttcaccaggttggccaggctggtctggaactcctgacctcaaatgatccacccaccttggcctcccaaagtgctgggattataagcatatgccacagtgcccaccctttattatttatttatttagattttttgagacagggtctcactctgtcacccaagctggagtgcagtggcatgattacggctcactgcagcttcaaccactcaggctcaatcgatcctcctaccttggcctcctgaataactgggactacaggtgtgcaccaccatgcccagctaatttttagattttttgtagagacaggggtcttgctatgttgcccaggctggtcttgaactcctggcctcaagcaatcctcccaccttggtcttccaaagtgctgggattacattcatgaatcactgtacctggcctaattgctctaacggggaaagtttcatcagctgtcagattgttagatctggactgacttgggttccgaagctcagttctatcttttatttttcctttggaaaccaaattctgtaggtagcagaggtttactcaaggccacggagatagttagcagccagtgctgggtggggccgactgtcccaggctagctggatggaggccaggagggcccgggcagcaagggtggaaggggcctgggtgggtgccggaggaagtcactaacgaatgttcttcaggcagaaagttcccagggtcccattgatacccctgcctgttcacatcctggcggcaggactgggatgaggcattttttaaaaagttggggagatatcagggctggtgtgccaaagttcaggctttgtccacccaaggtgagaggctgcaggggtgtgccgtgggcagagaggggatgaggtaggggtgggagggatgagctgcccagatgcacccgtggaggagttgatctggaatcctagccactgggtttaccctgcagtgtgtcccagcagcatcccaggcttctctccagcctttggcagggctttagcttccagcagctcaggtggaccccagcatatggactttgaaggcaggcagcccaggttcaaatcctggcttggccacttcccagttgggtgccttcaggcaagtgacttaccctctctgtgccttttttacttttctgtaaaatgaagttaataaaagtgtctcctacatagggcttttaaggtggcagaattggcacatgtcacacttcaaatagcagctcggcaagtgtcaagtaagggctggctgtttccaacatcttaaccggatagacacagacatagagagggatccatctgtcagcagtttgctgaaatgggaccatactatgcacgctttgctgcaccgtgggtttctcacgctgtcatcgcttgtccatgtcgtcctcagtcagctgggattttctggtgatgatcacgctctgattctctagatgcctggaaacgctccacggtgcggatgtaccacggctcgttctgccgttctcacgagggaggcatccagcttgtaaaataaagcagcccagtgtgttcttccaccaccacatcacccaaggttctattttcaaagattttattttgtaaaaacgggaagaaaagtacaagttgctgcatgtgcatcctctctctctcgttgctgagccttttgggtgcatcctaaggacaaggacgtccttctccatggccgtggaccacggccccactcaagaaggccacgaggatccacacctttaatctgacagtctgtgttcagattcccttacggccccaataacactttttgtcatagttgttcattaaaaaattttgaattggccgggcgtggtggctcacgcttgtaatcccagcactgtgggaggccgaggcgggtggatcacaaggtcaagagattgagaccatcctgggcagcatggtgaaaccctgtctctactaaaagtaaaaaaattagccaggcatggtggcgcacacctgtagtcccagctactcgggaggctgaggcaggagaatcgcttgaacccaggaggcggaggttgcagtgagccgagatcgtgccactgcactccagcctgggcgacagagtgagtctccgtctcagaaaaagaaaatttttttttgaatccaaggttgcatgctgtgtctggttgcctcatgtgtttttcctctttaatccagacctgttctccagcccttttctctttgctgggtctttcaggacactggcatctgtaaaggggccaggcgggtgcttttggcaggatggcccttgatttggatttgtctgattgctttctcaggagtgcacgtggtcttcccacagactcccacacaaaagcctcattgtgtgaatcagattggagcagagtgtttctgatgagccggggttgggtgcctggatcctcccccagctcccagcctggcagtcacagtgcaagtctgaacctcttgggttgaagaagcctgcaattcctcctggtcccttcctccacctgctttctaaaatttctggctctcattccttgcctttgagcggtgatgtgtccatccttcagcggccctcggccagcctctgcccaccccagtgccatcacatgggaagagatggcggatctgatccctgactcatcccctgggccaggaagattgtggaaaccctggggtctcagggctggtcgggtggaaacgggaggctggatggagtggcaggtgggggcttttcctgttttgcctctccctaactgggcctcccggcagccactagcccagatgcccaggcctcagagccttgtggaaattcttgtgccagatcatcttggccctgtctggcctcctggcaggcaggtggaggtcaaactggccttacttggccgagagcccattgcagccaggatcccttggcagctctgggaacagccaagtctcccctgtccttgggatttccttccactgtctgttcccaggctcctggactccacctgctgcaacccctcgctttgcactttccacccagagtcttggatttctgttttttgtgaaatttgctcactttgaggaaagtgtaagacatatgcaatcaaatatgtaactaagatgaacatgcatgcaatcaccccacgtgaagaagagggaaagctcatctgccccgaagcccctgcagaagacactgtgtttggtcccagcttgctttgctttttatagtatcactccctcatagccatccctaaacaccgggattagtttggtccggttttggacttaacttggatgcaggcatactctcagtatttttgtttggtttggtcttgctggtgttacttcatatggtatttgtgggatccacccacattgctgcgataagctgcactctattttcatggccatgtcatatcacagttcatgatgctaatcggaagaagctgagtatcacagttcatctgtctttttttttttttttttttttttttttttgagatggagtcttgctgtgtggccaggctagagtgcagtggcgcgatctcagctcactgcaacttctgccccccgggttcaagcaattctcctgcctcagcctcccaagtgcaccaccatgcccagctaatttttgtatttttagtagagatggggtttcactatgttggccaggatggtctcgacctcttgacctcatgatccgcctgcctcggcctcccaaagtgctgagattacaggcgtgagccaccacgcccggccagttcatctgttttaaccctgatgccggtgactgtgttcacttctgggttatgacacactcgtgtccctgcctgcaagggacatgtgcctgaatccctctagggcccgacagtcagagtagggggtctggtcatggcgtgcgtgtcttcacctgcatggagaagggccaagctcttgtccaaagtggttgcagcgtttccaatcctatcatttgtttttcatttaataatttattgagttcaaattcacataacatataattaatccttgtaaatcgaataattcagtgataccaccacttctacctagtttcaaaacatttccatccctcccaagtagaattgcccattctccactctttgcagctccccagcccctggcaatcactcattttctttctgtctctacagatgcacctgttacggacatttctttctttctttctttctttttttttgagacagagtcttgctctgtcgcccaggctggagtgcagtggtgcaatcttggctcactgcaacctccgcctcccgggttcaagcgattcttctgcctcagcctcccgagtagctgggactacaggcgcctgtcaccacacccggctaattttttttttttgtatttttagtagagacgggtttcaccatgttggccaggctagtctcaaactcctgacctcaggtcatccacctgccttggtcccaaagtgttgggattacaggcctgagccaccgtgcccggccctggacatttcttttttttttgaggcagagtcttgctgtgtcacccaggctggaatgcagtggcgtgatctcggctcactgcaacctccgcctcctgggttcaagcgattctcctgcctcagcctccccagtagctgggactacaggcgcccgccaccacgcccggctaattttttttttttttttttgtatttttaatatagacggggtttcacatgttggccaggctggtctcaaactcctgacctcaggtcaaacacctgcctcggtcccaaagtgttgggattacaggcctgagccaccgtgcccggccctgcacatttcttataaatgaaatcatgcaatgtgtggccttaagtgcctggctgcttccgcttagcgtcatgttttggggttcatccgtggtgtggcatgtggagtgctgctttcctttttgtagctgagcaggcctccacggtgcgggtgcactgcatgctgtttttccgcgcctctgtggatgggcgtctgggctggtggctgccgggagcggtgctgctgggagcgcgcatgttcgggaactcgcttggcttctgctttcagtggtctccttttatgccctccttcccctcacccctctcgctcttcagcccgccctgcccctgcgcccatcctctgtgtggctgcgtctgtaaacctgagtgtgtccttggaccttgtgcagtcctagattccggagcctgcaggtgtgggtccagatcccggccctgcctcctgctgctgtgtgaccatggacaggctcttcgagtctctggtctctggtgggtgtctgagtctgcatgggaggcagaggttgcagtgagccgagatcgtgccactgcactccagtctgggcaacagagcaggactctgtctcaaaaaaaaaaaaaaaagaaagaaagaaagaaaaaaatgtccataacaggtgcatgggccaaagaccatgtattcccgtacagttctggaggccggaagtctgagatcaggatgttggcagggctggcttctccggagacccctctcccggcctcatagacgccaccttctccctgtgtgcgcacatccccggtgactctctgcgggtcctaatctcctcttttaagggcgtcagtcagattggattagggctcaccccactgacctcatttaaacgtaatcaccgatttaaaggccctgccccccgcgccaatacagtcacattctgatgcaccgagggttagggctgcaacatgggaattttggggggacactgttcagctcataatagtagggtgcctataaaatgggctaaaaccatgccttcctataggattcagtgaggaccgagggagctagaggtggaagtgctttgagctgggggtggcacagggaggcccccttcatgtggcctgtcacgtctgaggagagggcggcttttccatgccatgcacggtaactcgtgtcaacattcctcgtgctgccctgtttaaaatctatccgtgttcctatgtgtgtatcaggcttattgcttccttctgctgcctggaccccctaattcatgttcatcctgttgtatttattcccttagcgacgggggtaccttggttatgtctgactcctgcctccacaaacagcaaatatgctggaccctgctgccttgtacttctccacaatttcctggggatgcgcaggagtggaattgctgtagggcagagggggagccattttcacccaacatgccttccagaagggccactgctgttcaccctcccagctctcacatcctctccagcacctgagacgatttagtcttcgagaatgtctggggtctcagtgagggtacgtggtgcagcattgttctcatgtgcttttctgaggtgtgaatttgagtcttcttcctttcataagtcctcaggtgggggtctgaggctcagagattttgagggccatgttggagggtcacagagcaaactggggctagaccccaccagcttgggcttggagggcctccagcttggatgtagggcccttcataaatccaggatgatctcatcttgggatccttaacttggtaacatctgtagagaccctatttttaaataaggccacattctgaggttctaggtgggcatgaatttggcaggggacatgcttcagaccagcactatgtgtgcaggtgcccaatcctcagatctgcagagctctgggttcaaatccaggtcccattacttgggtcctgtggaaactcacttttttcctaaaagccgatttccacctctgcacatggggccaatgttcttccatctcacaaaccagaagtcaaggccaggcgcagtggctcatgcctgtaatcccagcacttagcgaggcagaggtgggaggatcgcctgaggccaggagttcaagaccagcctgggtaacatagtaagacccccccccccatctccacagaaagtaagaaacaaaaacaaaacaaactggaggccaaggagagcagctgcggatggtcacgtgcctctccctgggtgtgcccagctcttacccagaaagccctggctggcattaaaaataaggaaggtaaaatagggatatgatcaaacaaattaaaagtgtacggaaaaagagtcagccttctggaaagatcacagcacgacactgctcccctggccccagctggtctttgctccagaggtgacttgggttaatagttccttgtttttccttcctgcaataaacatgtttgcgtccacagagggggacccacacccgtatccttcccgtccgtggcacacgtcccacaagggcacacggttcccatgcttttctgcctcttggctggggcacctaggtggatcttggcatctcttgttcccgcacagaacccatcctcatccgagccctgggcctctgtctcccagcaggctttgcaaataggaccttgtaaaggacagggctgtgtttcccaaaggccagagcagtgaagaggggcatgtggggtcgtggggcacacctctaggacattggggtccaggggtcctttccagtaagaccttgctgtgctggaaacacccctgtgtctttcagggctgttttcagagtcagggttgatgtgaccgccacgtttcctctgggcctgacagctgtaatctgtggcttgggagtgcagataagtgtgatgaagctgccagcaggcctgggcatctgtgtccttggtgggggcagggagaagagacagggactgggatgctggggccagggccagagccccgctgagatatgaggtccccttccaccctgggaggtgtgtggggaggagcttcctcgaacccctttagctgggacggatggaagaatgagcagctttgcatttccggcagcgtctccagctttcctggggctttctcggcttgtgttattgatgaactccagtgcgtgaggccattggagccttagaggcctgtttcccctggacaggtgactgcctgggccagaggcagcactggcatggtgccctcttgctcctggctgggagtgacacggggcgtggggcgctttgcattcatccccagctgtttgggcacatttacaaccctcattttacagatgggacaaaccccagtggagaatctgctccgggcagctgtttatgaaccacctgaatggggcacacaggactgtgggaggaggaggagggacccccagggctgaagtggcagggaaagctactcatcctttcggcccatgtttactgctcacaaattgtgggccaggtctcgtgccaagtgttggagctacagccgtggacaaaagagacctggtcctgctcacggcagctacgctttagcaggagggatgggccatggagaagcacatggaagaggtggggtggcgagaagagctaggaaggggacgaggcaggtgagctgacatggagccactgctgggaaggggactccttctgatgtgggagtcaggagggcctctcagaggaggggctctatgagctggggagcagctggcaaagagcacagcaggttcaaaggccggaggtgcgaaggagcctgctgtgttggaggaagagggagcaggccagtggggttgggagaagtgactggagggggtgaaaggggacaggcatgtgggggccttgcaggacacctggaggagttcagatttggttttcaattccatggagggttctgagcagggaagagccatgatctgactcagtattttttttttttttttttgagacagagtctttctctgtcgcccaggctggagtgcagtggcatggtatcagctcactgcaacctccgcctcccgagttcaagcaattctcttttttttttttttttgagacggagtcttgctctgttgcccaggcgggggagtgcagtggcgcaatctccgctcactgcaagctccgcctccctggttcatgccattctcctgcctcagcctcccgcgtagctgggactacaggcgcccgccaccacgcccggctaatttttttgtatttttagtagatacggggtttcaccgtgttagccagcatggtctcaatctcctgacctcgtgatccgcccacctcggcctcccaaagtgctgggattacaggcgtgagccaccgcgcccagcctcaagcaattttcttgcctcagccttctgagtagctgggattacacgtgcgtgccactacactcggctaatttttgtatttttcgtaaagatggtgtttcaccatgttgggcaggctggtcttgaacccctgacctcaggtgatccacctgccttggcctcccaaagggctgggattacaggtgtgagccaccacgcccagcctgactcagtcttgaagagctctctggctgctgtctggagaagggagtgtcgggggccaggctggaagcggggaggcagagaagatggtgatttgagccaggtgggggtagttttctaaatggaaagaagggaacatattcgagttttttttcagggagagtcaacaagataggtcagtggactggggtgcgagggaaggagaggaatcaagggtggttcctcaaggtttgaggagtttggtagatggtggagccattttccgagacgagaagatcgggggagggaggttgagagcagatggagtgtgggccttaactctggtcctaaaatcaagcgaggacaactcaacaacaagagtcagacaaccccattaaaacacggaccaagcacctgcaaagaagacatgcaggcagccaggcgcggtggctcacgcctgtaatcccagcactttgggaggccaaggtgggcggatcacaaggtcaggagttcgaggccagcctggccaagatggcgaaaccccgtctctactaaaaatacaaaaattagccgggtgtggtggcaggcgcctgtatgtaatcccagctactcaggaggctgaggcaggagaatcgtttgaacctgggaggtagaggttgcagtgagccaagactgtactccagcctgggcaacagagcaagactctgtcttaaaaaaaaaaaaaatgcagatagccaacggggacatggaagtgtgctcagcgacactcgctcatcgttagggaaatgagaatcagtgagacacactcatacccattaggatggtcacttgagaaaacagaaaatctcaaatattggtgaggatgcggagaaatcggggcccatgtgtgccgttggggtgagggtgcggagaaatcggggcccatgtgtgccgttggggtgagggtgcggagaaatcggggcccatgtgtgccgttggggtgagggtgcggagaaatcggggcccatgtgtgccgttggggtgagggtgcggagaaatcggggcccatgtgtgccgttggggtgagggtgcggagaaatcggggcccatgtgtgccgttggggtgagggtgcggagaaatcggggcccatgtgtgccgttggggtgagggtgcggagaaatcggggcccatgtgtgccgttggggtgagggtgcggagaaatcggggcccatgtgtgccgttggggtgagggtgcggagaaatcggggcccatgtgtgccgttgggaatgtaggatggtgcagctgctgtgtggaggcccccccaaaatttaacatagaattaccatatggtggggcaaccccacttctgggtatcaaagaaatggaagtgggaacttgaagaggaatctggccaccgcgttcattgcagcgctgttcacgatggcccaacggtgggagcaacccgagtgtccgctgatgggtggtgggtaagaagctggggcccatccacacgttggaataggattcagcctggaaaaggaaggacgtttggacgcacgctgcgacatgcacgggccttgaggaagttatgcgagtgaaataagccacaacaggacaaataccgtacgattccatttgcatgagctccctagagtagccagatccacagagacagaaaagagaatggtgggtgccaggggctgggggagagggggtgcagagctctttttcagtgggtacagggtttcacttttgcaagatgaaagagttctttagatggatgttgctgataattgcacaacattgcaaatgtccttaatgccactgaactgtacacttaaaaatggctaagttgggctgggcgtgtagtggctcatgcctgtaatcccagtacttttggagcccaaggtgggtggatcacgaggtcaggagttcaagaccagcctggccaagatggtgaaaccccgtctctactaaaaatacaaaaattagcctggcatggtggcaggcgcctgtaatcccagctacttaggaggctgaggcagagaatttcttgaacctgggaggcagaggttgcagtgagctgagatcacaccactgcactccagcctgggtgacagaccgagactccatctcaaaaaaaaaaaaaaaaaaggctaagttggtaaattttatgtttaccccaattaaaattaaaaactgtggcagggtttaacctgaagggagaaatggtaccggcccctcctgcttgaatgggttgggctgttggtttgtagatggtagaccagacggcccagcctgttggccagggtcaaattccaacatgctgagcctgttgcagaagacttagcctttgaagcaggcagccctgagcttctgattcctccaggcccaaaccaccgttgccctattttacaagaccttggctggcttcatgacgtgttcttgaactggagccagtggtgagctcagggaggctcctgtaacaatagttccctgaggcatgtttatagccgggtgccctcttcacaacagacacatgtcctgggtcatctcattctctctgcactgccacggggaggatggggaggcagtcacgtctccatgtggcatatcaggggaccggggtgggaggggctgactgagccgaggccagaactaggtctgcgccctggccctctccctaatggccagatccggcctcacccgtgaagctggcctggcaggggatggcccttcctgaatttctgtgtggtctaggctggggttggcatgcttttctgtacagagacagatagtcaatatgctcagctctgcaggctgcacgggttgtgtctcagctactcagccctgctgctgtgatgctgaggctgccattgacgacacagaaataagtcgaggtggccgctgtgggccagtgccactgcatttatgaacaccaaaatgtgaatttcatataactttcacatgtcatgaaatggcatttttctttcaattgtttgtttatttatttttaacatattttttggagacagggtctcactgtgtcacccaggctggggggcaggggcaccatcatggctcactgcagcctagacctcctgggctcgttatcctcccaccttactctcctgagtagctgggacaacaggcatgtgccaccacgcctggctcattttttttttttataggttggtgggtgggtgtgtagggggggtcctcactatgttgcccaggctcgtcttgaactcctggcctcaagtgaccctcccaccttggtctcccaaagtgttgggattacaggcgtgcagcactgcacccagctggctgacttttcttttcttttcttttcttttcttttcttttcttttcttttcttttcttttcttttctttctttttctttcctttcttttcttttttctcccttccttccttccttccttccttccttcctttctttctttctttccttctttctttcttttctttcttccctttcttcccctctttcccttctttccatttgaaaatgcaaaatgattgttagcttgtggggtgagtagaaataggcatcaggctggatttggtccacaggctgtagcttgttgaaacctttctaagaacagagaagaataatgtttttgcttgaaaacagccagctttattgaggttggggtgtttgaaagaaggcatattcactgtctgacttgtgtggaaacagtggggctgggatcagccctggtagaaattgtttttgtccaaaaaagcaagtagaggtgggctgcctggagttcccgtcatgcaggtgcaactggggagcaggatgaggccctgatttggcggggaaggagaggttgtgcttgattcatctctgtgcccggggaccaggactgtatgcaccttccagcattctcttcgctggtctgagctccttacttgccacgcctgtgctgggcctttaccaaggcagtgcccagcctttggtcagacccaaggtcaagactctgtgccaacattgggtttgtgctcttgggcaagcgagtggtttaatctccctgtacttcactttcttcatcagtaaaatggagacaaattattagctttattctattttattttatttcattttattttgagacagggtcttgctctgtcacccaggctggagtacagtggcacgatcatggctcactgcagcctctatctcctgggctcaagggatcctcctgcctcagccttccacgtacctgggtctacaggcatacactaccacatttggcaaatttttaaacattttttgtagagatggggctcactgtgttgcgcaggctggtctcgaactcctgggctccagtgatcctcctgccccagcctcccaaagtgctgggattacaggtgtgagccaccacacccagcctaattgttaggtttaaataaatgaaagcattgctttaaatgggatgctttgagctggaaacaacataaaacttgcctccagtggcttctgtaatgcagatgtttactgttgtgcttcctgagagatgggaggtggtggtttccagatgcgttcagcagttcagaacatcgtgctctgcctcagtagattttgcccagtggctctccgaggtggctgtactgatggacattctggccagtgacacatgcgcgagacctctggtgcttgccagtgcgtgccagtgaatacgtgtgatgtccctgagctcttcctactgggtacctgagtcacttgtgaattgcgtgtcctcttccccacttcctgctcagtccacgagggcaggaatcctgttggattaactttacccctgtagcactgatcgcccgctttggatgctcaggcagggttgaaatgaatgaatgactgagtgaatgaatgaatgaatgaatgccacgttctcatcgtgtgttactataggacagggattgcaaactgtgcaggaacatacatgggggaagggccctggcatgaggtgatagggagtggtggggcccgttgcaagctggagggcacaccccaactgtgaagcggctcctgctttgctccagcgggctgggccacgtggcatgcaggcccagagctggacatcacccaggttcccaaaagaagccagaaggcaggaatttacctgctggtttaagatactgtcaacagattcaaatattttgaaatatgtgattcaaaacccatctactggcaaaatctggcccatgggcccagtttgtggcatctgtttcaggaagagacaagacctagtttgtgttttttttagtgggggcggggaggatgcggtgtataagaaattttgagtgaattaaaaaaaatttatgaaaaatttcaagggctgggtgcagtgtctcacgttcgtaatccagctctttgggaggttgaagcgggagaattggtttaggccaggagtttcagaccagcctgggcaacatagcgagacccctacaaaaaataaaatctacaaaaaatgaaaaaattagctgagtatggtgacagaagctgctgtggtcccagctacaaggaggctgaggcaggaagatggtttgagcccaggagttcaaggctacagtgagctcggactgcaccagtgcactccagccagggtgacagagcaagaccctgtctcttaaaaaaaaaaaattcaaacatacataaaaatagaccggtgcgatggtcacgggccctgtaacttgctgaggagataccaagtcttgccttctgcacgtgcctccatacactttccccttttccacattgttctggggctcatgtcagacattgcctcatccgtcagtgtctctagtatagccacggcacttcatggtgcattggagtctggacgctggagggctcactaaaacacggatcaccaggccccagcccctgagtgtctgagtcggcagacctggcatggagcctgggaatctgcttttttatcgagttcccaagtgatgctgatgttgctggtttggagaccgccttttttttgggggggtggacagggtcttgctctgttgtgcagcctggagtgtagtggtgcaatcacagctcactgcagcctcgaccttctgggcccaagcaatcctcctgcctcagcctcccaagtagctgggactacaagtacatgccaccatgcctggctattgtaaaaaaattttgttctagagatgagatcttgctatgttgcccagactgttgaactcctgggctcaagtgatcctcctatctttgccttccaaagtgctgggattacaggcgtgagccaccacgctcagccagggaccacacttcaagaaccactgcccgaacaggtacctagtaaacaccaccacaatgataaagcaaaagaaaatccccaagcatgaccacagtactatgctcacaccaaaagtcgttctgtagggcagtaatgaggtttgaaaggtggacctgctccagtgttcagaaattgcccgagacctcatgcctggagtgggaggctctgcccggaccctcagccccttgttggttgggccccttttgggggcctcaggggctgtggctcagggaccagcacaagggactggcaccggtgacgtctgtttctggtcccggcccagcctggaagtttctgggaacctgaggaccctggagtggttctgacctctggctgcctccctgtccccccgtgtgtcccgtgggagctcactggcttgggaggcctgcttttgacaagacccctggctctggccttgcagggcccctcaggcccctgctggctgttgcctgcattcttcctcccatgtggctgaggatgaagctggcttagcccttttcagggcgggcctcggtttgtgtttctggacaccctcgtgtcactggcaggaggtctcgggagtcacgaggaggcagtgcaggcagcacgggcacgtccgcagcccagagggcttgcttttcagatcccagctgcaagaacaaagtgcccccgctgagctgcttgaaagcagctgcgggagagtgtcctgctccgcagatcattccccggcccgggtttctcttaggacagctcaggccaaaacacagcagggttcaaaagcgggggacgtgtgagcatcctgggcgtggagcttcccattaaagaggacactgggcttcctgccgggtccaccttgtttctagcgaagcctgccttctggccaagatagggctgtggggagaggaaggggtgcacggggcaaatgtcacactccctggtgcaggggtgttccctggggtggagggggctgtgtgggaggatgctctggggtctctgggtgggtgtggcaggctgaacaatgtccccaaaaggtgctggcgtcctgatccccaggacctgtgaatatgtgaccttcctcagcaaaagtggctttgtaggcgtgattaagttaaggatcttacaccaggcatgatggctcacacctgtaatcccagcactttgggaggccaaggtgggagatttttgagcccaggagttgaagaccagcttgggcaacatagtgagaccccatctctataaaaaatttaaaaattagccaggcgtggtggtgtatgcttgtggtcccagttatgtgggaggctgaggaaggaggattgcttcagcccgggagttcgaggctgcagtaagccgtgatcacaccactgcattccagcctggcaacagagtgagaccctgtctcaaaaaaaaaaaaaaagttaaggatcttgagatgggagatgaacttggattgtcccaggggccacaatgctttcacaaggatctttgacagagggaggcaggagggtcagagtaggagatgtgacctcagaagcaggggttggagtgataaggggacaggagccaaggaatgcggaggcctcttagaagctgggaaacggaaggcaatggattccccctgagctccagaaggaagcaccctgcctgcaccgtgactttcatcccatctcaggggcagtcccagtttggacttctccccccgagaactgtaagataataactttgtattgatctgagccagtgttaattgttaattcttacagcagcagtgggaagctaatacaatgaaaatgcttccattaaagccaggtcattaacatgatagtataatctggtttgggtgggcttaaataaaatatttaaacttaaaataatgttgtgttaatatgtgaacttttgaaattgtcagtgtttttacaaaaagaccttaatgttctggaaaaagtataaatatggggtgcacattttttccttttgcttgaagctgtaatatgattcggtgaggcactgatttggccctccctgtattgccgcgacagagcctgcttgttttaaaataactttctgttttagaacagtttttttttttttgagaccaagtctcactctgtcgcccaggctggagtgcagtggcacaatctcggctcactagaacctctgccttccaggttcaagtgattctccttcctcagcctcccaagtagctgggaccacaggtgcctgacaccacacctggctaatttttgtacttttagtagagtcgggatttcactatgttggccaggctagtctccaactcctgacctcaagtgatccacctgccttggcctcccaaagtgctaggattccacaggtgtgagccaccgcgcccggcctagaacagttcaactttcagaaaaattgggaagatagtgctgagttcccacatatcccacagccagtgtcccctgctattaacatcttaaaggaatgggatacatttgttgctatgaatgaaccagtgttgatacatcagtaataactaacatccagacaggattcacgtttccttcgtttctccttcgtatcattttctgtcccaggacccaatccagggccccacattgcgtttgtccctcacgttgccttaggatcctctggtctctgtgggttctcagactttcctggctttggatgacctggattgcgttcctgagcaccggctaggaattttgcaggacgcccctctgctgggatttgtctgttgttcttctattgattaggccctagttccgggttttggaaggaagatcgcagtgacgaagggctgttcccagcacattatgtcaacatgacctgtccctgctgcatgggtcctattctttattccttgttacaggtaaagaaacaggcacaggaagttgtatcaggcgcctgctggtaggtggaggatgggcagggggttgaggggacagggagtggacagagcccctttgctggtaaagaggggaccccagacctctcacgtctctccagggcccctggtggtctcctggctggcctcgctgctctgcttcagaaatcagatttgacggggcactgggaccctgattgggccttggtggctgtaggtgctctcttattagcgacaccacttgagggtgtattgaacatctgaagagtacccaaccctggatcaataattttttgctttaggccaggcgcagtggctcacgtctgtaatcccagcactttgggaggccaaggtgggccgatcacctgaggtcaggagttggagacctcagcctggccgacatggtaaaaccccatttctgtcaaaaatacaaaaatcagtccaggcgcggtggctcatgcctgtaatcctagcactttgggaggccgaggcaggcagatcacctgaggtcaggagtttgagaccagcctggccaatttggcaaaaccccgtctctacttaaaaaaatacaaaaattagccaggtgtggtggcgggcatctgtaatcccagctacttggggggctgaggcaggagaattgcttgaacccgggaggcagaggttgcagtgagccgagatcgcgccactgcactccagcctgggtgacagagcaaaactccgtctaaaaaaaaaaaaaaatcagctgggcatggtggcgtatgcctgtagtcccagctattctggaggctgaggcaggagaattgcttgaacctgggaggtggaggttgcagtgagctgagatcacaccactgcactccagcctgggcgacagagtgagactctgtctgaaaaaagaaaaaattttttttgctttaatttttttctttttaaaaattgaagtatcaaatgcattaagaaaagtgcacaatttcggttagaggttgatggattttcacctaggtatgcacgcttgtaatgaccccctataccaagtgtaaacgcctcccccgcacccctccctgtcagtatccacccctgaggtcctgctgcccccacctctatccccatggattggttttgccattccccgggacgtcacttatatggactcatgcacgtggtccctgtgtgtgatttaccctgtcattgtgtgtctcaattggtcattcccttttgtctctgggtagcattccatggtatgagtgtgtccccatttgtcatgcattcacttgcttgtagacatttgtgttgtttccagttttgggctgttatgggtacggctgctatgagcatttgtggataatctttgtgtgggtgcatgctttcatttttctgggacaaatactgggggtgggattgctatattgtagggtgaacgtccagctttggtagaaaaagtatattccacagtgactgcactaatgcacttccaccagcaatgtgtgagcactctggctgctccacatcctcaccagcacttgttattgtcagtctttttcattgtagccattctggtgtatggcatgggatctcactgtgagtccagtggacatttctctgatgactaagccacccagggctggtgggtggctccaggaagtcacaggaatcctttctcctatctcctattctgctccgcccttcttcatgtgtggctttcattcttaagggtgcctcatgatccaagatggctgctctagctccatccattgtgactgaattccgggcagcaggaagggggacttatcaacaactacatagaaatttcagatattgataatgtcagggagataaaacagggtgatgaatataatgtgatgggggaagggctgctttaggaagaatggtattttcatccattttgttcactattgaagtgcccaggcctagagcagagcctgccatacagtggggctcaatagagatggttaagggattgaaaattgctcagggaaggtctcacggagaaagtgactttagacttgagaagtgaggggctcacttttgtgggcatcaggggaaagattgcccctggaggagcaaagagtcagtgcaaaggccctgtggcagaaacttgcctggtggctcaggagcacagcaaggccagtgtggctggagcagagtgagcgaggcagataggaatgggggaggagactggagtgactatagggagctgggttgtgcaagacctcataggtcactgtcaggaatttagattctattctgagataaatagggaggcatgggggaggtttgagcagaatgacatgctctgactttaatcttaaaaggataacttggattctgtggggagaacggggcggggggcaagagcagaggcaggaaaacagcggggaaactacggcacccatccatacaagccaggatgggggccaggatggtgttggagagaagtggatggatttgaggcatgctttaaagcgagagcagcccagccttgctgatggagggtgctaggtgggaggaaaatggaggactcaggaatgactcaacctcttcgcttaagcaacttacaagcgtgggtttcttgtaaaaaccttttattttagtcttcttaccttagtcctttaaaaatcaaaagaagttggaacatgagccaagggtgtggtgttcccaagtcccagattggattagcctgtgtcgtagttgttcctgcatcttaaggccccatggagctggggatgcctggagccctgggattgagagtgtagtcagccactgactgctggtccagagtgggcagggcctggccttgtcccagaactaggcttcgtctgggcaaattggcttcttggctcatttggtcagaggtgtgtttcttggaagcctgttttctcttctcataggaggaggaattagaccttgcaattcagtggccaggtcaagttaatgattcatttgtgaagctcccgtgcaaggagcccagcggggatggcctaggagagaagcgtgagggcacaggcctagacgtggatacggagcctggggctctgtctctccctgctcctgtgaccttggtcaggcatttggctttggagcctcagtgtctgcttcgatgagatgagccattgagaatttaagaggtgaagtaggtggaagtgtttcgctacctgaacagggccgggcagatcgtccagacgattctgctcccctggtgtctgagggcaggggcgtctcagggcaggggccccatcgtgttcatccctgtgtctttaaattcctagcatagcatctagcatggggcggatgattgtacagggcatgaactgcaatcccaatgaggtcagagcagcctcactgtaggccgaggtcgttccagtgggcttcatggaggaggtggcacctgggcgagcggggtgttaggtttggaggtcaggggcctctccaggtgcaggggagagtgctgtgggtggtaggaagagaatgctaaaaattaatgtctatgaaatcttcatctcatccatcacacatttaccgagttgttgccccggcctaaacaggcggtactctctgtgtatttaaaaaaaaaaaaatcccttcttaagtatgagctgtttaaaacacacaaaggaggactagcgttaccacccgctagacatttccacgttgattatatggtttcaggtctcacttttgctcattcacccagaaaatatttacagagctcctgcaaggtgccaagagctgtcccaggccctgaggacagacacatcgccaaacaaagcagctgaggatcttgcccttgtgggtttatggccaggcaggtgatacagatggacagcaataaacctgataagctaattataccgtgtgctcgaaggccacaagcagcatggaaacaacacggcaggtcgggaggtcaggagttttaggggcaggttggatgagccttctgagaagggggcatgtaagcagagattcgaaggggtgagggatttgaccacgtgtttaggaggaagcagtgggtgcaaaggccctggggcaggagcgtggcctcttggacaggatagcaaggaggctgtggggctgggctgagtaacagacagtggttgtggaatgagaccaccgcttctgctgttgtccttcccagcttctccccagcctccccttttccctagtttataagacaggagaaaagggagaaagcaaaaagatggaaagaaacagaagtaagataaatagctagacgaccttggcgccaccacctggccctggtggttaaaataataataataataataatattaacccctgaccaaaactactggtgttatctgtaaattccagacgttgtatgagaaagcactgtaaaactttttgttctgttagctgatgtatgtagcccccagtcacgttcctcacacttactcgatctattatgaccctttcacatggaccccttagagttgtaagcccttaaaagggctaggaatttctttttcggggagctcggctcttaagacgcgagtctgccgatgctcccggccgaataaaaacctcttccttctttaatccggtgtctgaggagttttgtctgtgacttgtcctgctacagttggatgtggggccagagagaacacaggggccagaccaggcagaggggcttgtaggccaatgttcggacggtggctcgtatgttattttcaagaaataacatgttcacaatgacagttaaagctttccccacatccccagcccccagccctctcctgagattgtttgcaggtatcatttccctgaatgtttttctacatttccacatgggtttgtcttcagaaagaatatagagtattatttcatgtgtttggaaaattcacatcaagaggcccatgtcaattgtttctttctttagccttttcttcactcaagatttttttttctttaaaaataaaaattgaggccaggtgcagtggctgacacctataatcccagcactttgggaggctgaagcgagtggatctcttgagctcaggagttcaagaccagcctgggcaacatagtgagaccttgtctctactaaaagcaaaaaaataaaaaaataaaaaaatagccgggcgtggtggtatgtgccttgtagtcccagctactcaggaggctgaggtgagaggattgcttgatctggggagatcgggaagtcaaggctttccagcctgggcaactgagcgagactctgtttcaaaataaataaataataaaaattatgtatatttaaggcatccaaatgctgttttgctatatacacacacatagtgaaatgattactacagttaagcagattaacatatgtatctcctcatatagtcacctctttttttttttttttttttgagacagggtctcgctctgtcacccaggctagagtgcagtggggtgatcacggttcactgcagccttgaactctcaggctcaagtgatcctcccacttaagcctcctgagtagctgggaccataggtgcgcgccactgtgcctggctaatttttgtattttttatagagatgggctccctatgttgcccaggttgttctcaaactcctgacctcaagcgatcctcccacctcggcctcccaaagtgctgggattacaggcatgagtcccggccacctctttgtttttagtggtgagagcacctgaaatctgcttccagtatacagtacagtattaatcacagtcctcgtgttgtgcatgcaacctccagaccacccatcccacatgactgcagctttgcgccctgtgacctgcatctccccacctccccagcctccgccccgtaagcaccattcaactctctgtgtattcaatgtcttcagattcctcacctctaaaagacaaatctggaagtatttgtcctgcagtatttgtccttctgtcaagattctgcctttgagatatctgcaagtcgatggcgtttactcactgtgcagccaccgtgtgcagaagcccagcttatctttgactgagaacctggctgggctctctgttgtttttcaaggcagggtctcgctctgtcacccaggttggagtgcaatggtgtgatctcggctcactgcaacctccgcctcctgggctcaagcgattctcccacctcagcctcccgagtagctgggactacaggtgcatgtcaccgtgcttgggtaatttttgtagttttagtagagacggggttgcaccatgttggcccggctggtctggaactcctgacctcaagtgatccacccgcctcggcctcccaaagtgttgggatcacaggcgtgagcccccgtgcctggccctctgttctttttttgaggtctgacaattcttctggcgctttcttgcacacttaggagaatctttctagggcagagttttcaaactgtcagatgtgatcagattgtgggttgtgaaataagtggggtgggtggggatcagctttttttttttttttttttttttgagatggagtctcgctctgttgcccaggctggagtgcagtggcgcaattttggctcgctgcaacctccgcctcctggattcaagcgattctcctgcctcagccacccgagtagctgggactacaggcatctgccaccacgcctggctaatttttttgtattgttagtagagacagggtttcactgtgttggccaggatgatctcgatctcctgacctcatgatttgcccgccccagcctcccaaagtgctgggattacaggcgtgagccactgcacctggcctgggatcagcattttaaaaacagaatggaataggatagaataccagaggggcgaataatggagtcataacgtaagttgcatcatgctgggaaggtgcgtattccctcatgaggcttctggtgggtgtgcaggtcgtggccctagcctggcaggagctgggaagctggtgctctgcaccatgtctgaacagggaacccacagaccatggggctttgctcacacctgacccgattagaaactgtcccgactccccagtggcctgacccccgctgtgcacagcggtcctgtttccaaacatctttgcctgtagttgttttcttggatgtcctaatttttgctcatctactgggaaatgatatctcattttaatttggagttaatctttctttttgaaattgtgattaagatccagttaacgtaaaattcaccaaaaccatttacaaatgtacagtttagtggcatttagtgcgttcgcaaggttgcgcaatcgttaccacgatctagttccaaagcattctcattaccgtgtacccattaagcagtcacccaccattcctttctgccccagcccctggcaaccaccaatctgctgtctctggatttgcctattctgggcatttcgtaagtgtggtcagacactaagtgtggccttttgactggcttctttcactccatgtcaggtttttgaggtttgtccatgtggtggcatgtgtcagtgttgcattcctttctttttctttctttctttctcttttttttttttttttgagatgaaatctcggtctgttacccaggctggagtgcagcggcgcgatctcggctctcactgcaacctccgcctcccgggttcaagcgattctcctgcctcagcctcccgagtagctgggaatacagttggctgccaccatgcccagctaatttttgtatatattttttttttaatagaggcggggtttcaccatgttggccaggctggtcttgaactcctgacctcaggtgatccacttgcctcagcctcccaaagtgctgggattacagatgtgagccaccacacctggccgcattcctttctttaaaattattttttttggctctttaaatacatttcttttttacgcagtagtaaagagccaacccatgcattcctttttctaagtgagtcagatgccgctgtctggacagaccacactgcgttcagccattcatctgttgacggactcttgggtcgttttcgcccattgcgaacagtgctgctgtgcacattcttgtacgtgttgttttttacacccattttcagttcttcttgggtctgttctggggagtggagttgcagggtcatacggtaactttgcgtttgatcgtttgaggaacggtcagactggtttccacagtggctgttccacgttacacttctgccggcaatgcataaaggttccagttcccacacccttgccaacacttgctattttccatgaaaaaaaattctagccatcctggtgggtgagaagtggtggcccattgtgactttgatttgcatttccctgatgatgtcaagcaccttttcatgagcttgttggctatttgtgtattttctttggagcaaggtcttttcaagtcgtttgcccatttgcagttaatcttttatttttaaaatttcacattatagtttcaaaagggttttagtgaagtgtagcacacgtatagaaaagcccacatataaagggaataacaccatacatttttcagtgaacatagtcccgggtaaccagcaaccagatcaggaaacagaattttcacccgcttatcccccaacctcccaggtgacccctgttctggtttctattgatatgttagtcttgcctgttttagaacgtcacataaatgggattatatgtgtatgcacatatctgccttttgatcaacatgattgcattatattttaaaggtagtaatagtattcatttttaaatttttttttacttaaaatattctggagattgttccatagcggtaaatatagcatgtcatgggtggtggtgttgctgttgtttttttcagacagggtcttgctttgtggcccagactggagtgcaggcacatgatcacggctcactgcagccttgaactcctgggctcaagtgatcctcccagctcagtctcctgagtagctgggaccacaggtgtgcgccaccatgcccggctaatattttttgtatttttagtagagacacggtttcaccatgttgcccaggctggtcttgaactcctggcttcaagaaattctcctggcttggcctcccaaagtccttggattacaggcgtgagccaccatgcctggcctaatttgatgagttgcatcagatgtatacacttcacctatatggcaaccaaaatcaagatagctgggccagttcacgttagtcccctccctccatcccggtccccggcagccactggtctggcttctctctctagagtattccctttccccgatgtcgcggaaatggtgttgtgtagtatgattcttcttttcttttttttgcttggggtagtgcttctgaggctcacctgtgttgtggtgttcatccatggttggttctttttgattgccaggtagcattccattgtgtggatatgtggcaattcatttatctgttgatggatatttgagttgtttccaactatcaaatatggatatatcattccatatttgtttttggtgaacattcaggcaagtcacgctcttaaaggcttcccagcctttgcacaggctgtgtcctctgccaggcactcctccaccatcttcttttcctagcaaagcccagcttattttaaaattcccagctcagatgtcccctcctccaggcagccttcctgggctcaaagggtctatttcgccagatgggagggagtggcgtccaacaagggctcaccttggcattctatcccaactctgtgttccagggccatggtctttgaccttgggggtggtggggacagaatcatagaatcagccatgttaggcaaaaggccacagtgtccccaccactgttatctaatcagctgcctggtctagtgtggtggaaaaaagtcatcctgctgatcaaaagcagggcaaagtccatcccttaacccgggtggaggtggggcagaggaggaagggtcacttaatgactgcagggctgaccctctggtgcccggggtcttcagaccttgttctaagcactttacctccatggactcattcaaacctcacagcagcccatgaggcgacactctccttacgcccattgtatggttgaggaaactgagactcggagaaggtcagtgacccatccctgatcacccagttggacagggctgcgctgggtctctgaagattccctttgggcagtgctgaatgaatgggctgaaacccagggcctgaaagcctcttctttggggagagagagagagagagagagattgagacatggacacgcggccggatcccatgacttcttggtttagccctgattcatcttcagaaccaggccaagcggccaggggaggaggaaagcatggtgagattccaaaggagaaaggtatctccagcccagcagcctctgctgagaaaccaggtgggagagggaggctgcaaagggaaattgaggctgcgtgtgtgtgtgtgcgcgtgtgtgtgcgtgtgtgtgtgtgtgtgtgtgtgagatggagtctcgttctgttgcccaggctggagtgcagtggtgtgatcacagattgctgcaacctctgcttcctgggctcaagtcatcctcccacctcagcctcccaagcggctgggactataggcacctgccaccacggatggctgatttttctattttttgtagagatggagtctccatgctcaggctggtcttgaactcctgtgctcaagtgatctgcctgcctcggcccctcaaagtgctgggattacaggtgtgagccaccgcactgggccttgttttgcttttttaaaaaagcagctttattaaggtgtgattgatataaaacaatggcacatatttaatatatgcagtttgatgggctggtgtgtgcacaccgggaaacccgcccctgagatccaggtgctgaacatacctgtcacctctacggttccctcctgccccccactgttttctgtggtgagaacatatagttcgtgctctactccccgaacacattttccagggttctgggttttgatgctgtgttttctatggggggagcaccagcagctggatgtgtcccccaggctggtgaaggggtctggcctgtggtgagcagggtccccagggctgcagggtctgtcccagacagagggacctgcagtgcatgggctttgaggctgggtgaaggccaggactgaaggaagggctgggcgatcggggcctgagagctgggaggggctggagctggtgcctgtgggctgtggtcgcatggggtggtgggagccgccatggggtggggagggcgttaccgagggggcaggactgctctttgatccagctcacataccacctcccccaggaagacctcccggactcctcagtgccctccaactattggttgaatctctctccagccttagttgtgcctcagtttcctaatctgtaacatgggggtaataatagtggaggtggctgcctcaaagagtaatggtgctgttcagaggatgagaacacagtgaacagcatcaccaggctcatggcgcaccccggaaatgctcgcttctgttacctcattaatattcagtttatgcgccttcgtttgcacctatgggctgctcagaccataaggtgatccttctggtgctggcagctgggtaggaagacacgtttatcaataaacaattatggcacttgaaggccagtctctgatctcctgatttttccaagagaactctgaaatccagattttacattacatcttccagtttcttgacaaagaattcagcttcttcattgacacgccacgtaggtcaaactagacacagttgtagctacagtgtcactagctccttcgtacagatgagaaacttgaggttcacaggggctgacttgcccaagtggccccgtggagtgcgacccagggtctggctgcctccaaatctgctccttccgggggacatggggctggccctgactggccagctcagctgtctgcctcctgtagagcctctgcctgttggttctggctgtccttgtcttagaaggccatgttcaaaagagggtggaaggtgacgcagctgcctggctttgcgacatcagctctagtctaatgggctgggtcactcctgccgggctctgacgtggtgtgggcactgaggggcccgagatatgtcccggctgggttgagggtgggggtcaggtggactaggggggacctccctgtgacactggggcagcgaattatagatctagaggaggaaagctggcagaggagtccaagtggaccccaggcgtgtttcctatagtccctttaagaaaaccatgtttttgcaacatttttaaatcaggagatttcatatacatttctttatttctggttttcatggaaaacttctgtgttctcatcttccgactggctcactcagccacacagggaggctctctcccagcgtctccctaccacagggcctttgcacaggctgtggttgctgtggaatgtcccctgcgcctcaccttctagatttgtgagctcccttcctcactgccttctgttttgattttgggtctaggttgtgtttgtgtgtgttctttttctttctttcttttcttttttttttttttatgagacagggtgttgcttggacacccaggctggagtacaaatggcgccattacagctcactgcagcctcgacctccttggctcaagtgaccctcccatctcagcctcccaagtagctgggactacaggtgtgcagcaccatgcccagctactttttaaaaaattattttttataggccaggcacagtggctcatgcctgtaatcctggcactttgggaggccaaggcagacggatcacttgaggtcaggagaccagcctggccaacgtggtgaaaccccacttctactaaaacaaaaacaaaaactaaaacaaaaattaaccaggtatggtggtgggtgcctgtagtcccagctactcgggaggctgagggaggagaactgcttgaacccggaatatggaggtttcggtgagccgagatcgcgccactgcagtccaacctgggccacagagcaagactccctctcaaaaaaataaaataaaataaaaatacaaaaattagccaggcgtgatggtgggggcctgtagtcccagctactcaggaggctaaggcaggagaatcgcttgaacacagaagcagaggttgcagttagccaagatcatgccactgcactccagcctgggcaacagagtgagactccatctcaaaaaataaataaataaataaaattattctttatagagacagggtcttgctatgttgcccaggctggtctcaaactcttgggctctggtgatcctcctacctcggcctcccaaagttctgggattacaggcatgagccatggtgcctggcctatatttcacttacgtgatgactgtctcaaatcccagtctctccccaatccctgctgtgttttctctgtgctctgtcacgtggcatgcgttttacctgagttgtttggtgcctgcctttcctccgctatggtaaagccccacatagctgggcttttggtctgtcctggtcattctcccatccccagctcttagaacccgcctggcaggtggtagtgctcagtgcttgcttgtggaatgaatgaatgatccccctggccacacggagcccatgttcctgcatgaggctatccctgccctgagcccatccgctaccctgagctccggcgctccctttaggtggtgcatgggcccctgtttaccacagtcccttgactggtgtcacctgttgctgttaggacatattttaagatgcattatgacagcatggataggttgcctgtttaattccataagctccaggtgcgacagcctcatgtgcgaagggctttcccaccacctcctatcccaagctcccgccgaggagccccttccctggccgggctcgggcagctgttccggagccttgtggtggggcgtggggccctcatcactctcctcacaagcgtacttgtcccttcccctgcagaacgtgcgcatcgaccccagtagcctgtccttcaacatgtggaaggagatccctatccccttctatctctccgtctacttctttgacgtcatgaaccccagcgagatcctgaagggcgagaagccgcaggtgcgggagcgcgggccctacgtgtacaggtgaggctgtgtccaggtgagggtggaggggccggctgaggctgggcaggggaggggtctcagagtggacgggatggggaggctgctgactgagccccagagattgttccggaagcaggcaagtcatagtcggggtaagtgctagtcccagagaagtttttgttttagggttttttttttttttttttttttttttagagatgggatcttgctatgttgcccaggctggttttgaagtcctgggctcaagcgatcctccgcctcagcctctcaaagtgctgggattacaggtgcgaatcaccagacgttgcctagagaggttctttatggagcagggagggaccaatggtgtgcgtctgggtggagggtgcatgtgtgagttacacacatacatacacacacatacacatacacatacacacatacatacatacacacgtacatacacatacacacacacatacatacacacacatgtacctacacatgcatacacacatacacacacacacacacacacacacacacatgcatgaccaggagcagggaccgaccccccagaccctatctgggccagaggaccgggtgggtcagcacgggaaggggtcagctgtttgtggaacatgctggcccaaggaccacagagttgtgcctttgctttctgcttgtcctgtacctggctgtggcctaggggaagtgacttcattcctctgagcctcagtttgcccgtctgcagactggagagacacaagagccccttcatggggtcaccgggacactcaggatgcactcggagctctgagacggctggcggatgtgcctgttacaactcccttacctcctggcgttttcacagcacctcccctcctccacacccccacttcccagttcacagacaggggagctgacttgcccccggcacacggtgttccagggatggggcgggcagagggtgttcccgctgttggagacccacagtctggttctgggaaagccaagatgaaaacccagcaaatgtgcctgaggtttgggaatgggaaacatgaatcagctgctgcattccgttcactcattcactcattcgctcgttcatttaacaaatgtttactgagcacctgctaggtgttggctgccgttctaagtacaggggacccagcagtggacagaatgggcaaaaatgccaaagctctctatactccttcattctgtgagcactgactgagcacctcctgtgtgctgggggcgcaggcagaccaaggccctgcctcaccaagctgatgttctgatggggagagagtaaagaagtggacaaataaggggaaatcaggcagccatcagtattgtgcagacaaaacagtgtgaagccggagtgcaggagggagggtggccttggggctcggtctgactggagggtcgggagcacatctgcaaagggacactcactggacccgcatggtgggaagaggccctggggagacagagtcccgggcagagcatgtgcaaaggtctgaaggctgggacagaggcctgtgcggctggggttacgagggagggagacagtggagagacgaggcctgagccttgctgagggcctcagaggtcacgttaggagcctgattttaacctgcatgcaaagtggggttgtggtccagagcatggcacgatctgatttctacttttttttttgagacggagtctctctttcacccaggttggagtgcggtggcgcgatctcggctcactgcaacgtccgccccccaggttcaagcgattctcctgcctcagcctcccgagtagctgggattacaggcatctgccaccatgcccagctaatttttgtatttttagtagagacgtggtttcgccatgttgggcaggatggtcttgaactcctgacatcaggtgatctgcccacctcagcctcccaaagtgctgggtttacaggcatgagccactgctcccgacctgatttccacttttcaaagattcttctggatggtggagagtggcttggagagatgagagatcataaggacagcagcagcaacagtcacagcagctgatgtttacctcgtgctttctctgcacccggcggctgtgttgattgctttctgggtatctgattgcttaatccccacagctgccctgtgaagtagggcttgtgattacttccttttgtagatggagagacgatggccgtgttgggtgggggagagcagaacgaggccgggtgggcggcgacaccatgtcctgcagtgggcaggcggcgggagggacagacttggcgaaggggccgagctcagctttggctgtggggccggaggtgtgcacagacgtccagggcccctggttcccaggcaggcattgcaggcgagtagaagggaaacgtcccatgcagcggggcggggcgtctgacccactggcttcccccacagggagttcaggcacaaaagcaacatcaccttcaacaacaacgacaccgtgtccttcctcgagtaccgcaccttccagttccagccctccaagtcccacggctcggagagcgactacatcgtcatgcccaacatcctggtcttggtgaggctgccctgtggcccacgccgcctcgcaccctgacctcgtcccctgtctctcctcccgcctgccccttgtgcagagagcagtccctgaggtggtcggagcgtggggactcacgcctggtgggtggctttcggccctgtgctgtctccaccacccccagtgggttctgagtttcccaggtgggtccacctgtcttggtttggaagtcctggccaaagtacttttttttcccttttcaatttacatttctgagatctccaaaaggggctgtcttgttgagggctgagccacaggcccgcctctgggactggggctggagttcacttagcctgagtccagtggggtgcaagggggagaaggggttctcgggagcacatgtggccttggcactggaggagcagagggtggttctggtgtcccagatgccccacgtggccactccaggggcctcctgcaccccagcatttcccttcatgggctctttgctgtgaggcccagctggggccaagggaggatgggccagccacgtccagcctctgacactagtgtcccttcgccttgcagggtgcggcggtgatgatggagaataagcccatgaccctgaagctcatcatgaccttggcattcaccaccctcggcgaacgtgccttcatgaaccgcactgtgggtgagatcatgtggggctacaaggacccccttgtgaatctcatcaacaagtactttccaggcatgttccccttcaaggacaagttcggattatttgctgaggtacgtgtggcctggtgagaagccaaagattcaggcctgtgtcctgtcttcccctcacacagcctggacactggtcaccagcttgctttgtagctggctggggatctagtggctgtgggttgtaagtgactgagaacctgactcaaaccggcttgagtgaaatggggaatgttggggctcatagaactgaaaatgctagggttggattcaggtacagcttgatccaggctcaaatgatgtgactgggccttagcttagcaaattggaggctttgctggaggaagggggcatggctgctggggagtaatatcacaagctgactcttaatcttgactcttggcaacctggtagggtcactgactgggcttcggagccaacatctcgtccatggagggtctgaccctgaccttggctccctcaccgcaggtttcactgagtccttgggactgctccagcctcagccatctctttggtctctgctgatcagggtagagtgctgggggggatatcgtggtgctgcgtgtgatagatactcagtacgtttttgtcaagggtgacgggctcctgtcgtgtgggtaaatgagcaagtttgggctaagtggcatgatgctgtaaaggcatcttgtaaatcctgaagtgctttgcaaatgaaagttattacaaagtccctagtttagtataatctacgtttgactgcatataatgaaaacctcacaagaaaagtctagcggtaggctgttcagtgttggtgtggtggcttcatggtcttaagtgatctggaagccttctactactgtgctctgccatccttggcatgtggttttcatcctcaagataacctcattgtccaagttggctgctagagtgtcagcaatcacatcacattccaggcagcacagcaagaaggaaaaaatggtggggctttggggagcagatggaggaactgggggtaaaacagcgtgttccagtttcctccctcttagggaggaagaaaaccaatgtccacttcctttctattggccagaactgagtcacatggccacacctagctccaggggaagctggaaaggtagccttttagctgggtgtgctgatgctctgagtatggtttgggttttgtatgaaagagtaaggagagattgggctttgggtagtaactggcagtttctgatgctctccgtccattataatgatcatttaagtatcctattgggtgagcatttattaataataacaataataatgaaaataaagactatcattttgagctcttactgtgtccggtgcactgtacctggccgtttttgcacatgagtccctttaatgctcccctttgtagtggctctggttattcttggctccacatgggcacgtgacagacacacccgggatgctgggctggggccttgtgctgggtcctgctctggaggtctgtgatgggtgcagtcactcagccgggcaaccttggagctcctcagtgtgctccaccctgcaaagcacttctagatatttccttcagtcactcataccactttgtgctttgcatgtctttgggattagaaaccagcttctgggaaatgctctcggagcaccaaagcctgggccccactatcaagaacctcgatgggttaggacaggccaggttttgctgctgtgacaagcagccccggaatctccatggcttagaacaaccaagacttattcctttgcttgcactccacgtccagtgtgggtgactggggcacccgcgcttgtggtggtcacttggggatccaggctgagggaggctctgtcttgacacatacctccttggctgctatagcagcggcagagagcccggtgaaaggcgtgtgcacagtccttaaagcatccccccaagtgacatgccacttctcctttttcattgctcagggagctgacctggccatgctttattttatgctggagtagggtgtggtgggggtgatctgccatttacctgggaggaaagccagactctcctggagttaggaagggagggtggaacactgcagccacagaacccgggaccattcctcctagaaagctcccaagcctcctctcggtccccagacactgggcatttggcagtgaaccagatgctgggggccctgtccttctggtggagggggaggagggctcagcccagaatgttcagaccaggccggctcaatggcaggcctaagccttacgatgctgttccctgctgtgtctgtagctcaacaactccgactctgggctcttcacggtgttcacgggggtccagaacatcagcaggatccacctcgtggacaagtggaacgggctgagcaaggtgaggggcgagaggcgagggcccctgtcgccagggagaggggagggtgggcccggccatggctgctcgggagtggcagggaccagagagctccttcttcctttgtcgtgaagagggtgctgggaggatgaacactcttgaagttggaggagggattttacctctggttaaagcttgaccacccagaggggcaggtggggttagtgattgcttactgagcttactgagcacctggcatctgctgggaactgagtgctttcttcctttcgattctttacttacaaaaatgttttgaaggccgggcgtggtagttcatgcctgtaatcccagcactttgggaggccgaggtgggggggatcacctgaggtcaggagttcgagaccagccaggccaacatggcaaaaccctgtctctactaaaatacaaaaaattagctgggtgtggtgttgcatgcctgtaatcccagctactcatgagctgaggcaggagaatcgcttgaacacaggaggtgaaggttgcagtgagctgagatcacgcaatttcacttcagcctgggtgagaagagtgaaactcggtctccaaaaaaaagagaagagattcacattacaaaataaaacaagagagggagataagtgcccttagtatcacctcccacccctaccccttgccccagaggaaacccacaattatcagtttgggatatattcttcccatccatttttacccatgtgcacacctatatgtacgcagagaaatacggaattgtttcttctttgaacatcatgaaatcatactacgtgcattgttttgcaactggcttttttcaccttcgtgaccttttctcaatatgtgtgaaaatgactgtgtgacttcccactccccactccccgtggtagttactgcaaagaccctgagggctagggacaaatcgtaccccaaccggtgcccagagtggcaaaaatcaccatctgcaactggcagtagccattcacgatagatggagttcacattcattgtggcttgtatattccaatatactgtacatgtgcacgtgattttaaacatcctcatcatacagaagattggaaagtgagcagttggttttgtcgagtctgggtctcgctccccagagtcagttaacctgacaggttctgttttggggttgcccggtggtggccaccattatgattttaagtagtttgcctgtgtgccgattgttttcaggaggtgcctgttgacttccgctctgagggctgagcacagccgcgccccacctctcacctcacctgctcaccacacttcttttccagattcaggctggaaatgtgggtgataggcctattttgcagatgaaagaactgagtccctagcgaagttacacattcaggggtctgggagggggaagctgtggtaggcattctctctgtgtgtctacatagcctgccctcttcccaccgtgccagtattgggaattgagtggccgtgcgtgcaccagggtgagttaggtgtgcagcacctgagagggcttattaaggggccttggccctactgaggggtctagtctggatgcttccccccaggttgacttctggcattccgatcagtgcaacatgatcaatggaacttctgggcaaatgtggccgcccttcatgactcctgagtcctcgctggagttctacagcccggaggcctgccggtaatcactgggactcggggcctcctgggtttcctgggtagctcatggccaaattctgtggtgttggctgtgcacttggaaagcattttgactcatcgtggatttgactcagtagcccttggcaccagcttgaattctctttggtcacaccaccaaaagccaaactccagctgccaatgcctctacacagcccaaagagaggctgctggtggtggtttgcagccaggtggtgctgggctgggaggagggggcaggacatgtcaagtcacagggggccataaaatggaggcgagaggtgggctccctctggcgtctggctggtggcccccatgaccatctgggtgctgagacatcagctggctctgcttggccccttcaggctcagctctgtgtcccatggtcccagttgtggggtaagagtagacagtgggtggctcctacagcccttttctggaggccttgctgtgtcgtgttccattctggtcactctggaaaagtcctttacttaggaattcactcatgtggaccttgacttatctgccctcccagcaaaccctgtgaagccctactgtgtgccaggtgcccggtagtgagagggtgggccaagtctccgttccccaggaggctgtgtcctatcagggcaactcaggcaaatgcacaaacactagagtgtcagagagtgacagtgcttttgctctgtgtggccactttttactattttaagtttttacttatttatttatttaattttttagagatggggatctcgctatgttgcccaggctagtcttgaactcctagcctcaagggatcttcctgcttcagcctcccaaagtgctgggattgcaggtgtgagctactgtacccagctgactttaaaagctgaattttaaacttaaagaaaagttgcatgagaaatacaagtggcccccgtctccccttcacccaggttcactagccgtgttaagccccccaccccctcttctcgccgagtgtctttttccagaaccatcggagagtaggttggaggctgtatgcccccttacctccaaatactcagtgggtgtttttcagaaacgaggacattttcttacataaccataatgctgttatcaaaatgagaaaatttggcattgacataatactgttatctaagctggcactttcctataaatcaaaatatgcaaggcacacatgcaatttaaaattttttagtggctgcattaatacaagtaacaagaaattggtgaaattatttttaataatgtattttatttgacctcatatatcaaaaatgggatcatttcaagtgttattgatgaggttttttttttgttttttttgctagcaagtattagaaatctggaatgtattttatacttgcatcccaatttggactcaccccatgtcaagggcgggatggacagccatgcgtgctgagtggctgccgtggacagagtgggtagcacagtgcatgttcaagttcagaagtgcttttgagaaagtacaggagcatgaatggatggagggtggatggggtgggtgtgggggacggtgctgccacccgaggaagtgacagctgaactgagatctgactgaagggctgaagtctggtggatgaagatgccagaggagactgttcttaggcagagggagcagtgatgcaaagggcctggggcaggagtgagtgtgcttaggaactgcagggaggccctgggcacaggaagttggggcagggctgagcagagcttatgcaggggctagataaggtcaatgaatcctaatggtaggttagactagaaagggtcacagattttcagcaggggcatgatatattctgttctttttttaatttcataactttcaaaaacaattttatttttagagactgggtctcactatgttccccaggctggtctcacactcctgggctcaagtgatcctcccacctcggcctcccaaagtgctgggattacaggtgtgagccataatgcctggcctctgttcttttgtaaaactccatctggccacggctaatacattgatgtgggtggggaggcagagtcggagcggagaggcctgttaggaggtcgctgcagctccgcgggtgagagatgggggcggtttggacccgggaggtggtagcgcccgtggggagaagtggctggatctgggcagcctttggcagggcctggctctggccgccgggtctgggtgtcccctctcatcctgtctgtcccctgcagatccatgaagctaatgtacaaggagtcaggggtgtttgaaggcatccccacctatcgcttcgtggctcccaaaaccctgtttgccaacgggtccatctacccacccaacgaaggcttctgcccgtgcctggagtctggaattcagaacgtcagcacctgcaggttcagtacgtgccgtcccctgtgctgggatggcgggagggtgtgagggtggggccagctgagggtttatctgcccagtgctgtctgcttaatctctggcctctgtactcttgataatcccattaggcgaagataatgaggccgcaggaagataatctgaaaaggaagaaatgccaggtctattagttgctgtcacaatatattgccatgaacttcagggcttagaacaacacacactggttatcttgcagttctgtagtcagaagtctgcgtgggtctcaccgcgctcagaacaaggcggctgtgttccttctggaggctctaggagaagctgcgtctgccttttctggcttccagaggcccctctgtgtttcttggctcccgggcccttcctccacctttgaggccagcaacatggcatccttctgatccactctgtcctcatatctcttcctctcctccctcttctgccccctcttccagttctaaggacccttaagaccacatcgtgctctcctggataccccaggacaatctccccatcttgtttattattattttttttgagacagactctcactctgttacccaggttggagtgcagtggcgccatttcagctcactgcaacctccgcctcccaggttcaagtgatcctcccacctcagcctcccgagtagttgggaccacaggcgtgcaccaccacacctggctaattttttgtatttttggtagagatgggggtttcaccatgttgcccaggctggtcttgaactcctgacctcaagtgatctgcctgcctcggcctcccaaagtgctgggattataggtgtgagccaccgcgcccagcctatttttatattttgagacaaggtatcactctgtagcctggagctggagtatagtggtgcgattatagctcactgcagcctcgacctcatgggctcaagcgatcttcctgcctcagcctcctgagtagctgggaccgcaggtgtgggccatgatacctagctaattttatttttgtaaagtttttatagagatgttgtttcaccatgttgcccaggctggtctcaaacacctggcctcaagtgatccacctgcctctgcgtgttaaagcaatgggattacaggtgtgagccactgtgcccggcccaatttctggaccttcaaggcagctgctgagcaggcccagcgcccgtttaccctgtgacctgacatagtcatgggttctggggcttagggcatgtacgtgtcctaaatgacatgaaatgaggctggcgagggtctgtgtgttacgtaacatcttctcgttccgcagtgtgggtggctaggcagatgtgatgactgctgttggcgtcggtcccctgacattgttggtgtgtctcaattgtgtgccagtccctgagacacccccacctcaagttgctcagtttggaccctcgggcgtcagtttgtttccctgtcctcacccagctcttccagtgcgtcaacaagcccagatggctttacctccaggttgagtcctcactccttcctctctcttgctccccaccccaacccccgcctcattgtccaggccacggacacgtccttcctggtgatccagccctttcagtagcccatccctcaatgcagtgttgccgccttaccatccatcccctagaccatccagtgcggtcactgctggcctcttgcggctgtttaaattgaaattgatttaaacgaaatcaaattaaaaggcagttccttggttgcaccggccggaagttaaatgctcataagccatgtgtgtctagcagctactgtcttggatagcacagatagagaacatttccatcatcataggaagttctgtagcactcccgacagcagcggagggatcttttaacaatgcaggccgagtgtggtggttcacgcctgtaatcacaaccctgtaggaggccgaggtgggaggatcacttcaggccaagagttcgagaccagcctgggcaatatagcaagacctctctctggccaaaaaaaaaaaaaaaaaaaaaaaattagctaggcatggtggtgcatgcctgtagtcctagctacgtggaaagctggggtggaaggatggcctaagcccagaggtcaaggctgcagtgagctgtgatcaccccactgcactccagcctgggcaacagaggaagacccatctcgaaaaatatgcagatggccgccgtcctcttaaaatgtctggcagctctgtctgcattcatagtgaagtccaaactcccaccatggccctgggccctgcaggatccagcccctaccggcctctctgaaggatccagccccaccagcctctctgactccatttctccctacacgtcccctgttcattattctccagccctgatgccttcgccctttttctaaagtataccaaggtcagtgccttgcactgtggtttgcatgggttgcttcccctgcttggagagctctttgtctggatttctgagtggatcatccctcaccttcttttggcctgcatgcaaatgccacctcctccgggaggcctcctctgatttcctgttctaaagttgccccctctcccacctgctgctccctatggcttcctcttcttgtcttcttgtcttggtcttcttgtcttcctcttcttgtcttggtgctcattgctccctgaactcatggactgtggattttattcattgccccttccattcactgtcatccctgtaggcagggccccaagctcctcttgttggttttattcttagcttctagaactgtgccccgcatgtaatgggtactcatacagtatttgaatatagggatggtgagtgcgttctgtacttaggaaattgaggcctgatgctggatgcgaccaaggtcacagggttggtaggaccggaagcggccctctatttgactgtgtcttgccttctgggagcctttttggttgaagccccgtcctccagctggcaatggcgactctaaaggactggtctgcaacaggggtcaggaggaggggctgtcaacagattggatgctttcccctatttcaggcagaatggaacaacctgtgaatgaatggtttggagctggggcctgggacgcccgctgctgtctcctggtcctgtctggcccgtctgggcattggcctctgcttgcttcattctttgctggtatcggcggggatttctgctcctaaggttgaggatgttacacacccaataggtgtgctacagcaactgtcgcaagtggcagatgctggactccacctgatttaggccaaaagggcacttcttggtttgcgtaatgaaaaattccagggcagatctctgcattaggcacaaatccagaacttagatgttgtctggaagctgcctctgccaagtcccagctcagttttctgctggactggctttgctgccaagcaggctctccctcctggctgcctgtggaggctccaggctcacctccgcccagctccgagtcctgcagaaagggggccacctgtgtttccttcactccagcagaagttccagggtttgctctaattggtctgcctggctcccatgcccagctgtaaaccaatcacagtagccgaggtggactatgctgattggccaggcttgtgtcacatgctcagatggctcctgggtgggccggccccactccaagcacaaggctgaaagtagggaaggcttaaagtgggggtgtaggtaccaggagaagggggagaaacgacatgcccatcccctccaagggcgtgggacgaggtgtgtcccgccttctcttagcacatctgccaaaagaatttcaaaagcgcaaaggaattttaaaatctttatttaaatagagttttttcttatatttatttatatttttgagacagggtttctctgttgcccaggctcaagtgcagtggtgcaatcacagctcacagcagccttgacctcctgggcacaaatgattctcccacctcagcctcccaggtagctgggagcacaggtacacaccaccatacctggctaatttattttatcttatttcaattttttttttttttttttttttttttgtagaggtggggttgtgccgtcttgcccgggcttagtttttgcttctaaataaaatatacatagatacaaagaagttcccagatgactcatgcagtttttggggttttttgctttgttttttgagacagggtatcactgtcgcccaggctggagtgcagtggtgccatcatggctcacagcagtctgacctccatcactcaattgatcctcttaccccagcctcctgagtagctgggactacaagcggttgctaccatgcccagctaattcttaaattttttttaagagacggggtctcaccatattgcccaggctagtcttgaagtcctgggctcaagtgatcctcccgcccaaagtgctgggattacaggcatgaaccgctgcgcctggctgacacctgcagtttgaaacttcacaaagtcaacgcaccccgtggccagcaccctgactgagaaacagacaggacaggccctggatgccactcgcggtcctcccaccgccctgacatccagccccggagctcagctggtttgcatcttcatgtgaatggacccataatgctagactccttttcctccttttctggtccgtgcaccccttagcaccctggtgcaattcgtccgtgacgttgtgggtggtagcagcccctcctctccgttgctgtgtggtgttccatcgtgggtgccacctccatcttctccttccattgctcctaggcatccaagttcctgtttttcgaccgtgaccgtggtgcacacgtgtcgtttggtgcacctatgtctgtgtttctgtggcatgttatctaaagagtaaaggtaccatgcctttgcctttctgggccttgaaatctgttggactcagacaactccctcatctcaaacctccctgttgcgtgagggcatccgacttgcctcacagagccaactaattatcatcggtggcaatcctgggcaattatgtctggctagatatgccataaagctacaaattaattaccacttacagctgggaagatctctctcggcgtgcttggtaataacattggaagaaacacgtgcgtgggtccatgcaggctcggcctctgaagcaagggcgggggctggcaagttgttccatcccacctcggtctccacctggggctgtgggcaggccagggcttgtttgagattcctccagagttcggctccgtggctgcctcctccacggaggaggcacgagaggcagcgttttccagattgtggaggacactgtccctgcgggtgcttgggatggcttcagaaggtccaaggaggtgagagtgagcttctgggtccatgctcttttcctccttgccatcacctgcaggagaaggtctcttggtgttgagaggacttgaagagccttccacactcatcagccttttttgttttgttttttcttttaacagaaaaagagcagggctcccagttaaagcctcatgcagtaacaggttctagttagaacttacaacatcattattttccacggtattttttaactatgtcttttttttttttttttgagacacagtcttgctctgttgccaaggctggagtgcaatggtgcaatcttggctcaccacaacttctgcctcttgggttcaagcgattctcctgcctcagcctcctgagtagctgggattacaggtgtgcaccaccaggcccggctaattttgtatttttagtagagacggggtttctccatatcggtcaggctggtcgtgaactcccaacctcaactggtccacccgcctcggcctcctaaagtgctgggattacaggcgtgagccaccgcgcccggccctttattttattttattttttttgagacacagtcttgctcttttgccgaggctggagtgcattggtgcaatcttggcttactgcaacctccgaaaactattgtccaggctggagtgcagtgatgcgatttcagctccctgcagcctccgcaacctctgccgcccaggttcaagcaattctcctgcctcagcctcccgagtagctgggattacaggcacataccatgctcagctaatttttgtatttttagtagagacggggtctcaccatgttgcccaggctggtcttgaacttctgacctcaagtgatccacccgccttggcctcccaaagagttgggattacaggcatgagccaccgtgcctggcctctgattcaagtttttgttttgttttgttttgttttccttactgaggcaaaattcttgtaacataaaattaaccattttaaagtgaatgattcagtggcatttagcagggtcacaaggttgtgcagccatcacttgtatctcgttccagaacatttctgtcaccccaaaaggagacccagactcatcagaagtcactccccgccccctcccccagcccctggtcaccacccgttggctctgtctgtggattagcactgtctggatccttgtgagctttttaaaaaaaaattgtttttcttttgagacaggatcttgctctgttacccaggctggagtgcagtggcacggtcacggctcactgtagcctcaacctctgaggctcaagcgattttcctgcctcagcctcctgagtagctggaactacatgcaggtgccaccatgcccggctaatttttgtagagataggatcttgccatattgtccagcctggtctccaattcctgggtgcaagtcatctgcctgctgaggcctcccaaagtgttgggattacaggtgtaagccatggcgcctggccctcgtgagcttttgagagaggcccgctcgctgttccttgttaacttgtccttcaggcctgttgtccattgtcccgagggtgagctggtgtttgcctggtttggttggtcagtggcgaggttgagctcacatttgtagcaggtgtgtctacagggacacactcctgtggatgcagggagatctggccacaggtgcacacacctgagctccgatcattgctgcgtgtcctgcaggggacagtgtgggctgcttggcttgcgcttgagactcccctccccacacccaagttcaaatctggcgttgccttttccatccgtgcctccctagcacccttgtgcaccatgcatttccccctccccacgcttgcttctgccttcccttccacctggaatgcgcttcccgggcatccttcacctggctcctgtgaagaggcctcttccttcttttggtctctcctgtcagaatcagcggctcctgcctcaccccttctctggcgcagagcttgtccctcatcacagggcctggggctttttacagaatggaggaagggatcctctctgtctggttatcttgtcatcgccacgggggtgccctgcagaccacagctctgtgcagacctccggcctggcaggacctgccaatatactgtccttgtctgatgtcccctccctgcccctcttctaggtgcccccttgtttctctcccatcctcacttcctcaacgctgacccggttctggcagaagcggtgactggcctgcaccctaaccaggaggcacactccttgttcctggacatccacccggtgagcccctgccatcctctgtggggggtgggtgattcctggttggagcacacctggctgcctcctctctccccaggcagagagctgctgtgggctggggtggtgggaagcctggcttctagaatctcgagccaccaaagttccttacttcaccccgactccatagttcaaggtagttcaagggttttatgatccctgtactggtttctataaatgggctctaagacagtaaattaattagaacttatcagctgggatgtctcctacatgtgagctggaggcagccctctggatgtgtcaagataccataaagatctttaggtaccaagaagagcctggggtattttgcagataaatcacacagggaatttgtcccgtgtgaagttctgtctacaagcagggagctggacatgtggccctcccaaggacactccttcagcttgtggcctcttctcttggcacctccgttctctgtaaaatatgcccagttggtcttggtggctgctaccattgattcagaaatctgtggttttctgtttcctgggtgggcacttgtggtctatcccatgggcctttaacaaaattattattatttttgagacagggtctcactcggttgcccaggctggggtgcagtggcatgatcatggctcactgcagcttcgacctccctgggttcaggtgatcctcccacctcagcctccctaagtagccaggactacagacatgtaccaccatgcctggctaatttttatattttttgtagagacgcggttttgccatgttgcccagtctggtctcgaactcttgggctcaagcgatcctcctgtcttggcctcccaaagtaccgggattacaggcatgaaccgccgtgcccagccaaaattaattctctaaattgaagaagaaaagctgtctatttttgagtgtacaacatgatgttatgatgtatgtatataacttgtggaatacctaaatcaagccaattaacatatgcattacctcacatactgatgatgtattggtgctgagaacatttaaaatctactgtcagcaactttcaagcttacaatacattgctattcactctagtcactgtgttgtattgtatagtacaatacagtgactgttgctctatttcccagtaggtctcctaaacttactccccctgtctaactgaaattttgtatcctttgacggacttccccatccctgcccctggtagccagtattctaccctctgcttccatgaattcaactttttttcttttttttttctgagatggagtttcactctggttacccaggctggagtgcagtggcacgatctcggctcactgcaacctccgcctcccgggttcaagcaattctccctgcctcagcctctcaagtagctgggattacaggcacacaccaccacacccggctaatttttgtatttttagtagacacagggttttgccacgttggccatgctggtctcgaactcgtgacctcaggtgatccacccgcctcggactcccaggttgctgggattataggcgtgagccactacgcctggcctgaattcaactcttttagattccacatacaggtgaggtcatgcagggtttgctttcctgtgcctggcctattttacttcgcatagtgtccccaggtccgtcatgttgtcacaaataacaggatttccctgtttttatttatttattttttgagacagagtcttgctgcgtcacccaggctggagtgcagtggcatgatcgtggctccctgcaacctctgcctcctgggttcaagcaattctcctgcctcggcctccagagtagctgggattaaaggcacacaccaccacacctggctaatttttgtatttttagtggagacgggatttcaccatcctgaccaagctggtctggaactcctggcctcaagtgatccgtccacctcggcctcccaaagtgctgggattccaggcgtgagccgccgcgcccagctggatttccctctttttaaacgctgaacagtatcccactgtgtgtgcaccacgtttctgctgcccattcattcactgacggacacttcgcttgattccgtatcttggccacagcgtacatgggagtgcccctgtccctttgacacactgatttcatttcctctggggggatgcccggcagtgggatcactgatccgatggtagttctggttttcgttgtctgaggaagctccacacgtttgccacaatgctgtgcaccttaggcccttcccgaagctccatggcggatgctgttctgcaccatttttctttgtcctcccaggcctccttagccgctgactaggccctctcggccccggccccagccctggtctctgccttccctcttttagttaattcctcctccagcctctcttgctctgctggcttccggagaattccaccatgaacacagtctcctgtgcgctctcagtgatcgctttcctttcagggcacggattttctttcttcccctcacatcaagaaagaaggggttaagtttgggagcccagagagaggcaaacagctgttttgccgcagccactttagctgctgcgtccaattctacctccagatgagcttgaggccgacggggagaggaagtggcctttatttctttgttttttgagctgggcacgttggggagggggatcaggagatcagcttgctgcctgttggcctttagccttgaaaaacgaacgtgtgttggaagctgcttccgtgtggccacttttctgctcctgaaactgggggagggcatggggacgggggactcaacaggacgctgcttatggtggtatcacagctaagacttattgtaatgacagaattcaaagcaaaattagcaaagggaaaaagcaccttgggtgaagtcgggggaggccaggctggagcttccagagtcctcttccagggggttcctacaggatgcacttaatgtctttagcaatgagttgtgacaacatgtaggagatgttgtctagcagggaagctcattagaaattcagtgcccatggtgttttgctaggggccagtcatgtgggcacctctgcctggcaggtaccaaaattctaggctctgggaaggaaagcaggtgctgagcaagaaccatattgtttgtacaaataatttcggcccagtgaactactctcatcagttaggggatggtggggaccctcctgaaatctgggttcccagatgccagcagaattcctaaggagagcagcttcaggcctgtggcgtgaactctgttctactcacgggtgatctcatgcccatcccaccaggctctcgagggcaagcactccccagtcgtgggcgtaagtgaggcctttaatgagctcttctttgccacaggggcctggaaggtcctcagcatgctgggccataatgaagaaaacatcctttccctatagtgtacgtgataataacctaggcatttatggccctgcgtgtggctatcttgcataccttacttttgctcttgcaaaaattctgtcaggtcgggagagttctctctgttttacagatgaggacgcgactctcagttaagaagaaccaactgcagttctctgacctcaaatctagttttatttccactatcatttcctgacttcattctggaaccaccttcctgttgtttttgatgacacatgtgtgtcacttcggttatttacttaaaaaaaaaagtctcgttaaaaagagctgggtatagtggctcatggctgtaattccactgctttgggaggctgaggcaggaagatcacttgaggctaggagtttgacgccagcctgggcaacatagtgaaactctgtctctacaaaaataataatgataaataggctgggtgtggtggctcacgcctgtaatcccactttgggaggccaaggtgggcagttcgcttgctgccaggagtttgagaccagcctggtcaacatggtgaaaccctgtctttactaaaaatacaaaaattagccgagtgtgctggcgcacacctgtagtcccagctactggggaggctgaggcaggacaatcgcttgaactcaggaggcagaggttgcctgcactccagcctagatgactgagtaagactctgtctcaaaaagaaataaaaataaatttctgcacaaaggaagctttttcttactacctcaaataggaaattacaaaattctccaaaaaaagaagacaaccataaaaatgaacaaggaaaaaaatattaggctctatgtaaaatattgttgtctgtcagcaggagtgaacctggggccagctgtcagcaccattgttagaagtggagattttgtgtaggtgtattagaggggtattaaagaccatgcaaacacctggctgagacctactcttcaaaatgatcaggaggagttgggagtgaattgagaagggacttttgtctgttttattgaatgtggaggttgagagggagcatggacaaattgccattctgcactttgggaaacgctgtcttaggaggacaggtgaaagtgattaggtattttccaccctgagacagccttgctttatagaaggaacctctttgaggctgtgaacataactgtgtgggaagcacttgtttccctataaggggttagaatcaggagaggagatcccagtgcacctgcccagcatttctagaactgttgaccccccccagcctgtggcttgttttaggtaagatacaagcaagctccactgggcagttagctgggacgcccaccctcttgactgggaccagggagaggagggttgacggtgtccctggagcttgggggtggccagtctcctcactgtgtttgttgccgcaggtcacgggaatccccatgaactgctctgtgaaactgcagctgagcctctacatgaaatctgtcgcaggcattgggtgagtggggactgggagctggggctgcattgctcattgagagattaggtgctcagtgctccagtggtcccagactccagtgacataccccaggaaccagggcatggggaggggagagggtcctattgggggtggaatccagtccctgctgatcttctccctgaggggtcttggttttgagccaggtcctgacctgcatcctgatattccttccccagaacaccacttttctttctcttttttgttttgttttgttttgtttttgagatggagtctcgctcttgttgcccaggctggagtgcaagggcgggatattggctcaccacaacctccgcctcccaggttcaagcgattctcctgactcagcctcctgagtagctgggattacaggcatgtgccaccacgcccggctaatttggtatttttagtagagatggggtttcaccatgttggtcaggctggtctcaaactcctggcctcaagtgatccacccacctgggcctcccagcatgctgggattgcaggcgtgagccaccgtgcccggccttcacttttcttttttaacattcaccccctccctgccaattagccagagcttcaatccaagctgtttcctacacgacagagtggcagaaatgaaagcctggcaaaaccaaatgtttgaaaatggaaacgctgaggcaggagaatggcgtgaacccgggaggcggagcttgcagtgagccgagatcgcgccactgcactccagcctgggcgacagagcaagactccgtctcaaaaaaaaaaaaaaaaaagaatttttcaagcactgatttcctctttttgatttttaaaataaaacagctttatgcaaatataatgcatgtaccataaaactcacccctttaaaatgcacaattcaaaataaaaaaaataacccccctaataaaatgcacaattcagtgatttgcagtatattcacagaattgtgtatctatcaccacaatcaattttggaacattttcatcatccccaaaagaaaccccacactcattaacactcacttccctgtcccctcagccccaggcaaccgttcatccacttcctgtctctgcggacttacctattctgggcatttcatataaatggaatcatacactatgtggtcttttgcgactggcttctttcatttaggatcacgttttcaaggttcatccatgtcgtagcatggatcagtatattttatggctggataatattccattgtctggatagaccacatttatttatcggttgatggacgtttgagttgtttccacgcctgggccgttatgcataatgcagctgtaaatgttcttgtgtcagtcccagtgtggacatctgttctcgttttccttaggtatgtaccgaggggtggaatcgctgggtcctatgttaactctttgtttaaccgtttgaggaacagtcagacttgcacagcctctgccccccatccccattcttttttttttttttttttttttgagacggagtctcgctctgtcgcccaggctggagtgcagtggcgcgatctcggctcactgcaagctccgcctcctgggttcacgccattctcctgcctcagcctcccaagtagctgggactacaggcgcccgccaccgtgcccggctaattttttgtatttttagtagatacagggtttcaccatgttagccaggttggtctcgatctcctgacctcgtgatccgcccgcctcgggctcccaaagtgctgggattacaggcgtgagcaccgcgcccggccccatccccattcttagcagcagtgcagcagggttccagttcctccccacgcgcaccagcacttgctataacattacacgttgctagaacttggtgagggattatactgatttgttgctatgtcagtgtctgtacttcttagcatatctgaaatagtttcgttaaaaaaaattctcttagaaaaatccctgggttgcaggaatgtgagcatctattcagctttgtcaaatgcctctcggctggaaagaataacactttgtcagagcacggcagcgagtaataactgtgagctctcttccttcatcccgcccttgcattttatttttatattttgaggccacttagggaatttgttcttgatggatttgtgggtggggaaacagccccaggcatggaagaggcgtttgcagcccaagtcctccctctggttccaccgcgtggcacctgggctgctaactgggatgcaactggggccaagtgggtgaccagatagaagaggcgacctggggccgaggatacagccccttcccagcaccagctgactgtagccccatggaaatgcgggctcagtgtggccacatcctctgcatttttcaaaaggacttccaaatctgaattttaacaggagctctgtcaatttttacttattgggaggtaattcacattctttttttttttttttttttttttttgagacgaagtcttgccgtgtcacccaggccagagtgcagtggcatgatctcagctcactgcaacctctgcctcccgggttccagcgattctcctgcctcagcctctcaagtagctgggattacaggcgctcgctagtacacccagctaatttttgtatttttagtggagacaggatttcaccatgttggccaggctgttctcgaacttgtgacctcaggtgatccgcctcccagagtgctgggattataggcatgagccctgcgctgggcctaattcacattcttaacaaacagttcacgcgggcagctggattgtgcctgccagtgacctgtggaccggtcacccaacctctctgggccgcacagctgctgacctccctgtggactgggaacaaggcactccaggaaagtggtctcaacagcagatgtggagggccacgagggatggccgtgggaaaagtctagagacacagctgccgggaagcagagctgtctcgtgacctgtcggggaagcttctgtgctccttgctcgctaggtaaaggcagtggggttgcatgttacttgaatacaattcccattggttcttacaaagtctttcagagaaaagcagccaaagtaaatggtaccgtctgttgtctccccatcgtgtttcgtcctctgtagggtgatagtgtgagcccatttcactgtgcaataccctacttaacaaggggcctgagagacctccaataattgtgattgggggatttcagcctttttttttttttaagatggatcttgctcttgtcgcccaggctggagtacaatggcgcgatttcggctcactgcaacctccgcctcgggttcaagcgattctcctgcctcagcctcccgcgtagctgggattacaggtgcccgccaccacgcccagcaaatttttgtatttttagtagagacggggtttcactgtgttggccaggctggtctcaaactcctgacctcaggtgatcacctgcctcagcctcccaaagtgctgggattacaggcgtgagccactgcgcccggtctctgattgtggtatttgagccttttgtgctgactttcgtcaccgcccctcagtgacttccatctcccctccctactgctgtgcatggcaaagtgacagaatatttgtgtgtccatttctttgctgggatttaacaattataacaacaacaataataaatgtgccaagcacttttatatgccgggtaattttcgaagcactttacatgtattactgcattgaatcctccctagagtcctaggaagagttacgttataatatctcgcttatacagaagggacacaggctcagtagcacgcccaaggtcacagagctcgtaagtggctgtgtcaggggtcccaaggccaccgcaggctcgacgatttgctgggaagcctcaggacgaagcatatggttgtacacgtggctatgatttattacggtaaaaggattcaaagcaaatcagcaaagggaaaagccacacggggccaagtccagaggaaagcaggtgccagtttccagagccctctcccaggggagtcacccaattcttgctgcaaggagttgtgacaacccacgtgacatgtcatctggcagggaagcccgctagagagactgagtgcccagggtttttactaggggctggtcatgtggccaccctctgcctggcaggtactgaaattccagacccttagaaggcaagctggtggtcagtataaaccacattacttgtacagattgctcaggcacagtgagccatccttgtcagttagggagcaatgggaggcctcccgaatccaggctcccagacgccagctccgggccagcattgccagcagcctgtgtggggtggaagggctcgggcctgccacgtgagcacgtgcctgcacagcggcccaggtgggattcaaatccaggcagcgacgcttgagtgtgcactattaaccacctcgccacactgccttttctgctagacacctgttgtgggggggtctgtgtggggcaagtggtgagccagcccaccctgctgctccatactgagtgtgagttgctgtattcatttccttttgctgctgtcacaattttgccacaaatgtcatggctcacttttaaaaactatcttgcagtgttgggagtctgaagtctgaaaggaacacaacaaggctaagatcaaggtatctgcaggccttgttccttccagaggctccaggggagaacctgtgccttgccgtttacagcttctagaagctgcctactttccttagctcatgaccttaatcctagccctccctgttttgttgttgttgttgttgttgttgagacacagtctcgctctgctatctagactggagtacagtggcatgatctatagttcactgcagccttggcctcctgggcttaggcgatcctcccacctgagcctcctgagtagctgggactacaggcatgtgccaccataccctgctaatttttgtgttttttttgtagagacaggggtctcactatgttgcccaggctggtcttgaactccagggtgcaagtgatcctcccacctcagcctcccaaagtgctgggattacaggcgtgaaccactgcacttggcctctccctctttaaagccagcagtgtagcactgtccaatttctctctgcctctaacctcctgcttccctcttttttttttttttgagacggagtctcactctgtcgccaggctggagtgcaatggcatgatctccgctcactgcaacctctgactccctggttcaagtgattctcctgcctcagcctcccgagtagctgggattacaggcatgtgccaccacgtccggccaatttttatatttttagtagagatggggtttcaccatgttgcccaggatggtctcaatctcctgaccttgtgatccacctgcctcggcctcccaaagtgctgggattacaggcgtgagccaccacgccccagcccctctttttaaaaattaatttttctttttttagggttttaaaatttgtttttgttttttcgtttttctttctcttgcctgattgctctggctaggacttcccgcttctctctttaaggacttttgtgattctctggggcctacctaggtattccaggatactcttcccatctcagaaccttgatgtaaccatatctgcatggacccacctagatattccaggacgctctccccatctgagagccttgatgcagtcatgtctgcacggtccccttcgtcgtggcaggtcacagtttcccaggtgtcgcattggtcttccggtctccttaggcaccttttggctgtgacagtctctcatctctccttcattttgatgacagtgacacttttgagaagcactggtcaggtattttgtaggatgaccctctactgggattggtctggtgcttttctcatgattagactggggtgatgggttttgggaggaggaccgcagagagaaagttccagtttcagagcatcttgggtacattccgtcaacatactgtatcactgttgatgtcgaccttgatagcctggatgaggtcatgcctgtgacgtttctgcagtatcaagtttacttttattctccctttggattttattatctttttaacttgacgtgtaagttttaacttcacaggtaataagaacatatcttcctgaggggaagaatatggtttcctgtgactccccgccatccccagccgtacccaccatcatcaatttgctgtgcttccttccaggccttttcctacttatttacattcatatatatgtggccatagagaagatacatattgtcgtttgtctgtttgtctttgctagtccaagcaacactgaacatctttgtgcctctgtgtgcatgtgtgagcatctctagggcagtttctgaagagcgtaattgctgggtcgtgaggtatattggtggaattaggtttggcagtatagatcagtaaactcaacagttgtggctcaaatgggacaaaggtatattgttattttaaaaatcttcttttagaggatgtctggaggcagacagtccaagtatggcaagagacattcatagtccaagttccactcatctttctgttccattttcccaacacatggcttccatcctcaaggtcacctctggtcccagtgtctgcagagctccagcccttgcatctgcgtttcaggcatctgaaagggaggagggaaaaaaggttcacctcccagctgtgaaagcttctttttaagcagccctcccggccatcccacgtaacgcttctgcgaacatctcattaaccagaacttggtcacagagcctcacttgccacaagagaggctgaagtggagtctttaactgggtgcattgccatcctgagtaaaatttgggtcctgtttctaaaagggaccaatggattttggctgggtcactggtggtctctgccctgtaggagatgtgagtttttagtttaaaaagattattttcaagttgtcaagctctttgccaggggagattccttctggcagcacatcgaaacactggttccctcagcccatgccaatcaagtaccatggaccccttatccctgggggtatattccaagacccccagtggatgcctgaagctggggatagtactgaaccctgtatagcatatactatgcttcttcctatacacacacatctatgataaagttaatttataaattaggcatagccacaattaacaataactaagaatagggtaattataacaaatactataataaaagttatgtgaacatggtctctccctcaaaatatcttattgtactgtactgagggtaagaaatggcagaaatcaaaaccatggataaggagggatgactgagattgcagaatcaaatccctcctggcctcagagcttatgatcacaggttcaaagctgtgtgatgtcaaacagccctgtgggaaaacatctcaccttgtcaactaaagaaaaaaaaaaacaaaacgccttttaaagatttaaagttagtcttatttacaagtattattgagggctgtagactgagaccttcagcctggggacagttttgtcatatggctcctaaagtgtttcagctcattgtttatatttggtggtgagggtttagtgtgtgcaaaattatactaaacctgtttagatgttgtattcaagcagaattagatcaagtttgggtgtaagactttgtttcacacagctatgtcttgcttatttccagacaaactgggaagattgagcctgtggtcctgccgctgctctggtttgcagaggtaagggtgcgttgggcacagcgtcgggggcttttgttaagagccaatgtgggcatttgaggcaggaggcggggggagcagcttgtagaaagggagagggctgagccagggtaaccggactgtgacatggaccagcgtatcagaaacttcaccctgtccaagcaccctatgtcagttatcccaccaaggtgaaagggatccctagagatggggaagacagaagctgcatgaagaggtaaagtccctggccctgggtgtaaaataattttgttggggcatatgacctcttctcctgaaagtggggaagttggtttccaaggtgtcagcttttctcttaaacttacttctctcgcagcttttcagaattccttctggcctagcttgttttattttattattttattttattttatttactgtatttattgtttcttgcccacctcgttttaaatcagggctagctaatggccaggacaaacaagcctccgaatgtcaagtttacttcctactcacagccgaatctgaagtgggttgatggggaggcagagctctgctccctgcagtcatttagggttccagggccccggtggccccgccatcctggagagcctccattcagtggcagggaacgagatggagagtcacgtgaggccaggtccacctcccattggtcaggacacgatcatatggccctgcccaactgcaagggagtcggggaattgtagtctcaccttgtgtcctggagggaggaggtccctggcaggctccaacacatgctttagccgggaagcttgaggtggggaaaagctgaggcgggcacagaggaaggtgttgggtggcatctgcgctgtagcccgcagcctgcggccccagctcatgtgtttgtcattctgtctcctcagagcggggccatggagggggagactcttcacacattctacactcagctggtgttgatgcccaaggtgatgcactatgcccagtacgtcctcctggcgctgggctgcgtcctgctgctggtccctgtcatctgccaaatccggagccaagtaggtgctggccagagggcagcccgggctgacagccattcgcttgcctgctgggggaaaggggcctcagatcggaccctctggccaaccgcagcctggagcccacctccagcagcagtcctgcgtctctgccggagtgggagcggtcactgctgggggctgcgcagcacgcttgcgtcttttgcatgccgcgttgccactactctgcctgttctggaaggcctgggaccctcccttggagggggcacaggtgggctttgagtaatgagacctggtacttgcatcatccattcatcaagtcagcacccggggatgccaggttctgttaggggcgaggggacgtacagcagtagaggagacagctgagatccctgctcagggggattgaggggggctggcatcccagccggggagacagatgaaaaccaagtaaatcagcagaaaagataatttcactcatgataggagctgtgaggggttagagccaaatagaaatacagcgtgagccacgtgtgaggttttcagtttaaattttctaatagccacttaacagtcaaaggaaacaggtggaattaattttaatcttatttaacccaaatatatgcaaagtattatcacttcaacatgtaatcagtataaacggcatcaatatttttgcagtgtttttgcatgaagtctttgaaatcctgtgtgtacgatacatgtgcagcaggtctcattttggactggccccgtttcaagggctcaccaactgaatgcagcttccagactggagagtgcatgtctggagcaagtggggacaaggacagatggagcttcaggaaggcctctctgaagaggtagatagtgagctttgacatggaggccagggaggcactcagcacacagccacatgaggaatgctgcctggagcggccactgcaaaggccctgtggcagggacaggcaaggcacattggacggtcaggtagggccagggtggctggaggggagtgagggcagggacagaggtaggaggtggtgtctgagagtggacagggcgggccacccagggcctcgtggtgcacagtgagcagtttggaactgattctgggagtgacagaggcattggaggcttttaaagaggggaatcactaggtcagctgctggctggggaatgggccccgggctgggaggggtggaagcctgggcccagcggggaggccccagtgcgtgaggaagtcgatggcttgaacaggatggggcagctgctatcaggagggaacggaagtggggaagagctccagccctgggcttcagacctccagaagcagcaggaagaggggtgataacagtgtccctccttgcttcggggtcatttatttattcattcatgcattcactgcaaggccctttgcacaggtcaggggatgcagatgaccagaacataagatctgcagcaaagatgggcaaggcaggcctttaggcaagttactacagtcacacgaaggacaggagagctgtgatcacagagctcaaggggcagtggagatgggcgagtggctgccatggcaggggaagggatgctgcccactggcaggtcatcaggaaaggctgtttttggatgcatggaccagaaagccactctctgaggtggcccccacctgcatcatcctagggccacaggccgaggccttctgaggggccttggggctgccccgctgctctgctggtcttggctcctctgcagcacctgcttcctttagcactggtgaatgattcttcctcttcccctgtctcagggaaggcttgcctgcttccatgcttgcatgctgggtgacaataaagcattcctggttcttcaccttggctttcagacctgaaacccgacttcctttcaaaattcctgtcctcggggggaaaaggtttccacaatgtgagatggtagcctggacaccagctgtcatcatgttgctgctctagcgtttgagctcccgatgaacaatgaccattgggtccaaccagcccttttgggagcccccttctctgttactgccccagggggagccagaacccagcagggctgtgactccagcgagcaaaatggaactccctccagtttctagactgaattatgtagggaggacagaaggactcagaacccacactcccagaccatgaataccttcccgtggcagagcatcaccataggtgtccacaagtgacgacgcacagccacatgagggggtccctggggactggctgagggactgggccagaggaggccatgacagcctcatccctctgtctccttctgtctgtctccaaattgtccaccagaatggggacagggaaggagggtcaggaagcacagaaggtctaggctgtggctagggtccctttccctgccgcagcccccaaatcagcatcccaccctcaaatccagtaagaatgctacgatcggcagtgtggctccctccctgcaggtttcactggaggccacgtaagtgaaatttatcccattatgtgtgttttgatgtagcaggaccttaccagttttttttgtttttttatttttattttttttggtttcttttttgagatggagtcttgctgtgttgcccaggctggagtgcagtggcacaatcttggctcactgcaacctccacctcccgggttcaagtgggagcctcagcctcagcctcagcctcagcctcccgagcagttgggattacaggcacctgccaccatgcctggctaatttttgtatattgagtagaggcggggtttcaccatgttggccaggctggtcttgaactcctgacctcatgatctgccgccttggcctcccaaagttctgggattacaggtgtgagccactgtgcctggccggccttatcagtttttatgtaatttgtcctgatttaaaaaaaaaaaatcacatgcgggtaaactcaacatattttaatttctctttagagaaattttccacttgagccaggtatggtggctcttgcctgtaatctgagctactcgggaggttgaggtgggaggatcacttgagcccaggagttggaggctgcagtgagccatgatcgcaccattgcactccagcctggacaacagagtgagatcctgtctcaaaaattaaaaaaagcagtaattttttcttctcaagtcattcttttaaaataaatcaaaagtattttaacagaacaatctgttcctcgttgattttaaacagccccccagctaagtccactggttgtcggtctccatggcccatctccccaccttgcttcctcgctggcttaaattttcaagtggacgagcccttttcagcttggcttgtccacctcccaggtgtgtttcctgaagatgcttgtacttatgtctaagagcggcagctccccacatctcagccacctgcaatcgttgagggttgttggactctaaacttatgtgcctttcctgtttcctctttgccttttgcaaattgaagaaccgtgtaaaaccatttttatgtggcttcaacgtcaactataaattagcttggttatcttctaggagaaatgctatttattttggagtagtagtaaaaagggctcaaaggataaggaggccattcaggcctattctgaatccctgatgacatcagctcccaagggctctgtgctgcaggaagcaaaactgtaggtgggtaccaggtaatgccgtgcgcctccccgccccctcccatatcaagtagaatgctggcggcttaaaacatttggggtcctgctcattccttcagcctcaacttcacctggagtgtctacagactgaagatgcatatttgtgtattttgcttttggagaaactgcccttcctatgttctgagtgaatagcagtttttttgatcccagagggcaacttgtattctgtgggctggtgcctatttgcaaggtcacattagaaagacaggagcaaggcttaggcctgctgcttctggaatctcttgtgcaatagactcccctggggaagtctgctagaaatgcagatccctgggcagccttctagccttctggaataggatcatggtgggaccctggaatctgtattttaatggaccctaatgagattctgatgtagaaacatgtttgagaggcattgatctaaatctgggatgactggccaggtgcggtggctcacacctgtaatccagcactttgggaggccgaagcaggtggatcatttgaggtcaggagttcaagaccagcctggccaacatggtgaaaacctgtctctactaaaaatacaaaaaaaattagctgggtgtggtggcacacgcttgtaatcccagctactcaggaggctgaggcaggagaatcgcttgaacccgggagatggaggttgcagtgagccaagatcgtgccactgcactccagcctgggtgacagagtgagactccgtctcaaaaaaataaaactaaaataaataaataaataaatacatctgggatgactgaccaagaacaaagaatgtaggcatcaactgaacaccaactgtatacctgggactggatctgagggtaggattgccagattgagcacaaacaaacaaacgaaacacaagaaacaacagaagggtgcctgttaaatatgaatttctggtaagcaataagtaatttttattgtgttcctgtgcaatgataggacgcacgtatcctgaaaacctgtctgtagttcacctgaaattcacatttaactaggcgttctgattttatgtggccgccctatctgctgggaacataggctgatgcccctgggggttctgcgtttccttggccaggttcctgtagggctgaggtcatgggggagccgtggccagggatggtgtccttgccaggggaggtgacgggcagagctgcaggccctacaacttggggttgggcagggatgagtcgctctaccgtggtgcctctgataatagctgttattttagtttctgaaaaatgtgctgccctcaagagcttctgaggctaggcctacttgggtgaaaatagctttccagggagccacattgtggggacgtgaggcagcagccccactctttggggccacttggctgtctctaactcatctcacttgccagggctggacaaggagatgaaaaagaaaatgccaaggggtgggcaggcaggagcctgttggcgttggcaggcgctgccagccctgtgtcccagtcaagctctggggcgcccttcctgtccatctgggagtagggggtggagtctgaccatgttggggtggagtgttaatattaggagaggatcctccctgggacagggtccccaggcttgggggcaaggccttctgcctggacccccttcctttcctctctcccaggattcctccaggacaccgtagtgctgtgcacggtttggccccatcggtgtgcctgctccttccagccactgctcccttctgcagaaacttgcctgggattcccagggtgcaggggtgggggctggtcaggggctggtcagggcctggtcagtgtagagcagggagcttttcacttttagcatagatagagtggccagggccccatggctgcacctccctggacataaccaggatgtcctgtcatctgccccccaaacaaaactggggtctcccctttattttgccaacagaatccaatttttaggtgatgagacaagtgtttgacttgaaaagaccctcctgccctgcctcctggaaggtctggagcccagcccagcgccccggatcccctaggcatgtgaaacggggaatgcagtgtcctccagtggctggctgcagcttggggtgggcacctgggtggtggctggtggtccccctgcaggccactgctgccccctcttgggtcctgttctgagtctcaggcctgagtccctggggaaggccccacacctttgccctgtacctgggtatggggatggccctgaggggctgccttgcttgggagaaggggggaaaacagatatttttatataaataagaaaaagacagcccctctgggcagatgaggcctgctgtagggcagggggcttggtaaactgtgtgtccactggtctgtaacccttcagctgggtccttttgtgcagtgaacagcctggacaactgtccaggtggcccttcagggatatgttcctggacctttcttagcttctcaccgatctttcttccacgccaagtccatgttcctcagtcaaacaagtggcaaagaggaggaggaggaggggaaggaggaggaacctgcttcaccctctggcacctgcgggttttgttccacgtgtcttccttgggaaagacaaggggttcttttacagcctaggggtggtttcccctttctacttctgagtgagaccttctcaagtcacggctctttgggcccacatggctaaggtctagccacctgtgggtgtcctgtgctgcctcagtgggtggtgtgggggcggggctgacctcccccgcgcctggctcagtgcagcagaccctggcccctgtgcccgctctctgccctgagcaggagtttgattttcctaccccgtggtgaagcaagcacaggtgggcggaggtgggcagcggtttccagccccagccaccggggcagggaggcctctgttgctcagggcaagggaggcagggggcttagtgggacgaaggctcaccccacactcttatcattagcattatctgtatttttaatttttaagttgagaaattttctacaactgtcaaactagacaaatgacagtatccagttcccctcacccagacgcagccattgctgacagctgtcagtgttgaagatggccagcatctcatgcccttggtttcactcgttccctgggtggccctcgtccccctccccacgtcctgcgccccgcataccctgagcagccatggccaggaacttggcacgtgggtttgttttttctgaatctgcatgtgcgcagccatgagcatgaggtcactttttgaatgtgaccgaagctgcatcctgctgcgtgtattgctgtgtggctggctttcctccctgtcttctgttcaggaatactgttcaggctgaataccgtcctgtgacatgggtttaaaaacacaggccaggcatggtgtctcacgcctggaatcccagcgctttgggaggctgaggtgagaggatcgcctgagcccaggagttcgagaccagcctgggcaacatagacctgatctctacaaaaaattaaaaaatgagccgggcgtggtggtggcacgtgtcattgtcccggccacttgggaggccgaggtgggaggatcacttgagcccaggagttcaaggccgtggtgagccatgatggcaccaccgtactccagcctgggcgacagagagagaccgtctctaaaaaataaaaaataaatcaacacataaacatacgttctcctcctgagggacagagcttgccccggttgttccatgttgactgtgctgcacacaggccgccccccttgctctgcccctcactgtattaagtctctttgggcctcagtttctccatctctaaaggaggtcggggaggccaggcgggtgttgagtgagatgaagaggcccttggagtcattccaggagtctataggtgactccagtctcttctcacttctgacagcctggtggcgggggtggagtctctgctggggatgtgggtacagggctgtggtgggcggggatgagggtggggtttggtgacagggcgtgggtggtcacccagctttgcctttctccacagggtcctgaggacaccgtgagccagccaggcctggccgctgggcctgaccggccccccagcccctacaccccgcttctcccggactctcccagcggacagccccccagccccacagcctgagcctcccagctgccatgtgcctgttgcacacctgcacacacgccctggcacacatacacacatgcgtgcaggcttgtgcagacactcagggatggagctgctgctgaagggacttgtagggagaggctcgtcaacaagcactgttctggaaccttctctccacgtggcccacaggcctgaccacaggggctgtgggtcctgcgtccccttcctcgggtgagcctggcctgtcccgttcagccgttgggcccaggcttcctcccctccaaggtgaaacactgcagtcccggtgtggtggctccccatgcaggacgggccaggctgggagtgccgccttcctgtgccaaattcagtggggactcagtgcccaggccctggccacgagctttggccttggtctacctgccaggccaggcaaagcgcctttacacaggcctcggaaaacaatggagtgagcacaagatgccctgtgcagctgcccgagggtctccgcccaccccggccggactttgatccccccgaagtcttcacaggcactgcatcgggttgtctggcgcccttttcctccagcctaaactgacatcatcctatggactgagccggccactctctggccgaagtggccgcaggctgtgcccccgagctgcccccaccccctcacagggtccctcagattataggtgcccaggctgaggtgaagaggcctgggggccctgccttccgggcgctcctggaccctggggcaaacctgtgacccttttctactggaatagaaatgagttttatcatctttgaaaaataattcactcttgaagtaataaacgtttaaaaaaatgggatgcctgcctctgtgacagccttgtttgctgaggtcgtgggggtgggggcctctgggaagttccgggctcctcttctcttggtcaatagctcctttctggtggctgccaagagcgtctctcccagggccgggctgctggcttaccttcctgttgttttcaaatttcaaccttgtgcaatgttgagtttcatagaaatactgcatgagtacgcccttgtttagaagcagcagggtctgagtcccatcccacagcccccagtgcagacgcttttgccacttttgcatggggccccctggatgtgtttctgtgcatttatctacaaatcctggtgcccataggacatgccccgtgttgttctaggcctttgctttctgctcgttacataaatggtgaagaagagaagcgggtaagagaacagattggagccatctaaaagttctcatcttaagtgtagatcattgcaaaggatggaattttctccgattgtcatcatcgttgatgtttgaatatgagaccatttcatcagtatttaagtgtgcccgctgggatctaactaggagaggagagggtattcactgccgaacatttcaaagaatatacgaacaagctcttttatggtcagaaatgttagcccttttccccttaaacttgtatttcctttcttcttccctcctaaaattttatctaaatgatatttatatttgatgcttttaagccttttattaatcactcctatacttgcctgcaacataaatatataagttgaagcaaactttttttttttatttcttgtgaccatcaagggtaaatttttttaggtctggggctgccataacaaaataccatcaaggccgggcgtggtggcacacgcctgtaatcccagccctgtgggaggccaagaccggaggattgcttgatgccaggagttcaagactagtctgggtaatatggtgagaccctcatcaatacaaaaaaaatacaaaaattagccaggcgtggtggtgggcgcctgtggtcccaactactcaggaggctgaggtaggattgctggaagccaagaggttgaggctgcagtgagctgtgatcatgccactgcactccagcctgcgagacagagtgagacactgtctcaaaaagaaaaaataccatcaataggtggcttaaaccatttatttctcacagctctggaggctggaagtctgagaccagggttccagcagggttgggttctggtgaggcgtctcttcctggcttcagacggccgccttctctgtgtcctcacatggcctttcctcggtgtgaaagggggaagagagagggagagcgagagctctgtggtgtctcttataaggacactgatgctatcagatcagggcctcatttaattattttcttagaggccccatctccagacacagccacactggaggttagggcttcaacaggaattttgggtcaacacaaacatttagcctataccatctggattgcctttcaagtctcaggacacaattggtcaaaactatattactatgttttatgtgacatatcaaagtagcaaaattttattggaatgcatgtccttttggtatgcattttatatatctttttcatttacagattagagttgtgtgtgagtgtgtgtacaattccatgaattttgacatgtttatagagttgagtcattacccctacaatcaagacaaaacaatttcatcatcccccaagatttcctcatgtcttccctttgtttttgtttgttttacagtggg

SEQ ID NO: 2 - RNA, 129 nt, Homo sapiens
  source 1..129: mol_type = other RNA
gagaaatgctatttattttggagtagtagtaaaaagggctcaaaggataaggaggccattcaggcctattctgaatccctgatgacatcagctcccaagggctctgtgctgcaggaagcaaaactgtag

SEQ ID NO: 3 - RNA, 20 nt, synthetic construct
  source 1..20: mol_type = other RNA
aaataattgaattttaaata

SEQ ID NO: 4 - RNA, 21 nt, synthetic construct
  source 1..21: mol_type = other RNA
ggcctgaatggcctccttatc

SEQ ID NO: 5 - RNA, 20 nt, synthetic construct
  source 1..20: mol_type = other RNA
ggtacccacctacagttttg

SEQ ID NO: 6 - RNA, 20 nt, synthetic construct
  source 1..20: mol_type = other RNA
caaaataaatagcatttctc

SEQ ID NO: 7 - RNA, 19 nt, synthetic construct
  source 1..19: mol_type = other RNA
gtggcaacgcggcatgcaa

SEQ ID NO: 8 - RNA, 20 nt, synthetic construct
  source 1..20: mol_type = other RNA
agcatttctcctagaagata

SEQ ID NO: 9 - RNA, 20 nt, synthetic construct
  source 1..20: mol_type = other RNA
ttgagccctttttactacta

SEQ ID NO: 10 - RNA, 20 nt, synthetic construct
  source 1..20: mol_type = other RNA
gatgtcatcagggattcaga

SEQ ID NO: 11 - RNA, 20 nt, synthetic construct
  source 1..20: mol_type = other RNA
gcacagagcccttgggagct

SEQ ID NO: 12 - RNA, 20 nt, synthetic construct
  source 1..20: mol_type = other RNA
acctacagttttgcttcctg

SEQ ID NO: 13 - RNA, 21 nt, synthetic construct
  source 1..21: mol_type = other RNA
acataagtacaagcatcttca

SEQ ID NO: 14 - RNA, 20 nt, synthetic construct
  source 1..20: mol_type = other RNA
ggttttacacggttcttcaa

SEQ ID NO: 15 - RNA, 20 nt, synthetic construct
  source 1..20: mol_type = other RNA
gctgaaggaatgagcaggac

SEQ ID NO: 16 - RNA, 21 nt, synthetic construct
  source 1..21: mol_type = other RNA
gcatcttcagtctgtagacac

SEQ ID NO: 17 - RNA, 20 nt, synthetic construct
  source 1..20: mol_type = other RNA
ctgtctttctaatgtgacct

SEQ ID NO: 18 - RNA, 20 nt, synthetic construct
  source 1..20: mol_type = other RNA
cttgggagctgatgtcatca

SEQ ID NO: 19 - RNA, 25 nt, synthetic construct
  source 1..25: mol_type = other RNA
acagttttgcttcctgcagcacaga

SEQ ID NO: 20 - RNA, 15 nt, synthetic construct
  source 1..15: mol_type = other RNA
ccacctacagttttg

SEQ ID NO: 21 - RNA, 25 nt, synthetic construct
  source 1..25: mol_type = other RNA
agcccttgggagctgatgtcatcag

SEQ ID NO: 22 - DNA, 28 bp, synthetic construct
  source 1..28: mol_type = other DNA
ttactgaccgtacaccaaatttgcctgc

SEQ ID NO: 23 - DNA, 30 bp, synthetic construct
  source 1..30: mol_type = other DNA
cctggcagcgatcgctattttccatgagtg

SEQ ID NO: 24 - DNA, 22 bp, synthetic construct
  source 1..22: mol_type = other DNA
aacaagaaaggcctcacttcag

SEQ ID NO: 25 - DNA, 25 bp, synthetic construct
  source 1..25: mol_type = other DNA
ggtgttctctgacttatacatgcac

SEQ ID NO: 26 - DNA, 21 bp, synthetic construct
  source 1..21: mol_type = other DNA
nnnagatcggaagagcacacg

SEQ ID NO: 27 - DNA, 15 bp, synthetic construct
  source 1..15: mol_type = other DNA
cgtgtgctcttccga

SEQ ID NO: 28 - DNA, 27 bp, synthetic construct
  source 1..27: mol_type = other DNA
annnnnnnagatcggaagagcgtcgtg

SEQ ID NO: 29 - RNA, 18 nt, synthetic construct
  source 1..18: mol_type = other RNA
  modified_base 1..18: mod_base = OTHER; note = 2[prime]-O-methyl nucleotides and phosphorothioate internucleotide linkages
gcctacctaggttgttta

SEQ ID NO: 30 - RNA, 20 nt, synthetic construct
  source 1..20: mol_type = other RNA
  modified_base 1..20: mod_base = OTHER; note = 2[prime]-O-methyl nucleotides and phosphorothioate internucleotide linkages
accaattcaccttccaatat

SEQ ID NO: 31 - RNA, 20 nt, synthetic construct
  source 1..20: mol_type = other RNA
  modified_base 1..20: mod_base = OTHER; note = 2[prime]-O-methyl nucleotides and phosphorothioate internucleotide linkages
gccaccttacccggatgtat

SEQ ID NO: 32 - RNA, 20 nt, synthetic construct
  source 1..20: mol_type = other RNA
  modified_base 1..20: mod_base = OTHER; note = 2[prime]-O-methyl nucleotides and phosphorothioate internucleotide linkages
ttaaacttacttttccaaag

SEQ ID NO: 33 - RNA, 22 nt, synthetic construct
  source 1..22: mol_type = other RNA
  modified_base 1..22: mod_base = OTHER; note = 2[prime]-O-methyl nucleotides and phosphorothioate internucleotide linkages
acgcacctatagcttggcttct

SEQ ID NO: 34 - RNA, 20 nt, synthetic construct
  source 1..20: mol_type = other RNA
  modified_base 1..20: mod_base = OTHER; note = 2[prime]-O-methyl nucleotides and phosphorothioate internucleotide linkages
tactctggtccttaaggaaa

SEQ ID NO: 35 - RNA, 20 nt, synthetic construct
  source 1..20: mol_type = other RNA
  modified_base 1..20: mod_base = OTHER; note = 2[prime]-O-methyl nucleotides and phosphorothioate internucleotide linkages
aaataattgaattttaaata

SEQ ID NO: 36 - RNA, 20 nt, synthetic construct
  source 1..20: mol_type = other RNA
  modified_base 1..20: mod_base = OTHER; note = 2[prime]-O-methoxyethyl nucleotides and phosphorothioate internucleotide linkages
aaataattgaattttaaata

SEQ ID NO: 37 - RNA, 21 nt, synthetic construct
  source 1..21: mol_type = other RNA
  modified_base 1..21: mod_base = OTHER; note = 2[prime]-O-methyl nucleotides and phosphorothioate internucleotide linkages
ggcctgaatggcctccttatc

SEQ ID NO: 38 - RNA, 20 nt, synthetic construct
  source 1..20: mol_type = other RNA
  modified_base 1..20: mod_base = OTHER; note = 2[prime]-O-methyl nucleotides and phosphorothioate internucleotide linkages
ggtacccacctacagttttg

SEQ ID NO: 39 - RNA, 20 nt, synthetic construct
  source 1..20: mol_type = other RNA
  modified_base 1..20: mod_base = OTHER; note = 2[prime]-O-methyl nucleotides and phosphorothioate internucleotide linkages
caaaataaatagcatttctc

SEQ ID NO: 40 - RNA, 19 nt, synthetic construct
  source 1..19: mol_type = other RNA
  modified_base 1..19: mod_base = OTHER; note = 2[prime]-O-methyl nucleotides and phosphorothioate internucleotide linkages
gtggcaacgcggcatgcaa

SEQ ID NO: 41 - RNA, 20 nt, synthetic construct
  source 1..20: mol_type = other RNA
  modified_base 1..20: mod_base = OTHER; note = 2[prime]-O-methyl nucleotides and phosphorothioate internucleotide linkages
agcatttctcctagaagata

SEQ ID NO: 42 - RNA, 20 nt, synthetic construct
  source 1..20: mol_type = other RNA
  modified_base 1..20: mod_base = OTHER; note = 2[prime]-O-methyl nucleotides and phosphorothioate internucleotide linkages
ttgagccctttttactacta

SEQ ID NO: 43 - RNA, 20 nt, synthetic construct
  source 1..20: mol_type = other RNA
  modified_base 1..20: mod_base = OTHER; note = 2[prime]-O-methyl nucleotides and phosphorothioate internucleotide linkages
gatgtcatcagggattcaga

SEQ ID NO: 44 - RNA, 20 nt, synthetic construct
  source 1..20: mol_type = other RNA
  modified_base 1..20: mod_base = OTHER; note = 2[prime]-O-methyl nucleotides and phosphorothioate internucleotide linkages
gcacagagcccttgggagct

SEQ ID NO: 45 - RNA, 20 nt, synthetic construct
  source 1..20: mol_type = other RNA
  modified_base 1..20: mod_base = OTHER; note = 2[prime]-O-methyl nucleotides and phosphorothioate internucleotide linkages
acctacagttttgcttcctg

SEQ ID NO: 46 - RNA, 21 nt, synthetic construct
  source 1..21: mol_type = other RNA
  modified_base 1..21: mod_base = OTHER; note = 2[prime]-O-methyl nucleotides and phosphorothioate internucleotide linkages
acataagtacaagcatcttca

SEQ ID NO: 47 - RNA, 20 nt, synthetic construct
  source 1..20: mol_type = other RNA
  modified_base 1..20: mod_base = OTHER; note = 2[prime]-O-methyl nucleotides and phosphorothioate internucleotide linkages
ggttttacacggttcttcaa

SEQ ID NO: 48 - RNA, 20 nt, synthetic construct
  source 1..20: mol_type = other RNA
  modified_base 1..20: mod_base = OTHER; note = 2[prime]-O-methyl nucleotides and phosphorothioate internucleotide linkages
gctgaaggaatgagcaggac

SEQ ID NO: 49 - RNA, 21 nt, synthetic construct
  source 1..21: mol_type = other RNA
  modified_base 1..21: mod_base = OTHER; note = 2[prime]-O-methyl nucleotides and phosphorothioate internucleotide linkages
gcatcttcagtctgtagacac

SEQ ID NO: 50 - RNA, 20 nt, synthetic construct
  source 1..20: mol_type = other RNA
  modified_base 1..20: mod_base = OTHER; note = 2[prime]-O-methyl nucleotides and phosphorothioate internucleotide linkages
ctgtctttctaatgtgacct

SEQ ID NO: 51 - RNA, 20 nt, synthetic construct
  source 1..20: mol_type = other RNA
  modified_base 1..20: mod_base = OTHER; note = 2[prime]-O-methyl nucleotides and phosphorothioate internucleotide linkages
cttgggagctgatgtcatca

SEQ ID NO: 52 - RNA, 25 nt, synthetic construct
  source 1..25: mol_type = other RNA
  modified_base 1..25: mod_base = OTHER; note = 2[prime]-O-methyl nucleotides and phosphorothioate internucleotide linkages
acagttttgcttcctgcagcacaga

SEQ ID NO: 53 - RNA, 15 nt, synthetic construct
  source 1..15: mol_type = other RNA
  modified_base 1..15: mod_base = OTHER; note = 2[prime]-O-methyl nucleotides and phosphorothioate internucleotide linkages
ccacctacagttttg

SEQ ID NO: 54 - RNA, 20 nt, synthetic construct
  source 1..20: mol_type = other RNA
  modified_base 1..20: mod_base = OTHER; note = 2[prime]-O-methoxyethyl nucleotides and phosphorothioate internucleotide linkages
ggtacccacctacagttttg

SEQ ID NO: 55 - RNA, 18 nt, synthetic construct
  source 1..18: mol_type = other RNA
atgctgtgcatgtcgtaa

(19) United States
(12) Patent Application Publication
(10) Pub. No.: US 2026/0085322 A1
(43) Pub. Date: Mar. 26, 2026

(54) THERAPEUTIC AGENTS FOR METABOLISM-ASSOCIATED DISEASES

(71) Applicant: United Kingdom Research and Innovation, Swindon (GB)

(72) Inventors: Santiago VERNIA, Swindon, Wiltshire (GB); Helen PATERSON, Swindon, Wiltshire (GB); Sijia YU, Swindon, Wiltshire (GB); Michael MANOLAKAKIS, Swindon, Wiltshire (GB); Andrew JOBBINS, Swindon, Wiltshire (GB)

(21) Appl. No.: 19/109,304

(22) PCT Filed: Sep. 6, 2023
(86) PCT No.: PCT/EP2023/074396

§ 371 Date: Mar. 6, 2025

(30) Foreign Application Priority Data
Sep. 7, 2022 (EP) 22386061.0

(51) Int. Cl. C12N 15/113; A61P 3/06

(52) U.S. Cl. CPC C12N 15/1138; A61P 3/06; C12N 2310/11; C12N 2310/315; C12N 2310/321; C12N 2320/33

(57) ABSTRACT

Provided herein is a target for the treatment of metabolism-associated diseases, such as metabolism-associated fatty liver disease (MAFLD). Also provided are agents for the treatment of said diseases and methods of using said agents.

### FIELD OF THE INVENTION

Provided herein is a target for the treatment of metabolism-associated diseases, such as metabolism-associated fatty liver disease (MAFLD). Also provided are agents for the treatment of said diseases and methods of using said agents.

### BACKGROUND OF THE INVENTION

In mammals 95% of multi-exon genes undergo one or more alternative splicing (AS) events, generating multiple isoforms^{1, 2}, contributing to transcript diversity and complexity³. AS has been proposed to be critical for the establishment and maintenance of tissue-specific protein interaction networks⁴⁻⁶ and tissue identity⁶. In contrast, whether specific AS programs regulate physiological adaptations is less well characterised, and their role in metabolic disease is unclear. This is because of poor functional characterisation of specific isoforms and the technical challenges involved in identifying specific upstream regulatory splicing factors in vivo. As a consequence, there is limited information about networks of splicing involved in metabolic reprograming, at the level of both the splicing factors and the isoforms they regulate. As well as increasing our knowledge of metabolic regulation in health and disease, characterisation of splicing factors and isoforms involved in metabolic regulation could lead to the development of RNA-based therapeutics to enhance or antagonise specific isoforms. RNA-based therapeutics to inactivate specific genes (e.g. APOB or PCSK9) in the liver are emerging as promising therapeutic strategies for metabolic pathologies^{7, 8}. However, the development of splice-switching RNA therapeutics has yet to be explored.

In parallel with the global increase in obesity, metabolism-associated fatty liver disease (MAFLD) has become the most prevalent non-communicable liver pathology, affecting up to 25% of the population worldwide⁹. MAFLD ranges from pre-symptomatic hepatic steatosis (fatty liver) to non-alcoholic steatohepatitis (NASH), which is characterised by additional inflammation, hepatocellular injury and fibrosis, and that may progress to liver failure, cirrhosis and hepatocellular carcinoma (HCC)¹⁰.

While the exact mechanisms promoting the progression from steatosis to NASH are not fully understood, evidence suggests that chronic overnutrition and hypercaloric western-style diets play a role. This is in part due to dysregulation of bioactive and/or toxic lipid species such as phospholipids, saturated fatty acids, sphingomyelins, ceramides and cholesterol¹⁻¹³. In addition, there is evidence that MAFLD contributes to the pathogenesis of type 2 diabetes and cardiovascular disease, with coronary artery disease being the top cause of death in these patients.^{14, 15} Given MAFLD has unmet clinical needs, there is interest in understanding the molecular mechanisms linking MAFLD with increased cardiovascular risk and progressive liver damage, in order to identify novel therapeutic targets.

### SUMMARY OF THE INVENTION

In an aspect, there is provided an agent capable of inducing skipping of exon 12 of scavenger receptor class B type I (Scarb1). The agent may bind to the pre-mRNA of Scarb1. For instance, the agent may bind to a site within Scarb1 pre-mRNA that affects the splicing of exon 12. The agent may be a nucleic acid analogue or a nucleic acid. The agent may be an antisense oligomer.

In an aspect, there is provided an antisense oligomer capable of inducing skipping of exon 12 of scavenger receptor class B type I (SCARB1). The antisense oligomer may be capable of inducing skipping of exon 12 of human SCARB1. The antisense oligomer may be 10-45 nucleobases, 12-40 nucleobases, 15-35 nucleobases, 18-30 nucleobases, or 19-25 nucleobases in length.

The antisense oligomer may comprise a nucleobase sequence according to any one of SEQ ID NOs: 4, 5, 7, 8, 9, 10, 11, 12, 18, or 19, and may comprise 5, 4, 3, 2, 1, or fewer substitutions, deletions, or insertions. The antisense oligomer may comprise a nucleobase sequence according to any one of SEQ ID NOs: 4, 5, 7, 8, 9, 10, 11, 12, 18, or 19, and one or more nucleobase substituted for a modified nucleobase that is capable of base pairing with the same type of nucleobase. The antisense oligomer may comprise a nucleobase sequence according to any one of SEQ ID NOs: 4, 5, 7, 8, 9, 10, 11, 12, 18, or 19. The antisense oligomer may comprise a nucleobase sequence according to any one of SEQ ID NOs: 5, 8, 10, 11, 18, or 19. The nucleobase sequence of the antisense oligomer may be according to any one of SEQ ID NOs: 4, 5, 7, 8, 9, 10, 11, 12, 18, or 19.

The antisense oligomer may comprise at least one 2′-O-methyl nucleotide and/or at least one 2′-O-methoxyethyl nucleotide. The antisense oligomer may be a 2′-O-methyl nucleic acid oligomer and/or a 2′-O-methoxyethyl nucleic acid oligomer. The antisense oligomer may comprise at least one phosphorothioate internucleotide linkage. In some embodiments, all internucleotide linkages in the antisense oligomer are phosphorothioate internucleotide linkages.

In an embodiment, the antisense oligomer is an oligonucleotide and has a nucleobase sequence according to SEQ ID NO: 5, 8, 10, 11, 18, or 19, and wherein —O—CH₃ or —O—CH₂—CH₂—O—CH₃ is attached to the 2′ position of the sugar moiety of each nucleotide.

Provided herein is an antisense oligonucleotide with a sequence according to any one of SEQ ID NOs: 4, 5, 7, 8, 9, 10, 11, 12, 18, or 19, wherein the internucleotide linkages are phosphorothioate internucleotide linkages, and wherein —O—CH₃ or —O—CH₂—CH₂—O—CH₃ is attached to the 2′ position of the sugar moiety of each nucleotide.

Provided herein is a pharmaceutical composition comprising any antisense oligomer or antisense oligonucleotide as disclosed herein.

Provided herein is any antisense oligomer, antisense oligonucleotide, or pharmaceutical composition as disclosed herein, for use as a medicament. Provided herein is any antisense oligomer, antisense oligonucleotide, or pharmaceutical composition as disclosed herein, for use in a method of treating or preventing a metabolism-associated disease. Provided herein is any antisense oligomer, antisense oligonucleotide, or pharmaceutical composition as disclosed herein, for use in a method of treating or preventing a pathological effect of obesity and/or an obesogenic diet. The pathological effect may be liver inflammation, lipotoxicity, hepatocellular injury, and/or fibrosis. Provided herein is any antisense oligomer, antisense oligonucleotide, or pharmaceutical composition as disclosed herein, for use in a method of treating or preventing liver inflammation. The liver inflammation may be obesity-induced. Provided herein is any antisense oligomer, antisense oligonucleotide, or pharmaceutical composition as disclosed herein, for use in a method of treating or preventing metabolism-associated fatty liver disease (MAFLD). Provided herein is any antisense oligomer, antisense oligonucleotide, or pharmaceutical composition as disclosed herein, for use in a method of treating or preventing pre-symptomatic hepatic steatosis. Provided herein is any antisense oligomer, antisense oligonucleotide, or pharmaceutical composition as disclosed herein, for use in a method of treating or preventing non-alcoholic steatohepatitis (NASH). Provided herein is any antisense oligomer, antisense oligonucleotide, or pharmaceutical composition as disclosed herein, for use in a method of treating or preventing hepatocellular carcinoma (HCC) in a subject. The subject may have liver inflammation, pre-symptomatic hepatic steatosis, or NASH. Provided herein is any antisense oligomer, antisense oligonucleotide, or pharmaceutical composition as disclosed herein, for use in treating or preventing any one of, or a combination of, gallstone disease, type 2 diabetes, cardiovascular disease, and coronary artery disease.

Provided herein is any antisense oligomer, antisense oligonucleotide, or pharmaceutical composition as disclosed herein, for use in a method of treatment, wherein the method comprises lowering cholesterol levels in a subject in need thereof.

Provided herein is any method of increasing expression of scavenger receptor class B type I (Scarb1) isoform SR-BII relative to the isoform SR-BI in a cell, the method comprising contacting the cell with a composition comprising the antisense oligomer or antisense oligonucleotide as disclosed herein. The method may be in vivo and the composition may be administered to a subject in need thereof.

### BRIEF DESCRIPTION OF THE DRAWINGS

FIG. 1. Expression of pre-mRNA splicing machinery is regulated by nutritional inputs in the liver. A.—Schematic representation of experimental design. Livers from mice fed a HFD or a CD were harvested in fed (ad libitum) or fasted (16 h) state and processed for either high-throughput TMT/MS (isobaric mass tagging) proteomics (n=3) or RNAseq analysis (n=4). B.—Principal component analysis for TMT/MS (upper) and RNAseq (lower) analyses. C.—Gene ontology analysis of proteins differentially expressed between fasted vs. fed state (upper) and between HFD vs. CD livers (lower). D.—Analysis of differential AS events between CD fast vs. fed (black), HFD vs. CD (blue) and HFr vs. CD (red). Percentage of events changing within each comparison is represented by pie chart (right) (SE: Skipped Exon, MXE: Mutually Exclusive Exon, RI: Retained Intron, A5SS: Alternative 5′ Splice Site, A3SS: Alternative 3′ Splice Site). E-G.—Enrichment of eCLIP crosslinking surrounding conserved AS events, differentially regulated in each comparison, in HepG2 cells from ENCODE database. H.—Venn diagram showing the splicing factors differentially expressed in HFD vs. CD from RNAseq (yellow) and TMT/MS analyses (blue) and the overlap of splicing factors detected in primary hepatocytes (green).

FIG. 2. RBFOX2 is a splicing factor expressed in the liver. A.—Liver single-cell analysis of Rbfox2 expression. B.—Western blot of L^{WT} and L^{ΔRbfox2} liver lysates showing RBFOX2 expression in hepatocytes. C.—Western blot of C57BL6 liver lysates showing RBFOX2 expression in mice fed a CD, a HFD or a HFr diet (n=6 per condition, image shows 3 representative samples). Right: quantification of long/short RBFOX2 variants. D.—Expression of RBFOX2 containing full-length RRM motif as quantified by TMT/MS (n=3). Bar graphs are represented as mean±SEM. Statistical significance was determined by one-way ANOVA (C) or two-sided t-test (D) of biologically independent samples (*p-value<0.05; ***p-value<0.001).

FIG. 3. RBFOX2 controls AS in a cluster of lipid-regulatory genes in the liver. A.—Cartoon depicting the experimental strategy to identify RBFOX2-regulated AS programmes and isoforms for RNA therapeutics (the sequence depicted is SEQ ID NO: 55). B.—RBFOX2 motif enrichment relative to eiCLIP-RBFOX2 cross-linking positions in mouse hepatocytes (n=3). C.—RNA-maps showing normalised density of RBFOX2 eiCLIP crosslink sites relative to 5′SS and 3′SS of selected exons identified by RNAseq in liver from L^{WT} and L^{ΔRbfox2} mice. D.—GO Molecular function analysis of RBFOX2 cross-linked genes in mouse liver visualised with REVIGO. Bubble size corresponds to number of combined GO terms. Colour corresponds to combined score. E.—GWAS of human genes crosslinked by RBFOX2 in hepatocytes. F.—Schematic representation of Scarb1 gene showing RBFOX2 eiCLIP peak location surrounding exon 12 (arrow). Semi-quantitative PCR plus capillary electrophoresis analysis of Scarb1 exon 12 in liver and quantification of AS in L^{WT} and L^{ΔRbfox2} mice (bottom). G.—Analysis of RBFOX2 regulation of Pla2g6 exon 10, (H) Numb exon 3 and exon 9 and (I) Osbpl9 exon 6. PSI values are represented as mean±SEM (n=6-8). Statistical significance was determined by two-sided t-test of biologically independent samples.

FIG. 4. RBFOX2 controls lipid homeostasis in the liver. A.—Serum lipid analysis showing total cholesterol levels and (B.—) triglycerides in L^{WT} and L^{ΔRbfox2} mice fed a CD and a HFr diet. C.—PCA plot of liver lipid profiles of L^{WT} and L^{ΔRbfox2} mice fed a CD and a HFr determined by LC-MS. D.—LC-MS lipidomic analysis showing total levels of indicated species normalised to tissue mass and PUFA/non-PUFA TG ratio (n=8). E.—Cartoon depicting strategy for iPSC-derived human hepatocytes analysis. F.—RT-qPCR analysis showing knock-down of RBFOX2 in human hepatocytes (n=6). G.—Analysis of RBFOX2-mediated regulation of NUMB exon 3, (H) NUMB exon 9, (I) SCARB1 exon 12 (J) SEC31A exon 21 and (K) OSBPL9 exon 6. PSI values are represented as mean±SEM (n=6). (L) Lipidomics quantification of Cholesteryl ester and (M) sphingomyelin accumulation upon RBFOX2 knock down in human hepatocytes (n=5). Statistical significance was determined by two-sided t-test of biologically independent samples (*p-value<0.05; **p-value<0.01).

FIG. 5. Viral-mediated overexpression of RBFOX2-A6 and RBFOX2 WT in the liver. A.—Cartoon depicting the AAV backbones used to overexpress RBFOX2-A6 (with a truncated RNA binding motif RRM) or control GFP in the liver (top) and representative western blot showing expression levels in the liver. B.—RT-qPCR expression analysis of codon-optimised RBFOX2-A6 in the liver. C.—Quantification of percentage splice in (PSI) for Numb (exon 3), Osbpl9 (exon 6), Scarb1 (exon 12) and Sec31a (exon 21). D.—Cartoon depicting the adenoviral backbones used to overexpress RBFOX2 wild type or control GFP (top) and representative western blot showing expression levels in hepatocytes. E.—Capillary electrophoresis and quantification of percentage splice in (PSI) for Numb (exon 3), Osbpl9 (exon 6), Scarb1 (exon 12) and Sec31a (exon 21) after RBFOX2 overexpression in hepatocytes (n=6). F.—LC-MS lipidomic analysis showing total levels of free cholesterol, cholesteryl ester, sphingomyelin and ceramide normalised to liver tissue mass and PUFA/non-PUFA TG ratio in mice fed a HFr diet after transduction with pAd-RBFOX2 or pAd-GFP control (n=8-9). G.—Cholesterol levels quantified in the bile of mice fed a HFr diet after transduction with pAd-RBFOX2 or pAd-GFP control (n=8-9). H.—Serum lipid analysis showing total cholesterol, HDL-cholesterol and triglycerides in mice fed a HFr diet after transduction with pAd-RBFOX2 or pAd-GFP control (n=8-9). Results are represented as mean±SEM. Statistical significance was determined by two-sided t-test or Mann-Whitney test of biologically independent samples (***p<0.001).

FIG. 6. Transcriptional regulation of Rbfox2 in the liver. A.—CAGE-detected transcriptional start site (TSS) signal at the promoters of RBFOX2 transcripts in hepatocytes, aortic smooth muscle and hippocampus. Two transcript isoforms are shown with their respective promoters. Tag clusters are magnified and ChIP-seq signal for FOXA1, FOXA2, H3K4me3 and H3K27ac are shown. B.—RT-qPCR of Foxa1, Foxa2 and Rbfox2 in Hepa1-6 cells expressing a scramble shRNA (shC) or shRNA against Foxa1/2 (shF1/2) (n=6). C.—Microarray analysis of HepG2 cells with adenoviral FOXA1 overexpression (GSE30447)36. Results are represented as mean±SEM. Statistical significance was determined by two-sided t-test of biologically independent samples (***p<0.001).

FIG. 7. Scarb1 mediates lipidomic changes associated with RBFOX2-deficiency in hepatocytes and can be regulated with splice-switching oligos. A.—Mice fed a HFr diet were subcutaneously injected with SSO8.3, scramble (Scr) or saline during four consecutive weeks (top). No significant effect of injections on body weight was detected (bottom). B.—Semi-quantitative PCR analysis of Scarb1 exon 12 inclusion in liver (top) and quantification of overall AS caused represented as PSI (bottom). C.—Immunohistochemistry showing decreased macrophage (anti-MRC1) infiltration in the liver of SSO8.3-treated mice. Nuclei were stained with DAPI. D.—qPCR analysis in the liver of SSO8.3-treated mice (n=7-9). E-F.—LC-MS lipidomic analysis showing total levels of free cholesterol and sphingomyelin normalised to the tissue weight in mice fed a HFr diet upon SSO8.3 injection. G.—Quantification of cholesterol and phospholipids in the bile of SSO8.3- and Scr-treated mice. H.—Blood analysis of SSO8.3- vs Scr-treated mice showing total cholesterol and triglycerides I.—Analysis of blood VLDL, LDL and HDL lipoprotein composition. Samples were pooled in three replicates. J.—Cartoon depicting the analysis of Dil-HDL uptake in AML12 hepatocytes expressing codon-optimised SR-BI or SR-BII after targeted inactivation of endogenous Scarb1 with a specific siRNA or scramble control (left). Uptake as quantified as Dil-positive cells after 4 h incubation with 0.1 μg/ml Dil-HDL (n=6). K.—Quantification of major lipid species in HDL lipoproteins purified from L^{ΔRbfox2} vs L^{WT} mice fed a HFr diet and treated with SSO8.3 or Scr control. L.—Cholesterol levels quantified in the bile of L^{ΔRbfox2} vs L^{WT} mice treated with SSO8.3 or Scr control as indicated. Results are represented as mean±SEM (n=7-10); Statistical significance was determined by two-sided t-test of biologically independent samples (*p-value<0.05; **p-value<0.01, ***p-value<0.001).

FIG. 8. Splicing factors differentially expressed in specific metabolic conditions in the liver. A.—Volcano plot showing splicing factor (gold) expression in liver of mice in fed or fasted state as determined by TMT/MS analysis or B.—RNAseq analysis. C.—Splicing factor expression in mice fed a HFD vs CD as determined by TMT/MS analysis and D.—RNAseq analysis. E.—Overlap between AS events differentially regulated in feeding/fasting cycles in mice fed a CD or a HFD. F.—Splicing factor motif enrichment within and around cassette exons alternatively spliced in the liver of mice in fasted vs fed state and G.—in HFD vs. CD. Enrichment in non-AS exons was used as a background (dot lines).

FIG. 9. Single-cell RNAseq analysis of RNA binding protein gene expression in the liver. Liver single-cell RNAseq analysis of the expression of relevant AS factors.

FIG. 10. Analysis of RBFOX2-mediated AS regulation in the liver. A.—NuPAGE gel visualising protein-RNA complexes obtained in eiCLIP in hepatocytes. Lanes 1: No antibody, 2: No UV, 3: 0.2 U/ml RNase, 4: 0.1 U/ml RNase and 5: size-matched input. Region from which protein-RNA complexes were cut from the membrane is marked by a dashed box. B.—eiCLIP analysis confirms RBFOX2 crosslink to Ptbp2 and C.—Snrnp70 pre-mRNA transcripts. D.—Cartoon depicting positional effect on RBFOX2 regulation of AS. E.—Bubble-plot representing significant AS events between L^{ΔRbfox2} and L^{WT} livers. Significant events are represented by pie chart (right) as a percentage of total events. Selected transcripts with eiCLIP RBFOX2-crosslinking peaks are indicated. F.—eiCLIP track showing RBFOX2 crosslink (top) and semi-quantitative PCR showing that RBFOX2 promotes Sec31a exon 24 skipping. PSI values are represented as mean±SEM (n=6-8). Statistical significance was determined by two-sided t-test of biologically independent samples. G.—RBFOX2 crosslink in human hepatocyte samples to PLA2G6, H.—SEC31A, I.—NUMB and J.—SCARB1 pre-mRNA transcripts.

FIG. 11. RBFOX2 is involved in the regulation of cholesterol homeostasis. A.—Bodyweight gain over time of L^{WT} and L^{ΔRbfox2} animals fed a CD, (B.—) a HFD and (C.—) a HFr diet. D.—Glucose tolerance test of L^{WT} and L^{ΔRbfox2} fed a CD, (E.—) a HFD and (F.—) a HFr diet. G.—Blood analysis of L^{ΔRbfox2} vs. L^{WT} female mice showing cholesterol levels, and H.—triglycerides. I.—H&E staining of liver from L^{ΔRbfox2} vs. L^{WT} mice fed a HFr diet. Scale bar 50 μm. J-M.—LC-MS/MS analysis of the specified lipids in the liver of L^{ΔRbfox2} vs. L^{WT} mice fed a HFD. N.—Blood cholesterol and triglyceride levels in L^{ΔRbfox2} vs. L^{WT} mice fed a HFD. Results are represented as mean±SEM (n=8-9). Statistical significance was determined by two-way ANOVA (A-F) or two-sided t-test (G-N) of biologically independent samples (*pvalue<0.05; **p-value<0.01).

FIG. 12. RBFOX2 regulates lipid metabolism in human hepatocytes. A.—RT-qPCR analysis of ASGPR2, SERPINA1 and SERPINA2 in human hepatocytes upon RBFOX2 targeted knockdown (n=6) B.—PCA plot of lipidomic analysis of human hepatocytes upon RBFOX2 targeted knockdown as determined by LC-MS. (n=5). C.—RT-qPCR analysis of genes involved in cholesterol and bile acid homeostasis, in the liver of L^{ΔRbfox2} and L^{WT} mice fed a HFr diet (n=18-20). D.—Representative western-blot showing APOB and ABCA1 expression in the liver of L^{ΔRbfox2} vs. L^{WT} mice (n=8). E-K.—Bile acids levels in the liver of L^{ΔRbfox2} vs. L^{WT} mice fed a HFr diet or (L-U) a HFD diet as determined by LC-MS/MS. Samples were normalised by tissue weight and internal standard (n=8). Results are represented as mean±SEM; Statistical significance was determined by two-sided t-test of biologically independent samples (*p-value<0.05; **p-value<0.01; ***p-value<0.001).

FIG. 13. Quantification of AS of direct RBFOX2 targets in mice fed a CD, a HFD or a HFr diet. A.—Quantification of percentage splice in (PSI) for Pla2g6 (exon 10), (B.—) Scarb1 (exon 12), (C.—) Numb (exon 3), (D.—) Numb (exon 9), (E.—) Sec31a (exon 21) and (F.—) Osbpl9 (exon 6) in L^{WT} and L^{ΔRbfox2} mice fed a CD, a HFD or a HFr diet (n=7-10). G.—Representative western-blot showing RBFOX2 expression levels in the liver of mice after transduction with pAd-RBFOX2 or pAd-GFP control (left) and quantification by PCR/capillary electrophoresis of percentage splice in (PSI) for Scarb1 (exon 12) (right) (n=7-9). H.-ChIPseq signal for FOXA1 in mouse Rbfox2 promoter in liver. I.—Volcano-plot showing LC-MS lipidomic analysis of L^{ΔRbfox2} vs L^{WT} hepatocytes (n=5-6). J.—PCA plot of L^{ΔRbfox2} vs. L^{WT} hepatocytes and relevant SSO treatment as determined by LC-MS lipidomic analysis. Results are represented as mean±SEM. Statistical significance was determined by two-way ANOVA or two-sided t-test of biologically independent samples (**p-value<0.01; ***p<0.001).

FIG. 14. Role of RBFOX2-downstream targets in lipid metabolism in hepatocytes. A.—Capillary electrophoresis and quantification of percentage splice in (PSI) for Numb (exon 9), in L^{WT} and L^{ΔRbfox2} hepatocytes upon treatment with SSO7.8 or Scr control. B.—Heatmap showing LC-MS metabolomic analysis of L^{WT} and L^{ΔRbfox2} hepatocytes treated with SSO7.8 or Scr. C.—Capillary electrophoresis and quantification of percentage splice in (PSI) for Sec31a (exon 21), in L^{WT} and L^{ΔRbfox2} hepatocytes upon treatment with SSO6.2 or Scr control. D.—Heatmap showing LC-MS metabolomic analysis of L^{WT} and L^{ΔRbfox2} hepatocytes treated with SSO6.2 or Scr. Results are represented as mean±SEM. Statistical significance was determined by two-sided t-test of biologically independent samples (n=5-6) (***p<0.001).

FIG. 15. Role of Osbpl9 and Pla2g6 isoforms in lipid metabolism. A.—Capillary electrophoresis and quantification of percentage splice in (PSI) for Osbpl9 (exon 6), in L^{WT} and L^{ΔRbfox2} hepatocytes upon treatment with SSO11.1 or Scr control. B.—Heatmap showing LC-MS metabolomic analysis of L^{WT} and L^{ΔRbfox2} hepatocytes treated with SSO11.1 or Scr. C.—Western blot showing PLA2G6 expression in wild type and RBFOX2-deficient hepatocytes (top). Cryo-EM maps of PLA2G6 dimers showing tight interaction of the catalytic domains (orange) of each monomer with ankyrin repeats (purple) oriented outward from the core. Ankyrin repeats face ‘claw-like’ towards membrane phospholipids. Insert: 90-degree rotation of the structure detailing the region corresponding to exon 10 of Pla2g6L, a 55-amino intrinsically disordered proline-rich region at the interface of the ankyrin repeats and the catalytic domain. D.—Diagram showing design of splice-switching oligos targeting Pla2g6 alternative splicing at exon 10. SSO5.1 is designed to promote exon skipping (top) as validated by semi-quantitative PCR analysis (bottom). E.—Quantification of the effect of SSO5.1 on the described lipid species as determined by LC-MS. Results are represented as mean±SEM (n=5-6). Statistical significance was determined by two-sided t-test of biologically independent samples (*p-value<0.05; ***p<0.001).

FIG. 16. Role of Scarb1 splicing variants in lipid metabolism. A.—Splice-switching oligonucleotide (SSO8.3) promotes skipping of Scarb1 exon 12 in primary hepatocytes as determined by semi-quantitative PCR analysis. B.—Volcano-plot showing LC-MS lipidomic analysis of L^{ΔRbfox2} hepatocytes treated with 100 nM SSO8.3 or Scr for 16 h. (n=5-6). C.—Metabolomics analysis showing levels of total ceramide, D.—PUFA/non-PUFA TG ratio, E.—total sphingomyelin, and F.—total triglycerides (n=5-6). G-I.—Heatmap showing LC-MS metabolomic analysis of L^{WT} and L^{ΔRbfox2} hepatocytes treated with SSO8.3 or Scr. J.—Western-blot showing SR-BI/II levels in the liver after SSO8.3 treatment. K.—RT-qPCR expression analysis in liver of mice treated with SSO8.3 or Scr of potential off-targets genes such as Dscc1, (L.—) Kcnj16 and (M.—) Rab10. N.—Effect of SSO8.3 injection on circulating ALT and O.—AST levels. P.—Liver/body weight ratio upon SSO8.3 injection (n=7-10). Results are represented as mean±SEM. Statistical significance was determined by one-way ANOVA or two-sided t-test of biologically independent samples (*p-value<0.05; **p-value<0.01; ***p-value<0.001).

FIG. 17. In vivo treatment with SSO8.3 promotes lipoprotein remodelling. A.—Quantification of major lipid species in purified VLDL, B.—LDL and C.—HDL lipoproteins from mice fed a HFr diet and treated with SSO8.3 or Scr control. Samples were pooled in three replicates. D.—Representative western blot analysis of lipogenic proteins in liver of L^{WT} and L^{ΔRbfox2} mice treated with SSO8.3 or Scr (n=9-10). E.—Quantification of liver triglyceride content normalised to liver weight in mice injected with SSO8.3 or Scr control (n=8-9). F.—RT-qPCR expression analysis of endogenous Scarb1 gene knockdown in AML12 hepatocytes treated with siRNA to Rbfox2 or Scr control (n=5). G.—Absolute RT-qPCR expression analysis of codon-optimised Scarb1 isoforms in AML12 cells (n=5). H.—Capillary electrophoresis and quantification of percentage splice in (PSI) for Scarb1 (exon 12) in L^{WT} and L^{ΔRbfox2} liver upon treatment with SSO8.3 or Scr control (top) and western-blot analysis of protein levels (bottom) (n=8-9). I.—Quantification of liver triglyceride content normalised to liver weight in L^{WT} and L^{ΔRbfox2} mice upon treatment with SSO8.3 or Scr control (n=9-10). J.—Total blood cholesterol level in L^{ΔRbfox2} vs. L^{WT} mice fed a HFr diet and treated with SSO8.3 or Scr control as indicated. K.—Quantification of major lipid species in purified LDL and (L) VLDL lipoproteins. Results are represented as mean±SEM; Statistical significance was determined by two-sided t-test of biologically independent samples (*p-value<0.05; **pvalue<0.01, ***p-value<0.001).

FIG. 18. Activity of SSO8.4, SSO8.5, SSO8.6 and SSO8.7 in human hepatocytes. A.—Cartoon showing the hybridization site of the tested SSOs within exon 12 locus. B.—Human hepatocytes were transfected for 6 h with 100 nM SSO with lipofectamine 2000. 24 h after transfection RNA was extracted and activity was quantified as PSI of SCARB1 exon 12. Scramble oligos were used as controls. Results are represented as mean±SEM (n=3).

FIG. 19. Activity of SSO8.5, SSO8.8, SSO8.9, SSO8.10, SSO8.11 and SSO8.12 in human hepatocytes. A.—Cartoon showing the hybridization site of the tested SSOs within exon 12 locus. B.—Human hepatocytes were transfected for 6 h with 100 nM SSO with lipofectamine 2000. 24 h after transfection RNA was extracted and activity was quantified as PSI of SCARB1 exon 12. Scramble oligos were used as controls. Results are represented as mean±SEM (n=3).

FIG. 20. Activity of SSO8.5, SSO8.10, SSO8.11, SSO8.12, SSO8.13, SSO8.14, SSO8.15, SSO8.16, SSO8.17, SSO8.18, SSO8.19, SSO8.20, SSO8.21 in Huh7 or HepG2 human hepatocytes. A.—Cartoon showing the hybridization site of the tested SSOs within exon 12 locus. B.—Cartoon depicting the chemical modifications introduced in the SSOs tested. C.—Huh7 human liver cells were transfected for 24 h with 100 nM SSO with lipofectamine 2000. 24 h after transfection RNA was extracted and activity was quantified as PSI of SCARB1 exon 12. D.—HepG2 human liver cells were transfected for 24 h with 100 nM SSO with lipofectamine 2000. 24 h after transfection RNA was extracted and activity was quantified as PSI of SCARB1 exon 12. Scramble oligos were used as controls. Results are represented as mean±SEM (n=3).

FIG. 21. Analysis of efficacy and toxicity of SSO8.19 in human liver microtissues. A.—PCR and capillary electrophoresis analysis of SCARB1 exon 12 inclusion/skipping. B.—Quantification of exon 12 inclusion/skipping represented as Percentage Spliced In (PSI=exon 12 included/(exon12 included+exon 12 skipped). C-D.—Liver toxicity evaluated as LDH release caused by increasing doses of SCR (C), SSO8.18 (D) and chlorpromazine (E). Results are represented as Mean±SEM.

FIG. 22. Anti-inflammatory effect of SS8.18. A.—Cartoon depicting the repeated treatment (20 μM) experimental setup. B.—Effect of SSO8.18 on CXCL10 (IP10) levels. C.—Cartoon depicting the experimental setup with single treatment (7.5 μM) for 48 h (top). PCR and capillary electrophoresis analysis of SCARB1 exon 12 inclusion/skipping in these conditions (bottom). D.—Liver toxicity evaluated as LDH release single treatment (7.5 μM). E.—Effect of SSO8.18 on CXCL10 (IP10) levels. Results are represented as Mean±SEM. Pair-wise statistical comparisons between SCR and SSO8.18 were evaluated with the student's t test (***P<0.001).

### DETAILED DESCRIPTION

The inventors have found that components of the pre-mRNA alternative splicing machinery are selectively regulated by metabolic inputs in the liver. They identify RNA-binding Fox protein 2 (RBFOX2) is a key splicing factor in the liver, regulating AS in a cluster of genes involved in lipid homeostasis. This includes the scavenger receptor class B type I (Scarb1), the phospholipase A2 group VI (Pla2g6), the clathrin vesicle adapter Numb, the component of the COPII vesicle trafficking system Sec31a and the oxysterol binding protein-like 9 (Osbpl9). The inventors reveal that in addition to promoting or antagonising the expression of specific AS variants in the liver, RBFOX2 regulates AS in response to obesogenic diets. They also show that this RBFOX2-regulated AS network can be targeted therapeutically. Specifically, splice-switching oligonucleotides (SSOs) modulating Scarb1 splicing revert the accumulation of lipotoxic species in RBFOX2-deficient mouse hepatocytes, alleviate liver inflammation associated with diet-induced obesity in vivo and promote an anti-atherogenic lipoprotein profile in the blood. These findings highlight the potential of isoform-specific RNA therapeutics for metabolic pathologies.

In an aspect, there is provided an agent capable of inducing skipping of exon 12 of scavenger receptor class B type I (Scarb1). The agent may bind to the pre-mRNA of Scarb1. For instance, the agent may bind to a site within Scarb1 pre-mRNA that affects the splicing of exon 12. The agent may be a nucleic acid analogue or a nucleic acid. The agent may be an antisense oligomer.

A “nucleic acid analogue” is a compound that has an arrangement of nucleobases that mimics the arrangement of nucleobases in nucleic acids containing a 2′ deoxyribose 5′ monophosphate or ribose 5′ monophosphate backbone, wherein the nucleic acid analogue is capable of base pairing with a complementary nucleic acid. Examples of backbone moieties include amino acids as in peptide nucleic acids, glycol molecules as in glycol nucleic acids, threofuranosyl sugar molecules as in threose nucleic acids, morpholine rings and phosphorodiamidate groups as in morpholinos, and cyclohexenyl molecules as in cyclohexenyl nucleic acids.

In an aspect, there is provided an antisense oligomer capable of inducing skipping of exon 12 of scavenger receptor class B type I (SCARB1).

An “antisense oligomer”, in the context of the present disclosure, is a molecule comprising subunits that comprise moieties capable of binding to nucleobases. Hence, antisense oligomers can be designed to be capable of hybridising to specific nucleic acid sequences. The subunits may be monomers, each monomer comprising a moiety capable of binding to a nucleobase. The moieties capable of binding to a nucleobase may bind by base-specific hydrogen bonding, such as Watson-Crick base pairing.

The antisense oligomer may be referred to as an antisense compound, particularly where the compound is not necessarily synthesised from monomers.

The term “antisense” refers to molecules that are at least partially complementary to a region of a sense strand of a nucleic acid. The antisense oligomers of the present disclosure are at least partially complementary to a region of Scarb1 pre-mRNA and are capable of binding by hybridisation to said region. The degree of complementarity may not be exact, as long as the antisense oligomer and the pre-mRNA can hybridise under physiological conditions. In some examples, the antisense oligomer and the pre-mRNA may be complementary apart from 5, 4, 3, 2, or 1 mismatches. In some examples, the antisense oligomer is perfectly complementary to a region of the pre-mRNA. In other examples, the antisense oligomer comprises a region that is perfectly complementary to a region of the pre-mRNA, wherein the complementary regions are of a sufficient length to allow binding by hybridisation.

Physiological conditions are the conditions (such as temperature, pH, concentration of various ions, etc) found in natural, in vivo, situations, or conditions that correspond such a situation. For instance, the antisense oligomer may bind by hybridisation in intracellular conditions, such as the intracellular conditions of human cells. The physiological conditions may be those during pre-mRNA splicing in cells found in the liver, for instance the intracellular conditions of human hepatocytes. Confirmation of hybridisation in physiological conditions may be performed in vitro, for instance at a temperature, pH, and salt concentration that approximates intracellular conditions. In a particular example, the antisense oligomer may hybridise to Scarb1 pre-mRNA at 37° C., pH 7.4, and phosphate buffered saline (e.g. containing 137 mM NaCl, 2.7 mM KCl, 10 mM Na₂HPO₄, and 1.8 mM KH₂PO₄). In all embodiments, the degree of hybridisation is capable of inducing exon skipping during splicing of the target gene in the subject.

The binding of the antisense oligomers disclosed herein to Scarb1 pre-mRNA is capable of inducing skipping of exon 12 during splicing. The antisense oligomers disclosed herein may therefore be referred to as splice-switching oligonucleotides (SSO) due to this activity. The SSOs disclosed herein, upon binding to Scarb1 pre-mRNA, increase the possibility of exon 12 being skipped during splicing. When exon 12 is skipped, it is not included in the resultant mRNA and the polypeptide region encoded by exon 12 will not be present in the translated protein. This effect is important because inclusion of exon 12 of Scarb1 generates the canonical SR-BI isoform, while skipping this exon generates an alternative receptor variant with a different adapter carboxy-terminal domain, named SR-BII.

Splice-switching activity may be detected by an assay to determine if the candidate antisense oligomer is capable of increasing the ratio of mRNA-not-comprising-Scarb1-exon 12 to mRNA-comprising-Scarb1-exon 12 within a cell. The antisense oligomers of the present disclosure may increase the ratio of SR-BII to SR-BI expressed by the cell. These effects may be quantified as a percentage splice (PSI), which may be expressed as a percentage of non-exon-12-skipped Scarb1 mRNA relative to the total Scarb1 mRNA. The relevant calculation is therefore: PSI=exon 12 included/(exon 12 included+exon 12 skipped). A PSI of 100% represents the situation where all Scarb1 mRNA comprises exon 12 (i.e. no skipping is induced). Methods for the measurement of PSI are disclosed in the Examples herein. For example, the PSI may be measured by transfection of a candidate splice-switching oligomer into Huh7 human liver cells, extraction of RNA after 24 hours, and the quantification of exon 12-included Scarb1 mRNA and total Scarb1 mRNA (see the Examples for further information).

In some examples, the antisense oligomer of the present disclosure induces skipping of Scarb1 exon 12 so that the PSI is less than or equal to 90%, 80%, 70%, 60%, 50%, 40%, 30%, 20%, 10%, or 5.7%. In a particular embodiment, the PSI is less than 40% (i.e. the antisense oligomer has induced a 60% decrease in PSI of SCARB1 exon 12).

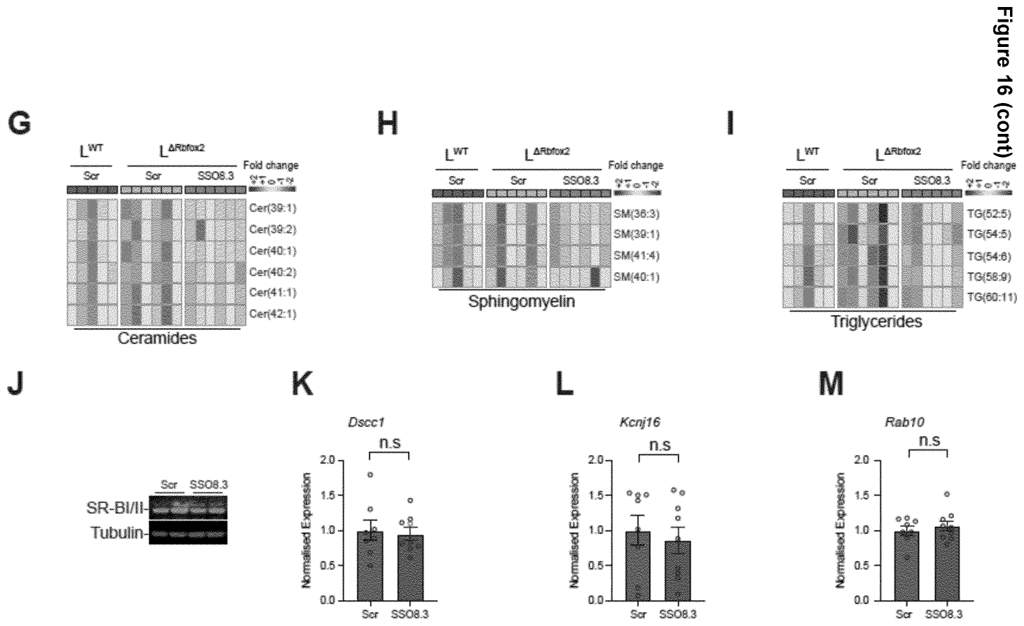

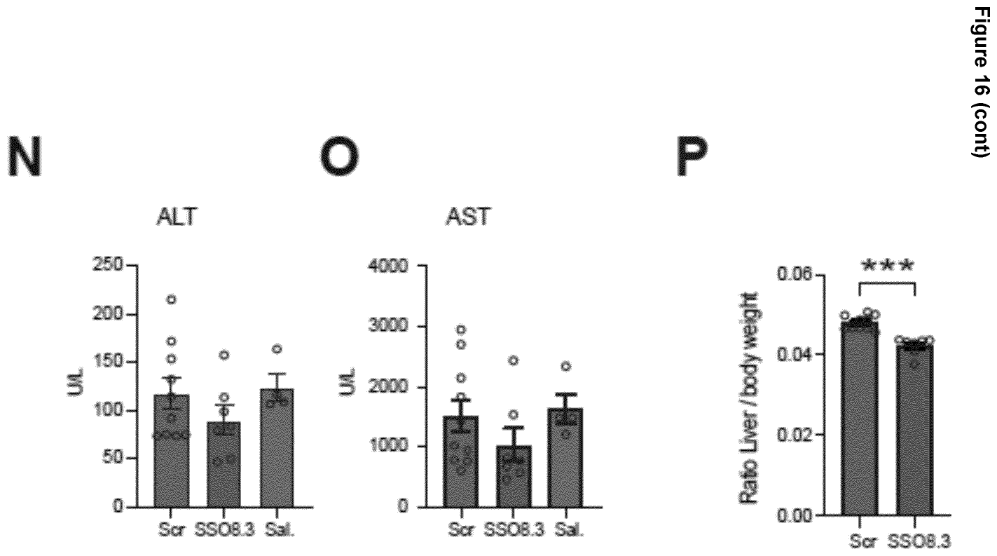

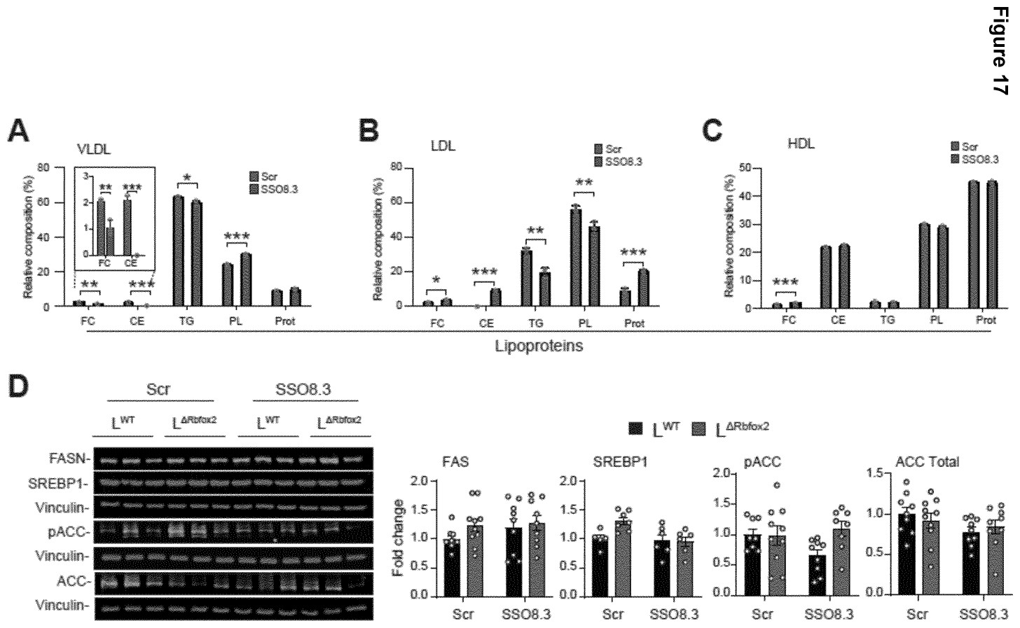

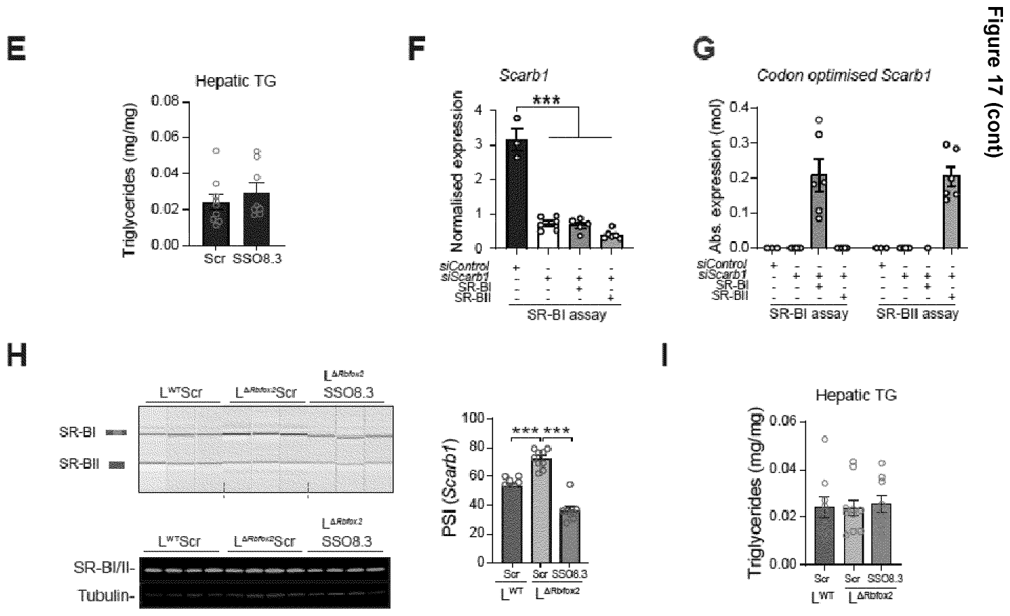

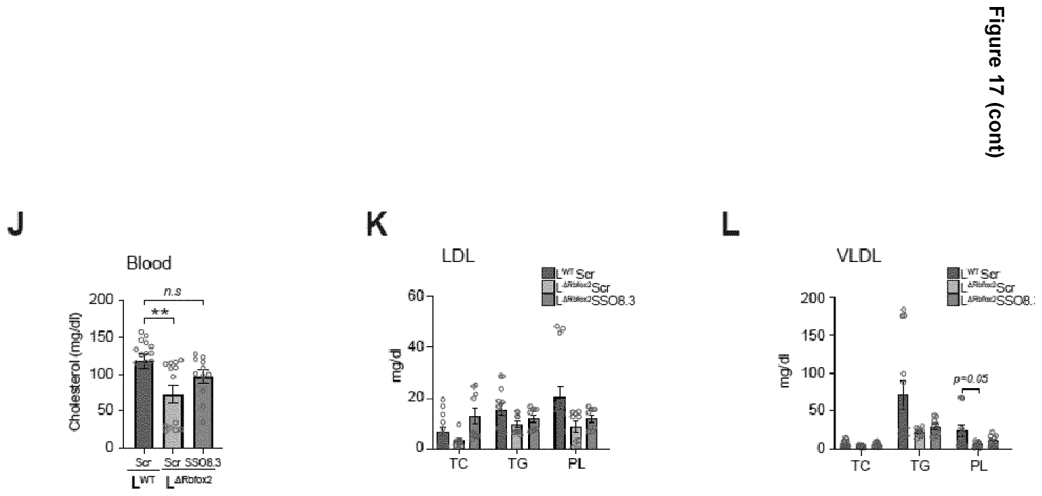

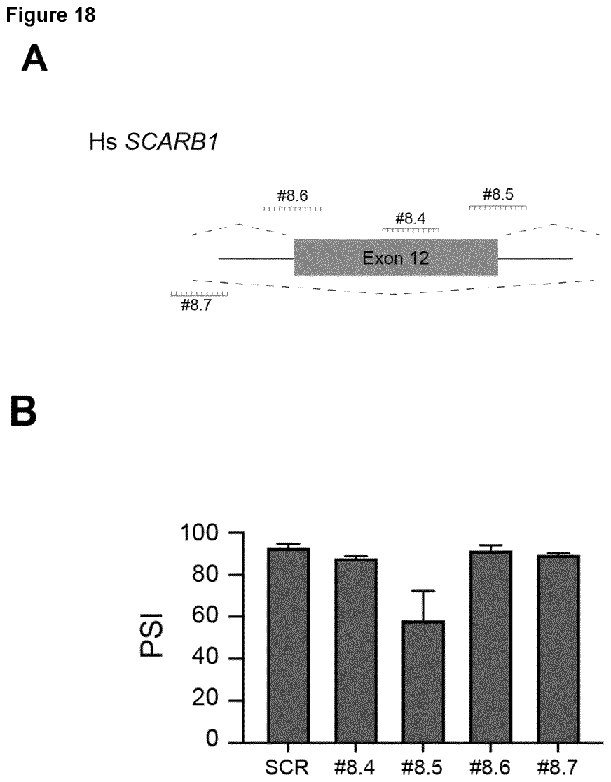

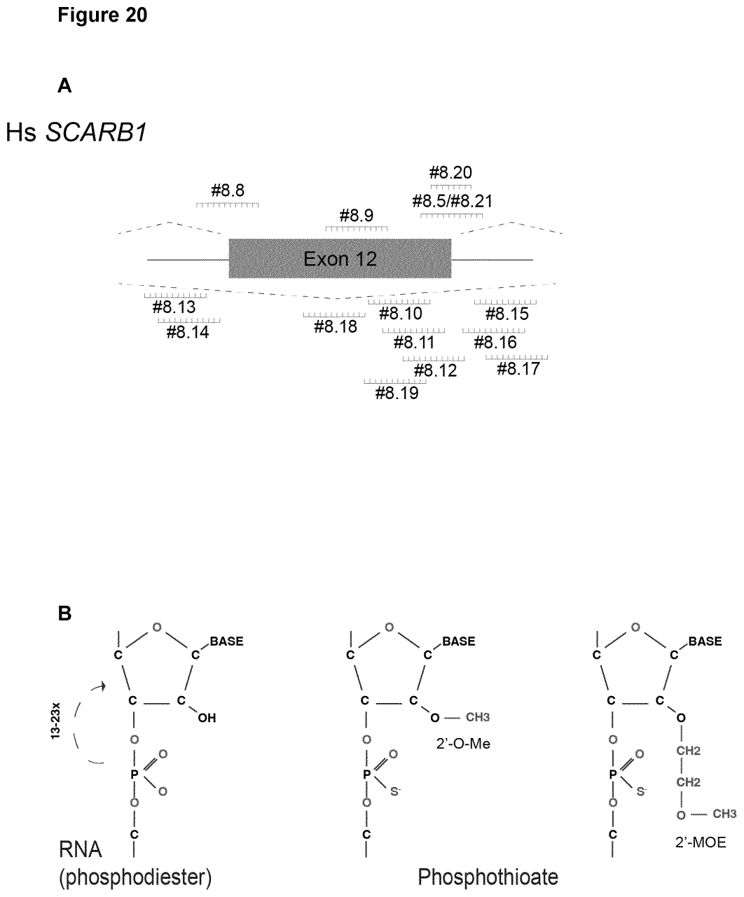

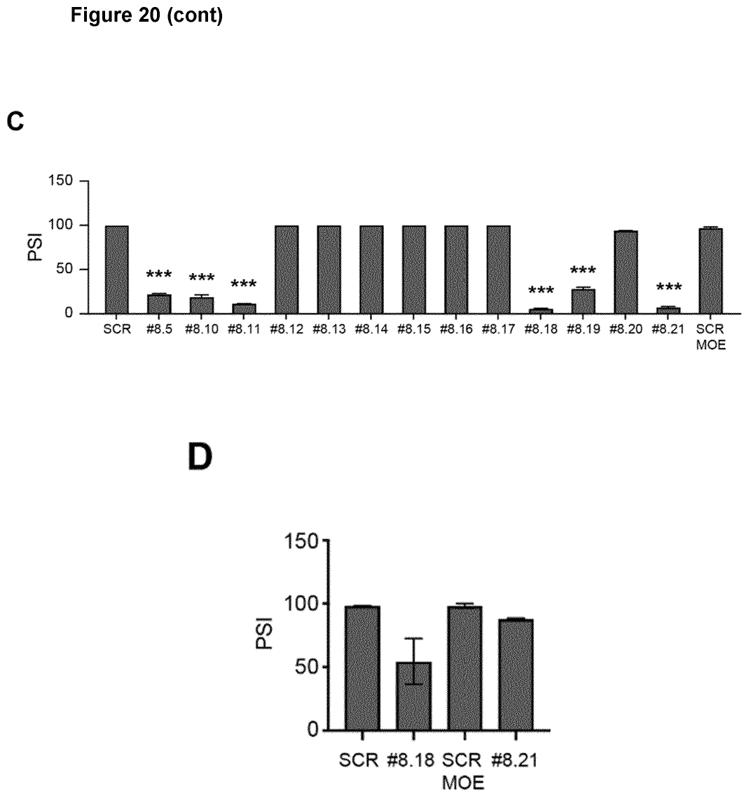

The antisense oligomer may target cis-regulatory regions within Scarb1 pre-mRNA (see FIG. 18A). In some examples, the antisense oligomer is targeted to the 3′ splice site of Scarb1 exon 12. For instance, the antisense oligomer may be complementary to a region comprising the 3′ end of exon 12 in Scarb1 pre-mRNA. SSO8.5 and SSO8.21 provide examples of such antisense oligomers (see FIG. 20). In other examples, the antisense oligomer may be targeted to a region within exon 12, and so is capable of hybridising to a region of exon 12 that does not comprise the 5′ or 3′ ends. SSO8.18 provides an example of such an antisense oligomer (see FIG. 20). The antisense oligomer may be capable of hybridising to a region that overlaps with the region to which SSO8.18 is complementary. For instance, the antisense oligomer may be complementary to 5, 10, 15, 16, 17, 18, 19, or all 20 bases to which SSO8.18 is complementary.

The sequence of the SCARB1 gene may be as described for Gene ID: 949 on the NIH identification page and updated on 12 Aug. 2022. The gene may be as described by Ensemble gene: SCARB1 ENSG00000073060.17.

The sequence of the SCARB1 pre-mRNA may be the sequence of human SCARB1 pre-mRNA. The sequence of the SCARB1 pre-mRNA may be according to SEQ ID NO: 1.

The sequence of Scarb1 exon 12 may be:

(SEQ ID NO: 2)
GAGAAATGCTATTTATTTTGGAGTAGTAGTAAAAAGGGCTCAAAGGATAA
GGAGGCCATTCAGGCCTATTCTGAATCCCTGATGACATCAGCTCCCAAGG
GCTCTGTGCTGCAGGAAGCAAAACTGTAG

The antisense oligomer may comprise nucleobases capable of base pairing with nucleobases of target nucleic acids. The antisense oligomer may comprise nucleotides. The antisense oligomer may be, or may comprise, a nucleic acid and/or nucleic acid analogue. The antisense oligomer may be, or may comprise, an oligonucleotide or a polynucleotide. The antisense oligomer may be, or may comprise, a phosphorodiamidate morpholino oligomer (PMO). The antisense oligomer may be, or may comprise, a peptide nucleic acid (PNA).

In some examples, the antisense oligomer comprises at least 10, 12, 13, 14, 15, 16, 17, 18, 19, 20, or 21 nucleobases. In some examples, the antisense oligomer comprises no more than 45, 40, 35, 30, 29, 28, 27, 26, 25, 24 or 21 nucleobases. The antisense oligomer may comprise 10-45, 12-40 nucleobases, 15-35 nucleobases, 18-30 nucleobases, or 19-25 nucleobases. The antisense oligomer may be 14-30, 15-29, 16-28, 17-27, 18-26, or 19-25 nucleobases in length. The antisense oligomer may be 14-24, 15-24, 16-24, 17-24, 18-24, or 19-24 nucleobases in length. In a particular embodiment, the antisense oligomer is 21 nucleobases in length.

The antisense oligomer may comprise a nucleoside or nucleosides that have been modified, for instance the modifications may be to the sugar moiety. In some examples, the 2′-position of the sugar moiety may be modified. A modification may be to any moiety that is not “—H” for DNA or not “—OH” for RNA. Examples of such 2′ modifications are —O—CH₃ or —O—CH₂—CH₂—O—CH₃, and further examples are provided herein. In some examples, the 4′ position of the sugar moiety made be modified, for instance to result in a bridge between the 2′ position and the 4′ position. The antisense oligomer may comprise one, two, three, four, or more types of nucleoside. The antisense oligomer may comprise a combination of modified nucleosides and unmodified nucleosides. The antisense oligomer may comprise only modified nucleosides. The nucleotides of the antisense oligomer may all be modified in the same manner or may be modified in two or more different manners.

The antisense oligomer may comprise a modification to one or more internucleoside linkages. For instance, the antisense oligomer may comprise one or more phosphorothioate linkages. In some embodiments, all of the internucleotide linkages within the antisense oligomer are phosphorothioate linkages. The antisense oligomer may be or may comprise an oligonucleotide phosphorothioate. The antisense oligomer may comprise one or more phosphorodiamidate linkage. In some embodiments, all of the intermonomer linkages within the antisense oligomer are phosphorodiamidate linkages.

The antisense oligomer may comprise one or more of a deoxyribonucleotide, a ribonucleotide, an arabinonucleotide, a 2′-Fluoroarabinonucleotide (FANA), a 2′-O-methyl (2′OMe) nucleotide, a phosphorothioate 2′-O-methyl (PS-2′OMe) nucleotide, a 2′-O-methoxyethyl (MOE) nucleotide, a phosphorothioate 2′-O-methoxyethyl (PS-MOE) nucleotide, a phosphorodiamidate morpholino monomer, a locked nucleotide, a P-alkyl phosphonate nucleotide, a threose nucleotide, a hexitol nucleotide, a 2′ hydroxy-hexitol nucleotide, a cyclohexene nucleotide, a 3′ deoxi-DNA (2′-5′) nucleotide, a peptide nucleic acid (PNA) residue, a 2′-O,4′-C-ethylene-bridged nucleotide, or any combination thereof.

The antisense oligomer may be or may comprise a DNA oligomer, an RNA oligomer, an arabinonucleic acid (ANA) oligomer, a 2′-Fluoroarabinonucleic acid (FANA) oligomer, a 2′-O-methyl ribonucleic acid (2′OMe) oligomer, a phosphorothioate 2′-O-methyl ribonucleic acid (PS-2′OMe) oligomer, a 2′-O-methoxyethyl (MOE) nucleic acid oligomer, a phosphorothioate 2′-O-methoxyethyl (PS-MOE) nucleic acid oligomer, a phosphorodiamidate morpholino oligomer (PMO), a locked nucleic acid (LNA) oligomer, a P-alkyl phosphonate nucleic acid (phNA) oligomer, a threose nucleic acid (TNA) oligomer, a hexitol nucleic acid (HNA) oligomer, a 2′ hydroxy-hexitol (AtNA) oligomer, a cyclohexene nucleic acid (CeNA) oligomer, a 3′ deoxi-DNA (2′-5′) oligomer, a peptide nucleic acid (PNA) oligomer, a 2′-0,4′-C-ethylene-bridged nucleic acid (ENA) oligomer, or any combination thereof.

The antisense oligomer of the present disclosure may include one or more naturally occurring nucleobase and/or one or more modified nucleobase. A modified nucleobase is a nucleobase that is capable of base pairing with a nucleobase of a nucleic acid, but is structurally different from a naturally occurring nucleobase. An example of a modified nucleobase is 5-methylcytosine.

Any of the antisense oligomers of the present disclosure may be present as a pharmaceutically acceptable salt, ester, salt of said ester, or hydrate of said antisense oligomer, and references to an antisense oligomer encompass such compounds. The antisense oligomer of the present disclosure may be present as a prodrug.

The sequence of the scrambled oligonucleotide used in Examples 8 is:

(SEQ ID NO: 3)
AAAUAAUUGAAUUUUAAAUA

Examples of sequences that are complementary to Scarb1 pre-mRNA include:

(SEQ ID NO: 4)
GGCCUGAAUGGCCUCCUUAUC
(SEQ ID NO: 5)
GGUACCCACCUACAGUUUUG
(SEQ ID NO: 6)
CAAAAUAAAUAGCAUUUCUC
(SEQ ID NO: 7)
GUGGCAACGCGGCAUGCAA
(SEQ ID NO: 8)
AGCAUUUCUCCUAGAAGAUA
(SEQ ID NO: 9)
UUGAGCCCUUUUUACUACUA
(SEQ ID NO: 10)
GAUGUCAUCAGGGAUUCAGA
(SEQ ID NO: 11)
GCACAGAGCCCUUGGGAGCU
(SEQ ID NO: 12)
ACCUACAGUUUUGCUUCCUG
(SEQ ID NO: 13)
ACAUAAGUACAAGCAUCUUCA
(SEQ ID NO: 14)
GGUUUUACACGGUUCUUCAA
(SEQ ID NO: 15)
GCUGAAGGAAUGAGCAGGAC
(SEQ ID NO: 16)
GCAUCUUCAGUCUGUAGACAC
(SEQ ID NO: 17)
CUGUCUUUCUAAUGUGACCU
(SEQ ID NO: 18)
CUUGGGAGCUGAUGUCAUCA
(SEQ ID NO: 19)
ACAGUUUUGCUUCCUGCAGCACAGA
(SEQ ID NO: 20)
CCACCUACAGUUUUG

In the sequence listing, all “U”s have been replaced with “T”s as per the requirements of WIPO ST.26. The antisense oligomers of the present disclosure may comprise uracil or thymine in said positions. In some embodiments, the nucleobases are as recited above, including the respective uracils.

The antisense oligomer of the present disclosure may comprise the sequence of or may be according to the sequence of any one of SEQ ID NOs: 4-20, wherein the sequence comprises modifications including extensions, deletions, insertions, substations, and wherein the antisense oligomer is capable of inducing skipping of exon 12 of Scarb1. The antisense oligomer may be complementary to 5, 10, 15, 16, 17, 18, 19, 20, or all bases to which an antisense oligomer comprising any one of SEQ ID NOs: 4-20 is complementary.

In an embodiment, the antisense oligomer of the present disclosure may comprise the sequence or may be according to the sequence of any one of SEQ ID NOs: 4, 5, 7, 8, 9, 10, 11, 12, 18, or 19, wherein the sequence comprises 5, 4, 3, 2, 1, or no substitutions, deletions, or insertions. In particular, the sequence may comprise 3, 2, 1, or no substitutions. In an embodiment, the antisense oligomer of the present disclosure may comprise the sequence or may be according to the sequence of any one of SEQ ID NOs: 4, 5, 7, 8, 9, 10, 11, 12, 18, or 19. The antisense oligomer may comprise a nucleotide comprising a 2′-O-methyl sugar moiety and/or a 2′-O-methoxyethyl sugar moiety. The antisense oligomer may comprise only 2′-O-methyl nucleotides and/or 2′-O-methoxyethyl nucleotides. The antisense oligomer may comprise one or more phosphorothioate linkages. In some examples, all of the linkages within the antisense oligomer are phosphorothioate linkages.

In some embodiments, the antisense oligomer does not comprise any additional nucleic acid sequence, or analogous nucleobases, beyond that recited in any one of SEQ ID NOs: 4, 5, 7, 8, 9, 10, 11, 12, 18, or 19. In other embodiments, the antisense oligomer may comprise 1, 2, 3, 4, 5, 10, 15, 20, 25, 30, 35, 40, 45, or more nucleobases in addition to those recited in: i) any one of SEQ ID NOs: 4-20, ii) any one of SEQ ID NOs: 4, 5, 7, 8, 9, 10, 11, 12, 18, or 19, or iii) any one of SEQ ID NOs: 5, 8, 10, 11, 18, or 19.

In particular embodiments, the antisense oligomer of the present disclosure may comprise the sequence or may be according to the sequence of any one of SEQ ID NOs: 5, 8, 10, 11, 18, or 19, wherein the sequence comprises 5, 4, 3, 2, 1, or no substitutions, deletions, or insertions. In particular, the sequence may comprise 3, 2, 1, or no substitutions. In particular embodiments, the antisense oligomer of the present disclosure may comprise the sequence or may be according to the sequence of any one of SEQ ID NOs: 5, 8, 10, 11, 18, or 19. The antisense oligomer may comprise a 2′-O-methyl nucleotide and/or a 2′-O-methoxyethyl nucleotide. The antisense oligomer may comprise only 2′-O-methyl nucleotides and/or 2′-O-methoxyethyl nucleotides. The antisense oligomer may comprise one or more phosphorothioate linkages. In some examples, all of the linkages within the antisense oligomer are phosphorothioate linkages. In some embodiments, the antisense oligomer does not comprise any additional nucleic acid sequence, or analogous nucleobases, beyond that recited in any one of SEQ ID NOs: 5, 8, 10, 11, 18, or 19.

The antisense oligomer of the present disclosure may comprise the sequence or may be according to the sequence of any one of SEQ ID NOs: 4, 5, 7, 8, 9, 10, 11, 12, 18, or 19, wherein one of more of the nucleobases has been substituted for a modified nucleobase. For instance, one of more of the nucleobases of any one of SEQ ID NOs: 4, 5, 7, 8, 9, 10, 11, 12, 18, or 19 may be replaced with a modified nucleobase that retains the base pairing capability of the replaced nucleobase. As an example, the cytosines of any one of SEQ ID NOs: 4, 5, 7, 8, 9, 10, 11, 12, 18, or 19 may be replaced with 5-methylcytosine. Additionally, any uracil in a sequence disclosed herein may be exchanged for a thymidine.

The antisense oligomer may be complementary to 5, 10, 15, 16, 17, 18, 19, 20, or all bases to which an antisense oligomer comprising any one of SEQ ID NOs: 4, 5, 7, 8, 9, 10, 11, 12, 18, or 19 is complementary. The antisense oligomer may be complementary to 5, 10, 15, 16, 17, 18, 19, 20, or all bases to which an antisense oligomer comprising any one of SEQ ID NOs: 5, 8, 10, 11, 18, or 19 is complementary.

In an embodiment, the antisense oligomer is not according to, or does not comprise, the sequence

(SEQ ID NO: 21)
AGCCCUUGGGAGCUGAUGUCAUCAG (US 2005/0244851A1).

Examples of antisense oligonucleotides are provided below.

| | | |
|---|---|---|
| SCR | [A*A*A*U*A*A*U*U*G*A*A*U*U*U*U*A*A*A*U*A] | (SEQ ID NO: 35) |
| SCR MOE | <A*A*A*U*A*A*U*U*G*A*A*U*U*U*U*A*A*A*U*A> | (SEQ ID NO: 36) |
| SSO8.4 | [G*G*C*C*U*G*A*A*U*G*G*C*C*U*C*C*U*U*A*U*C] | (SEQ ID NO: 37) |
| SSO8.5 | [G*G*U*A*C*C*C*A*C*C*U*A*C*A*G*U*U*U*U*G] | (SEQ ID NO: 38) |
| SSO8.6 | [C*A*A*A*A*U*A*A*A*U*A*G*C*A*U*U*U*C*U*C] | (SEQ ID NO: 39) |
| SSO8.7 | [G*U*G*G*C*A*A*C*G*C*G*G*C*A*U*G*C*A*A] | (SEQ ID NO: 40) |
| SSO8.8 | [A*G*C*A*U*U*U*C*U*C*C*U*A*G*A*A*G*A*U*A] | (SEQ ID NO: 41) |
| SSO8.9 | [U*U*G*A*G*C*C*C*U*U*U*U*U*A*C*U*A*C*U*A] | (SEQ ID NO: 42) |
| SSO8.10 | [G*A*U*G*U*C*A*U*C*A*G*G*G*A*U*U*C*A*G*A] | (SEQ ID NO: 43) |
| SSO8.11 | [G*C*A*C*A*G*A*G*C*C*C*U*U*G*G*G*A*G*C*U] | (SEQ ID NO: 44) |
| SSO8.12 | [A*C*C*U*A*C*A*G*U*U*U*U*G*C*U*U*C*C*U*G] | (SEQ ID NO: 45) |
| SSO8.13 | [A*C*A*U*A*A*G*U*A*C*A*A*G*C*A*U*C*U*U*C*A] | (SEQ ID NO: 46) |
| SSO8.14 | [G*G*U*U*U*U*A*C*A*C*G*G*U*U*C*U*U*C*A*A] | (SEQ ID NO: 47) |
| SSO8.15 | [G*C*U*G*A*A*G*G*A*A*U*G*A*G*C*A*G*G*A*C] | (SEQ ID NO: 48) |
| SSO8.16 | [G*C*A*U*C*U*U*C*A*G*U*C*U*G*U*A*G*A*C*A*C] | (SEQ ID NO: 49) |
| SSO8.17 | [C*U*G*U*C*U*U*U*C*U*A*A*U*G*U*G*A*C*C*U] | (SEQ ID NO: 50) |
| SSO8.18 | [C*U*U*G*G*G*A*G*C*U*G*A*U*G*U*C*A*U*C*A] | (SEQ ID NO: 51) |
| SSO8.19 | [A*C*A*G*U*U*U*U*G*C*U*U*C*C*U*G*C*A*G*C*A*C*A*G*A] | (SEQ ID NO: 52) |
| SSO8.20 | [C*C*A*C*C*U*A*C*A*G*U*U*U*U*G] | (SEQ ID NO: 53) |
| SSO8.21 | <G*G*U*A*C*C*C*A*C*C*U*A*C*A*G*U*U*U*U*G> | (SEQ ID NO: 54) |

Code:
[2′oMe]
<2′MOE>
*Phosphorothioate

In embodiments, the antisense oligomer of the present disclosure may comprise or may be an antisense oligonucleotide illustrated above as SSO8.4, SSO8.5, SSO8.7, SSO8.8, SSO8.9, SSO8.10, SSO8.11, SSO8.12, SSO8.18, SSO8.19, or SSO8.21. The antisense oligomer may be or may comprise any one of SEQ ID NOs: 35-54. Optionally, any of these antisense oligonucleotides may comprise 5, 4, 3, 2, 1, or no substitutions, deletions, or insertions to the sequence. In particular, the sequence may comprise 3, 2, 1, or no substitutions. Optionally, the antisense oligonucleotides may comprise 10, 9, 8, 7, 6, 5, 4, 3, 2, 1, or no modifications, for instance one or linkages between nucleotides may differ from a phosphorothioate linkage, or one or more nucleotides may have a sugar moiety that differs from the respective 2′-O-methyl nucleotide or 2′-O-methoxyethyl nucleotide. In particular embodiments, the antisense oligomer of the present disclosure may comprise or may be an antisense oligonucleotide illustrated above as SSO8.5, SSO8.8, SSO8.10, SSO8.11, SSO8.18, SSO8.19, or SSO8.21. Within said antisense oligonucleotides, any uracil may be replaced by a thymidine.

In some examples, the antisense oligomer of the present disclosure is present in an isolated form. An isolated antisense oligomer does not comprise further nucleobases that are complementary to the target pre-mRNA. However, the isolated antisense oligomer may be associated additional components, such as peptides, lipids, sugar moieties, or modifications to the 5′ or 3′ ends of the antisense oligomer. The association between the antisense oligomer and any of said components may be covalent or non-covalent. The antisense oligomer may be purified.

The antisense oligomer may be conjugated to one or more sugar moiety, for instance a monosaccharide, disaccharide, or oligosaccharide. The antisense oligomer may be glycosylated or glycated. In an example, the antisense oligomer is conjugated to at least one N-acetylgalactosamine (GalNac). The GalNac may be associated with targeting the antisense oligomer to the liver of a subject.

The antisense oligomer of the present disclosure may be associated with one or more other agents, for instance an agent that promotes the delivery of the antisense oligomer to the relevant site. The antisense oligomer may be associated with an agent for the delivery of the antisense oligomer to a target organ, such as the liver or the gallbladder. The antisense oligomer may be associated with an agent for the delivery of the antisense oligomer into cells, for instance for promoting the transfer of the antisense oligomer across a membrane. The antisense oligomer may be non-covalently or covalently bound to said one or more agent.

The antisense oligomer of the present disclosure may be present as part of extracellular vesicle composition. For instance, the antisense oligomer may be loaded into the lumen of an exosome or associated with a component of an exosome. The antisense oligomer may be associated with monolayers, micelles, bilayers, lipid vesicles, or liposomes.

The antisense oligomer may be included in a nanoparticle, for instance a lipid nanoparticle composition. The antisense oligomer may be covalently or non-covalently bound to a peptide that promotes cell entry, such as a cell-penetrating peptide. The peptide for cellular entry may be a polycationic peptide, may be an amphipathic peptide, may be a hydrophobic peptide, may be a stapled peptide, or may be a stitched peptide.

The antisense oligomer may be part of, or encoded by, a vector. Hence the antisense oligomer may be delivered as a part of a vector or by transcription from said vector. The vector may be within an extracellular vesicle, such as an exosome, or a nanoparticle. And so, in an embodiment, there is provided an extracellular vesicle or nanoparticle comprising a vector encoding an antisense oligomer of the present invention.

In an aspect, there is provided a pharmaceutical composition comprising an antisense oligomer as disclosed herein. The pharmaceutical composition may comprise a pharmaceutically acceptable vehicle, a pharmaceutically acceptable carrier, a pharmaceutically acceptable excipient, a pharmaceutically acceptable stabilizer, or a pharmaceutically acceptable preservative, or any combination thereof. To be pharmaceutically acceptable, a substance or combination of substances must be suitable for the formulation of pharmaceutical compositions or a medicament.

The pharmaceutical composition may comprise a therapeutically effective amount of the antisense oligomer of the present disclosure. The phrases “therapeutically effective amount” and “effective amount” and the like, as used herein, indicate an amount necessary to administer to a subject, or to a cell, tissue, or organ of a subject, to achieve a therapeutic effect, such as an ameliorating or alternatively a curative effect. The effective amount is sufficient to elicit the biological or medical response of a cell, tissue, system, animal, or human that is being sought by a researcher, veterinarian, medical doctor, or clinician.

In an aspect, there is provided an antisense oligomer or pharmaceutical composition of the present disclosure for use as a medicament.

In an aspect, there is a method of treatment comprising administering a therapeutically effective amount of an antisense oligomer or pharmaceutical composition of the present disclosure to a subject in need thereof.

In an aspect, there is provided use of an antisense oligomer or pharmaceutical composition of the present disclosure for the manufacture of a medicament.

The antisense oligomer or pharmaceutical composition of the present disclosure may be for use in treating or preventing a metabolism-associated disease. For instance, the antisense oligomer may be for treating or preventing a pathological effect of obesity and/or an obesogenic diet. The pathological effect may be liver inflammation. The pathological effect may be lipotoxicity. In some examples, the antisense oligomer of the present disclosure is for use in reducing hepatocellular injury and/or fibrosis in a subject in need thereof.

The antisense oligomers or pharmaceutical composition of the present disclosure may be used to reduce levels of lipotoxic species in hepatocytes. For instance, the lipotoxic species may be ceramides, cholesterol and/or sphingomyelins. In some examples, the antisense oligomers of the disclosure may be used to promote an anti-atherogenic lipoprotein profile in the blood. The anti-atherogenic lipoprotein profile may be associated with a reduction in plasma VLDL and triglycerides.

In some examples, the antisense oligomer or pharmaceutical composition of the present disclosure is for use in treating or preventing liver inflammation. For instance, obesity-induced liver inflammation. The antisense oligomer of the present disclosure may be for use in treating or preventing metabolism-associated fatty liver disease (MAFLD). The antisense oligomer of the present disclosure may be for use in treating or preventing pre-symptomatic hepatic steatosis (fatty liver), non-alcoholic steatohepatitis (NASH), liver failure, or hepatocellular carcinoma (HCC). In a particular embodiment, the antisense oligomer of the present disclosure is for use in treating or preventing NASH, and a therapeutically effective amount of the antisense oligomer is administered to a subject in need thereof. In another embodiment, the antisense oligomer may prevent, delay, or reduce the severity of HCC, where the HCC is associated with liver inflammation, for instance NASH. In an example, the subject may have NASH and be at risk of HCC, and the antisense oligomer may reduce the risk of developing HCC. Alternatively, the subject may have NASH and HCC, and the antisense oligomer may reduce the severity of at least one symptom.

The antisense oligomer or pharmaceutical composition of the present disclosure may be for use in treating or preventing gallstone disease. The antisense oligomer may be used to lower the levels of cholesterol in bile. The levels of cholesterol in bile may be lowered to an extent that reduces the risk of gallstone disease or the severity of gallstone disease.

The antisense oligomer or pharmaceutical composition may be for use in treating or preventing any one of, or a combination of, type 2 diabetes, cardiovascular disease, and coronary artery disease. The treatment of said pathological conditions may be due to the reduction or prevention of metabolic changes in the liver. For instance, the treatment of MAFLD may reduce the risk of, or reduce the severity of, type 2 diabetes, cardiovascular disease, or coronary artery disease.

The antisense oligomer or pharmaceutical composition may be for use in lowering cholesterol levels in a subject in need thereof. For instance, the subject may have hypercholesterolemia or MAFLD. The cholesterol levels may be total cholesterol levels, circulating cholesterol levels, free cholesterol levels, liver cholesterol levels, intrahepatic cholesterol levels, and/or bile cholesterol levels.

In an aspect, there is provided a method of increasing the expression of SR-BII relative to SR-BI in a cell. The method may comprise the contacting of a cell with a composition comprising an antisense oligomer of the present disclosure or may comprise the administration of an antisense oligomer to a subject in need thereof. The levels of SR-BII may be increased on hepatocytes. The method may be in vitro or in vivo.

In a particular embodiment, there is provided an antisense oligonucleotide with a nucleobase sequence according to SEQ ID NO: 5, for use in a method of treatment. The oligonucleotide may comprise modified nucleobases (e.g. 5-methylcytosine instead of cytosine, etc). The oligonucleotide may comprise at least one modified internucleotide linkage, such as a phosphorothioate internucleotide linkage. The oligonucleotide may comprise one or more nucleotide with a modified sugar moiety, such as —O—CH₃ or —O—CH₂—CH₂—O—CH₃ attached to the 2′ position of the sugar moiety. The method of treatment may be for the treatment of liver inflammation. The method of treatment may be for NASH.

In a particular embodiment, there is provided an antisense oligonucleotide with a nucleobase sequence according to SEQ ID NO: 11, for use in a method of treatment. The oligonucleotide may comprise modified nucleobases (e.g. 5-methylcytosine instead of cytosine, etc). The oligonucleotide may comprise at least one modified internucleotide linkage, such as a phosphorothioate internucleotide linkage. The oligonucleotide may comprise one or more nucleotide with a modified sugar moiety, such as —O—CH₃ or —O—CH₂—CH₂—O—CH₃ attached to the 2′ position of the sugar moiety. The method of treatment may be for the treatment of liver inflammation. The method of treatment may be for NASH.

In a particular embodiment, there is provided an antisense oligonucleotide with a nucleobase sequence according to SEQ ID NO: 18, for use in a method of treatment. The oligonucleotide may comprise modified nucleobases (e.g. 5-methylcytosine instead of cytosine, etc). The oligonucleotide may comprise at least one modified internucleotide linkage, such as a phosphorothioate internucleotide linkage. The oligonucleotide may comprise one or more nucleotide with a modified sugar moiety, such as —O—CH₃ or —O—CH₂—CH₂—O—CH₃ attached to the 2′ position of the sugar moiety. The method of treatment may be for the treatment of liver inflammation. The method of treatment may be for NASH.

The antisense oligomer or pharmaceutical composition of the present disclosure may be administered to a subject by any suitable means. Suitable means for administering antisense oligomers, such as oligonucleotides, nucleic acids, and nucleic acid analogues, are known in the art. For instance, the antisense oligomer or pharmaceutical composition may be administered intravenously or subcutaneously.

A pharmaceutical composition of the present disclosure may be formulated for administration to any subject in need thereof. A “subject”, as used herein, may be a vertebrate, mammal, or domestic animal. Most preferably, the subject is a human.

A suitable dosing regimen may be used depending on the organism to be treated. In non-limiting examples the dosing may be 1 mg/kg to 100 mg/kg, 20 mg/kg to 60 mg/kg, or 40 mg/kg. It will be appreciated that antisense oligomers or pharmaceutical compositions according to the present disclosure may be used in a monotherapy. Alternatively, antisense oligomers or pharmaceutical compositions according to the present disclosure may be used as an adjunct to, or in combination with, known therapies. The antisense oligomers or pharmaceutical compositions may be for administration before, during or after onset of the pathological condition.

The terms “treat”, “treating”, “treatment” and the like, as used herein, unless otherwise indicated, refers to reversing, alleviating, inhibiting the process of, or preventing the disease, disorder or condition to which such term applies, or one or more symptoms of such disease, disorder or condition and includes the administration of any of the antisense oligomers, pharmaceutical compositions, or dosage forms described herein, to prevent the onset of the symptoms or the complications, or alleviating the symptoms or the complications, or eliminating the disease, condition, or disorder. For example, treatment is curative or ameliorating. As used herein, “preventing” means preventing in whole or in part, or ameliorating or controlling, or reducing or halting the production or occurrence of the thing or event, for example, the disease, disorder or condition, to be prevented.

The terms “administering”, “administer”, “administration” and the like, as used herein, refer to any mode of transferring, delivering, introducing, or transporting a therapeutic agent to a subject in need of treatment with such an agent.

It is understood that wherever embodiments are described herein with the language “comprising,” otherwise analogous embodiments described in terms of “consisting of” and/or “consisting essentially of” are also provided.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made to the Examples, which are not intended to limit the invention in any way.

### EXAMPLES

### Summary

RNA alternative splicing (AS) expands the regulatory potential of eukaryotic genomes. The liver is transcriptionally a highly complex organ. However, the mechanisms regulating liver-specific AS profiles and their contribution to liver function are poorly understood. Here, we explore the links between diet, AS and metabolic flexibility in the liver. We identify a key role for the splicing factor RNA-binding Fox protein-2 (RBFOX2) in maintaining cholesterol homeostasis in an obesogenic environment. Using enhanced individual-nucleotide resolution UV-crosslinking and immunoprecipitation (eiCLIP), we identify physiologically relevant targets of RBFOX2 in mouse liver, including the scavenger receptor class B type I (Scarb1). Our findings demonstrate that specific AS programmes actively maintain liver physiology, and underlie the lipotoxic effects of obesogenic diets when dysregulated. Using splice-switching oligonucleotides targeting this network alleviates obesity-induced inflammation in the liver and promotes an anti-atherogenic lipoprotein profile in the blood, underscoring the potential of isoform-specific RNA therapeutics for treating metabolism-associated diseases.

### Example 1—Nutrition-Promoted Changes in the Liver Splicing Machinery

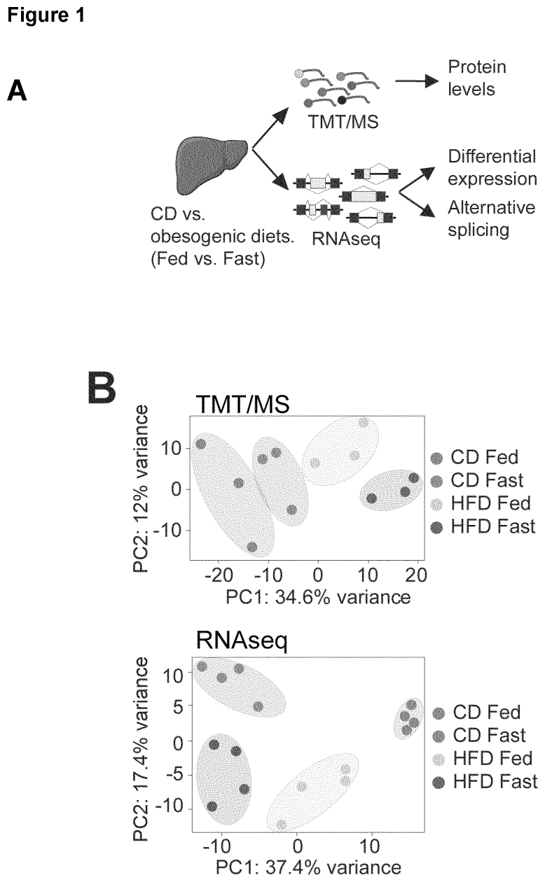

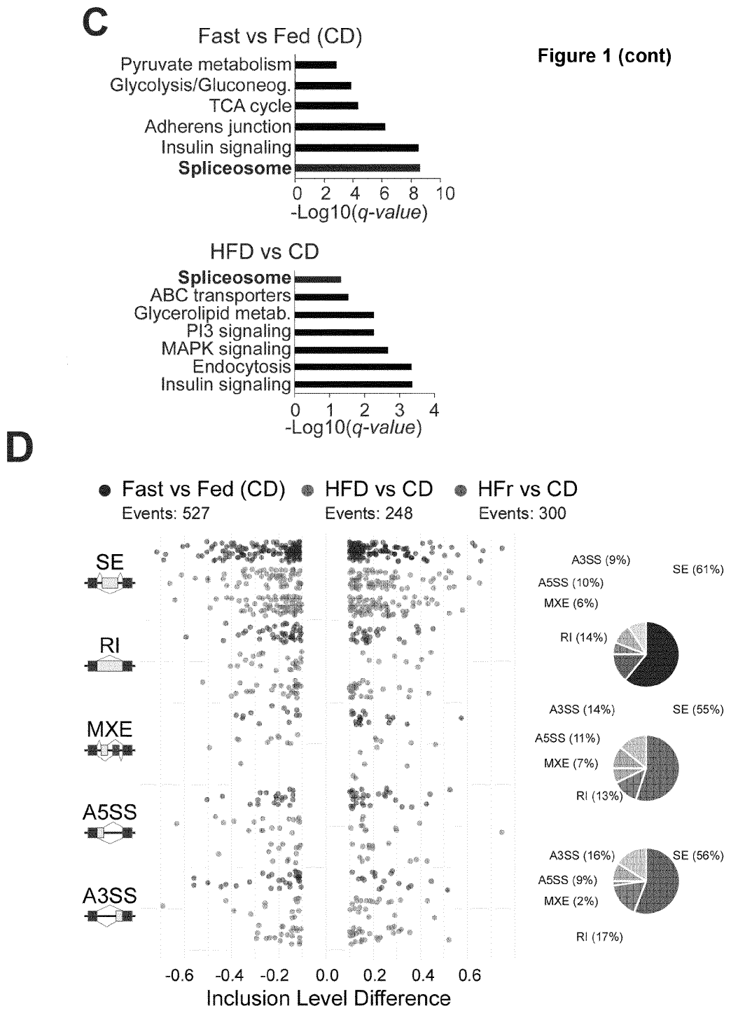

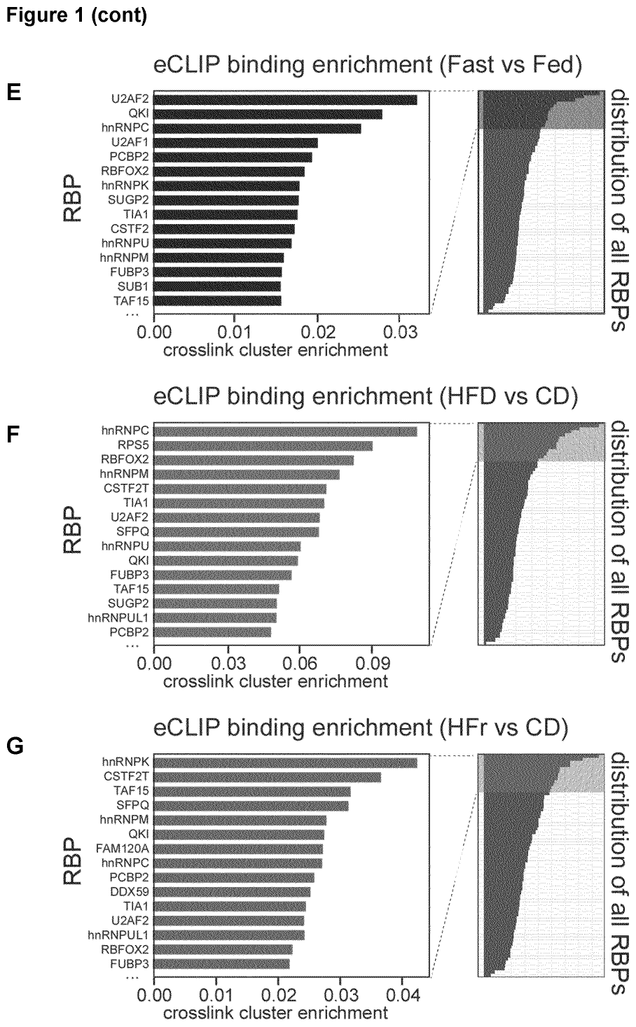

To investigate the molecular mechanisms involved in liver metabolic plasticity during health and disease, we conducted unbiased analyses of the liver transcriptome (RNAseq) and proteome (tandem mass tag; TMT/MS) of mice fed either control (CD) or high-fat (HFD) diets, in both fed and starved conditions (FIG. 1A). Proteomic analysis identified 5999 proteins in all experimental conditions (Suppl. table 1—see Paterson et al. Nature Metabolism). Principal component analysis confirmed the effect of the dietary interventions in the liver proteome (FIG. 1B, top) and transcriptome (FIG. 1B, bottom). Gene ontology analysis showed that feeding/fasting cycles specifically modify the expression of ‘spliceosome’ proteins involved pre-mRNA splicing (FDR=2.43*10⁻⁹), as well as core metabolic categories such as ‘insulin signalling’ and ‘TCA cycle’ (FIG. 1C top, FIG. 8A-B). Similarly, expression of spliceosome proteins in the liver was modified by consuming a HFD (FDR=4.72*10⁻²) (FIG. 1C bottom, FIG. 8C-D). Thus, components of the pre-mRNA splicing machinery are selectively regulated by metabolic inputs in the liver, suggesting a potential effect on pre-mRNA splicing and/or alternative splicing (AS).

Direct analysis of AS profiles by RNAseq identified significant changes associated with feeding/fasting cycles in mice fed a control diet, and with HFD (FIG. 1D). AS changes promoted by HFD included skipped exons (SE), the most abundant category (55%), followed by retained introns (RI; 13%), alternative 3′splice sites (A3SS; 14%), alternative 5′splice sites (A5SS; 11%), and mutually exclusive exons (MXE; 7%). Increased sugar consumption is a significant contributor to diet-induced liver disease and associated cardiometabolic disease. We investigated AS events promoted by a high-fructose (HFr) diet as an alternative model of diet-induced obesity¹⁶. AS changes associated with HFr diet included skipped exons (SE), the most abundant AS event identified in these conditions (56%), followed by retained introns (RI; 17%), alternative 3′ splice sites (A3SS; 16%), alternative 5′ splice sites (A5SS; 9%) and mutually exclusive exons (MXE; 2%), (FIG. 1D). AS changes promoted by feeding/fasting cycles in CD mice were strongly attenuated in diet-induced obesity (FIG. 8E) suggesting that perturbation of liver AS networks contributes to the reduced metabolic plasticity observed in obesity. Collectively, these results reveal specific changes in pre-mRNA AS programs in physiological (feeding/fasting cycles) and pathophysiological (HFD- and HFr-induced obesity) adaptations.

### Example 2—Splicing Factor RBFOX2 is Modulated by Diet in the Liver

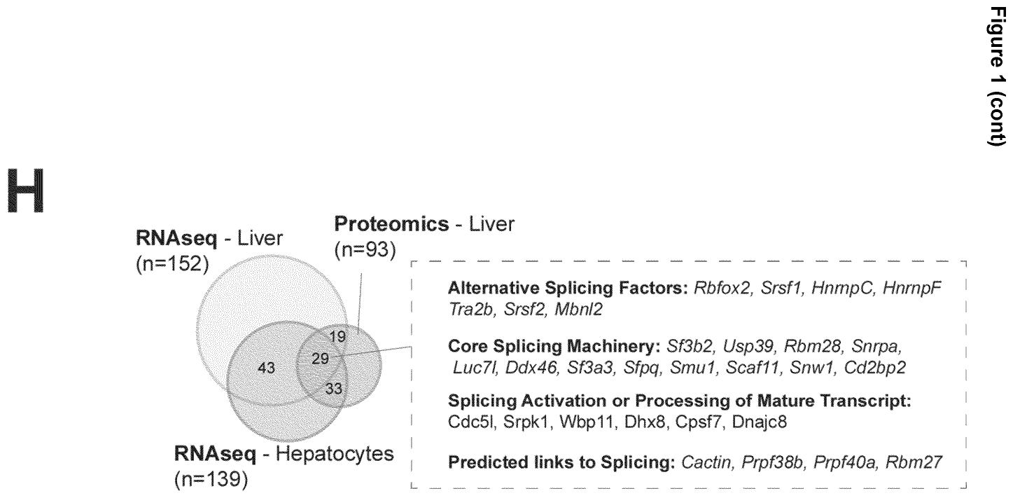

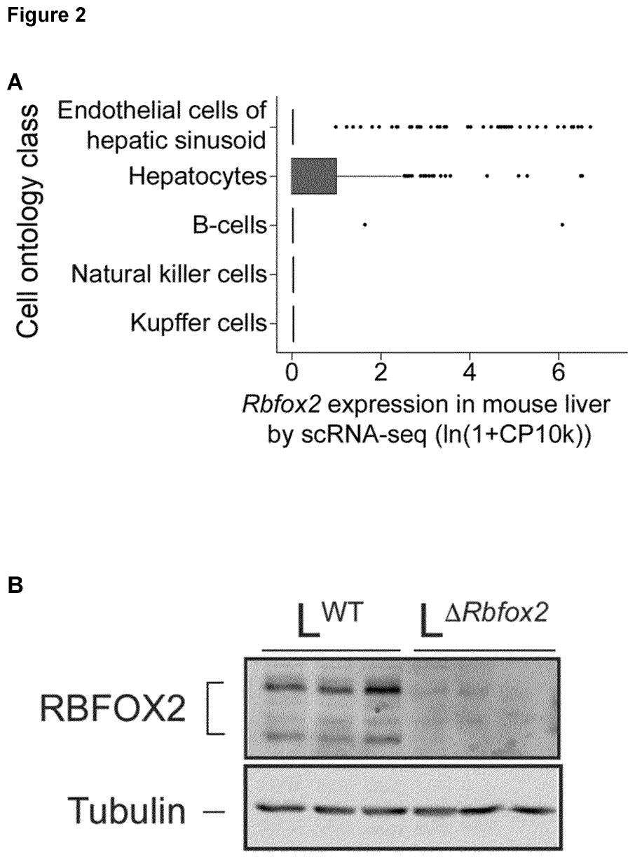

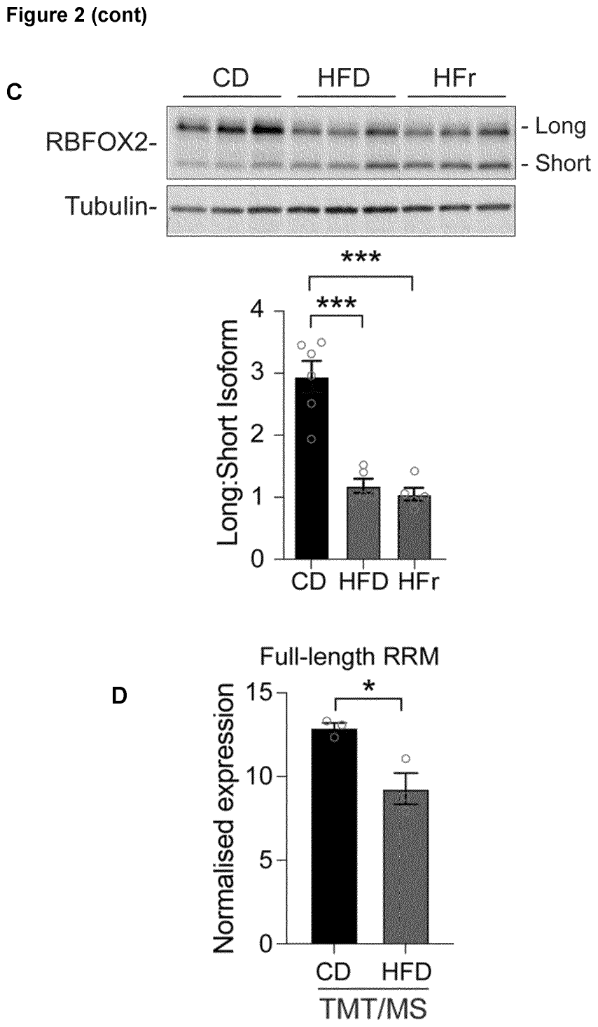

We investigated which splicing factors (SFs) were promoting the changes detected in liver AS profiles in different nutritional states. We reasoned that SFs controlling splicing networks in the liver should (a) show detectable expression in liver and/or hepatocytes and (b) have enriched binding at regions within or surrounding the alternatively spliced exons in the liver. We thus performed an unsupervised motif enrichment analysis of the sequences within and surrounding alternatively spliced exons in physiological (feeding/fasting cycles) and pathological (diet-induced obesity) states in the liver. This revealed a significant enrichment of SF binding motifs including RBFOX2, CUGBP2, SRSF1, PTBP1, and MBN1 (FIG. 8FG). Analysis of SFs with conserved crosslinking peaks within and surrounding AS exons in human liver cells¹⁷ identified eight SFs (U2AF2, RBFOX2, QKI, hnRNPC, PCBP2, TIA1, hnRNPM, and TAF15) as the top 20% ranking factors in all three comparisons analysed: feeding/fasting cycles (FIG. 1E), HFD-induced obesity (FIG. 1F), and HFr-induced obesity (FIG. 1G). Additional analysis confirmed that RBFOX2 is expressed in the liver (FIG. 1H). Furthermore, analysis of a mouse single-cell RNAseq dataset¹⁸ showed that hepatocytes account for most of the Rbfox2 expression in the liver, although Rbfox2 is also detected in endothelial cells (FIG. 2A). Other splicing factors potentially involved in AS regulation, showed a broader expression across other liver-resident cell populations (FIG. 9). We generated Alb_cre⁻Rbfox2^{LoxP/LoxP} (L^{WT}) and Alb_cre⁺Rbfox2^{LoxP/LoxP} (L^{ΔRbfox2}) mice to inactivate the Rbfox2 gene selectively in hepatocytes. Western blot analysis showed that RBFOX2 is undetectable in the liver of L^{ΔRbfox2} mice, confirming that hepatocytes account for most of its expression in the liver (FIG. 2B). These results suggest a relevant role for RBFOX2 in regulating AS in hepatocytes.

Our proteomic and transcriptomic analysis showed that Rbfox2 is regulated at the transcriptional level by feeding/fasting cycles in the liver (FIG. 8A-B). The use of alternative promoters and AS can generate multiple RBFOX2 isoforms with different splicing activity¹⁹ including a dominant negative form, characterized by a truncated RNA-recognition motif (RRM). Western blot analysis showed that both HFD- and HFr-diet-induced obesity are associated with decreased expression of the main RBFOX2 isoform in the liver (FIG. 2C). Moreover, TMT/MS proteomic analysis shows that these changes are associated with decreased levels of full-length active RBFOX2 (FIG. 2D) suggesting a potential RBFOX2 loss-of-function in the liver in diet-induced obesity. Collectively, these data suggest that RBFOX2 could play a specific role in coordinating dynamic AS changes in response to physiological and pathological metabolic signals.

### Example 3—RBFOX2 Controls Cholesterol-Regulating Genes Via AS

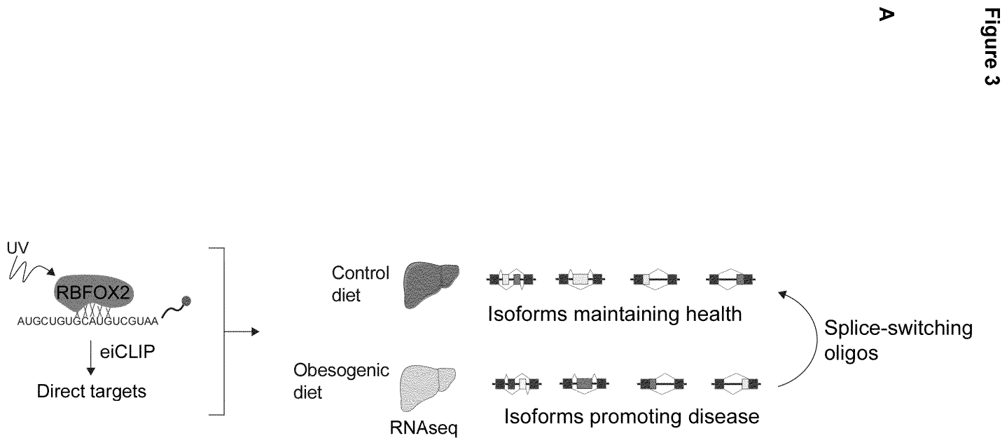

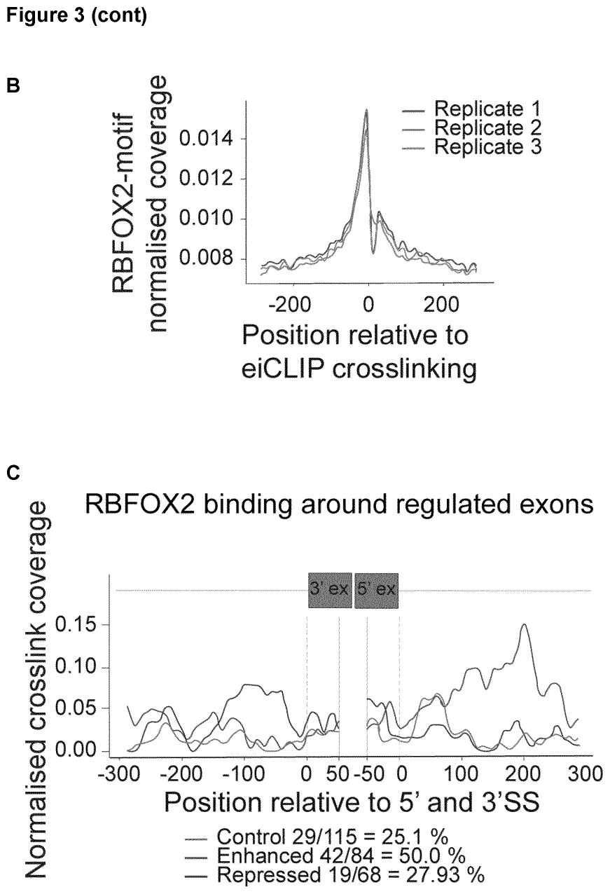

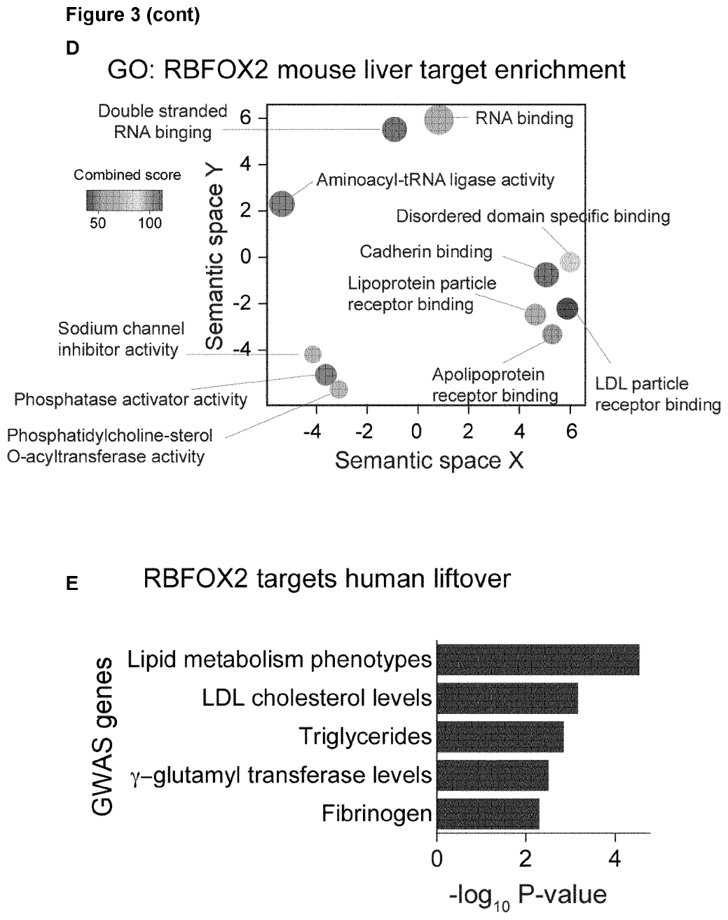

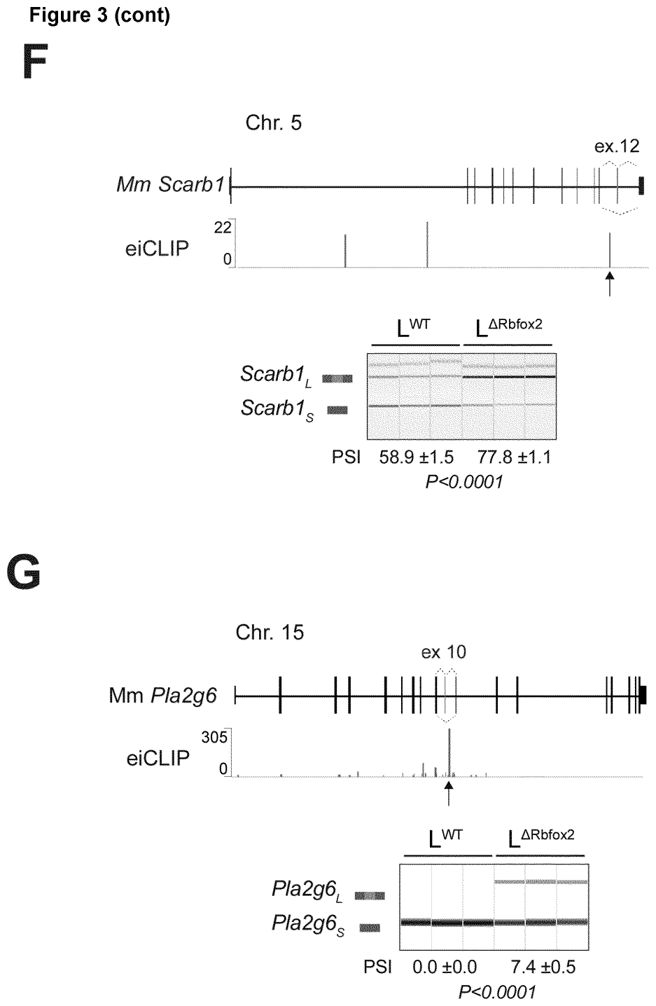

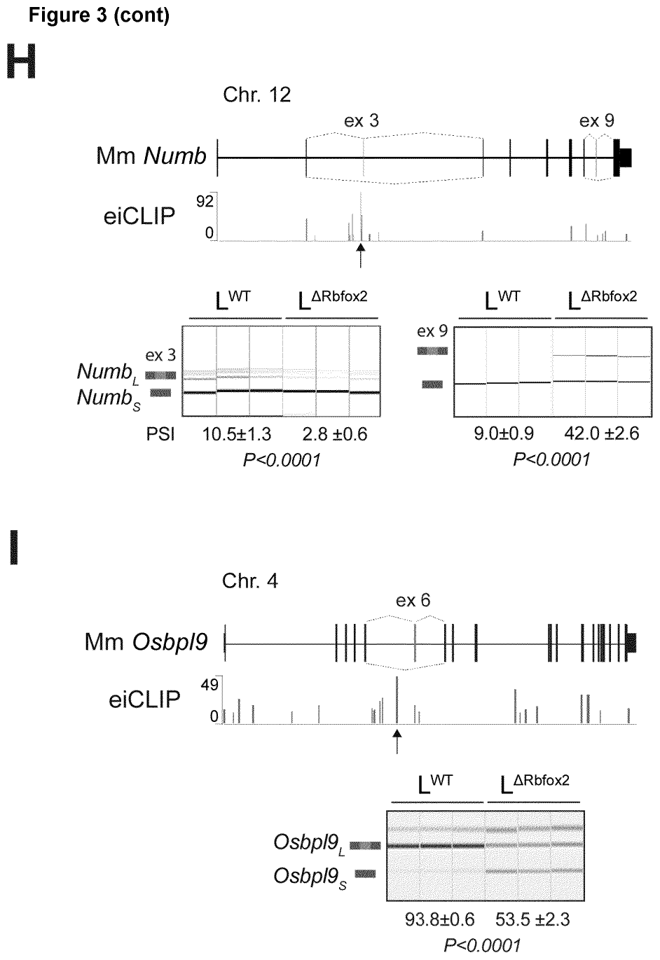

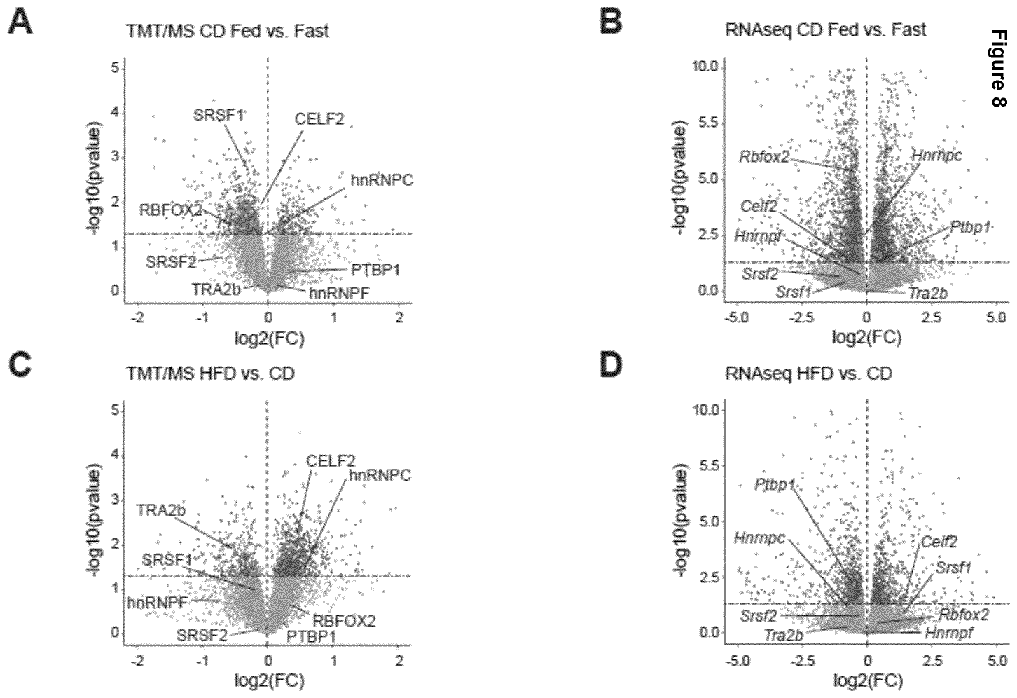

Splicing factors frequently regulate AS through interconnected cis- and trans-mediated effects^{20, 21}. However, identification of direct targets for endogenous splicing factors in the liver has been hampered by rapid pre-mRNA degradation during crosslinking and immunoprecipitation (iCLIP) analysis in liver samples. For this reason, there is a very limited information about AS programmes in adult liver that contribute to maintain or perturb homeostasis. To overcome this problem, we employed an ‘enhanced individual nucleotide-resolution iCLIP’ (eiCLIP) protocol with an expedited and improved library preparation workflow (see methods) to significantly enhance the recovery of RBFOX2-crosslinked premRNA products (FIG. 3A; 10A). The specificity of the signal was confirmed by peak analysis, showing an enrichment in the previously described RBFOX2 consensus (U)GCAUG binding motif²² (FIG. 3B). Additionally, our analysis revealed that in hepatocytes, direct RBFOX2 targets included previously described bona fide targets such as Ptbp2 and Snrnp70²⁰ (FIG. 10B-C).

The RBFOX2 protein promotes or represses AS in a position-dependent manner^{20, 22-27} (FIG. 10D). Analysis of RBFOX2 crosslinking positions upstream or downstream AS exons (identified by RNAseq in L^{WT} and L^{ΔRbfox2} mice; FIG. 10E) showed that in the liver, this positional effect is more robust in enhanced exons (50.0%) than in repressed exons (27.9%) compared to control exons (25.1%) (FIG. 3C).

Gene ontology analysis showed that RBFOX2-crosslinked clusters are highly enriched in transcripts encoding for proteins involved in phosphatidylcholine-sterol-O-acyltransferase activity (adj. p-value=3.66*10⁻²), lipoprotein particle receptor binding (adj. p-value=1.13*10⁻²), apolipoprotein receptor binding (adj. p-value=2.61*10⁻³), and LDL particle receptor binding, suggesting a role for RBFOX2 in controlling lipid metabolism (FIG. 3D). In addition, other targets are involved in functions including cadherin binding (adj. p-value=7.42*10⁻¹⁶), disordered domain specific binding (adj. p-value=2.72*10⁻⁴) and also RNA binding proteins (RBPs) (adj. p-value=3.72*10⁻¹⁴), underscoring a role for RBFOX2 in modulating additional layers of transcriptional regulation²⁰. The list of RBFOX2-targets contributing to each gene ontology category are included in Suppl. Table 2 (see Paterson et al. Nature Metabolism).

The human orthologous genes of the mouse RBFOX2 targets detected by eiCLIP are highly enriched in genes implicated by GWAS in human lipid metabolism phenotypes, LDL cholesterol levels or triglycerides (FIG. 3E). These include the Scarb1 gene, which encodes the class B scavenger receptor SR-BI, a HDL receptor that mediates cholesterol uptake and modifies plasma HDL and bile cholesterol^{28, 29}, Pla2g6 which is a phospholipase A2 group VI³⁰, Sec31a, a core component of the COPII vesicle trafficking system involved in SREBP1 activation³¹ and processing of ApoB-containing lipoprotein³², the oxysterol-binding protein Osbpl9, and Numb an adaptor protein involved in clathrin-dependent reverse cholesterol transport from bile³³.

Further analysis of eiCLIP profiles at the Scarb1 pre-mRNA transcript revealed that RBFOX2 binds upstream of exon 12. PCR analysis of the livers of L^{ΔRbfox2} mice confirmed that RBFOX2 promotes Scarb1 exon 12 skipping (FIG. 3F). In addition, RBFOX2 promotes skipping or inclusion of specific exons in Pla2g6 (FIG. 3G), Numb (FIG. 3H), Osbpl9 (FIG. 3I) and Sec31a (FIG. 10F). Analysis of RBFOX2 binding in human hepatoma cells confirmed the conservation of RBFOX2 crosslinking within alternatively spliced exons in the orthologous human transcripts (FIG. 10G-J). Collectively, these results reveal that RBFOX2 directly regulates AS of a network of genes involved in lipid homeostasis in the liver.

### Example 4—RBFOX2 Regulates Cholesterol Metabolism in Obesogenic Diets

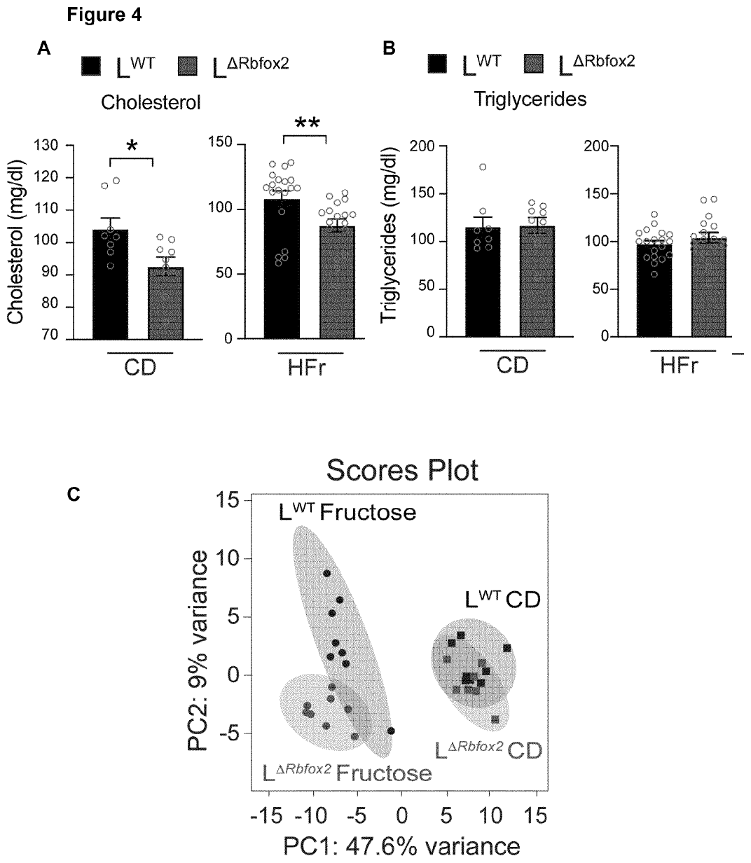

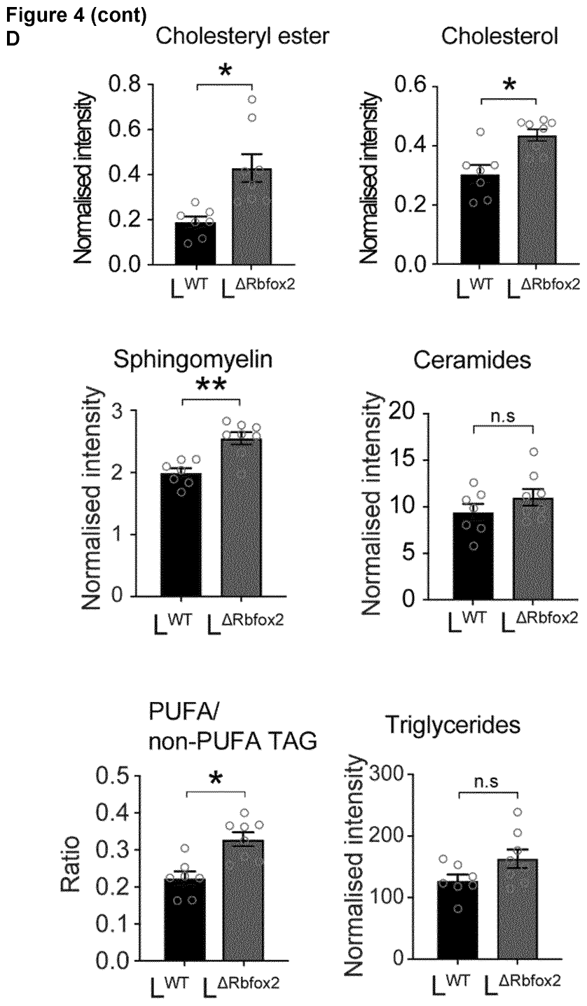

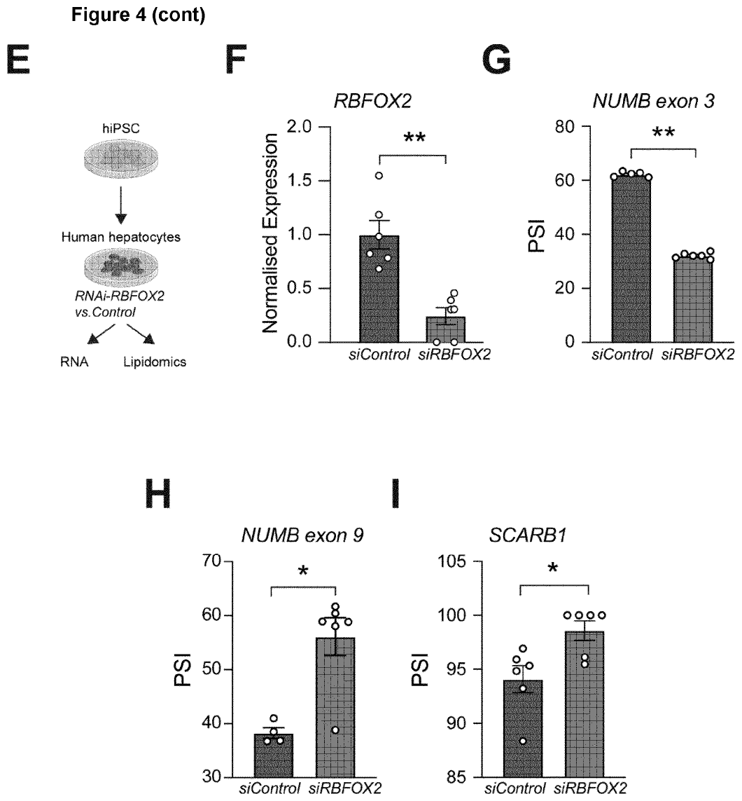

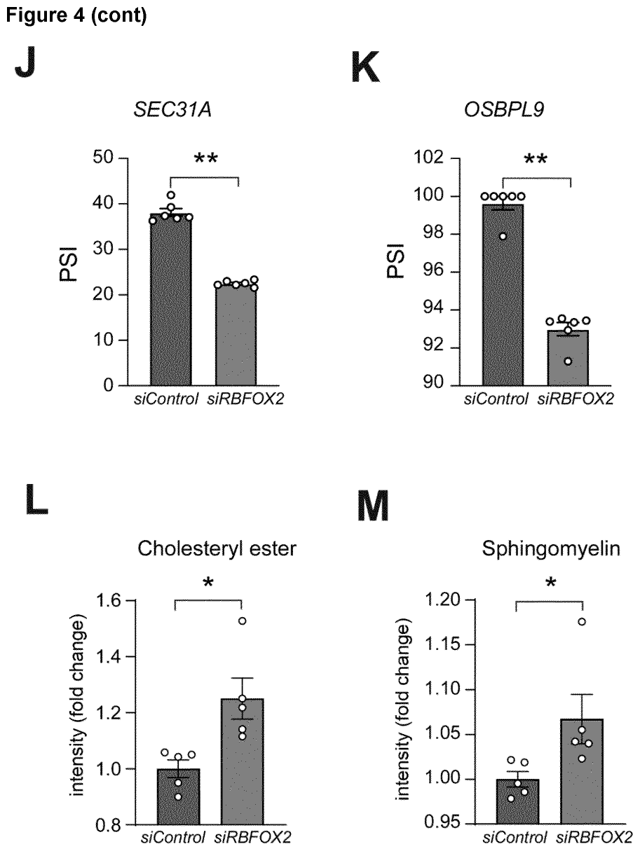

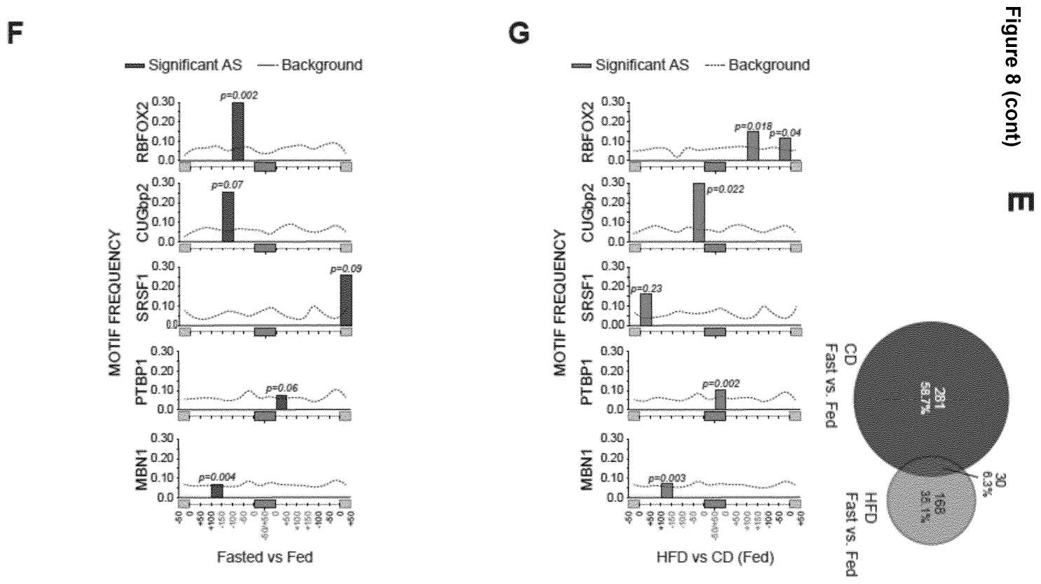

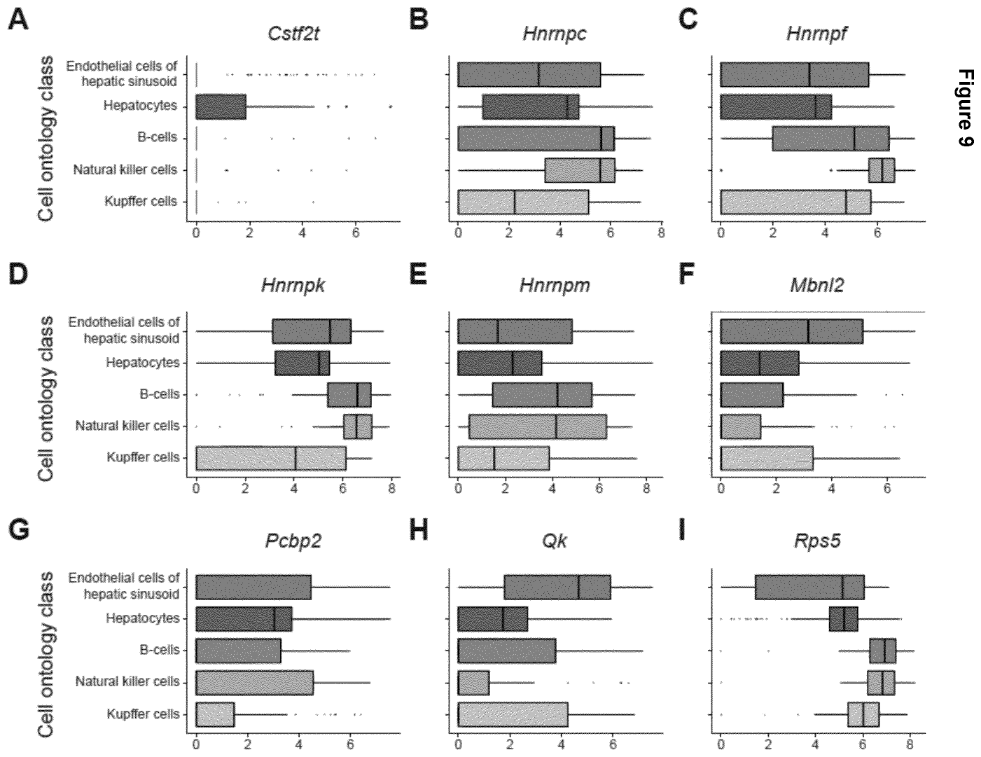

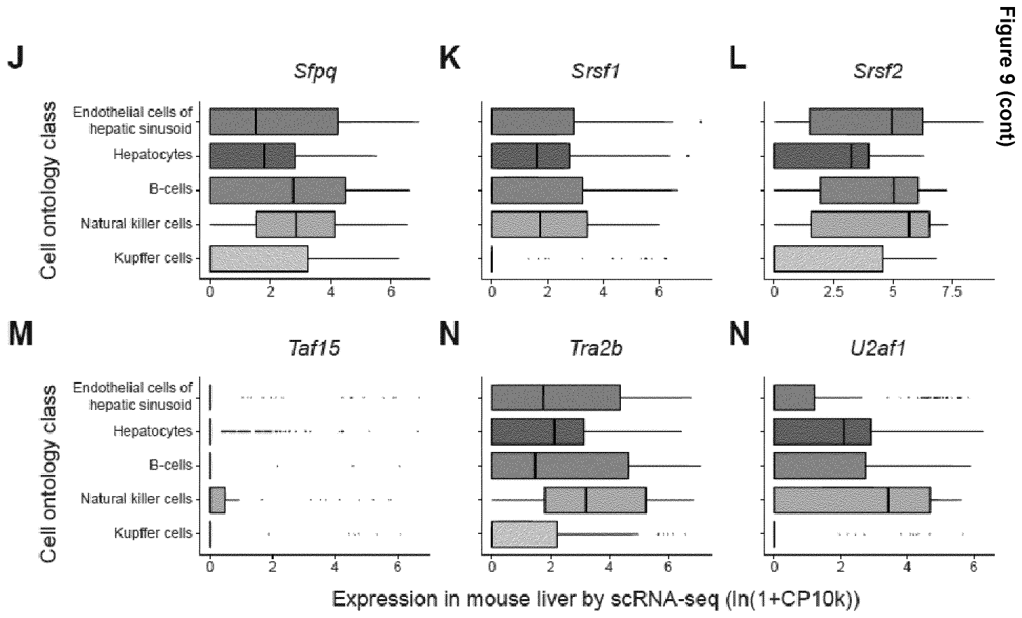

L^{ΔRbfox2} mice are viable and are born at expected Mendelian ratios. When fed control chow, a HFD or a HFr diet, L^{ΔRbfox2} mice did not show significant changes in body weight (FIG. 11A-C) or glucose tolerance (FIG. 11D-F) suggesting that RBFOX2 ablation does not interfere with normal liver development. In contrast to the observed normal glucose homeostasis, blood lipid profile analysis revealed that L^{ΔRbfox2} male mice showed a significant decrease in total cholesterol (FIG. 4A) but not in triglycerides (FIG. 4B), when consuming a HFr diet. Similar results were obtained when studying L^{WT} and L^{ΔRbfox2} female mice (FIG. 11G-H).

The liver plays a central role in lipid homeostasis. To investigate the metabolic changes associated with this altered blood lipid profile, livers from L^{WT} and L^{ΔRbfox2} mice were analysed by untargeted LC/MS lipidomics. Principal component analysis showed that the HFr diet was associated with marked changes in the overall metabolomic profile compared to CD samples (FIG. 4C). In mice fed a HFr diet, ablation of RBFOX2 was associated with an increase in hepatic lipid content, particularly cholesteryl esters (CE), total cholesterol and sphingomyelins (FIG. 4D). While we did not observe differences in steatotic patterns (FIG. 11I), we noted a change in triglyceride (TG) composition favouring longer polyunsaturated fatty acyl chains (as denoted by the PUFA/Non-PUFA TG ratio) in L^{ΔRbfox2} mice (FIG. 4D). When fed a HFD, L^{ΔRbfox2} mice also showed an increase in intrahepatic total cholesteryl esters. However, contrary to mice fed a HFr diet, they showed no overall changes in cholesterol or sphingomyelins (FIG. 11J-M), nor changes in blood cholesterol (FIG. 11N), suggesting a partial phenotype that is exacerbated when L^{ΔRbfox2} mice are fed a pro-lipogenic HFr diet.

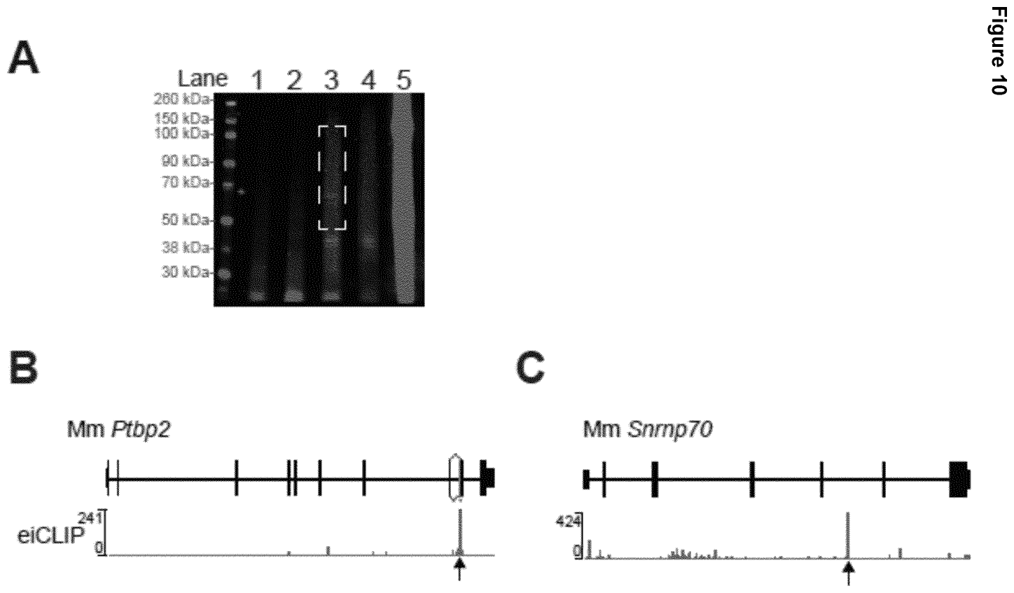

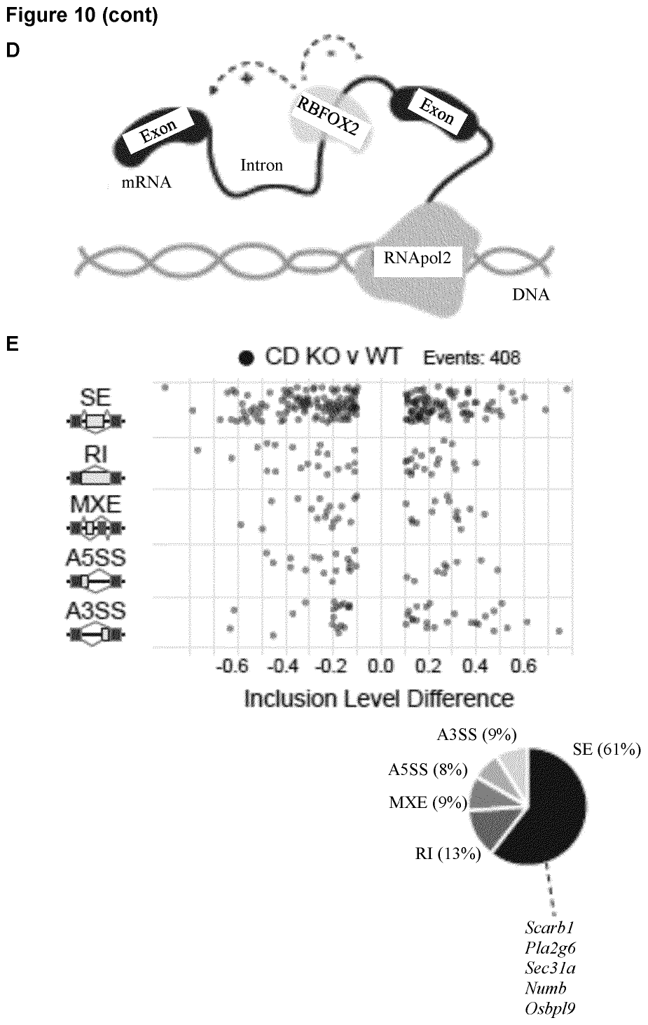

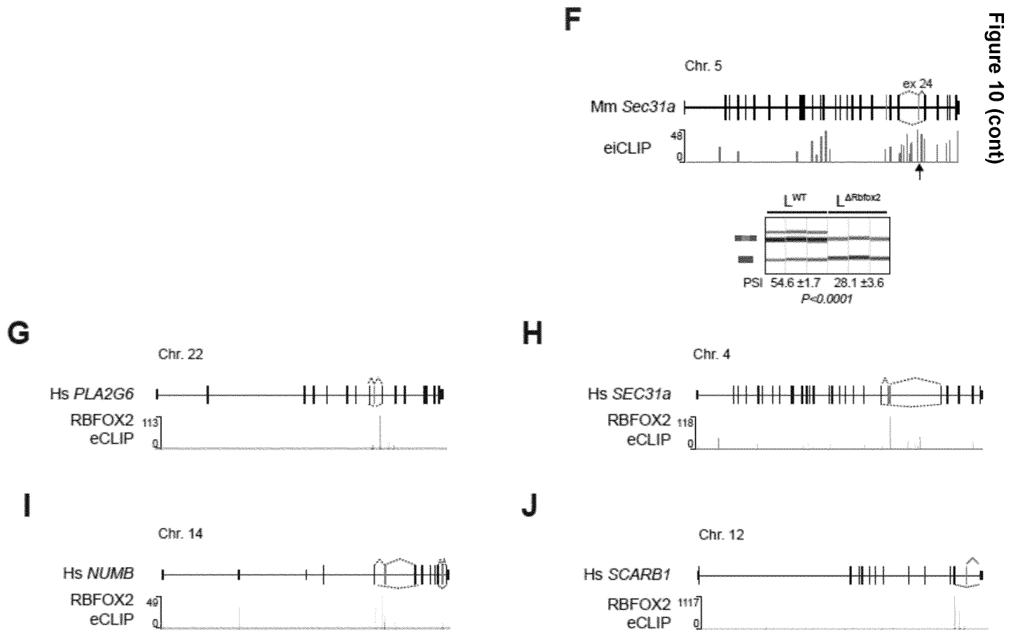

CLIP-seq analysis suggests a conservation in RBFOX2 targets in humans (FIG. 10G-J). To interrogate if RBFOX2 is involved in lipid metabolism in human hepatocytes, we obtained iPSC-derived hepatocytes and used siRNA to silence RBFOX2 (FIG. 4E). Efficient RBFOX2 silencing was confirmed by qPCR analysis (FIG. 4F). RBFOX2 silencing did not affect the expression of mature hepatocyte/differentiation markers such as ASGPR2, SERPINA1 and SERPINA2 (FIG. 12A). Consistent with human RBFOX2 binding to pre-mRNA, the effect of RBFOX2 deficiency in alternative splicing of SCARB1, SEC31A, OSBPL9 and PLA2G6 and NUMB is maintained in human hepatocytes (FIG. 4G-J), confirming the conservation of this regulatory network. Moreover, lipidomics analysis of human hepatocytes showed that RBFOX2 silencing promotes changes in lipid composition (FIG. 12B) including the accumulation in CE and SM (FIG. 4L-M). These results further support the role of RBFOX2 in controlling a conserved AS network involved in lipid metabolism.

The simultaneous accumulation of cholesterol in the liver of L^{ΔRbfox2} mice and the decrease in cholesterol in the plasma suggested that RBFOX2 plays a specific role in the regulation of hepatic cholesterol uptake, trafficking and/or efflux. To gain insight into these mechanisms, we used RNAseq to compare the transcriptome of L^{WT} and L^{ΔRbfox2} mice fed a HFr diet. Ablation of RBFOX2 in the liver led to changes in the cholesterol biosynthesis (ratio=0.138; p-value=1.7*10⁻⁵), the mevalonate pathway (ratio=0.214; p-value=5.25*10⁻⁵), the LXR pathway (ratio=0.02; p-value=2.75*10⁻²) and the FXR pathway (ratio=0.02; p-value=3.09*10⁻²), consistent with a role of RBFOX2 in lipid and cholesterol metabolism. Additional changes were found in oxidative phosphorylation (ratio=0.138; p-value=2.51*10⁻¹⁷) and mitochondrial dysfunction (ratio=0.0877; p-value=2.51*10⁻¹⁴).

Cholesterol metabolism is controlled by a feedback mechanism involving sterol regulatory element-binding proteins (SREBPs), an ER-resident family of transcription factors³⁴. An ad hoc qPCR analysis of genes involved in cholesterol homeostasis showed that increased intrahepatic cholesterol is not associated with increased expression of key biosynthetic genes including Srebf2, Hmgcs, and Hmgcr (FIG. 12C), suggesting that this feedback regulation remained intact. Increase in the expression of Abca1, Abcg8 and Nr1h2 (LXR beta) and Nr1h3 (LXR alpha) genes further suggests a compensatory increase in cholesterol efflux pathways caused by cholesterol accumulation upon RBFOX2 deficiency (FIG. 12C). Consistent changes for ApoB were also observed at protein level, although the difference between L^{WT} and L^{ΔRbfox2} for ABCA1 was not statistically significant (FIG. 12D).

Reverse HDL-cholesterol uptake and conversion into bile acids in the liver is a major pathway for cholesterol excretion through the bile. We hypothesised that decreased cholesterol in blood of L^{ΔRbfox2} mice and simultaneous increase in intrahepatic cholesterol could lead to an increase in bile acid levels. Consistent with this hypothesis, LC-MS/MS analysis showed an increase in bile acids such as taurocholic, taurodeoxycholic, tauroursodeoxycholic, and cholic acids (FIG. 12E-K). When fed a HFD, L^{ΔRbfox2} mice also showed an increase in intrahepatic bile acids compared to L^{WT} control mice (FIG. 12L-U). Altogether, these results show that liver-specific ablation of Rbfox2 leads to cholesterol accumulation in the liver, increased conversion to bile acids and subsequent activation of LXR and FXR pathways.

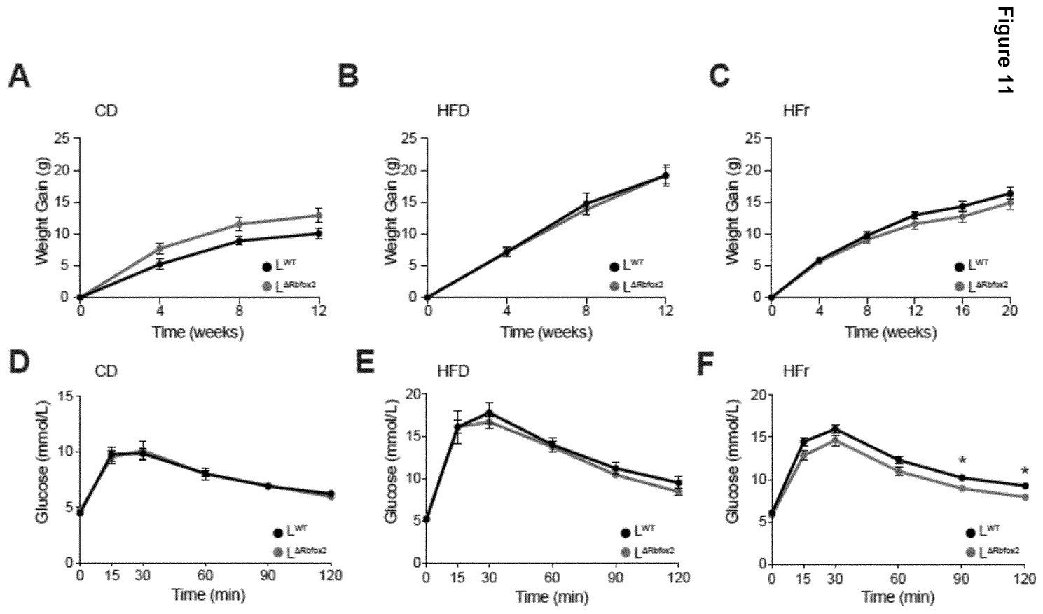

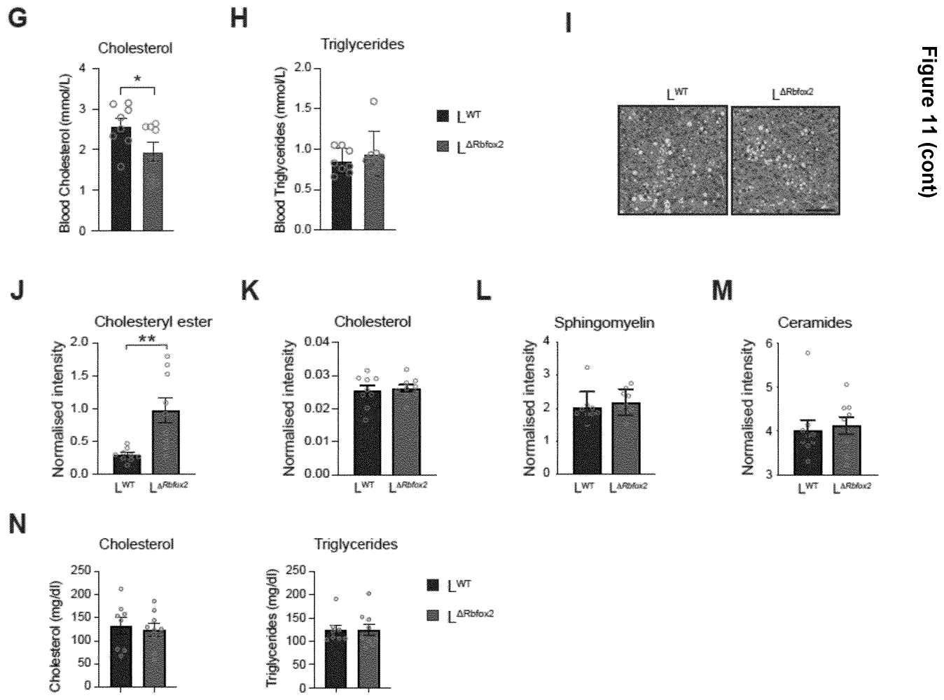

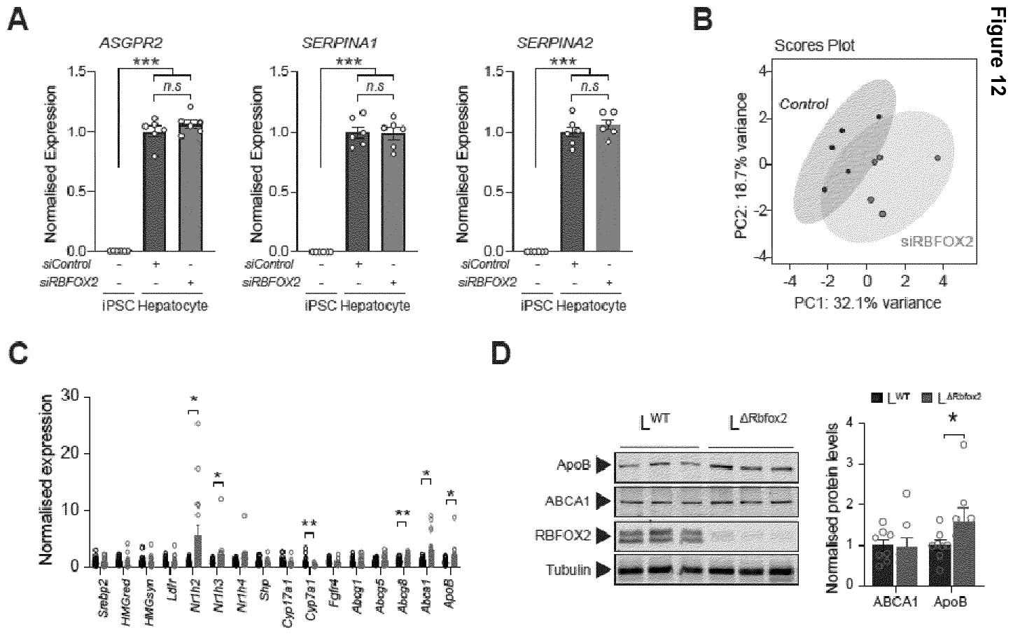

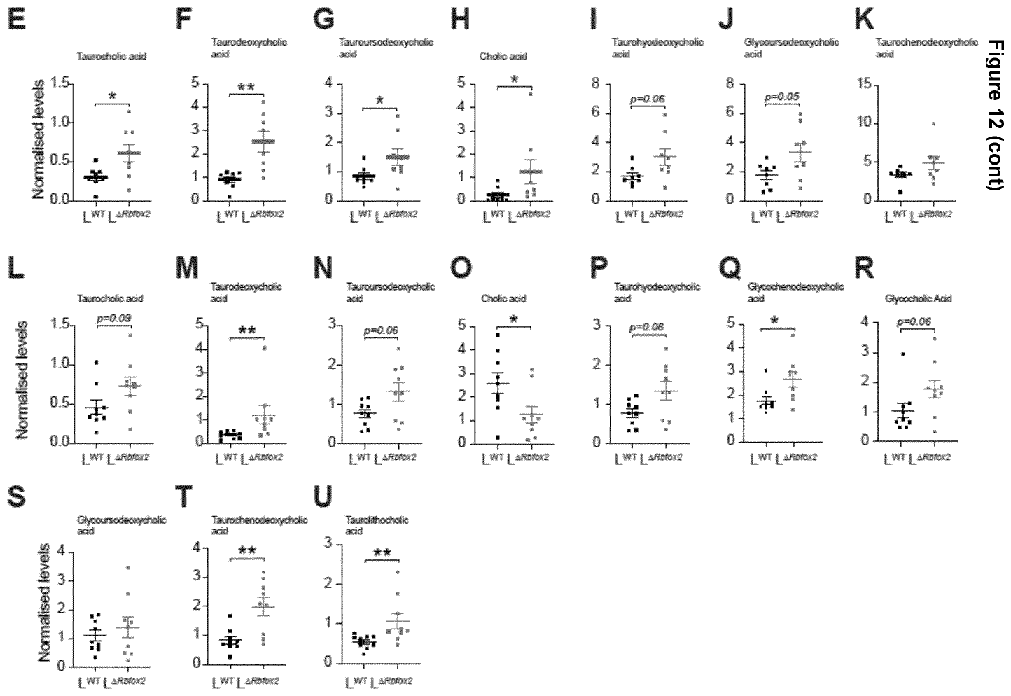

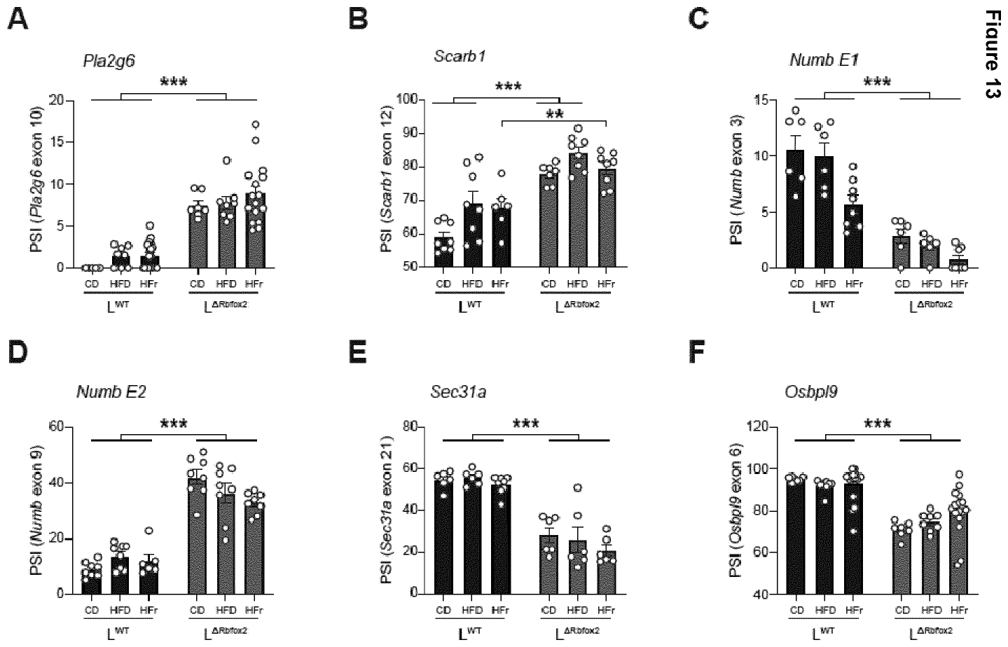

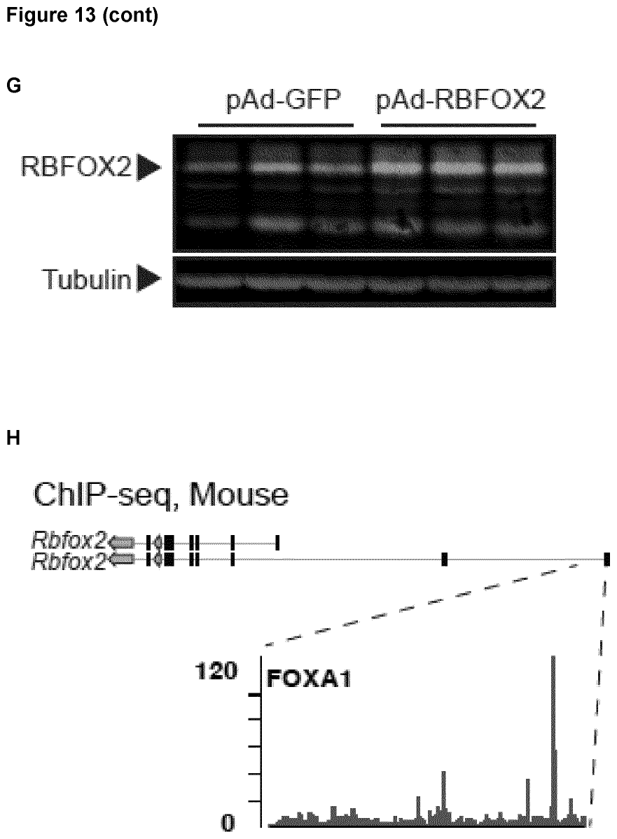

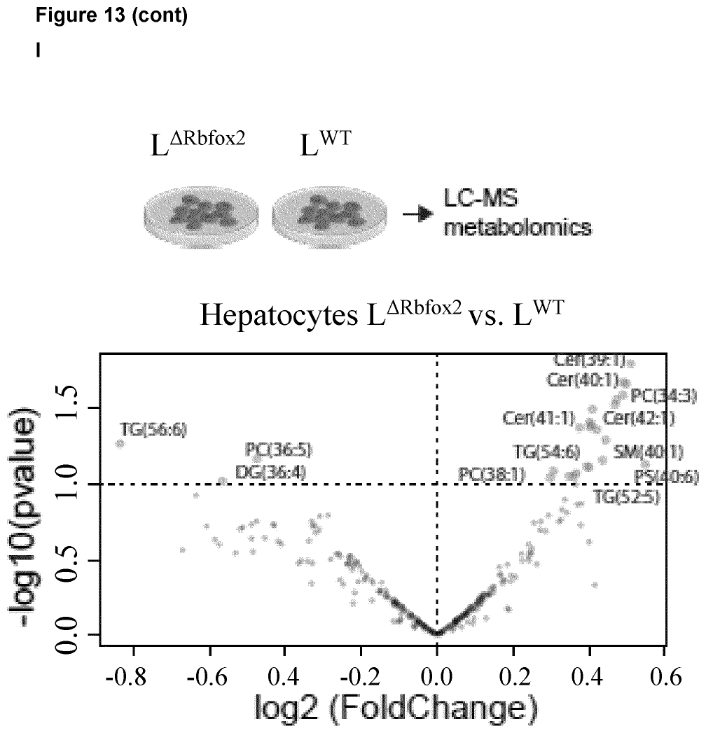

Next, we hypothesized that changes in RBFOX2 activity could coordinate changes in this downstream AS network in response to diet. To test this idea we analysed AS changes in Scarb1, Pla2g6, and Numb in L^{WT} and L^{ΔRbfox2} mice fed a control or an obesogenic diet. Consistently with our hypothesis, HFr promotes significant AS changes in L^{WT} mice, and L^{ΔRbfox2} mice fail to induce obesity-specific AS events at Pla2g6 (FIG. 13A), Scarb1 (FIG. 13B), and Numb (FIG. 13C). Analysis of other targets such as Sec31a and Osbpl9 confirmed the strong regulation by RBFOX2 (FIG. 13E-F), however, the effect of the diet was not detected, suggesting that other factors might play a role in the regulation of these genes under obesogenic conditions.

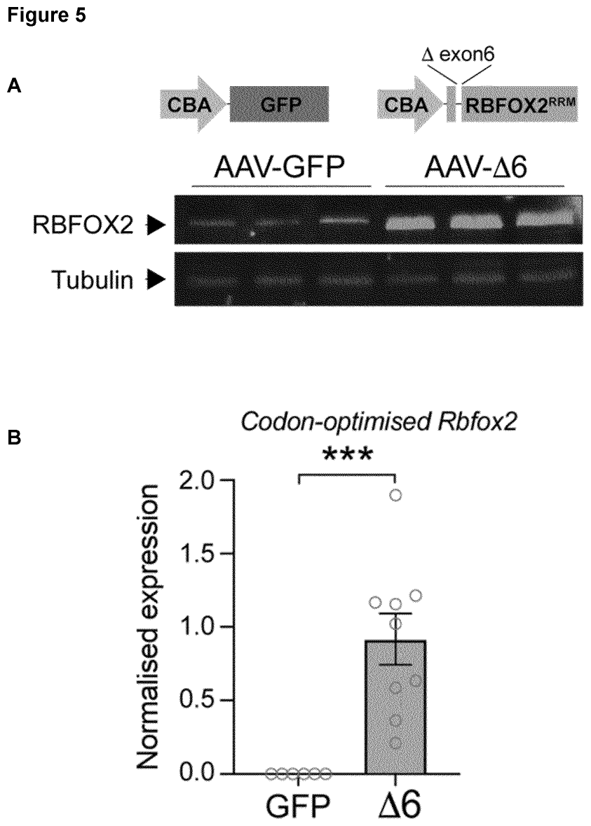

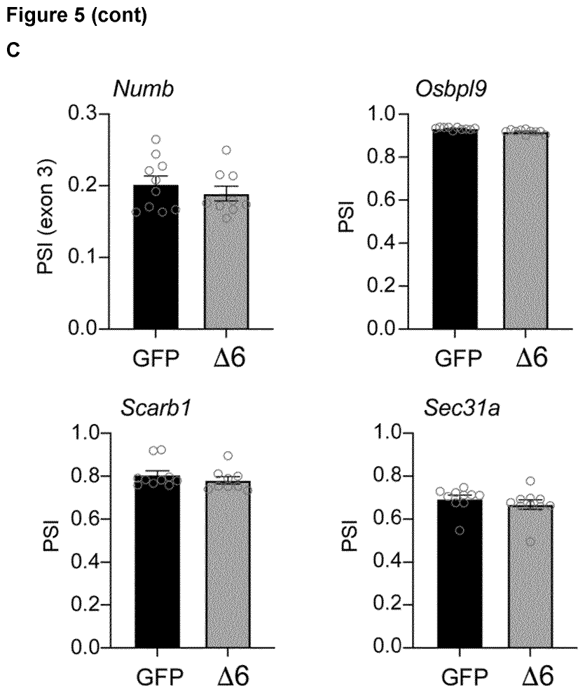

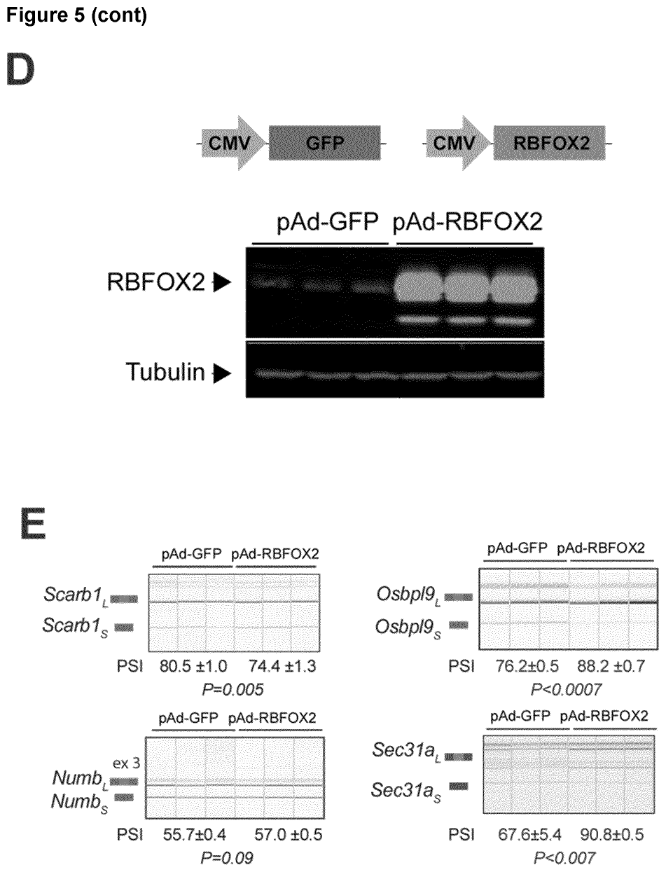

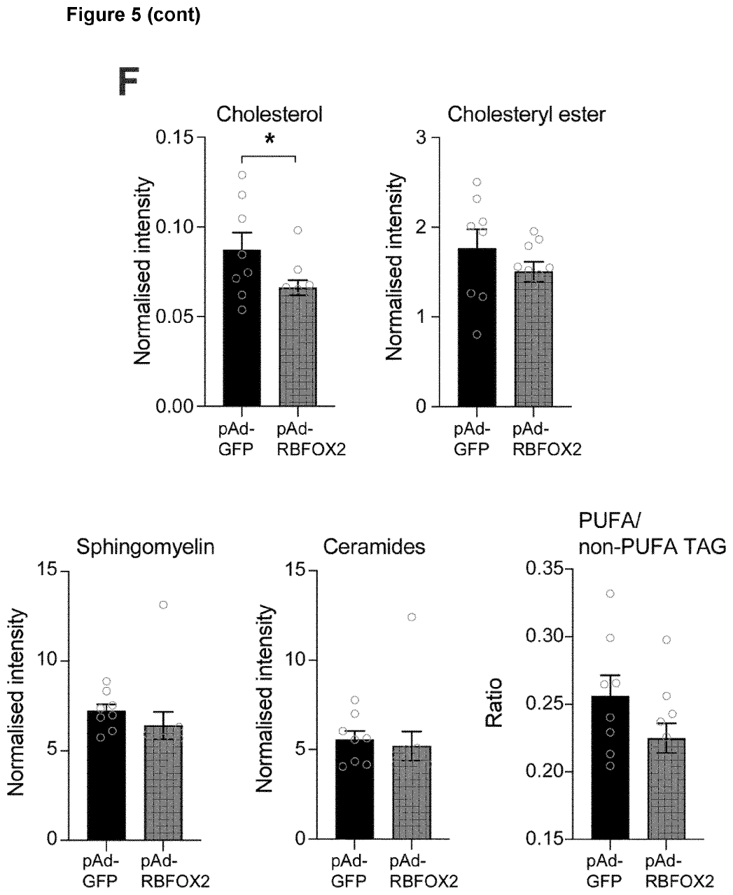

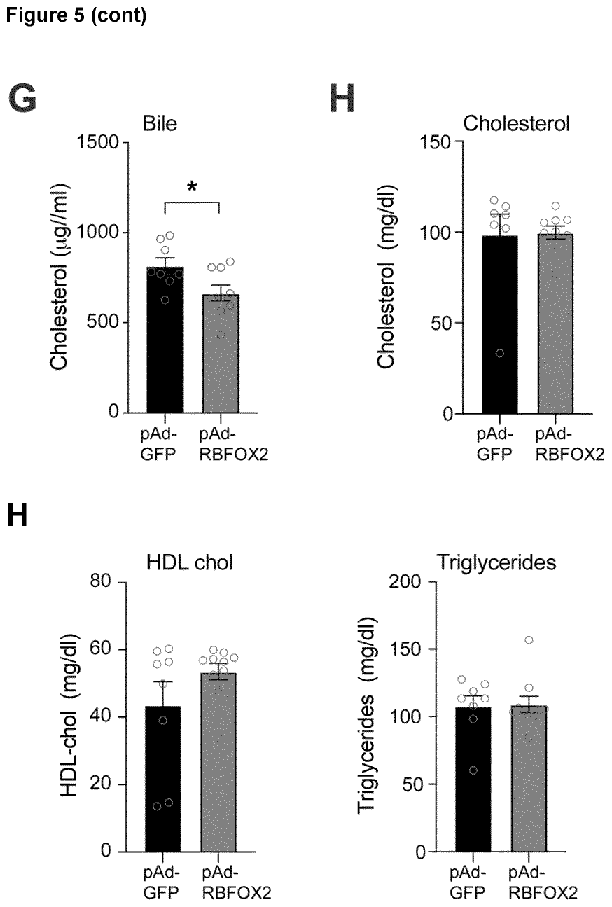

In order to get further mechanistic insights into the regulation of this AS network by RBFOX2 we interrogated if alternative splicing changes are associated with the decreased levels of active RBFOX2 or increased levels of the inactive (lacking RRM motif) protein (FIG. 2C-D). To investigate this point we generated an AAV vector expressing a RBFOX2 lacking the RRM motif (RBFOX2-A6) (FIG. 5A). Expression was confirmed by western blot and qPCR (FIG. 5A-B). However, overexpression of RBFOX2-A6 did not mimic the AS changes associated with RBFOX2-inactivation in mouse and human hepatocytes (FIG. 5C). We next generated a vector overexpressing full-length RBFOX2 (containing RRM motif) (FIG. 5D; 13G). Notably, overexpression of active RBFOX2 promotes AS changes in the opposite direction to RBFOX2-deficiency (FIG. 5E; 13G), suggesting that the levels of active RBFOX2 are more relevant for AS regulation in the liver, than the expression of the dominant negative form.

LC-MS/MS lipidomics analysis showed that RBFOX2 overexpression is associated with a decrease in cholesterol in the liver (FIG. 5F) and a decrease in cholesterol in the bile (FIG. 5G). The transient overexpression of RBFOX2 was associated with a mild increase in blood HDL-cholesterol that didn't reach statistical significance (FIG. 5H). Collectively these results confirm that RBFOX2 regulates a network of genes involved in lipid metabolism, and changes in full-length RBFOX2 expression modify cholesterol distribution under a lipogenic diet.

### Example 5—Liver Expression of RBFOX2 is Controlled by FOXA1/2

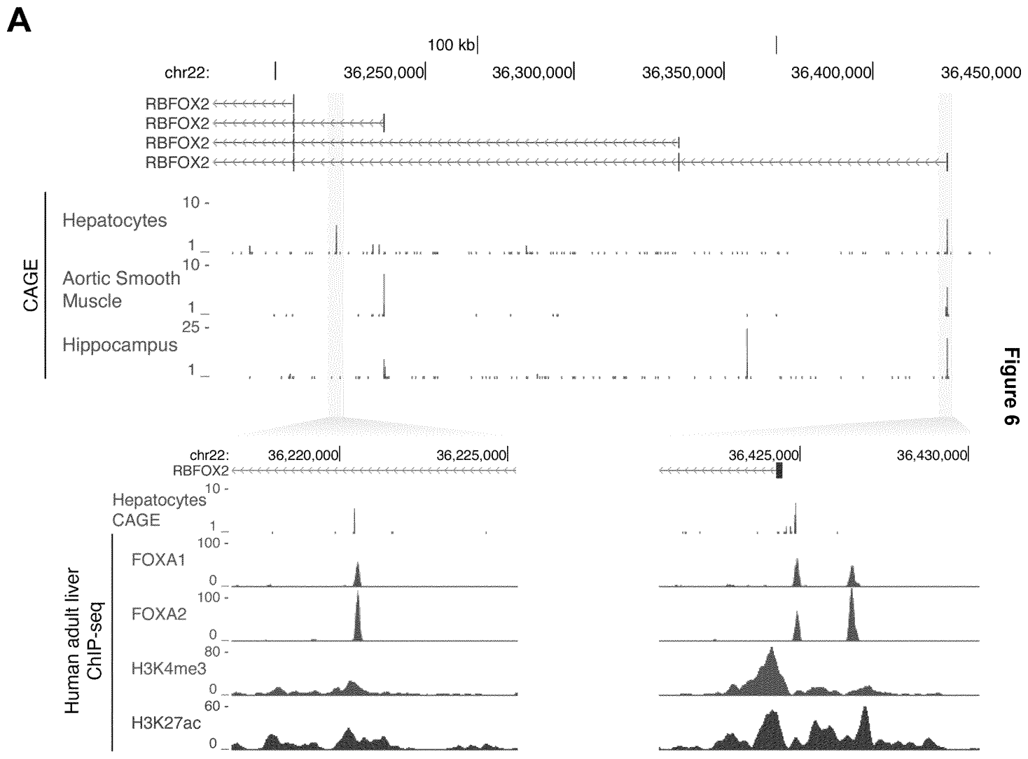

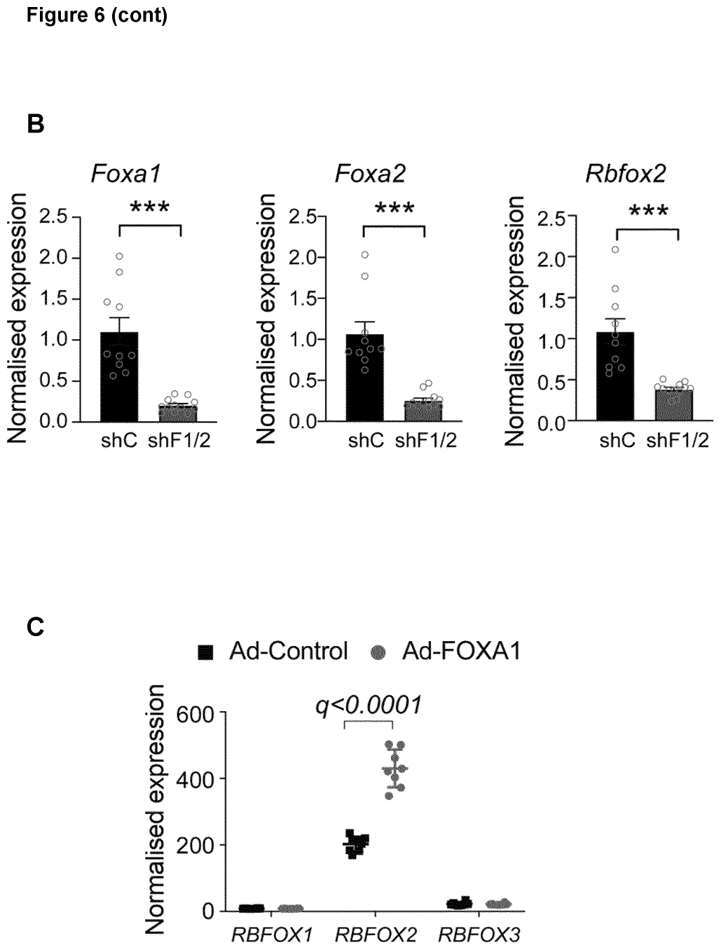

To characterise the upstream regulators of RBFOX2 in the human liver, we used FANTOM5 CAGE datasets of transcription initiation sites, as the RBFOX2 gene has a complex architecture with multiple promoters¹⁹ (FIG. 6A). This analysis concluded that the expression of RBFOX2 in the human hippocampus or aortic smooth muscle is driven by two promoters, a proximal promoter and a distal one, whose activity levels are similar. On the other hand, in hepatocytes it is the distal promoter that predominantly controls the expression of RBFOX2 (3-fold change compared to the proximal promoter). CAGE data detected a third promoter in human hepatocytes that does not overlap with previously annotated RBFOX2 promoters (FIG. 6A). This and the common distal promoter account for most of the RBFOX2 transcription in human hepatocytes. Both show enrichment of H3K4me3 and H3K27ac by ChIP-seq in adult human liver, consistent with their role as active hepatic promoters.

Further analysis of liver ChIP-seq datasets showed that the RBFOX2 promoters are bound by FOXA1/2 in humans (FIG. 6A, bottom insets) and mouse (FIG. 13H). FOXA1/2 are winged helix transcription factors implicated in bile acid metabolism and protection from liver cholestasis³⁵. To validate the role of FOXA1/2 in Rbfox2 regulation, we knocked down Foxa1 and Foxa2 in mouse hepatoma Hepa1-6 cells, which led to a significant decrease in Rbfox2 expression (FIG. 6B). Conversely, FOXA1 overexpression in HepG2 cells³⁶ was associated with a significant increase in RBFOX2 expression, but not RBFOX1/3 (FIG. 6C). Altogether, these results identify the active promoters of RBFOX2 in the liver and demonstrate that the transcription factors FOXA1/2 are upstream regulators of RBFOX2 in human and mouse liver.

### Example 6—Scarb1 is an RBFOX2 Target with Therapeutic Potential

To elucidate the molecular mechanisms underlying the role of RBFOX2 in lipid homeostasis we performed a systematic design of splice-switching oligonucleotides regulating alternative splicing of RBFOX2 downstream targets. The efficacy of the SSOs was tested by PCR followed by capillary electrophoresis. Next, SSOs showing potent activity for each splicing event were further used in lipidomic analyses in primary hepatocytes (Table 1).

TABLE 6
Suppl. - List of splice switching oligonucleotides used in this study

| | | | | SEQ ID |
|---|---|---|---|---|
| Name | Sequence (5'-3') | Target | Purpose | NO. |
| Scr | [A*A*A*U*A*A*U*U*G*A*A*U*U*U*U*A*A*A*U*A] | — | Control | 35 |
| SSO#5.1 | [G*C*C*U*A*C*C*U*A*G*G*U*U*G*U*U*U*A] | Pla2g6 | Promote skipping | 29 |
| | | | of exon 10 | |
| SSO#6.2 | [A*C*C*A*A*U*U*C*A*C*C*U*U*C*C*A*A*U*A*U] | Sec31a | Promote skipping | 30 |
| | | | of exon 21 | |
| SSO#7.8 | [G*C*C*A*C*C*U*U*A*C*C*C*G*G*A*U*G*U*A*U] | Numb | Promote skipping | 31 |
| | | | of exon 9 | |
| SSO#7.9 | [U*U*A*A*A*C*U*U*A*C*U*U*U*U*C*C*A*A*A*G] | Numb | Promote skipping | 32 |
| | | | of exon 3 | |
| SSO#8.3 | [A*C*G*C*A*C*C*U*A*U*A*G*C*U*U*G*G*C*U*U*C*U] | Scarb1 | Promote skipping | 33 |
| | | | of exon 12 | |
| SSO#11.1 | [U*A*C*U*C*U*G*G*U*C*C*U*U*A*A*G*G*A*A*A] | Osbpl9 | Promote skipping | 34 |
| | | | of exon 6 | |

LC/MS metabolomic analysis revealed that RBFOX2-deficient hepatocytes showed increased accumulation of lipids (FIG. 13I), confirming the role for RBFOX2 in lipid metabolism in hepatocytes. While SSO7.9, promoting changes in Numb exon 3, was not associated with significant lipid remodelling (FIG. 13J), SSOs targeting Numb exon 9, Sec31 exon 21, Pla2g6 exon 10, Osbpl9 exon 6 and Scarb1 exon 12 were associated with specific changes in lipid composition (FIG. 13J), suggesting that these isoforms mediate the effect of RBFOX2.

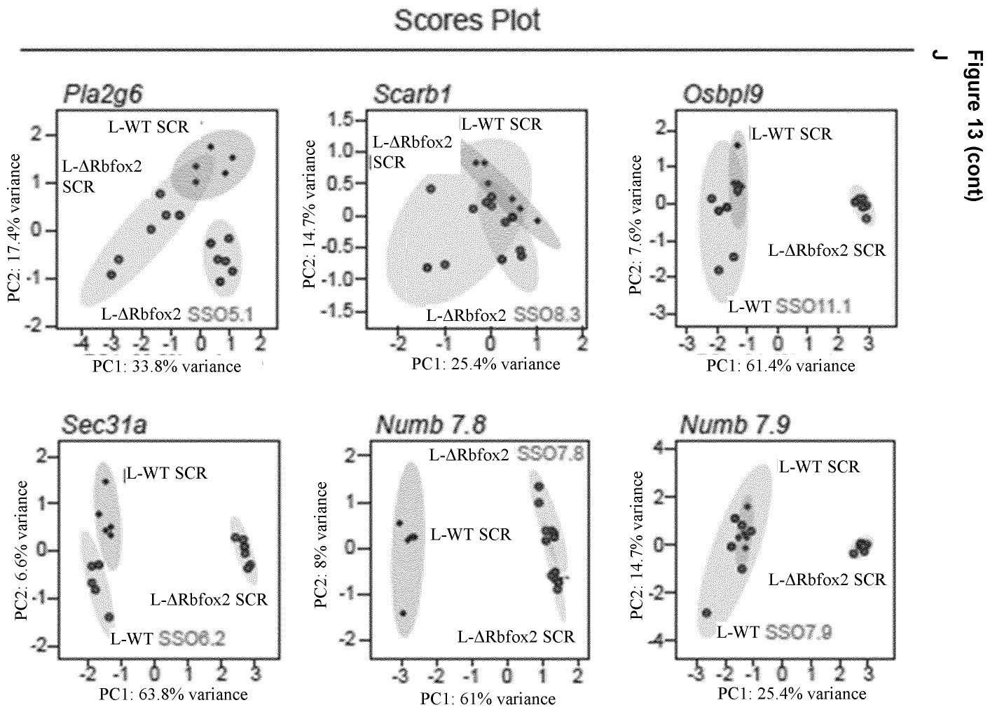

SSO-induced skipping of exon 9 in Numb transcript (FIG. 14A), reverted the accumulation of a number of lipid species including phospholipids PC(36:3), PC(40:7), PE (38:5), PC(38:4), PC(36:2), PC (40:5), triglycerides such as TG(58:8), TG(62:13), TG(58:8), TG(56:7), TG(56:2) or TG(54:2) and others (FIG. 14B). Expression of the short Sec31a isoform (skipping exon 21) is associated with increased levels of particular lipid species such as PC(36:2), TG(51:1), PC(32:0), PC(34:0), DG(38:4) or lysoPC(22:6) (FIG. 14C-D). Expression of the short Osbpl9 isoform (skipping of exon 6) in wild type cells promotes an increase in specific species such as Cer (40:2), TG (50:1), TG (51:1) PC (32:0) PC (34:0), DG (38:4), PE (40:7) resembling the RBFOX2-deficient hepatocytes and suggesting a previously uncharacterized role in lipid metabolism (FIG. 15A-B). Expression of the long Pla2g6 isoform including exon 10 (FIG. 15C-D) was associated with minor changes in lipid composition (FIG. 15E).

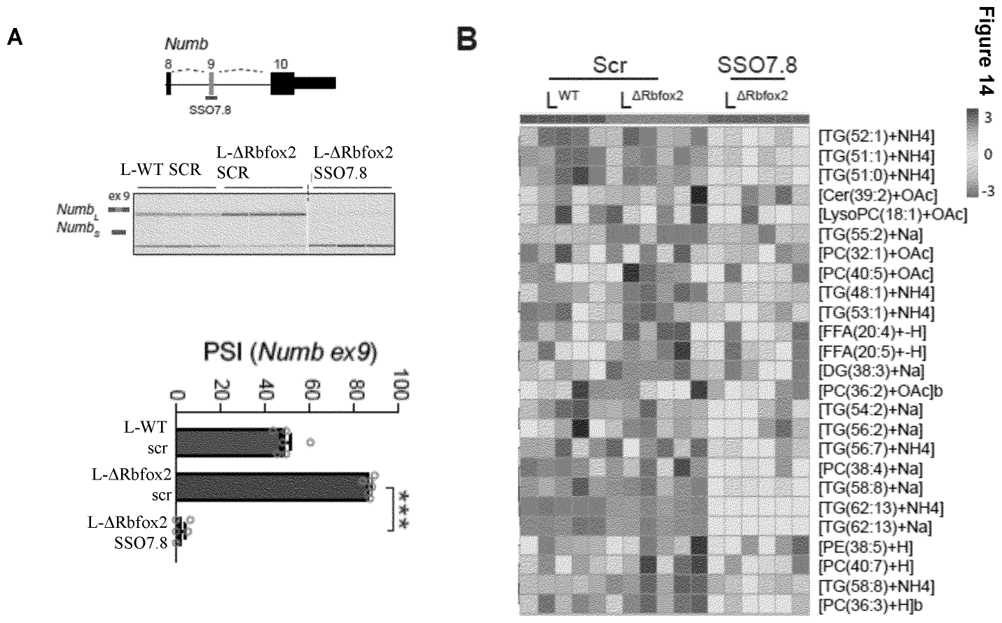

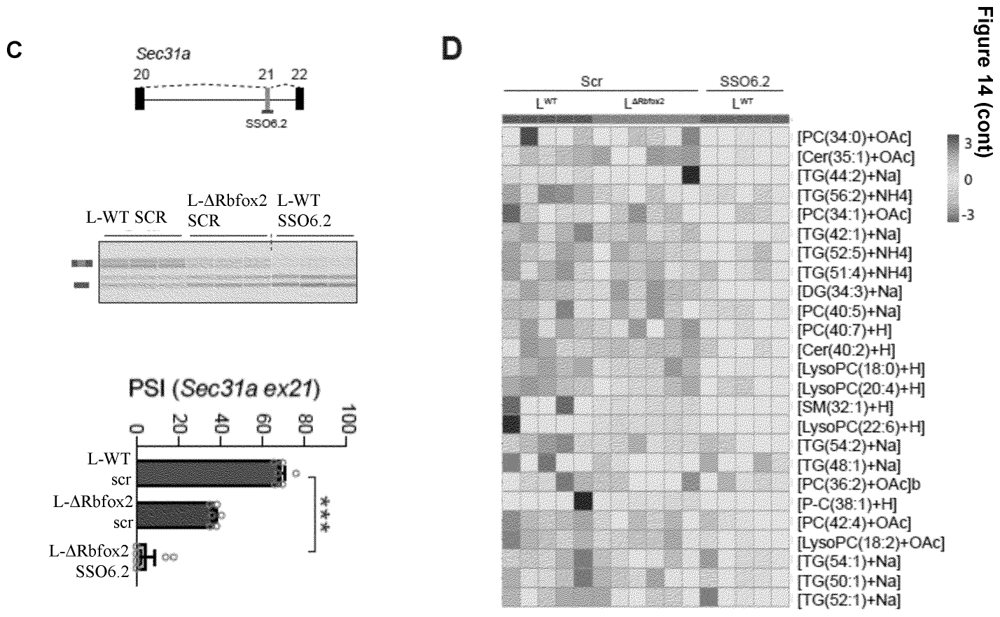

Inclusion of Scarb1 exon 12 generates the canonical SR-BI isoform, while skipping this exon generates an alternative receptor variant with a different adapter carboxy-terminal domain, named SR-BII¹³⁷. SR-BI/II, is a scavenger receptor for multiple ligands including very low (VLDL) and high-density lipoproteins (HDL), involved in the transport of cholesterol, cholesteryl esters, phospholipids-PC, sphingomyelins, lysoPC and other lipid species³⁸. However, the overall impact on liver lipidomics and the pathophysiological significance of these splicing variants has not been established. SSO8.3, showed significant activity in repressing exon 12 inclusion in primary hepatocytes (FIG. 16A). LC/MS analysis showed that this treatment was associated with substantial changes in lipid composition in L^{ΔRbfox2} hepatocytes (FIG. 16B), partially reverting some of the changes associated with RBFOX2 deficiency, such as increased total ceramides (FIG. 16C), PUFA/non-PUFA TG ratio (FIG. 16D) and a number of SMs and TGs (FIG. 16G-T).

Increased intrahepatic levels of cholesterol, ceramides, sphingomyelins, and other lipotoxic species have been implicated in the pathogenesis of obesity-induced steatohepatitis^{13, 38}.

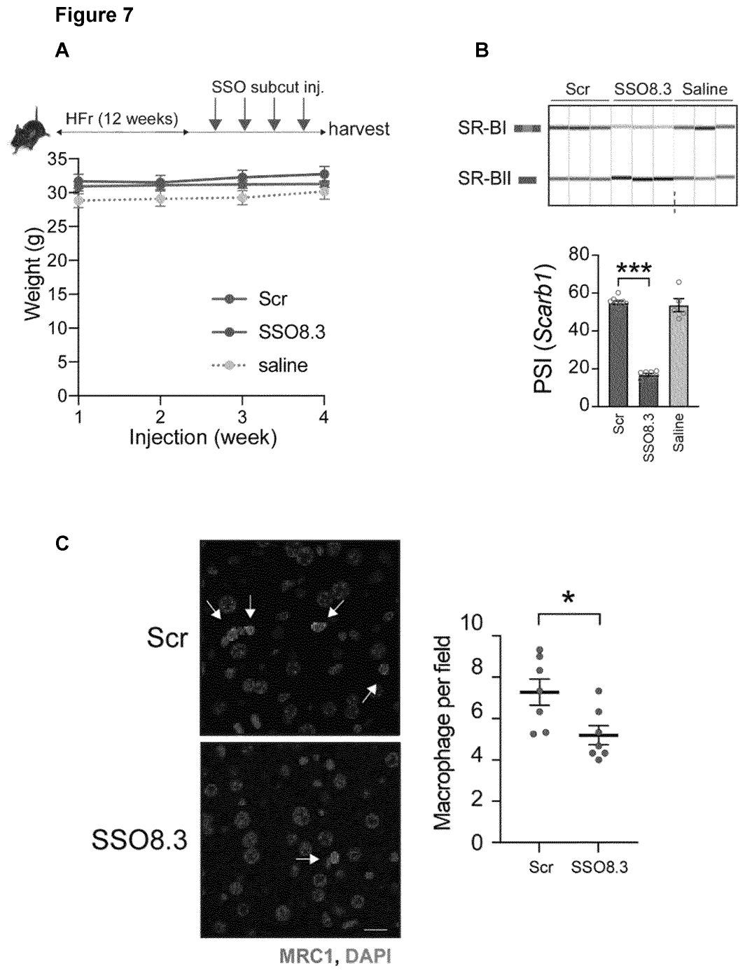

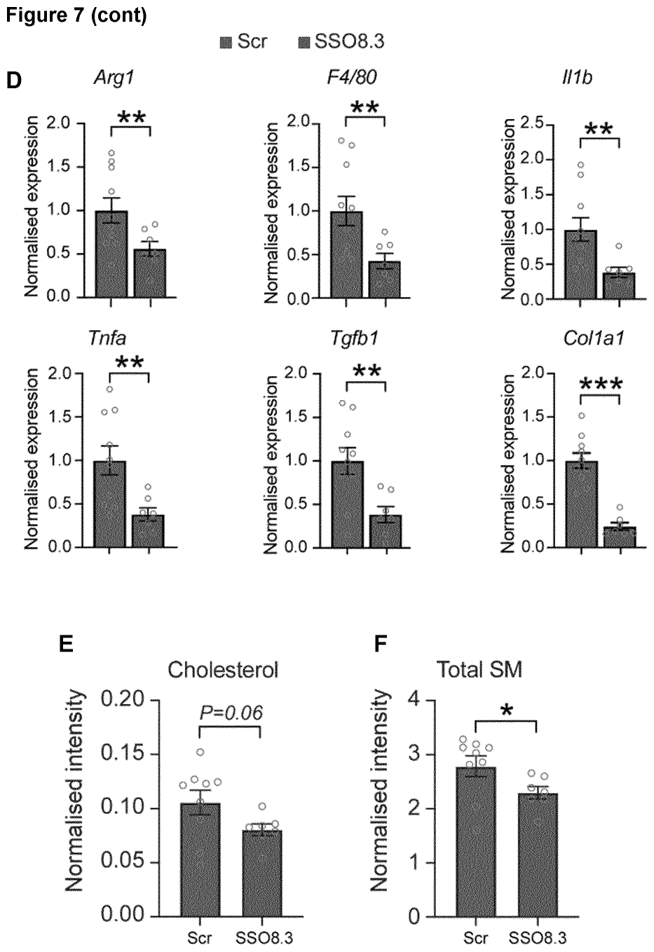

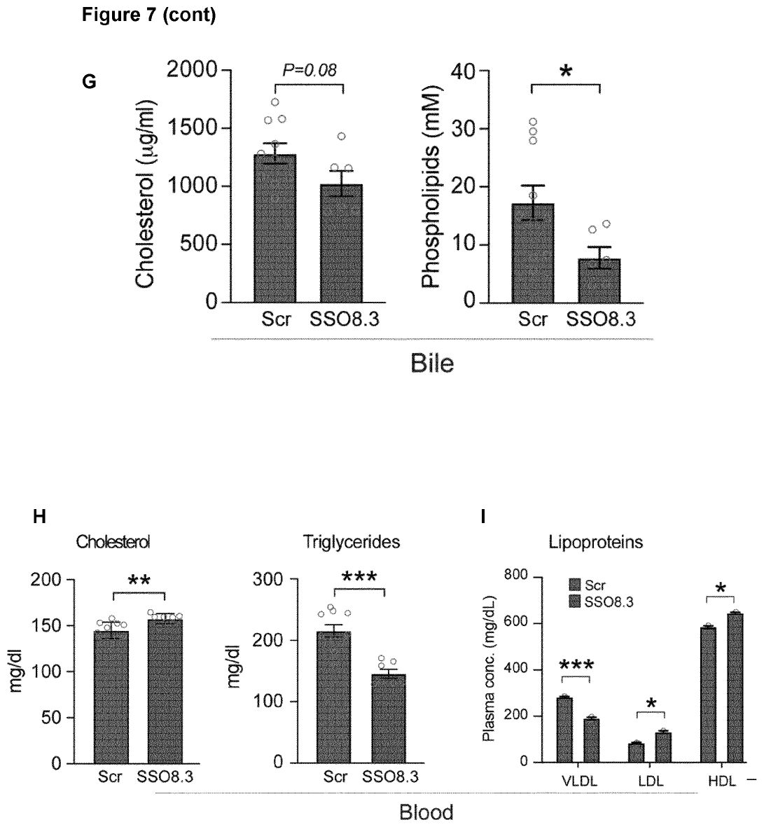

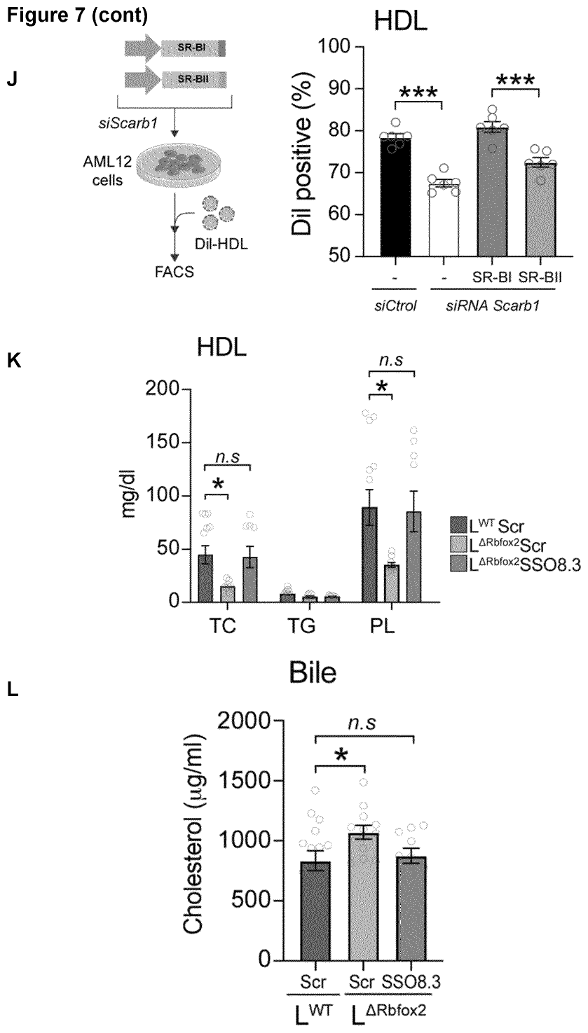

While SCARB1 is a complex therapeutic target, the effect of SSO8.3 in decreasing the levels of some of these lipid species in hepatocytes suggest that strategies aimed to promote the SR-BII isoform could contribute to alleviating obesity-induced inflammation in vivo. To test this, we injected SSO8.3, a scrambled control SSO (Scr) or saline into mice fed an obesogenic HFr diet (FIG. 7A, top). SSO8.3 showed a potent activity antagonising Scarb1 exon 12 inclusion in vivo in the liver (FIG. 7B, 16J). SSO8.3 was designed to avoid potential off-target effects. This specificity was confirmed by evaluating potential expression changes in the top three potential off-target genes by qPCR (FIG. 16K-M). No body weight loss (FIG. 7A, bottom) or increase in transaminases (FIG. 16N-0) were detected, indicating that the RNA injections were not associated with toxic side-effects. IHC analysis of liver sections showed decreased macrophage infiltration in liver from mice injected with SSO8.3 (FIG. 7C). qPCR analysis showed that expression of inflammatory (Arg1, F4/80, and Tnfa) and fibrotic (Tgfb1 and Col1a1) markers was significantly downregulated in mice treated with SSO8.3 (FIG. 7D). Consistent with a decreased lipid load in hepatocytes (FIG. 7E-F), SSO8.3-treated mice showed a decreased liver/total body weight ratio (FIG. 16P) and decreased cholesterol and phospholipid secretion into the bile (FIG. 7G) suggesting decreased reverse cholesterol transport into the liver.

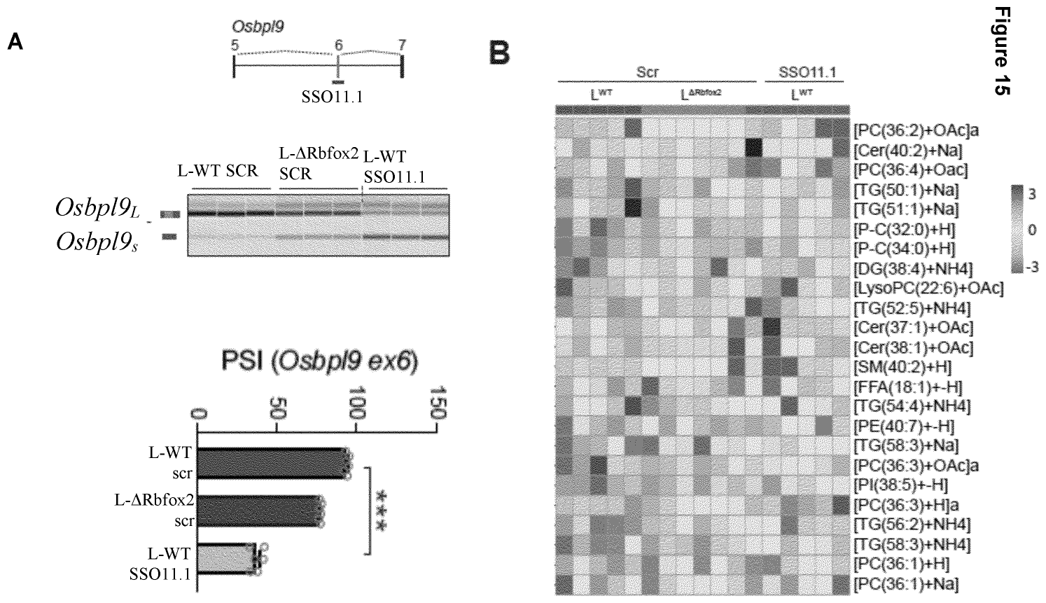

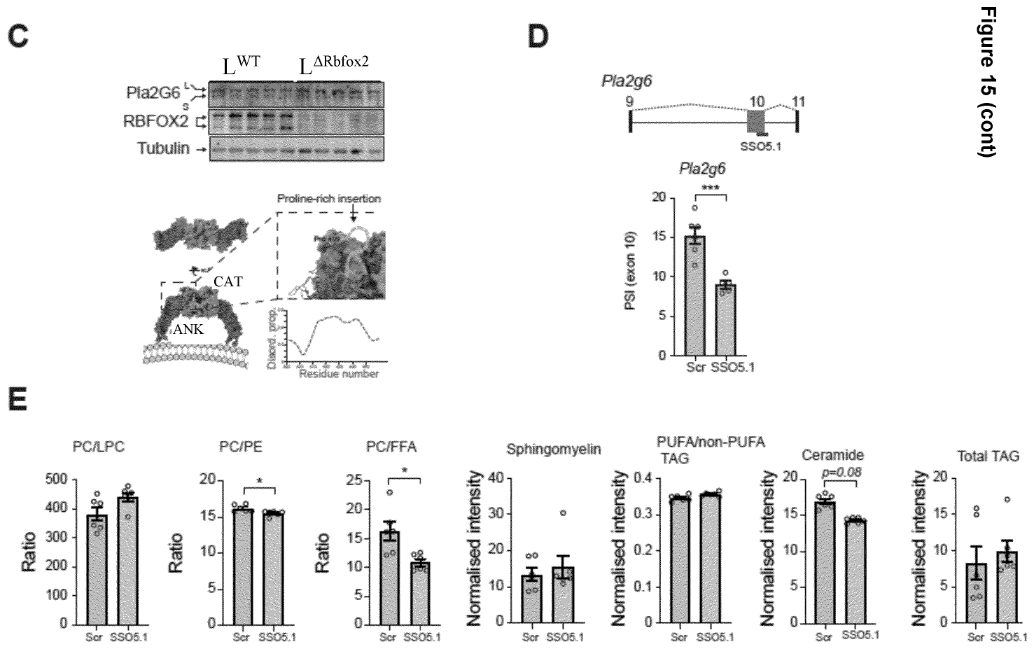

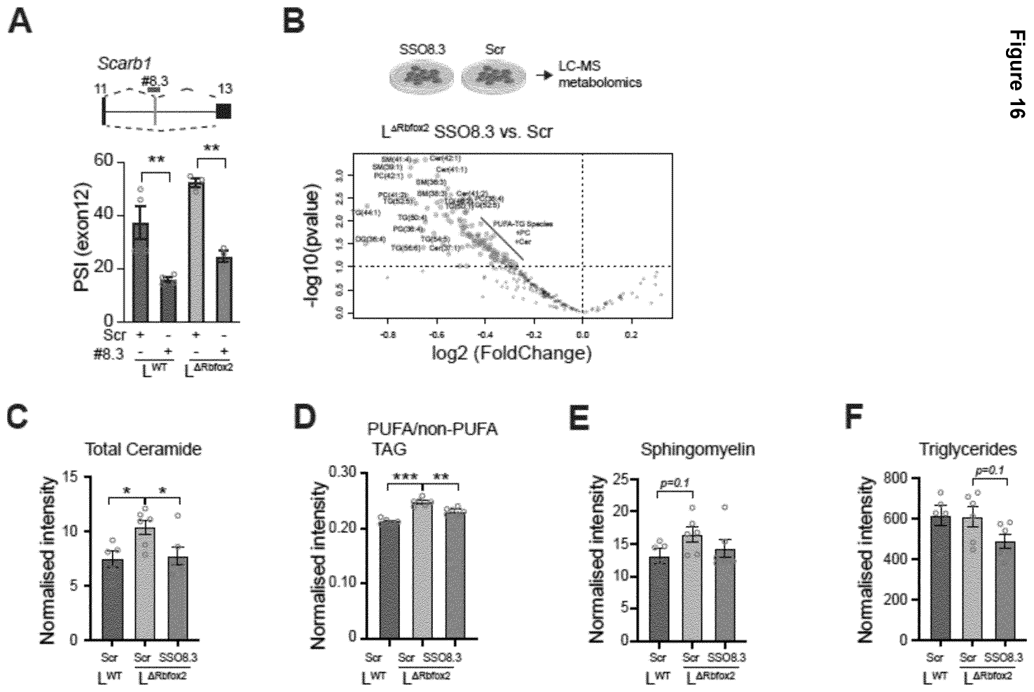

Genetically modified mouse models have shown that complete³⁹ or liver-specific⁴⁰ inactivation of Scarb1 is associated with increased VLDL, LDL, and HDL levels, and increased atherosclerosis, while over-expression of SR-BI has the opposite effect²⁸. SSO8.3 treatment caused a mild but significant increase in total cholesterol levels and a decrease in blood triglycerides (FIG. 7H). These results suggest that by increasing SR-BII isoform expression, SSO8.3 treatment leads to specific effects on VLDL and HDL lipoproteins. Consistent with this hypothesis, lipoprotein analysis showed a strong decrease in VLDL lipoproteins and increased HDL and LDL levels (FIG. 7I). To further confirm these findings, lipid species content was directly quantified in isolated lipoproteins. This analysis revealed major alterations in lipoprotein composition including a drop in the cholesterol content of VLDL whereas that of HDL and LDL was increased upon SSO8.3 treatment (FIG. 17A-C). SSO8.3 treatment was not associated with changes in lipogenic genes (FIG. 17D) or overall triglyceride content in the liver (FIG. 17E). For these reasons, while we cannot exclude a contribution from decreased VLDL secretion, the decreased total, VLDL and LDL triglyceride blood level suggests that SSO8.3 accelerates VLDL lipolysis and remnants formation. However, confirmation of this mechanism will require further investigation.

Collectively, these results show that RBFOX2 coordinates an alternative splicing network in the liver that promotes specific changes in lipid metabolism and collectively regulates the homeostasis of lipid species including cholesterol, sphingomyelins and phospholipids. In particular the SR-BIII splice switch can be targeted therapeutically to decrease liver inflammation and modify lipid distribution.

### Example 7—Scarb1 AS Mediates the Effect of RBFOX2 in Cholesterol Metabolism

RBFOX2 deficiency is not associated with changes in cholesterol biosynthesis nor lipogenesis (FIG. 12C; 17D). Moreover, our data supports that increased SR-BI/II-mediated lipid uptake contributes to the increased accumulation in cholesterol and other lipids upon RBFOX2 deficiency. We hypothesised that this mechanism could also underlie the changes in blood cholesterol associated with RBFOX2-deficiency, through increased reverse cholesterol uptake and excretion into the bile. To explore this possibility, we first engineered AML12 hepatocytes with targeted inactivation of endogenous Scarb1 with siRNA or control siRNA, that simultaneously expressed either codon-optimised SR-BI or SR-BII (FIG. 7J, 17F). Comparable expression levels of SR-BI and SR-BII were confirmed by absolute qPCR quantification (FIG. 17G). FACS analysis showed that expression of SR-BI isoform, including exon 12, is associated with increased lipid uptake from Dil-HDL lipoproteins (FIG. 7J).

Next, we investigated the contribution of SR-BII isoforms to the role of RBFOX2 in vivo. We treated L^{WT} and L^{ΔRbfox2} mice with Scr control or SSO8.3 to antagonise SR-BII isoform in L^{ΔRbfox2} mice. Notably, SSO8.3 efficiently reverted SR-BI/SR-BII ratio in L^{ΔRbfox2} mice liver (FIG. 17H). Blood lipoproteins were isolated and lipid composition was quantified. Cholesterol and phospholipid content in HDL was decreased in L^{ΔRbfox2} mice and these differences were abolished by reverting SR-BI/SR-BII ratio, confirming the contribution of this isoform switch and cholesterol transport to RBFOX2 role in the liver (FIG. 7K). Further analysis confirmed that cholesterol in the bile was increased upon RBFOX2 inactivation in the liver (FIG. 7L), while acute adenovirus-mediated RBFOX2 overexpression causes the opposite effect (FIG. 5G). Moreover, switching Scarb1 isoform expression by SSO8.3 treatment reverted the cholesterol excretion in the bile (FIG. 7L) and total blood cholesterol levels (FIG. 17J), further confirming that SR-BI/II-mediated reverse cholesterol transport underly the role of RBFOX2 in cholesterol metabolism. Purification and analysis of blood LDL and VLDL showed that RBFOX2 deficiency was associated with changes in cholesterol and phospholipids that did not reach statistical significance, suggesting a major role for RBFOX2 in cholesterol HDL uptake (FIG. 17K-L). Collectively, these results uncover a novel role for RBFOX2 in the control of lipid metabolism and show that the SR-BI/SR-BII isoform switch plays a key role in this mechanism by regulating HDL lipoprotein homeostasis.

### Discussion of Examples 1 to 7

In the liver, pre-mRNA alternative splicing (AS) has been largely regarded as a housekeeping mechanism involved in tuning the transcriptome to maintain cellular identity. While several splicing factors have been shown to play a role in this regulation⁴¹⁻⁴⁵, the contribution of specific AS networks (splicing factors and downstream isoforms) to fluctuating metabolic demands remains to be characterised.

Here we describe a role for RBFOX2 in regulating genes involved in lipid metabolism in the liver. This AS network is modulated by an obesogenic diet, and RBFOX2 is critical for this regulation. In mammals, the Rbfox family includes three paralogs: Rbfox1, Rbfox2, and Rbfox3. Rbfox1 is expressed in neurons, heart, and muscle; Rbfox3 expression is restricted to neurons; and Rbfox2 has a broader expression profile46. We have found that Rbfox2 is mainly expressed in hepatocytes in the liver, and ablation of Rbfox2 gene in hepatocytes leads to a cholesterol decrease in the blood and an increase in intrahepatic content of cholesterol, bile acids and other lipids, uncovering a role for RBFOX2 in controlling lipid distribution.

Increased circulating and intrahepatic cholesterol levels contribute to metabolism-associated fatty liver disease (MAFLD) and promote coronary artery disease^{14, 15}. Liver cholesterol overload is also recognised as a key contributor to progression of liver damage and inflammation^{13, 38, 47}. Cholesterol levels are tightly regulated to ensure a constant supply to tissues, while preventing the detrimental effects of excessive accumulation. The characterisation of this AS network in the liver illustrates that additional layers of complexity are involved in cholesterol homeostasis in health and disease.

We demonstrate that RBFOX2-mediated regulation of splicing variants in Scarb1, Pla2g6, Numb, Sec31a or Osbpl9 transcripts is conserved in humans and has specific roles in controlling lipid composition. While the coordinated activity of this splicing network underlies the effect of RBFOX2 in lipid metabolism, individual components can be targeted with splice-switching oligonucleotides to trigger specific changes in hepatocyte lipid content. Two different canonical receptors, SR-BI and SR-BII, are generated through the inclusion/skipping of exon 12 of the Scarb1 gene. SR-BI has increased activity in HDL binding and promotes the selective import of cholesteryl esters^{37, 48}. However, the regulation and the biological significance of this splicing event had not been described. HDL cholesterol levels in plasma are inversely correlated with atherosclerosis risk in humans and in some murine models⁴⁹. For this reason there has been considerable interest in SR-BI as a therapeutic target to modify lipid metabolism by boosting HDL-C levels. However, human genetic studies⁵⁰ together with gain-²⁸ and loss-of-function³⁹ mouse models have shown that SR-BI activity is atheroprotective, underscoring that HDL cholesterol flux is more important than the steady state levels. We find that by promoting SR-BII expression, RBFOX2 prevents reverse cholesterol flux from HDL lipoproteins, a mechanism that ensures appropriate distribution and prevents excessive cholesterol loss. Consistently, RBFOX2 inactivation, is associated with decreased total and HDL cholesterol in the blood, and increased cholesterol in the liver and consequent excretion in the bile under a lipogenic diet.

Moreover, by promoting SR-BII expression, the splice-switching oligonucleotide SSO8.3 substantially reduces the accumulation of lipotoxic species such as ceramides, cholesterol and sphingomyelins, and this effect is associated with decreased expression of inflammatory markers in the liver.

Treatment with SSO8.3, to promote the expression of the SR-BII isoform in vivo, is associated with a substantial reduction in plasma VLDL and triglycerides. These results show that rather than a loss- or a gain-of-function, expression of the SR-BII isoform promotes an anti-atherogenic lipoprotein profile, potentially by accelerating VLDL catabolism. While our results clearly establish that the RBFOX2-SR-BI/II axis plays a key role in controlling cholesterol homeostasis, the contribution of the SR-BI/II splice switch to triglycerides homeostasis seems to be more complex as RBFOX2 inactivation is not associated with changes in total circulating triglycerides. The clarification of this point warrants further investigation.

RNA-based drugs such as inclisiran⁸ and Mipomersen⁷ significantly reduce cholesterolemia by targeting PCSK9 and APOB mRNAs, respectively. Recent improvements in the design and pharmacokinetics of RNA-based oligonucleotides should enable the development of isoform-specific therapeutics for common metabolic pathologies. However, this avenue is underexplored due to the limited characterisation of the key isoforms maintaining health or promoting disease. Our work provides a proof-of-principle for the potential of RNA therapeutics targeting individual isoforms in the liver.

### Example 8—Development of Humanized Versions of SSOs Targeting SCARB1 Gene

Examples 1 to 7 identified scavenger receptor class B type I (Scarb1) as a potential new target in non-alcoholic steatohepatitis (NASH) to modulate lipid metabolism and liver inflammation. Scarb1 has two AS variants: SR-BI including exon 12/SR-BII skipping exon 12. High fructose diet in mice increases expression of SR-BI that is involved in the transport of cholesterol and other lipid species. By increasing SR-BI expression, the body promotes uptake of cholesterol and other lipids by the liver leading to inflammation and liver toxicity. Conversely, splicing switching oligonucleotides (SSO) specifically targeted to promote the expression of the isoform skipping exon 12 (SR-BII) lead to a decrease in lipid loading in the liver in mice.

The results in mice have shown that treatment with a 21 nt oligonucleotide, SSO8.3 is associated with significant changes in the liver and circulating lipids with clear therapeutic effects: 1) decreased liver inflammation that could be beneficial in NASH, 2) decreased blood triglycerides, 3) decreased intrahepatic cholesterol, and 4) decreased cholesterol levels in the bile which is relevant to gallstone disease. No toxic side effects have been detected.

In addition, we have developed humanized versions of SSOs targeting SCARB1 gene which could be used as effective therapeutic agents to target the underlying pathological processes associated with the above-described pathologies such as NASH in humans.

An initial set of splice-switching oligos (SSO) was designed. The rationale for the initial round of design was the targeting of cis-regulatory regions in exon 12 in human SCARB1 gene. These SSOs were tested in human hepatocytes (derived from induced pluripotency stem cells—iPSCs), by transfecting 100 nM oligo with lipofectamine 2000. RNA was extracted 24 h after transfection and activity was evaluated by calculating percentage splice in (PSI=exon 12 included/(exon12 included+exon12 skipped).

As shown in FIG. 18, SSO8.5 targeting the 3′ region of exon 12 showed a stronger activity in antagonising exon 12.

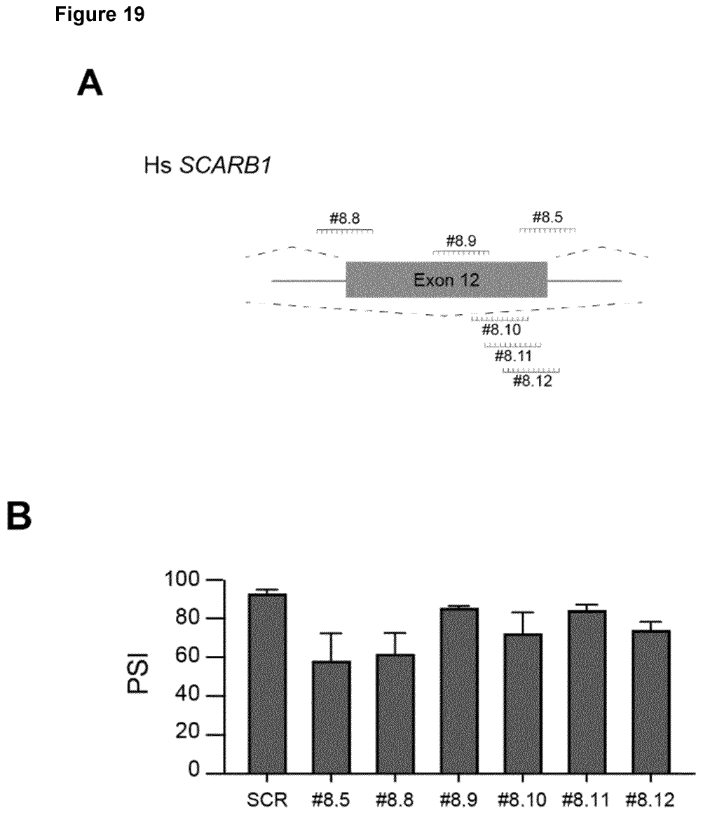

In order to increase the chances of finding the best molecules, we explored other genomic regions, and a second round of SSOs was designed. The rationale for the second round of design was the targeting of alternative regions in exon 12 in human SCARB1 gene. These SSOs were tested in iPSCs-derived hepatocytes, by transfecting 100 nM oligo with lipofectamine 2000. RNA was extracted 24 h after transfection and activity was evaluated by calculating PSI. As shown in FIG. 19, SSO8.5 was still the oligo with higher activity.

After this, the most promising candidates from round 1 and round 2 were further modified by genomic micro-walk. The rationale for the third round of design was 1) to test the effect of subtle sequence modifications in the oligos previously showing some activity, 2) target intronic regulatory elements identified. In addition, 3) chemical modifications (2′MOE) were introduced in the one of the candidates (SSO8.5) leading to the new oligo SSO8.21, to improve activity and/or pharmacokinetics. To note, human hepatocytes or HepG2 liver cells have low transfection potential while Huh7 liver cells have high transfection efficiency. To maximise the activity 13 hSSOs (2′OME modification: SSO8.5, SSO8.10, SSO8.11, SSO8.12, SSO8.13, SSO8.14, SSO8.15, SSO8.16, SSO8.17, SSO8.18, SSO8.19, SSO8.20 or 2′MOE modification: SSO8.21) were tested in Huh7 human liver cells by transfecting 100 nM with lipofectamine 2000. RNA was extracted 24 h after transfection and activity was evaluated by calculating PSI.

Cells were transfected for 6 h with 100 nM SSO with lipofectamine 2000. 24 h after transfection RNA was extracted and activity was quantified as PSI of SCARB1 exon 12. Scramble oligos were used as controls. Results are represented as mean±SEM (n=3). Statistical comparison was performed with Student's T test (***p<0.001).

As shown in FIG. 20, SSO8.5, SSO8.10, SSO8.11, SSO8.18 and SSO8.21 promote a more than 60% decrease in PSI of SCARB1 exon 12 (threshold initially established in the DGF application). In particular, both SSO8.18 and SSO8.21 showed a very high activity antagonising SCARB1 exon 12 (promoting SR-BII isoform). To further test these candidates, we transfected HepG2 cells (an alternative human liver cell line with a much lower transfection efficiency than Huh7 cells). In this system, SSO8.18 showed a higher activity, suggesting that this oligo have a consistently higher activity across different cell types and hence has been selected as lead hSSO for the liver-on-a chip experiments (FIG. 20D).

### Example 9—Liver-On-A-Chip Experiments

A Liver-On-A-Chip is a human micro-tissue system containing most of the key cell types involved in the development of NASH, including hepatocytes, stellate cells, Kupffer cells, and liver endothelial cells. Use was made of a media, developed by Insphero (Zurich, Switzerland), that promotes the development of NASH and fibrosis, as evaluated by gene expression, closely resembling human disease.

An oligonucleotide aimed to promote the expression of the SR-II isoform (SSO8.18), and a scramble control (SCR) were used in a Liver-On-A-Chip model system. Efficacy and toxicity were evaluated by treating liver microtissues with different doses (1 to 20 μM) of these SSOs for 3-10 days.

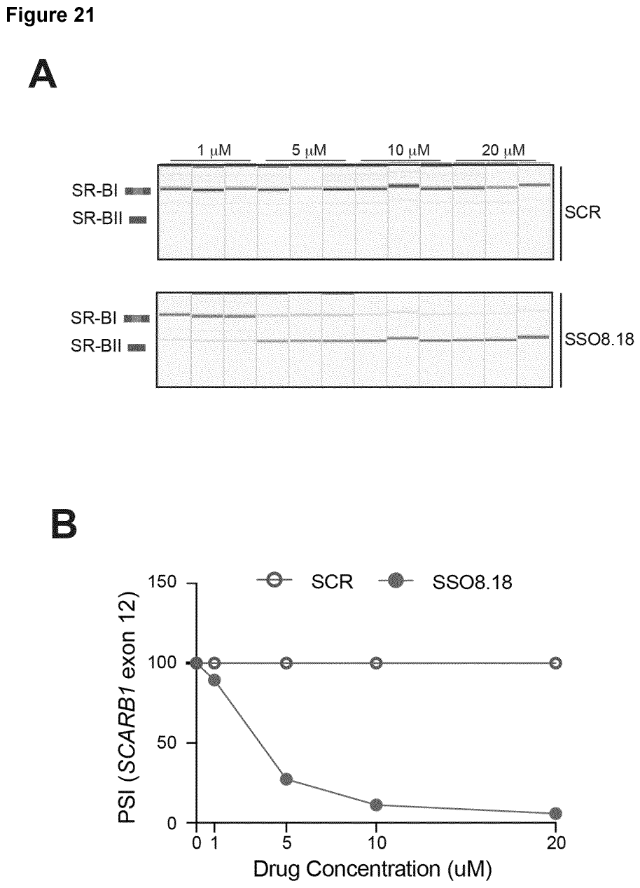

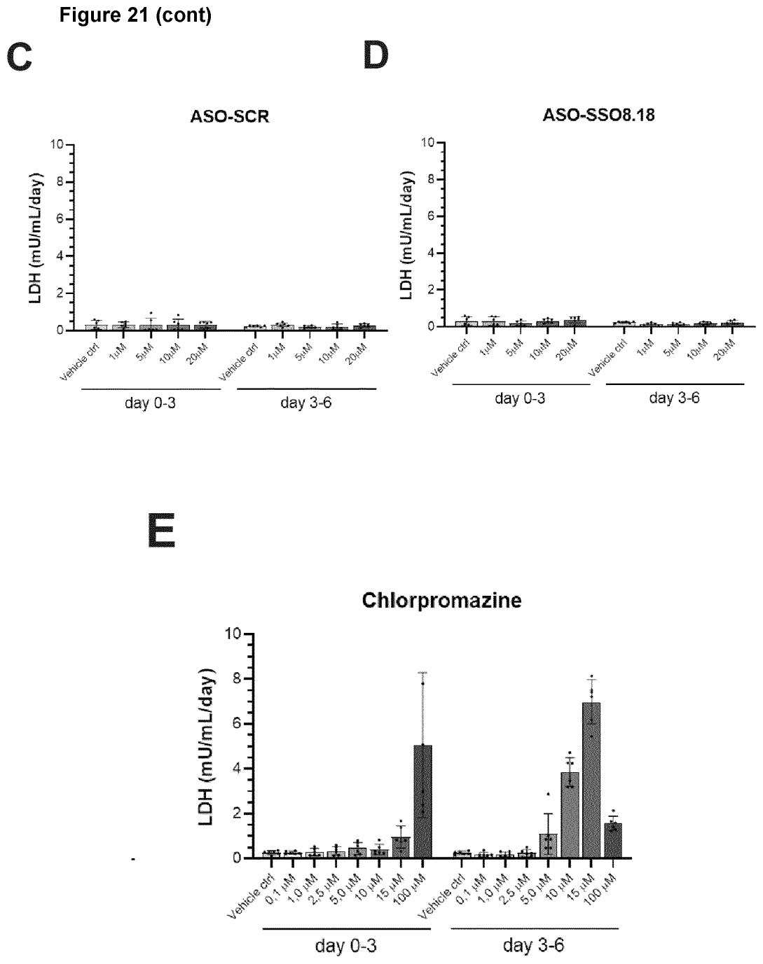

5-6 microtissues were pooled into 3 biological replicates. RNA was isolated with a RNAeasy micro kit, and cDNA was obtained by reverse transcription. Expression of SR-BI (including exon 12) and SR-BII (skipping exon 12) were analysed by PCR and capillary electrophoresis (FIG. 21A). This analysis confirmed a potent effect of SSO8.18 in triggering a dose-dependent skipping of exon 12 (FIG. 21B). Toxicity was evaluated by quantifying LDH release and even the higher doses of either SCR or SSO8.18 oligos were not associated with significant toxicity (FIG. 21C-D). A dose response curve with chlorpromazine was used as a positive control (FIG. 21E).

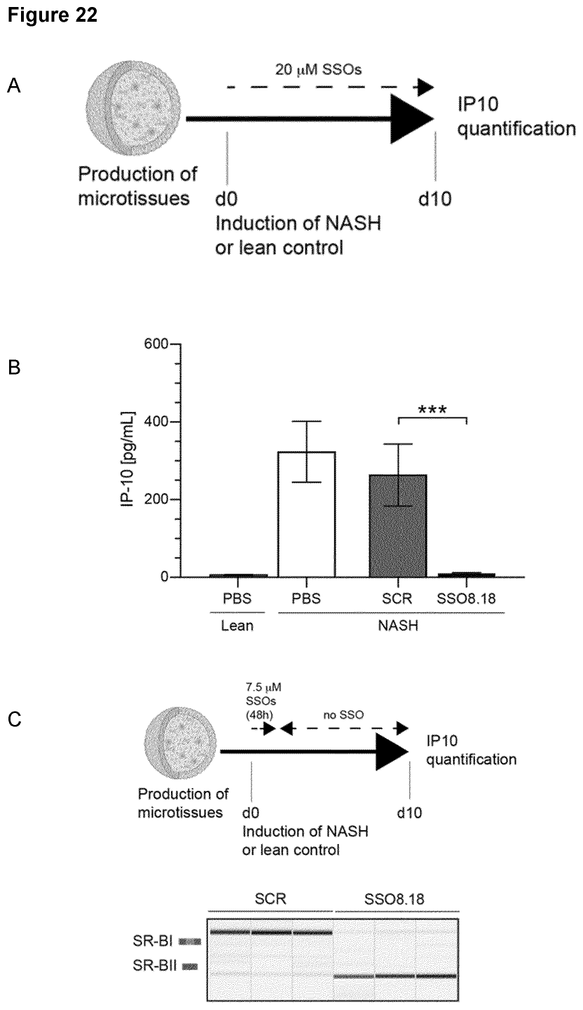

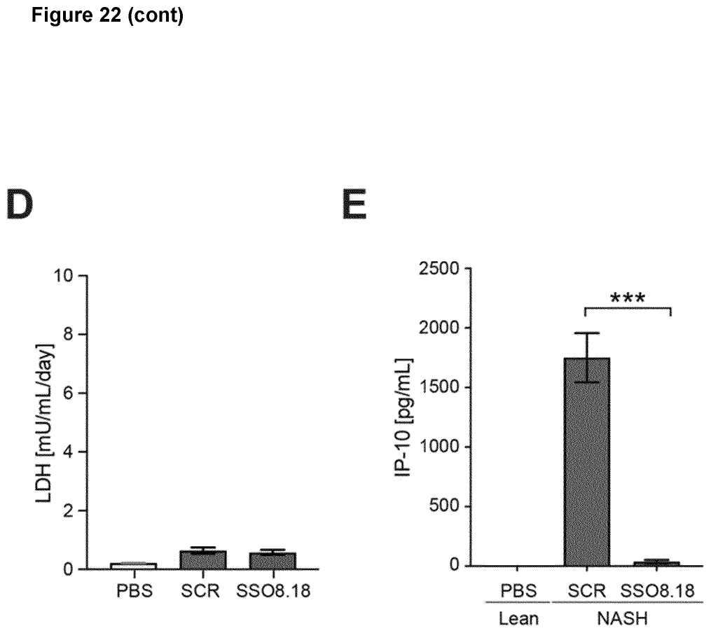

The chemokine CXCL10 (IP-10) has been shown to play a key role in the development of liver inflammation. Increased SR-BII expression has been associated with decreased expression of inflammatory markers in vivo. For this reason, we tested the effect of SSO8.18 in the expression of IP10 in the liver microtissues (FIG. 22A). As shown in FIG. 22B, SSO8.18 is able to promote a potent decrease in IP10 levels, confirming the anti-inflammatory activity.

RNA oligo therapeutics provide multiple pharmacokinetic advantages for the treatment of liver pathologies. 2′ modifications confer a high stability to oligonucleotides. For this reason, we tested the effect of a single lower dose in the activity of SSO8.18 in this system. Microtissues were treated with 7.5 μM SSO8.18 or SCR control during 48 h. After this time, SSOs were washed out and microtissues were maintained with media without SSOs for the rest of the experiment (FIG. 22C, top). In these conditions, SSO8.18 was able to trigger a potent splicing change, promoting SCARB1 exon 12 skipping, confirming the efficacy of our oligo (FIG. 22C, bottom).

Moreover, consistent with our previous experiments, while no toxicity was detected, as evaluated by LDH release (FIG. 22D), treatment with SSO8.18 caused a strong decrease in IP10 levels (FIG. 22E).

### Methods for Examples 1 to 7

### Mice

C57BL6/J (stock number 000664), Rbfox2^{loxP/loxP} (stock number 014090)⁵¹ and Albumin-cre (stock number 003574)⁵2 were obtained from the Jackson Laboratory. 8-weeks old mice were randomly assigned to experimental groups and fed a CD, a high-fat diet (60% Kcal from fat, Bioserve) or a high-fructose diet containing 30% (w/v) fructose, administered in the drinking water for 16 to 22 weeks. Mice were housed in pathogen-free barrier facilities under a 12 hour light/dark cycle at 22° C. with free access to food and water. The presence of the Cre recombinase and Rbfox2 LoxP sites was determined by PCR analysis of genomic DNA and the following primers: CreF1>TTACTGACCGTACACCAAATTTGCCTGC (SEQ ID NO: 22) and CreR1>CCTGGCAGCGATCGCTATTTTCCATGAGTG (SEQ ID NO: 23), Rbfox2F1> AACAAGAAAGGCCTCACTTCAG (SEQ ID NO: 24) and Rbfox2R1>GGTGTTCTCTGACTTATACATGCAC (SEQ ID NO: 25). All in vivo work was approved by the animal welfare and ethical review board at Imperial College London and in accordance with the United Kingdom Animals (Scientific Procedures) Act (1986).

### Adeno and Adeno-Associated Viruses

The adenoviral vector driving mouse RBFOX2 expression (Ad-m-RBM9) and pAd-GFP were obtained from Vector Biolabs (Malvern, PA, USA). These viruses were purified by using the AdEasy virus purification kit (Agilent technologies). 8×10⁹ GC were i.v injected in mice fed a HFr diet and mice were harvested 7 days post-injection. Codon-optimised RBFOX2 lacking the RRM was obtained by gene block synthesis (IDT) and cloned in to the AAV-CBA-GFP vector. AAV2/8 were produced and purified by iodixanol gradients and 5×10¹¹ GC were i.v injected in mice fed a HFr diet. Mice were harvested 12 weeks post-injection.

### Tissue and Blood Harvesting

Biopsies were flash frozen in liquid nitrogen and kept at −80° C. Sections for histology were fixed in 10% formalin and subsequently embedded in paraffin, or immersed in OCT and isopentane, for cryosections. Paraffin sections were stained with hematoxylin & eosin, or used for IHC analysis of macrophage infiltration with anti-MRC1 antibody (ab64693) and DAPI (Sigma, D9542) for nuclear staining. Serum was obtained by centrifugation at 5000 g for 10 minutes at 4° C. and analyzed by St. Mary's hospital pathology department unless otherwise indicated.

### Quantitative Proteomics (TMT/MS, Tandem Mass Tag)

Flash frozen livers were lysed using a homogenizer with SDS lysis buffer (2.5% SDS, 50 mM HEPES pH 8.5, 150 mM NaCl, 1×EDTA-free protease inhibitor cocktail (Roche), 1× PhosSTOP phosphatase inhibitor cocktail (Roche)). Lysates were clarified by centrifugation at 13,000 rpm for 15 min and protein concentration was measured by Pierce BCA assay (Thermo scientific). 20 mg of protein was reduced with 5 mM TCEP for 30 mins, then alkylated with 14 mM iodoacetamide for 30 mins, and finally quenched with 10 mM DTT for 15 mins. All reactions were performed at RT. Proteins were chloroform-methanol precipitated and the pellet resuspended in 8 M urea, 50 mM EPPS pH 8.5. To help with the resuspension, protein precipitates were passed 10 times through a 22G needle and protein concentration was measured again. Before protein digestion, 5 mg of protein was collected, and urea concentration diluted to 1 M with 50 mM EPPS pH 8.5. Then, LysC was added at 1:100 (LysC:protein) and digested for 12 hours at RT. Samples were further digested for 5 hours at 37° C. with trypsin at 1:100 (trypsin:protein). To stop the digestion 0.4% TFA (pH<2) was added to the samples. Digested samples were clarified by centrifugation at 13,000 rpm for 10 min. Peptide concentration was measured using a quantitative colorimetric peptide assay (Thermo scientific). 25 μg of peptides were desalted using 10 mg SOLA HRP SPE Cartridges (Thermo scientific). To allow the comparison of both TMT, 2 bridge channels were prepared and processed in parallel. For that, 1.39 μg of each sample was added for to each bridge channel. Then, dried peptides from all 20 samples were resuspended in 200 mM EPPS pH 8.5 and labelled with TMT-10plex following the protocol described in⁵³. After labelling, both bridge channels were combined and split again to ensure homogeneity. Finally, samples were mixed in equal amounts. After combining, both TMT were desalted using the tC18 SepPak solid-phase extraction cartridges (Waters) and dried in the SpeedVac. Next, desalted peptides were resuspended in 5% ACN, 10 mM NH₄HCO₃ pH 8. Both TMT were fractionated in a basic pH reversed phase chromatography using a HPLC equipped with a 3.5 μm Zorbax 300 Extended-C18 column (Agilent). 96 fractions were collected and combined into 24. 12 of these were desalted following the C18 Stop and Go Extraction Tip (STAGE-Tip)⁵⁴ and dried down in the SpeedVac. Finally, samples were resuspended in 3% ACN, 1% FA and run in an Orbitrap Fusion running in MS3 mode⁵⁵ as described previously⁵³. RAW data were converted to mzXML format using a modified version of RawFileReader and searched using the search engine Comet56 against a mouse target-decoy protein database (Uniprot, Jun. 11, 2019) that included the most common contaminants. Precursor ion tolerance was set at 20 ppm and product ion tolerance at 1 Da. Cysteine carbamidomethylation (+57.0215 Da) and TMT tag (+229.1629 Da) on lysine residues and peptide N-termini were set up as static modifications. Up to 2 variable methionine oxidations (+15.9949 Da) and 2 miss cleavages were allowed in the searches. Peptide-spectrum matches (PSMs) were adjusted to a 1% FDR with a linear discriminant analysis⁵⁷ and proteins were further collapsed to a final protein-level FDR of 1%. TMT quantitative values we obtained from MS3 scans. Only those with a signal-to-noise>100 and an isolation specificity>0.7 were used for quantification. Each TMT was normalised to the total signal in each column. To allow the comparison of both TMT, those proteins quantified in both TMT, data was normalised using the bridge channels present in each TMT. Quantifications included in Supplementary Table 1 (see Paterson et al. Nature Metabolism) are represented as relative abundances. Newly generated proteomic datasets are publicly available as described below.

iPSC-Derived Human Hepatocytes

Human induced pluripotent stem cells (iPSC) CGT-RCiB-10 (Cell & Gene Therapy Catapult, London, U.K.) were maintained on Vitronectin XF (STEMCELL Technologies) coated Corning Costar TC-treated 6-well plates (Sigma-Aldrich) in Essential 8 Medium (Thermo Fisher Scientific) and passaged every 4 days using Gentle Cell Dissociation Reagent (STEMCELL Technologies).

Hepatocyte differentiation was carried out as previously described^{58, 59} in Essential 6 Medium (Thermo Fisher Scientific; days 1-2), RPMJ-1640 Medium (Sigma-Aldrich; days 3-8) and HepatoZYME-SFM (Thermo Fisher Scientific; day 9 onward) within TC-treated 182 cm² flasks (VWR). The following growth factors and small molecules were supplemented into the media for hepatocyte differentiation: 3 μM CHIR9901 [Day 1](Sigma-Aldrich), 10 ng/ml BMP4 [Day 1-2](R&D Systems), 10 μM LY29004 [Day 1-2](Promega, Madison, WI), 80 ng/ml FGF2 [Day 1-3](R&D Systems), 100 ng/ml [Day 1-3] and 50 ng/ml [Day 4-8]Activin A (Qkine), 10 ng/ml OSM [Day 9 onwards](R&D Systems) and 50 ng/ml HGF [Day 9 onwards](PeproTech). After 21 days iPSC-derived hepatocytes were dissociated into a single-cell suspension using TrypLE Express Enzyme (10×), no phenol red (Thermo Fisher Scientific) and seeded into multi-well plates coated with type-1 collagen from rat tail (Sigma-Aldrich). Silencing of human RBFOX2 was performed by transfecting 100 nM smart pool to RBFOX2 or a mock control (Horizon) with RNAimax reagent (Invitrogen) in Optimem (Invitrogen).

### AML12 Hepatocytes and Dil-HDL Uptake

AML12 were cultured as previously described⁶⁰. Silencing of Scarb1 was performed by transfecting 100 nM smart pool to Scarb1 or a mock control (Horizon) with RNAimax reagent (Invitrogen) in Optimem (Invitrogen). Expression of SR-BI and SR-BII was obtained by cloning codon-optimised SR-BI and SR-BII (generated by gblock synthesis at IDT) into pLV (PGK)-GFP Neo vector. Third generation lentivirus were generated in HEK-293T cells and purified by high-speed centrifugation. Viruses were resuspended in media supplemented with polybrene and added to the cells. When indicated cells were incubated with 100 ng/ml Dil-HDL for 4 h and lipid uptake was quantified by FACS using a FACSAria III cell sorter system (BD biosciences).

### RNA Isolation

Cells or tissues were homogenized in TRIzol (Thermo Fisher Scientific) and RNA was extracted following the manufacturer instructions. For RNA sequencing, after homogenization with TRIzol, RNA was extracted with a RNeasy kit column (Qiagen) following the manufacturer's instructions, including DNase I treatment.

### RNAseq Sequencing and Analysis

RNA was quality controlled with a 2100 BioAnalyser (Agilent CA, US). Poly(A) enrichment of samples with RIN>8, was performed and libraries were prepared using the NEBNext Ultra II RNA Library Prep Kit for Illumina and multiplexed using NEBNext Multiplex Oligos for Illumina (NEB, E7760S and E7335S). Sequencing was carried out with 100 bp paired end reads with HiSeq 2500 (Illumina). Newly generated RNAseq datasets are publicly available as described below. Previously published datasets of mice fed a high fructose diet were obtained from GSE123896¹⁶. Reads were aligned to Ensembl mouse genome (GRCm38) using Tophat2 (2.0.11)⁶¹ with argument “--library-type fr-firststrand”. Reference sequence assembly and transcript annotation were obtained from Illumina iGenomes (https://support.illumina.com/sequencing/sequencing_software/igenome.html). Gene-based read counts were obtained using featureCounts function from Rsubread Bioconductor package⁶². Normalisation and differential expression analysis were performed using DEseq2⁶³ or edgeR-voom-limma^{64, 65-67} bioconductor packages. Alternative splicing was primarily analysed with rMATs⁶⁸. Differentially spliced sites were kept for data visualisation if passed the following threshold: p<0.05; FDR<0.1 and absolute(IncLevelDifference)>0.1 or <−0.1. Gene Ontology analysis was performed by using GOseq Bioconductor package⁶⁹. List of differentially expressed genes with adjusted p-value of less than or equal to 0.05 were selected as input for the Ingenuity Pathway Analysis (IPA; http://www.ingenuity.com/index.html). No cut-off was applied for fold change of differential expression. Enrichment of binding motifs for different SFs were tested using binomTest function from edgeR bioconductor package⁷⁰.

### Lipidomics Analysis

Tissue was pulverized using a cyroPREP Dry Pulverizer (Covaris). Adapted from Folch and colleagues⁷¹. Approximately 30 mg of frozen liver powder was weighed into a weighed Eppendorf. Tissue was homogenised in a TissueLyzer (20 Hz, 3-5 mins×2) using a stainless steel ball and 1 ml chloroform:methanol (2:1). The stainless-steel ball was removed and 400 ul HPLC grade water was added, samples were vortexed for 20 seconds and centrifuged for 15 minutes, 13,200×g at room temperature. Both the organic and the aqueous layers were removed. The protein pellet was re-extracted in 500 ul 2:1 chloroform:methanol and 200 ul of HPLC grade water, samples were vortexed and centrifuged, and the respective fractions were combined.

Lipid profiling was performed by liquid chromatography high-resolution mass spectrometry (LC-HRMS) using a Vanquish Flex Binary UHPLC system (Thermo Scientific) coupled to a benchtop hybrid quadrupole-Orbitrap Q-Exactive mass spectrometer (Thermo Scientific). Chromatographic separation was achieved using an Acquity UPLC BEH C18 column (Waters, 50×2.1 mm, 1.7 μm) held at a temperature of 55° C. and flow rate of 0.5 mL/min. For the positive ion mode, mobile phase was composed of 60:40 (v/v) acetonitrile/water plus 10 mM ammonium formate (solvent A) and 90:10 (v/v) isopropanol:acetonitrile plus 10 mM ammonium formate (solvent B). For the negative ion mode, mobile phase was composed of 60:40 (v/v) acetonitrile/water plus 10 mM ammonium acetate (solvent A) and 90:10 (v/v) isopropanol:acetonitrile plus 10 mM ammonium acetate (solvent B). The gradient elution program was performed for both ion modes according to the Suppl. Table 4 (see Paterson et al. Nature Metabolism), yielding a total run time of 10 min per sample. The injection volume for the positive and negative ion mode was 5 and 10 μL, respectively. The ionization was performed using a heated electrospray ionization source (HESI) and the parameters for positive/negative mode are as follows: capillary voltage 3.5/−2.5 KV, heater temperature 438° C., capillary temperature 320° C., S-lens RF level 50, sheath, auxiliary and sweep gas flow rate are 53, 14 and 1 unit, respectively. The mass accuracy was calibrated prior to sample analysis for both ion modes. High-resolution mass spectrometric (70,000 at m/z 200) data were acquired in profile mode using the full scan setting (m/z 200-2000). Automatic gain control (AGC) was set to 1e6 and maximum MS1 injection time at 200 ms. Lipidomics data acquisition was performed with Xcalibur software (version 4.1). Peak picking was performed using XCMS⁷², and features normalised to isotopically labelled internal standard and dry tissue weight. Lipid identification was performed by accurate mass using an in-house database. Bile acids analysis was performed using liquid chromatography tandem mass spectrometry (LC-MS/MS) in an Acquity I-Class binary UPLC system (Waters) coupled to a triple quadrupole Xevo TQ-XS mass spectrometer as previously described (https://www.waters.com/webassets/cms/library/docs/7200006261en.pdf) (Waters). Chromatographic separation was performed on a CORTECS T3 column (Waters, 30×2.1 mm, 2.7 μm) held at a temperature of 60° C. and flow rate of 1.3 mL/min. Mobile phase consisted of 0.2 mM ammonium formate plus 0.01% (v/v) formic acid (solvent A) and 50:50 (v/v) isopropanol/acetonitrile plus 0.01% (v/v) formic acid and 0.2 mM ammonium formate. The elution gradient program starts with 20% of B holding for 0.1 minute and ramping to 55% of B over 0.7 minutes, followed by a 0.9 minute column wash at 98% of B. The column was re-equilibrated to initial conditions, yielding a total run time of 1.71 min per sample. The injection volume was 10 μL. Data was acquired using multiple reaction monitoring (MRM) in the negative ion mode according to Suppl. Table 5 (see Paterson et al. Nature Metabolism). The source parameters are as follows: −2.0 kV capillary voltage, 60 V cone voltage, desolvation temperature 600° C., cone and desolvation gas flow rate were 150 and 1000 L/hr, respectively. Data was acquired by MassLynx software (version 4.2) and processed with TargetLynx XS (Waters).

### Quantification of Liver Triglycerides

Liver (50-200 mg) was incubated overnight at 50° C. added in 350 μl ethanolic KOH (2 Ethanol (100%):1 KOH (30%)). Following incubation, samples were vortexed and 650 μl of ethanol (50%) was added followed by centrifugation at full speed for 5 minutes. 900 μl of the of the supernatant was mixed with 300 μl Ethanol (50%) and 200 μl of the samples were mixed with 215 μl of 1M MgCl2, and incubated on ice for 10 minutes. Subsequently, samples were centrifuged at full speed for 5 minutes and 10 μl of the supernatant was assayed for glycerol content using free glycerol reagent (Sigma).

### Protein Analysis

Tissue was homogenized in Triton Lysis Buffer (12.5 mM HEPES pH 7.4, 50 mM NaCl, 500 μM EDTA, 5% glycerol, 0.5% Triton X-100, 50 mM Sodium vanadate, 50 mM PMSF, 5 mM aprotinin, 5 mM, Leupeptin) using a TissueLyser II Homogenizer (Qiagen) before undergoing centrifugation at 10,000 rpm for 10 minutes at 4° C. The supernatant was transferred to a new tube and protein quantified with Pierce BCA Protein Assay Kit (Thermo Fisher Scientific) and analyzed by western blot by incubating with anti-RBFOX2 (Bethyl Laboratories), anti-PLA2G6 (Santa Cruz), anti-SREBP1 (Pharmigen), anti-vinculin, anti-APOB, anti-ABCA1, anti-ACC, anti-pS79 ACC, anti-FASN (Cell signaling) and anti-Tubulin (Santa Cruz) primary antibodies, and imaged with an Odyssey infra-red scanner (LICOR).

### Glucose Tolerance Tests

For glucose tolerance tests animals are fasted for 16 h and i.p injected with 1 g of glucose/kg. Blood glucose was measured using Contour XT glucometers (Roche).

### Lipoprotein Fractionation and Characterization

Major lipoprotein fractions namely very low-density lipoprotein (VLDL, d<1.019 g/ml), low density lipoprotein (LDL, d: 1.019-1.063 g/ml) and high density lipoprotein (HDL, d: 1.063-1.21 g/ml) were successively isolated from plasma by sequential ultracentrifugation at 100,000 rpm at 15° C. using a Beckman Optima Max-TL centrifuge following periods of centrifugation of 1 h30, 3 h30 and 5 h30 respectively. After isolation, lipoprotein fractions were analyzed for their lipid and protein content with a calibrated AutoAnalyzer (Konelab 20) by using Commercial kits. Total cholesterol, free cholesterol and phospholipids were measured using reagents from Diasys. Cholesteryl ester (CE) mass was calculated as (TC-FC)×1.67 and thus represents the sum of the esterified cholesterol and fatty acid moieties. Triglycerides were quantified with a commercial kit (Thermo Electron). Bicinchoninic acid assay reagent (Pierce, Thermo Fisher Scientific) was utilized for protein quantification. Lipoprotein mass was calculated as the sum of the mass of the individual lipid and protein components for each lipoprotein fraction.

### Primary Hepatocytes

Livers were perfused using liver perfusion buffer (HBSS, KCl 0.4 g/L, glucose 1 g/L, NaHCO₃ 2.1 g/L, EDTA 0.2 g/L) and then digested using liver digest buffer (DMEM-GlutaMAX 1 g/L glucose, HEPES 15 mM pH7.4, penicillin/streptomycin 1%, 5 mg/mouse Collagenase IV (C5138 Sigma). After excision, livers were placed on ice in plating media (M199, FBS 10%, Penicillin/Streptomycin 1%, Sodium pyruvate 1%, L-glutamine 1%, 1 nm insulin, 1 mM dexamethasone, 2 mg/ml BSA). Tissue was homogenized using forceps and then filtered in plating media. Cells were then washed twice in plating media and then subjected to a 1:3 Percol Gradient (Sigma Aldrich). Cells were plated on collagen coated plates (ThermoFisher Scientific) in plating media. Media was changed after 3 hours to maintenance media (DMEM 4.5 g glucose/L, penicillin/streptomycin 1%, L-Glutamine 1%, 100 nM Dexamethasone, 2 mg/ml BSA) for 12 h, and hepatocytes were treated as indicated.

Generation of Cell Lines Expressing pGIPZ Lentiviral shRNA

Lentiviral shRNA (sh1-Foxa1—v2lmm14620, sh4-Foxa2—v2lmm71498) clones were recovered from the pGIPZ library following the manufacturers protocol (ThermoFisher) and used to obtain lentiviruses and produce stable Hepa1-6 cells.

### Antisense Oligonucleotides

RNA splice-switching oligonucleotides (SSO) were synthetized with 2′O-ME modifications and phosphorothioate backbone (Eurogentec). A list of SSO sequence is provided in Table 1. 100 nM SSO were transfected by incubating cells with Lipofectamine2000 in OptiMEM (Thermo Fisher scientific). For in vivo studies, 40 mg/kg/week of oligonucleotides or saline were weekly injected subcutaneously over four consecutive weeks.

### CAGE-Seq and ChIP-Seq Data Processing

Mapped CAGE-supported transcriptional start sites (CTSSs) from the FANTOM5 project⁷³⁻⁷⁵ were imported to R (http://www.R-project.org/) as CTSS tables. Replicate samples were merged and normalised using the standard workflow within the CAGEr package⁷⁶. Processed bigwig files corresponding to human adult liver ChIP-seq signal p-value were obtained from the ENCODE portal⁷⁷: FOXA1 (ENCFF058DKS)⁷⁸, FOXA2 (ENCFF902TMK)⁷⁸, K3K4me3 (ENCFF610REU)⁷⁹, and H3K27ac (ENCFF012XAP)⁷⁹. Mouse liver FOXA1 ChIP-seq data (GSE106379) was retrieved from⁸⁰.

### RT-PCR Analysis

RNA was reverse transcribed using High-Capacity cDNA Reverse Transcription Kit (Thermo Fisher Scientific). Taqman Gene Expression Assays (Thermo Fisher Scientific) with probes from the Roche Universal Probe Library or Fast SYBR-Green Mix (Thermo Fisher Scientific) were used for quantification on QuantStudio 7 Flex Real-Time PCR system (Thermo Fisher Scientific). All data was analyzed using a relative standard curve or the delta CT method. Ribosomal 18S RNA was used to normalize samples in all cases. Alternative splicing analysis was carried out using primers designed for detecting more than one mRNA isoform. Targets were amplified by PCR and were analyzed by capillary electrophoresis by using the QIAxcel Advanced System (Qiagen). AS was calculated as percentage spliced in (PSI) of a specific splicing event across samples. (PSI=(Long isoform)/(long isoform+short isoform)*100). Probes and primers used in this analysis are described in Suppl. table 3 (see Paterson et al. Nature Metabolism).

Enhanced Individual Nucleotide Resolution UV Cross-Linking and Immunoprecipitation (eiCLIP)

A revised version of the previously described non-isotopic individual nucleotide resolution UV cross-linking and immunoprecipitation (iCLIP) workflow⁸¹ was carried out with novel modifications to enhance speed and efficiency. Specifically, a shortened Cy5.5 labelled adapter was incorporated (/5Phos/A[XXXXXX]NNNAGATCGGAAGAGCACACG/3Cy55Sp/) (SEQ ID NO: 26), high concentration T4 RNA ligase (New England Biolabs) was used to enhance adapter ligation, the RecJf exonuclease (New England Biolabs) was used to remove un-ligated adapter prior to SDS-PAGE, SDS-PAGE was visualised in the 700 nm channel, reverse transcription was carried out with a biotinylated primer homologous to the adapter (/5BiotinTEG/CGTGTGCTCTTCCGA) (SEQ ID NO: 27), un-incorporated RT-primer was removed by exonuclease III (New England Biolabs) after annealing to the reverse complement, cDNA was captured by MyOne streptavidin C1 magnetic beads (Thermo Fisher Scientific), bead bound cDNA was ligated to a 3′ adapter (/5Phos/ANNNNNNNAGATCGGAAGAGCGTCGTG/3ddC/) (SEQ ID NO: 28) instead of carrying out the previous used intramolecular ligation, and cDNA was eluted from the streptavidin beads using nuclease and cation free water at high temperature. A 5% size matched input was prepared by capturing the cellular proteome on SeraMag carboxylic beads (Sigma Aldrich) and proceeding through the eiCLIP protocol in parallel with RBFOX2 immunoprecipitated complexes. RBFOX2 eiCLIP was performed using 1 μg/μl RBM9 Antibody (A300-A864A, Bethyl Laboratories) using isolated primary hepatocytes—total protein was quantified at 4 μg/μl and antibody was added 8 μg/ml. Samples from three independent hepatocyte cultures each obtained from two mice were sequenced with paired end reads using a MiSeq system (Illumina).

Mapping and Identification of Crosslink Clusters from eCLIP and eiCLIP Experiments

For mapping eCLIP and eiCLIP RBFOX2 sequencing data we used GENCODE assembly annotation version ‘GRCm38.VM20’ for mouse and ‘GRCh38.p7’ for human samples. For the eCLIP samples a double adapter removal was used following the recommended ENCODE eCLIP pipeline: (https://www.encodeproject.org/pipelines/NCPL357ADL/). For the adapter removal of eiCLIP sequencing samples we also used ‘cutadapt’ tool (https://cutadapt.readthedocs.io/en/stable) with the following parameters: ‘cutadapt -f fastq - -match-read-wildcards --times 1 -e 0.1 -O 1 --quality-cutoff 6 -m 18 -a AGATCGGAAG $INPUT.fastq>$OUTPUT.adapterTrimfastq 2>$OUTPUT.adapterTrim.metrics’. Both eCLIP and eiCLIP samples were aligned by STAR alignment tool (version 2.4.2a) (https://github.com/alexdobin/STAR) with the following parameters: ‘STAR -runThreadN 8 - runMode alignReads -genomeDir GRCh38 Gencode v25 -genomeLoad LoadAndKeep - readFilesIn read1, read2, -readFilesCommand zcat -outSAMunmapped Within -outFilterMultimapNmax 1 -outFilterMultimapScoreRange 1 -outSAMattributes All - outSAMtype BAM Unsorted -outFilterType BySJout -outFilterScoreMin 10 -alignEndsType EndToEnd -outFileNamePrefix outfile’.

For the overamplification correction of eCLIP samples we used a barcode collapse python script ‘barcode_collapse_pe.py’ available on GitHub (https://github.com/YeoLab/gscripts/releases/tag/1.0). And for the eiCLIP samples we used a custom python script to swap random barcodes from the first 7 nts of the FASTQ sequence line to the FASTQ header line. Uniquely mapped reads with the same genomic positions and the same random barcode were then removed as PCR duplicates.

To identify binding clusters we used cDNA-starts as crosslinking positions and as the input for False Discovery Rate clustering tool available by iMaps (https://imaps.genialis.com/iclip).

The clusters were identified by using default parameters and Paraclu clustering algorithm (http://cbrc3.cbrc.jp/˜martin/paraclu/). Semantic space for RBFOX2 eiCLIP targets was visualised with REVIGO⁸².

Motif Enrichment Relative to eCLIP and eiCLIP Crosslink Sites

To identify the enrichment of RBFOX2 binding motifs relative to crosslinking sites we used density plot of already known (U)GCAUG binding motif^{22, 83} relative to eiCLIP cDNA-starts from mouse liver samples and eCLIP cDNA-starts of HepG2 samples from ENCODE. Each position on the graph was normalised by the total number of mapped cDNAs from all three replicates.

### Comparison of Human and Mouse RBFOX2 Binding Sites

For the lift over of RBFOX2 crosslink clusters from mouse (mm10) to human (hg38), we used UCSC online tools (https://genome.ucsc.edu/cgi-bin/hgLiftOver). Overlap analysis between binding sites was performed with pybedtools^{84, 85}.

### Enrichment Analysis of Orthologous RBFOX2 Target Genes

The human orthologous genes of the mouse RBFOX2 targets detected by eiCLIP were retrieved from BioMart⁸⁶ and investigated for enrichment for genes associated with common traits/diseases using EnrichR^{87, 88}.

### RNA-Maps Around RBFOX2-Regulated Exons

Alternatively spliced and control exons were selected from L^{WT} and L^{ΔRbfox2} liver RNAseq samples analysed by ‘junctionSeq’ Bioconductor package (http://www.bioconductor.org/packages/release/bioc/html/JunctionSeq.html) from the Splicing Events table MATS.SE with the following parameters:
Up regulated exons: p-value<0.05, FDR<0.1, IncLevelDifference>0.2
Down regulated exons: p-value<0.05, FDR<0.1, IncLevelDifference<−0.2
Control exons: p-value<0.05, FDR<0.1, absolute(IncLevelDifference)<0.1

For the splicing regulation analysis of RBFOX2 eiCLIP, we used previously published RNAmaps approach⁸⁹ Chakrabarti, 2018 #12611. Density graphs were plotted as distribution of cDNA-starts of RBFOX2 eiCLIP samples relative to 5′ and 3′ splice sites. All three replicates were grouped together and each group of exons was normalised by the total number of exons per group.

### Statistical Analysis

Differences between dietary groups and gene targets were analyzed for statistical significance using a one-way or two-way ANOVA test. Pairwise comparisons were analysed with Mann-Whitney U test or two-sided student's t-test when applicable. Results are represented as mean±SEM.

### Data Availability:

The mass spectrometry data have been deposited to the ProteomeXchange Consortium via the PRIDE partner repository⁹⁰ with the dataset identifier PXDxx. RNAseq data and eiCLIP data generated for this study have been deposited at GEO under accession number GSE151753.

### Code Availability

The code corresponding to FIG. 1E-G and FIG. 3B,C is publicly available at the GitHub repository (https://github.com/nebo56/RBFOX2-data_analysis).

### REFERENCES

1 Pan, Q., Shai, O., Lee, L. J., Frey, B. J. & Blencowe, B. J. Deep surveying of alternative splicing complexity in the human transcriptome by high-throughput sequencing. Nat Genet 40, 1413-1415 (2008). https://doi.org:10.1038/ng.259
2 Wang, E. T. et al. Alternative isoform regulation in human tissue transcriptomes. Nature 456, 470-476 (2008). https://doi.org:10.1038/nature07509
3 Blencowe, B. J. Reflections for the 20th anniversary issue of RNA journal. RNA 21, 573-575 (2015). https://doi.org:10.1261/rna.051003.115
4 Buljan, M. et al. Tissue-specific splicing of disordered segments that embed binding motifs rewires protein interaction networks. Mol Cell 46, 871-883 (2012). https://doi.org:10.1016/j.molcel.2012.05.039
5 Ellis, J. D. et al. Tissue-specific alternative splicing remodels protein-protein interaction networks. Mol Cell 46, 884-892 (2012). https://doi.org:10.1016/j.molcel.2012.05.037
6 Yang, X. et al. Widespread Expansion of Protein Interaction Capabilities by Alternative Splicing. Cell 164, 805-817 (2016). https://doi.org:10.1016/j.cell.2016.01.029
7 Raal, F. J. et al. Mipomersen, an apolipoprotein B synthesis inhibitor, for lowering of LDL cholesterol concentrations in patients with homozygous familial hypercholesterolaemia: a randomised, double-blind, placebo-controlled trial. Lancet 375, 998-1006 (2010). https://doi.org:10.1016/S0140-6736(10)60284-X
8 Ray, K. K. et al. Inclisiran in Patients at High Cardiovascular Risk with Elevated LDL Cholesterol. N Engl J Med 376, 1430-1440 (2017). https://doi.org:10.1056/NEJMoa1615758
9 Eslam, M., Sanyal, A. J., George, J. & International Consensus, P. MAFLD: A Consensus-Driven Proposed Nomenclature for Metabolic Associated Fatty Liver Disease. Gastroenterology 158, 1999-2014 e1991 (2020). https://doi.org:10.1053/j.gastro.2019.11.312
10 Friedman, S. L., Neuschwander-Tetri, B. A., Rinella, M. & Sanyal, A. J. Mechanisms of NAFLD development and therapeutic strategies. Nat Med 24, 908-922 (2018). https://doi.org:10.1038/s41591-018-0104-9
11 Turpin-Nolan, S. M. & Bruning, J. C. The role of ceramides in metabolic disorders: when size and localization matters. Nat Rev Endocrinol 16, 224-233 (2020). https://doi.org:10.1038/s41574-020-0320-5
12 Hall, Z. et al. Lipid zonation and phospholipid remodeling in nonalcoholic fatty liver disease. Hepatology 65, 1165-1180 (2017). https://doi.org:10.1002/hep.28953
13 Ioannou, G. N. The Role of Cholesterol in the Pathogenesis of NASH. Trends Endocrinol Metab 27, 84-95 (2016). https://doi.org:10.1016/j.tem2015.11.008
14 Corey, K. E. & Chalasani, N. Management of dyslipidemia as a cardiovascular risk factor in individuals with nonalcoholic fatty liver disease. Clin Gastroenterol Hepatol 12, 1077-1084; quiz e1059-1060 (2014). https://doi.org:10.1016/j.cgh.2013.08.014
15 Collaborators, G. B. D. O. et al. Health Effects of Overweight and Obesity in 195 Countries over 25 Years. N Engl J Med 377, 13-27 (2017). https://doi.org:10.1056/NEJMoa1614362
16 Zhang, G. et al. Differential metabolic and multi-tissue transcriptomic responses to fructose consumption among genetically diverse mice. Biochim Biophys Acta Mol Basis Dis 1866, 165569 (2020). https://doi.org:10.1016/j.bbadis.2019.165569
17 Van Nostrand, E. L. et al. Robust transcriptome-wide discovery of RNA-binding protein binding sites with enhanced CLIP (eCLIP). Nat Methods 13, 508-514 (2016). https://doi.org:10.1038/nmeth.3810
18 Tabula Muris, C. et al. Single-cell transcriptomics of 20 mouse organs creates a Tabula Muris. Nature 562, 367-372 (2018). https://doi.org:10.1038/s41586-018-0590-4
19 Damianov, A. & Black, D. L. Autoregulation of Fox protein expression to produce dominant negative splicing factors. RNA 16, 405-416 (2010). https://doi.org:10.1261/rna.1838210
20 Jangi, M., Boutz, P. L., Paul, P. & Sharp, P. A. Rbfox2 controls autoregulation in RNA-binding protein networks. Genes Dev 28, 637-651 (2014). https://doi.org:10.1101/gad.235770.113
21 Irimia, M. & Blencowe, B. J. Alternative splicing: decoding an expansive regulatory layer. Curr Opin Cell Biol 24, 323-332 (2012). https://doi.org:10.1016/j.ceb.2012.03.005
22 Jin, Y. et al. A vertebrate RNA-binding protein Fox-1 regulates tissue-specific splicing via the pentanucleotide GCAUG. EMBO J 22, 905-912 (2003). https://doi.org:10.1093/emboj/cdg089
23 Yeo, G. W. et al. An RNA code for the FOX2 splicing regulator revealed by mapping RNA-protein interactions in stem cells. Nat Struct Mol Biol 16, 130-137 (2009). https://doi.org:10.1038/nsmb.1545
24 Zhang, C. et al. Defining the regulatory network of the tissue-specific splicing factors Fox-1 and Fox-2. Genes Dev 22, 2550-2563 (2008). https://doi.org:10.1101/gad.1703108
25 Underwood, J. G., Boutz, P. L., Dougherty, J. D., Stoilov, P. & Black, D. L. Homologues of the Caenorhabditis elegans Fox-1 protein are neuronal splicing regulators in mammals. Mol Cell Biol 25, 10005-10016 (2005). https://doi.org:10.1128/MCB.25.22.10005-10016.2005
26 Nakahata, S. & Kawamoto, S. Tissue-dependent isoforms of mammalian Fox-1 homologs are associated with tissue-specific splicing activities. Nucleic Acids Res 33, 2078-2089 (2005). https://doi.org:10.1093/nar/gki338
27 Modafferi, E. F. & Black, D. L. A complex intronic splicing enhancer from the c-src pre-mRNA activates inclusion of a heterologous exon. Mol Cell Biol 17, 6537-6545 (1997). https://doi.org:10.1128/MCB.17.11.6537
28 Kozarsky, K. F. et al. Overexpression of the HDL receptor SR-BI alters plasma HDL and bile cholesterol levels. Nature 387, 414-417 (1997). https://doi.org:10.1038/387414a0
29 Acton, S. et al. Identification of scavenger receptor SR-BI as a high density lipoprotein receptor. Science 271, 518-520 (1996). https://doi.org:10.1126/science.271.5248.518
30 Dennis, E. A., Cao, J., Hsu, Y. H., Magrioti, V. & Kokotos, G. Phospholipase A2 enzymes: physical structure, biological function, disease implication, chemical inhibition, and therapeutic intervention. Chem Rev 111, 6130-6185 (2011). https://doi.org:10.1021/cr200085w
31 Han, J. et al. The CREB coactivator CRTC2 controls hepatic lipid metabolism by regulating SREBP1. Nature 524, 243-246 (2015). https://doi.org:10.1038/nature14557
32 Gusarova, V., Brodsky, J. L. & Fisher, E. A. Apolipoprotein B100 exit from the endoplasmic reticulum (ER) is COPII-dependent, and its lipidation to very low density lipoprotein occurs post-ER. J Biol Chem 278, 48051-48058 (2003). https://doi.org:10.1074/jbc.M306898200
33 Temel, R. E. et al. Hepatic Niemann-Pick C1-like 1 regulates biliary cholesterol concentration and is a target of ezetimibe. J Clin Invest 117, 1968-1978 (2007). https://doi.org:10.1172/JCI30060
34 Horton, J. D., Goldstein, J. L. & Brown, M. S. SREBPs: activators of the complete program of cholesterol and fatty acid synthesis in the liver. J Clin Invest 109, 1125-1131 (2002). https://doi.org:10.1172/JCI15593
35 Bochkis, I. M. et al. Hepatocyte-specific ablation of Foxa2 alters bile acid homeostasis and results in endoplasmic reticulum stress. Nat Med 14, 828-836 (2008). https://doi.org:10.1038/nm.1853
36 Moya, M. et al. Foxa1 reduces lipid accumulation in human hepatocytes and is downregulated in nonalcoholic fatty liver. PLoS One 7, e30014 (2012). https://doi.org:10.1371/journal.pone.0030014
37 Webb, N. R. et al. SR-BII, an isoform of the scavenger receptor BI containing an alternate cytoplasmic tail, mediates lipid transfer between high density lipoprotein and cells. J Biol Chem 273, 15241-15248 (1998). https://doi.org:10.1074/jbc.273.24.15241
38 Puri, P. et al. A lipidomic analysis of nonalcoholic fatty liver disease. Hepatology 46, 1081-1090 (2007). https://doi.org:10.1002/hep.21763
39 Trigatti, B. et al. Influence of the high density lipoprotein receptor SR-BI on reproductive and cardiovascular pathophysiology. Proc Natl Acad Sci USA 96, 9322-9327 (1999). https://doi.org:10.1073/pnas.96.16.9322
40 Huby, T. et al. Knockdown expression and hepatic deficiency reveal an atheroprotective role for SR-BI in liver and peripheral tissues. J Clin Invest 116, 2767-2776 (2006). https://doi.org:10.1172/JCI26893
41 Sen, S., Jumaa, H. & Webster, N. J. Splicing factor SRSF3 is crucial for hepatocyte differentiation and metabolic function. Nat Commun 4, 1336 (2013). https://doi.org:10.1038/ncomms2342
42 Wei, N. et al. SRSF10 Plays a Role in Myoblast Differentiation and Glucose Production via Regulation of Alternative Splicing. Cell Rep 13, 1647-1657 (2015). https://doi.org:10.1016/j.celrep.2015.10.038
43 Benegiamo, G. et al. The RNA-Binding Protein NONO Coordinates Hepatic Adaptation to Feeding. Cell Metab 27, 404-418 e407 (2018). https://doi.org:10.1016/j.cmet.2017.12.010
44 Pihlajamäki, J. et al. Expression of the splicing factor gene SFRS10 is reduced in human obesity and contributes to enhanced lipogenesis. Cell Metabolism 14, 208-218 (2011). https://doi.org:10.1016/j.cmet.2011.06.007
45 Nikolaou, K. C. et al. The RNA-Binding Protein A1CF Regulates Hepatic Fructose and Glycerol Metabolism via Alternative RNA Splicing. Cell Rep 29, 283-300 e288 (2019). https://doi.org:10.1016/j.celrep.2019.08.100
46 Kuroyanagi, H. Fox-1 family of RNA-binding proteins. Cell Mol Life Sci 66, 3895-3907 (2009). https://doi.org:10.1007/s00018-009-0120-5
47 Min, H. K. et al. Increased hepatic synthesis and dysregulation of cholesterol metabolism is associated with the severity of nonalcoholic fatty liver disease. Cell Metab 15, 665-674 (2012). https://doi.org:10.1016/j.cmet.2012.04.004
48 Eckhardt, E. R. et al. High density lipoprotein endocytosis by scavenger receptor SRBII is clathrin-dependent and requires a carboxyl-terminal dileucine motif. J Biol Chem 281, 4348-4353 (2006). https://doi.org:10.1074/jbc.M513154200
49 Emerging Risk Factors, C. et al. Major lipids, apolipoproteins, and risk of vascular disease. JAMA 302, 1993-2000 (2009). https://doi.org:10.1001/jama.2009.1619
50 Zanoni, P. et al. Rare variant in scavenger receptor BI raises HDL cholesterol and increases risk of coronary heart disease. Science 351, 1166-1171 (2016). https://doi.org:10.1126/science.aad3517
51 Gehman, L. T. et al. The splicing regulator Rbfox2 is required for both cerebellar development and mature motor function. Genes Dev 26, 445-460 (2012). https://doi.org:10.1101/gad.182477.111
52 Postic, C. et al. Dual roles for glucokinase in glucose homeostasis as determined by liver and pancreatic beta cell-specific gene knock-outs using Cre recombinase. J Biol Chem 274, 305-315 (1999). https://doi.org:10.1074/jbc.274.1.305
53 Nguyen, A. T. et al. UBE2O remodels the proteome during terminal erythroid differentiation. Science 357 (2017). https://doi.org:10.1126/science.aan0218
54 Rappsilber, J., Ishihama, Y. & Mann, M. Stop and go extraction tips for matrix assisted laser desorption/ionization, nanoelectrospray, and LC/MS sample pretreatment in proteomics. Anal Chem 75, 663-670 (2003). https://doi.org:10.1021/ac026117i
55 McAlister, G. C. et al. MultiNotch MS3 enables accurate, sensitive, and multiplexed detection of differential expression across cancer cell line proteomes. Anal Chem 86, 7150-7158 (2014). https://doi.org:10.1021/ac502040v
56 Eng, J. K., Jahan, T. A. & Hoopmann, M. R. Comet: an open-source MS/MS sequence database search tool. Proteomics 13, 22-24 (2013). https://doi.org:10.1002/pmic.201200439
57 Huttlin, E. L. et al. A tissue-specific atlas of mouse protein phosphorylation and expression. Cell 143, 1174-1189 (2010). https://doi.org:10.1016/j.cell.2010.12.001
58 Blackford, S. J. I. et al. Validation of Current Good Manufacturing Practice Compliant Human Pluripotent Stem Cell-Derived Hepatocytes for Cell-Based Therapy. Stem Cells Translational Medicine 8, 124-137 (2019). https://doi.org:10.1002/sctm.18-0084
59 Jobbins, A. M. et al. Dysregulated RNA polyadenylation contributes to metabolic impairment in non-alcoholic fatty liver disease. Nucleic Acids Res (2022). https://doi.org:10.1093/nar/gkac165
60 Wu, J. C., Merlino, G. & Fausto, N. Establishment and characterization of differentiated, nontransformed hepatocyte cell lines derived from mice transgenic for transforming growth factor alpha. Proc Natl Acad Sci USA 91, 674-678 (1994). https://doi.org:10.1073/pnas.91.2.674
61 Kim, D. et al. TopHat2: accurate alignment of transcriptomes in the presence of insertions, deletions and gene fusions. Genome Biol 14, R36 (2013). https://doi.org:10.1186/gb-2013-14-4-r36
62 Liao, Y., Smyth, G. K. & Shi, W. featureCounts: an efficient general purpose program for assigning sequence reads to genomic features. Bioinformatics 30, 923-930 (2014). https://doi.org:10.1093/bioinformatics/btt656
63 Love, M. I., Huber, W. & Anders, S. Moderated estimation of fold change and dispersion for RNA-seq data with DESeq2. Genome Biology 2014 15:1215, 1-21 (2014). https://doi.org:10.1186/S13059-014-0550-8
64 Robinson, M. D., McCarthy, D. J. & Smyth, G. K. edgeR: a Bioconductor package for differential expression analysis of digital gene expression data. Bioinformatics 26, 139-140 (2010). https://doi.org:10.1093/bioinformatics/btp616
65 Law, C. W., Chen, Y., Shi, W. & Smyth, G. K. voom: Precision weights unlock linear model analysis tools for RNA-seq read counts. Genome Biol 15, R29 (2014). https://doi.org:10.1186/gb-2014-15-2-r29
66 Ritchie, M. E. et al. limma powers differential expression analyses for RNA sequencing and microarray studies. Nucleic Acids Res 43, e47 (2015). https://doi.org:10.1093/nar/gkv007
67 Huber, W. et al. Orchestrating high-throughput genomic analysis with Bioconductor. Nat Methods 12, 115-121 (2015). https://doi.org:10.1038/nmeth.3252
68 Shen, S. et al. rMATS: robust and flexible detection of differential alternative splicing from replicate RNA-Seq data. Proc Natl Acad Sci USA 111, E5593-5601 (2014). https://doi.org:10.1073/pnas.1419161111
69 Young, M. D., Wakefield, M. J., Smyth, G. K. & Oshlack, A. Gene ontology analysis for RNA-seq: accounting for selection bias. Genome Biol 11, R14 (2010). https://doi.org:10.1186/gb-2010-11-2-r14
70 McCarthy, D. J., Chen, Y. & Smyth, G. K. Differential expression analysis of multifactor RNA-Seq experiments with respect to biological variation. Nucleic Acids Res 40, 4288-4297 (2012). https://doi.org:10.1093/nar/gks042
71 Folch, J., Lees, M. & Sloane Stanley, G. H. A simple method for the isolation and purification of total lipides from animal tissues. J Biol Chem 226, 497-509 (1957).
72 Smith, C. A., Want, E. J., O'Maille, G., Abagyan, R. & Siuzdak, G. XCMS: processing mass spectrometry data for metabolite profiling using nonlinear peak alignment, matching, and identification. Anal Chem 78, 779-787 (2006). https://doi.org:10.1021/ac051437y
73 Lizio, M. et al. Gateways to the FANTOM5 promoter level mammalian expression atlas. Genome Biol 16, 22 (2015). https://doi.org:10.1186/s13059-014-0560-6
74 Noguchi, S. et al. FANTOM5 CAGE profiles of human and mouse samples. Sci Data 4, 170112 (2017). https://doi.org:10.1038/sdata.2017.112
75 Lizio, M. et al. Update of the FANTOM web resource: expansion to provide additional transcriptome atlases. Nucleic Acids Res 47, D752-D758 (2019). https://doi.org:10.1093/nar/gky1099
76 Haberle, V., Forrest, A. R., Hayashizaki, Y., Carninci, P. & Lenhard, B. CAGEr: precise TSS data retrieval and high-resolution promoterome mining for integrative analyses. Nucleic Acids Res 43, e51 (2015). https://doi.org:10.1093/nar/gkv054
77 Sloan, C. A. et al. ENCODE data at the ENCODE portal. Nucleic Acids Res 44, D726-732 (2016). https://doi.org:10.1093/nar/gkv160
78 Dunham, I. et al. An integrated encyclopedia of DNA elements in the human genome. Nature 489, 57-74 (2012). https://doi.org:10.1038/nature11247
79 Bernstein, B. E. et al. The NIH Roadmap Epigenomics Mapping Consortium. Nat Biotechnol 28, 1045-1048 (2010). https://doi.org:10.1038/nbt1010-1045
80 Grimm, S. A. et al. DNA methylation in mice is influenced by genetics as well as sex and life experience. Nat Commun 10, 305 (2019). https://doi.org:10.1038/s41467-018-08067-z
81 Sibley, C. R. Individual Nucleotide Resolution UV Cross-Linking and Immunoprecipitation (iCLIP) to Determine Protein-RNA Interactions. Methods Mol Biol 1649, 427-454 (2018). https://doi.org:10.1007/978-1-4939-7213-5_29
82 Supek, F., Bosnjak, M., Skunca, N. & Smuc, T. REVIGO summarizes and visualizes long lists of gene ontology terms. PLoS One 6, e21800 (2011). https://doi.org:10.1371/journal.pone.0021800
83 Auweter, S. D. et al. Molecular basis of RNA recognition by the human alternative splicing factor Fox-1. EMBO J 25, 163-173 (2006). https://doi.org:10.1038/sj.emboj.7600918
84 Quinlan, A. R. & Hall, I. M. BEDTools: a flexible suite of utilities for comparing genomic features. Bioinformatics 26, 841-842 (2010). https://doi.org:10.1093/bioinformatics/btq033
85 Dale, R. K., Pedersen, B. S. & Quinlan, A. R. Pybedtools: a flexible Python library for manipulating genomic datasets and annotations. Bioinformatics 27, 3423-3424 (2011). https://doi.org:10.1093/bioinformatics/btr539
86 Smedley, D. et al. BioMart—biological queries made easy. BMC Genomics 10, 22 (2009). https://doi.org:10.1186/1471-2164-10-22
87 Chen, E. Y. et al. Enrichr: interactive and collaborative HTML5 gene list enrichment analysis tool. BMC Bioinformatics 14, 128 (2013). https://doi.org:10.1186/1471-2105-14-128
88 Kuleshov, M. V. et al. Enrichr: a comprehensive gene set enrichment analysis web server 2016 update. Nucleic Acids Res 44, W90-97 (2016). https://doi.org:10.1093/nar/gkw377
89 Haberman, N. et al. Insights into the design and interpretation of iCLIP experiments. Genome Biol 18, 7 (2017). https://doi.org:10.1186/s13059-016-1130-x
90 Vizcaino, J. A. et al. 2016 update of the PRIDE database and its related tools. Nucleic Acids Res 44, D447-456 (2016). https://doi.org:10.1093/nar/gkv1145

1. An antisense oligomer capable of inducing skipping of exon 12 of scavenger receptor class B type I (SCARB1).

2. The antisense oligomer of claim 1, wherein the antisense oligomer is capable of inducing skipping of exon 12 of human SCARB1.

3. The antisense oligomer of claim 1 or claim 2, wherein the antisense oligomer is 10-45 nucleobases, 12-40 nucleobases, 15-35 nucleobases, 18-30 nucleobases, or 19-25 nucleobases in length.

4. The antisense oligomer of any one of claims 1 to 3, wherein the antisense oligomer comprises a nucleobase sequence according to any one of SEQ ID NOs: 4, 5, 7, 8, 9, 10, 11, 12, 18, or 19, and comprises 5, 4, 3, 2, 1, or fewer substitutions, deletions, or insertions.

5. The antisense oligomer of any one of claims 1 to 4, wherein the antisense oligomer comprises a nucleobase sequence according to any one of SEQ ID NOs: 4, 5, 7, 8, 9, 10, 11, 12, 18, or 19, and one or more nucleobase is substituted for a modified nucleobase that is capable of base pairing with the same type of nucleobase.

6. The antisense oligomer of any one of claims 1 to 3, wherein the antisense oligomer comprises a nucleobase sequence according to any one of SEQ ID NOs: 4, 5, 7, 8, 9, 10, 11, 12, 18, or 19.

7. The antisense oligomer of any one of claims 1 to 3, wherein the antisense oligomer comprises a nucleobase sequence according to any one of SEQ ID NOs: 5, 8, 10, 11, 18, or 19.

8. The antisense oligomer of claim 1 or claim 2, wherein the nucleobase sequence of the antisense oligomer is according to any one of SEQ ID NOs: 4, 5, 7, 8, 9, 10, 11, 12, 18, or 19.

9. The antisense oligomer of any preceding claim, wherein the antisense oligomer is complementary to 5, 10, 15, 16, 17, 18, 19, 20, or all bases to which an antisense oligomer according to any one of SEQ ID NOs: 4, 5, 7, 8, 9, 10, 11, 12, 18, or 19 is complementary.

10. The antisense oligomer of any preceding claim, wherein the antisense oligomer is complementary to 5, 10, 15, 16, 17, 18, 19, or all 20 bases to which SSO8.18 (SEQ ID NO: 51) is complementary.

11. The antisense oligomer of any preceding claim, wherein the antisense oligomer comprises at least one 2′-O-methyl nucleotide and/or at least one 2′-O-methoxyethyl nucleotide.

12. The antisense oligomer of any preceding claim, wherein the antisense oligomer is a 2′-O-methyl nucleic acid oligomer and/or a 2′-O-methoxyethyl nucleic acid oligomer.

13. The antisense oligomer of any preceding claim, wherein the antisense oligomer comprises at least one phosphorothioate internucleotide linkage.

14. The antisense oligomer of any preceding claim, wherein all internucleotide linkages in the antisense oligomer are phosphorothioate internucleotide linkages.

15. The antisense oligomer of any preceding claim, wherein the antisense oligomer is an oligonucleotide and has a nucleobase sequence according to SEQ ID NO: 5, 8, 10, 11, 18, or 19, and wherein —O—CH₃ or —O—CH₂—CH₂—O—CH₃ is attached to the 2′ position of the sugar moiety of each nucleotide.

16. An antisense oligonucleotide with a sequence according to any one of SEQ ID NOs: 4, 5, 7, 8, 9, 10, 11, 12, 18, or 19, wherein the internucleotide linkages are phosphorothioate internucleotide linkages, and wherein —O—CH₃ or —O—CH₂—CH₂—O—CH₃ is attached to the 2′ position of the sugar moiety of each nucleotide.

17. The antisense oligomer of any preceding claim, wherein the antisense oligomer is or comprises SSO8.5 (SEQ ID NO: 38), SSO8.8 (SEQ ID NO: 41), SSO8.10 (SEQ ID NO: 43), SSO8.11 (SEQ ID NO: 44), SSO8.18 (SEQ ID NO: 51), SSO8.19 (SEQ ID NO: 52), or SSO8.21 (SEQ ID NO: 54).

18. The antisense oligomer of any preceding claim, wherein the antisense oligomer is or comprises SSO8.18 (SEQ ID NO: 51).

19. A pharmaceutical composition comprising the antisense oligomer or antisense oligonucleotide of any one of claims 1 to 18.

20. An antisense oligomer, antisense oligonucleotide, or pharmaceutical composition of any one of claims 1 to 19, for use as a medicament.

21. An antisense oligomer, antisense oligonucleotide, or pharmaceutical composition of any one of claims 1 to 19, for use in a method of treating or preventing a metabolism-associated disease.

22. An antisense oligomer, antisense oligonucleotide, or pharmaceutical composition of any one of claims 1 to 19, for use in a method of treating or preventing a pathological effect of obesity and/or an obesogenic diet.

23. The antisense oligomer, antisense oligonucleotide, or pharmaceutical composition for use of claim 22, wherein the pathological effect is liver inflammation, lipotoxicity, hepatocellular injury, and/or fibrosis.

24. An antisense oligomer, antisense oligonucleotide, or pharmaceutical composition of any one of claims 1 to 19, for use in a method of treating or preventing liver inflammation.

25. The antisense oligomer, antisense oligonucleotide, or pharmaceutical composition for use of claim 24, wherein the liver inflammation is obesity-induced.

26. An antisense oligomer, antisense oligonucleotide, or pharmaceutical composition of any one of claims 1 to 19, for use in a method of treating or preventing metabolism-associated fatty liver disease (MAFLD).

27. An antisense oligomer, antisense oligonucleotide, or pharmaceutical composition of any one of claims 1 to 19, for use in a method of treating or preventing pre-symptomatic hepatic steatosis.

28. An antisense oligomer, antisense oligonucleotide, or pharmaceutical composition of any one of claims 1 to 19, for use in a method of treating or preventing non-alcoholic steatohepatitis (NASH).

29. An antisense oligomer, antisense oligonucleotide, or pharmaceutical composition of any one of claims 1 to 19, for use in a method of treating or preventing hepatocellular carcinoma (HCC) in a subject.

30. The antisense oligomer, antisense oligonucleotide, or pharmaceutical composition for use of claim 29, wherein the subject has liver inflammation, pre-symptomatic hepatic steatosis, or NASH.

31. An antisense oligomer, antisense oligonucleotide, or pharmaceutical composition of any one of claims 1 to 19, for use in treating or preventing any one of, or a combination of, gallstone disease, type 2 diabetes, cardiovascular disease, and coronary artery disease.

32. An antisense oligomer, antisense oligonucleotide, or pharmaceutical composition of any one of claims 1 to 19, for use in a method of treatment, wherein the method comprises lowering cholesterol levels in a subject in need thereof.

33. A method of increasing expression of scavenger receptor class B type I (Scarb1) isoform SR-BII relative to the isoform SR-BI in a cell, the method comprising contacting the cell with a composition comprising the antisense oligomer or antisense oligonucleotide of any one of claims 1 to 18.

34. The method of claim 33, wherein the method is in vivo and the composition is administered to a subject in need thereof.
